(19) United States
(12) Patent Application Publication
(10) Pub. No.: US 2025/0368716 A1
(43) Pub. Date: Dec. 4, 2025

(54) DELTA T-CELL OR GAMMA T-CELL RECEPTOR CHAINS OR PARTS THEREOF THAT MEDIATE AN ANTI-TUMOUR OR ANTI-INFECTIVE RESPONSE

(71) Applicant: GADETA B.V, Utrecht (NL)

(72) Inventors: Haakan Norell, Vallentuna (SE); Stefania Gobessi, Utrecht (NL)

(21) Appl. No.: 18/568,179

(22) PCT Filed: Jun. 7, 2022
(86) PCT No.: PCT/EP2022/065382

§ 371 Date: Dec. 7, 2023

(30) Foreign Application Priority Data
Jun. 7, 2021 (EP) 21178061.4

(51) Int. Cl. C07K 14/725; G01N 33/50

(52) U.S. Cl. CPC C07K 14/7051; G01N 33/5011; C07K 2317/565

(57) ABSTRACT

We identified several novel δT-cell and γT-cell receptor chains mediating anti-tumour or anti-infective responses as well as methods for identifying same.

### FIELD OF THE INVENTION

The present invention relates to δT-cell (or γT-cell) receptors chains or parts thereof or γδTCRs or parts thereof or cells comprising or expressing them and mediating an anti-tumour or anti-infective response as well as to methods for identifying same.

### BACKGROUND OF THE INVENTION

Our immune system utilizes different lines of defences to protect us from infections as well as cancer. In order to cover the magnitude of potential invaders and internal threats our adaptive immune system has the possibility to raise up to 10¹⁶ αβTCR combinations as well as 10¹¹ variations in immunoglobulins (Chien Y H, et al, 2014. Annu.Rev.Immunol.).

Among all immune receptor chains, TCR bs have even the highest potential diversity in the CDR3 loop (approximately 10¹⁶ combinations for murine TCR δ) owing to the presence of multiple D gene segments (two in mice, three in human, and up to five in cattle) that can join together. Each D gene segment can be read in all three open reading frames, and N nucleotides can be inserted into the junctions of the joining segments. Thus, despite the limited diversity at the VJ junctions of TCR γ-chains, the potential diversity generated at the combined CDR3 junctions (approximately 10¹⁸ combinations) is still higher than that of αβTCRs (˜10¹⁶) and immunoglobulins (˜10¹¹) (Chien Y H et al, 2014. Annu Rev. Immunol.). TCRδ and TCRγ chains may be particularly useful for immunotherapeutics against cancer and infections.

Accordingly, there is still a need for improved γT- and δT-cell receptor chains, and γδT-cell receptors. There is still a need for identifying new γT- and δT-cell receptor chains, and γδT-cell receptors, that will mediate an anti-tumour response. There is still a need for identifying new γT- and δT-cell receptor chains, and γδT-cell receptors, that will mediate an anti-infective response. There is still a need for improved treatments utilizing γT- and δT-cell receptor chains, and γδT-cell receptors.

### DESCRIPTION OF THE FIGURES

FIG. 1. Map of pLenti6.3

FIG. 2A-2B. γδTCR clone 1 does not display tumour reactivity. MZ1851 RC, RKO, MDA-MB-231 and HT29 tumour cell lines were co-cultured for 48 hr with TEGs expressing γδTCR clone 1 (CDR3 regions represented by SEQ ID NOs: 1, 4) or untransduced matched αβT-cells (negative control), at effector to target (E:T) ratio of 1:1. Cytotoxicity (FIG. 2A) was measured by xCELLigence and plotted as percentage of cytolysis relative to maximum cytolysis induced by treatment of the target cells with the detergent Triton-X-100. Bars represent mean±SD of triplicates in a single experiment. The levels of IFN-γ released into the supernatants were measured by ELISA (FIG. 2B). Graphs describe one representative TEG batch of two analyzed.

FIG. 3A-3B. γδTCR clone 2 displays potent and broad tumour reactivity. Fifteen tumour cell lines were co-cultured for 48 hr with TEGs expressing γδTCR clone 2 (CDR3 regions represented by SEQ ID NOs: 7, 10) or untransduced matched αβT-cells (negative control), at effector to target (E:T) ratio of 1:1. Cytotoxicity (FIG. 3A) was measured by xCELLigence and plotted as percentage of cytolysis relative to maximum cytolysis induced by treatment of the target cells with the detergent Triton-X-100. Bars represent mean±SD of triplicates in a single experiment. The levels of IFN-γ released into the supernatants were measured by ELISA (FIG. 3B). Graphs describe one representative TEG batch of two analyzed.

FIG. 4A-4B. γδTCR clone 3 displays potent and broad tumour reactivity. Fifteen tumour cell lines were co-cultured for 48 hr with TEGs expressing γδTCR clone 3 (CDR3 regions represented by SEQ ID NOs: 13, 16) or untransduced matched αβT-cells (negative control), at effector to target (E:T) ratio of 1:1. Cytotoxicity (FIG. 4A) was measured by xCELLigence and plotted as percentage of cytolysis relative to maximum cytolysis induced by treatment of the target cells with the detergent Triton-X-100. Bars represent mean±SD of triplicates in a single experiment. The levels of IFN-γ released into the supernatants were measured by ELISA (FIG. 4B). Graphs describe one representative TEG batch of two analyzed.

FIG. 5A-5B. γδTCR clone 4 displays clear and broad tumour reactivity. Fifteen tumour cell lines were co-cultured for 48 hr with TEGs expressing γδTCR clone 4 (CDR3 regions represented by SEQ ID NOs: 19, 22) or untransduced matched αβT-cells (negative control), at effector to target (E:T) ratio of 1:1. Cytotoxicity (FIG. 5A) was measured by xCELLigence and plotted as percentage of cytolysis relative to maximum cytolysis induced by treatment of the target cells with the detergent Triton-X-100. Bars represent mean±SD of triplicates in a single experiment. The levels of IFN-γ released into the supernatants were measured by ELISA (FIG. 5B). Graphs describe one representative TEG batch of two analyzed.

FIG. 6A-6B. 41 BBL-OX40 (one of the chimeric bidirectional signaling transmembrane proteins disclosed herein, SEQ ID NO: 83) enhances the anti-tumour activity of TEGs expressing two tested γδTCRs of the invention. TEGs expressing γδTCR clone 2 (CDR3 regions represented by SEQ ID NOs: 7, 10) with or without 41BBL-OX40 protein (FIG. 6A) or γδTCR clone 4 (CDR3 regions represented by SEQ ID NOs: 19, 22) with or without the 41BBL-OX40 protein (FIG. 6B), were repetitively co-cultured with MDA-MB-231 tumour cell line ectopically expressing luciferase-tdTomato, at effector to target (E:T) ratios of 1:1, 1:2, and 1:4, for three rounds of stimulation (Stim). Cytotoxicity was assessed by measuring the luciferase activity from target cells and plotted as percentage of cytolysis relative to tumour target cells only calculated as the following formula: 100x [1-(Luminescence from target cells in co-culture with effector T-cells/Luminescence from target cells cultured alone)]. Bars represent mean±SD of triplicates in a single experiment. Graphs describe one representative TEG batch of two analyzed. **** P<0.0001; *** P<0.001; * P<0.05 (TEG γδTCR+41 BBL-OX40 vs TEG γδTCR).

FIG. 7A-7D. TEGs expressing the newly identified γδTCR of clone 2, clone 3, or clone 4 display more potent tumor reactivity compared to previously characterized γδTCRs. TEGs expressing γδTCR of clone 2, γδTCR of clone 3, γδTCR of clone 4, γδTCR of clone Zi11, or γδTCR of clone Fe11, were co-cultured with Luciferase-transduced HT-29 colon carcinoma cell line (FIG. 7A and FIG. 7B) or with Luciferase-transduced NCI-226 lung carcinoma cell line (FIG. 7C and FIG. 7D) at effector to target (E:T) ratio of 1:1 for 48 hrs. Untransduced αβT cells (UNTR) were used as negative control T cell effectors. Cytotoxicity towards target cells (FIG. 7A and FIG. 7C) was measured by decreased Luciferase activity relative to target cells cultured alone and plotted as % of cytotoxicity. IFNγ levels (FIG. 7B and FIG. 7D) were measured by ELISA. Bars represent mean±SD of triplicates in a single experiment. n.s: not significant; *P<0.05; **P<0.01; ***P<0.001 ****P<0.0001, multiple t-test.

FIG. 8. γδTCRs comprising a Cγ1 constant region result in higher γδTCR cell surface expression in a TEG setting compared to γδTCRs comprising a Cγ2 constant region. Flow cytometry analysis of TEGs expressing γδTCRs of clones 2, 3, and 4 comprising a Cγ1 constant region or comprising a Cγ2 constant region was performed to evaluate the surface expression of the γδTCRs, using antibodies specific to αβTCR (clone IP26) and γδTCR (clone IMMU510). Acquisition was performed with BD FACS Fortessa and data analysized with FlowJo v10. Displayed plots show CD3+ gated cells.

FIG. 9A-9F. TEGs expressing γδTCRs comprising a Cγ1 constant region display a more potent tumour response compared to TEGs expressing γδTCRs comprising the same variable regions but comprising a Cγ2 constant region. TEGs expressing either Cγ1-constant region-comprising or Cγ2-constant region-comprising variants of the γδTCR of clone 2, clone 3, or clone 4 were co-cultured with Luciferase-transduced MDA-MB-231 breast cancer cell line at effector to target (E:T) ratio 1:1 for 48 hrs. At the end of the co-cultures, effectors were harvested and re-seeded on new MDA-MB-231 tumour cells for a second stimulation. For each stimulation round, target cell cytotoxicity (FIG. 9A, FIG. 9C and FIG. 9E) was measured by decreased Luciferase activity relative to target cells cultured alone and plotted as % of cytotoxicity. IFNγ levels (FIG. 9B, FIG. 9D and FIG. 9F) were measured by ELISA. Bars represent mean±SD of triplicates in a single experiment (representative of n=2 experiments with 2 αβT-cell donors). n.s: not significant; *P<0.05; **P<0.01; ***P<0.001, multiple t-test.

FIG. 10A-10E. γδTCRs comprising a Cγ1 constant region result in higher γδTCR cell surface expression in a TEG setting compared to γδTCRs comprising a Cγ2 constant region and TEGs expressing γδTCRs comprising a Cγ1 constant region display a more potent tumour response compared to TEGs expressing γδTCRs comprising the same variable regions but comprising a Cγ2 constant region. (FIG. 10A) Flow cytometry analysis of TEGs expressing either Cγ1-constant region-comprising or Cγ2-constant region-comprising variants of the γδTCR of c15 or C132 (described in WO2017/212074) was performed to evaluate the surface expression of the γδTCRs, using antibodies specific to αβTCR (clone IP26) and γδTCR (clone IMMU510). Acquisition was performed with BD FACS Fortessa and data analysized with FlowJo v10. Displayed plots show CD3+ gated cells. (FIG. 10B to FIG. 10E) TEGs expressing either Cγ1-constant region-comprising or Cγ2-constant region-comprising variants of the γδTCR of c15 or C132 were co-cultured with RKO colon carcinoma cell line (FIGS. 10B and 10C) or with HT-29 and SW480 colon carcinoma cell lines (FIG. 10D and FIG. 10E) at effector to target (E:T) ratio 1:1 for 48 hrs. For TEG expressing γδTCR of clone 5, co-cultures were performed in the presence (+) or absence (−) of N-bisphosphonate Pamidronate (10 μM final concentration). Target cell cytotoxicity (FIG. 10B and FIG. 10D) was measured by xCELLigence and plotted as percentage of cytolysis relative to maximum cytolysis induced by treatment of the target cells with the detergent Triton-X-100. IFNγ levels (FIG. 10C and FIG. 10E) were measured by ELISA. Bars represent mean±SD of triplicates in a single experiment (representative of n=2 experiments with 2 αβT-cell donors). n.s: not significant; *P<0.05; **P<0.01; ***P<0.001 ****P<0.0001, multiple t-test.

FIG. 11A-11D. Soluble γδTCR-CD3 bispecific engagers mediate a strong anti-tumour response, particularly when comprising a Cγ1 constant region part. Increasing concentrations (1, 3 or 10 μg/well/200 μl) of soluble γδTCR clone 3-CD3 bispecific engagers comprising either a Cγ1 or Cγ2 region part were added to co-cultures of PBMC-derived αβT-cells and Luciferase-transduced RKO colon carcinoma cell line (FIG. 11A and FIG. 11B) or Luciferase-transduced MDA-MB-231 breast carcinoma cell lines (FIG. 11C and FIG. 11D) (effector to target ratio 1:1). Co-cultures were maintained for 48 hrs at 37° C. after which target cell cytotoxicity (FIG. 11A and FIG. 11C) was measured by decreased Luciferase activity relative to target cells cultured alone and plotted as % of cytotoxicity. IFNγ levels (FIG. 11 B and FIG. 11D) were measured by ELISA. Bars represent mean±SD of duplicates in a single experiment. n.s: not significant; *P<0.05; **P<0.01; ****P<0.0001, multiple t-test.

FIG. 12A-12C. Schematic representation of the γδTCR heterodimer (FIG. 12A). Each γ- and δ-chain contains a variable region (antigen-binding and recognition) and a constant region (cell membrane anchoring and TCR/CD3 complex formation and signaling). The γ-chain constant region has allelic variants (Cγ1 or Cγ2) which differ by the presence or absence of a interchain Cys-bound, respectively, and an extra-amino acid sequence present in the Cγ2 only. FIG. 12B demonstrates an overview of the human TRG locus chromosomal localization (adapted from IMGT@, http://www.imgt.org). V:variable genes; JP, JP1, J1, JP2, J2: Joining genes; C1, C2: Constant genes. FIG. 12C provides a schematic representation of TRGC1 and TRCG2 genes encoding for Cγ1 or Cγ2 regions of human γTCR. The TRGC2 gene can include a duplication (EX2R and EX2) or a triplication (EX2T, EX2R and EX2) of Exon 2, which translates into an incorporation of 16 extra amino acids (Ex 2 (2x)) or 32 extra amino acids (Ex 2 (3x)).

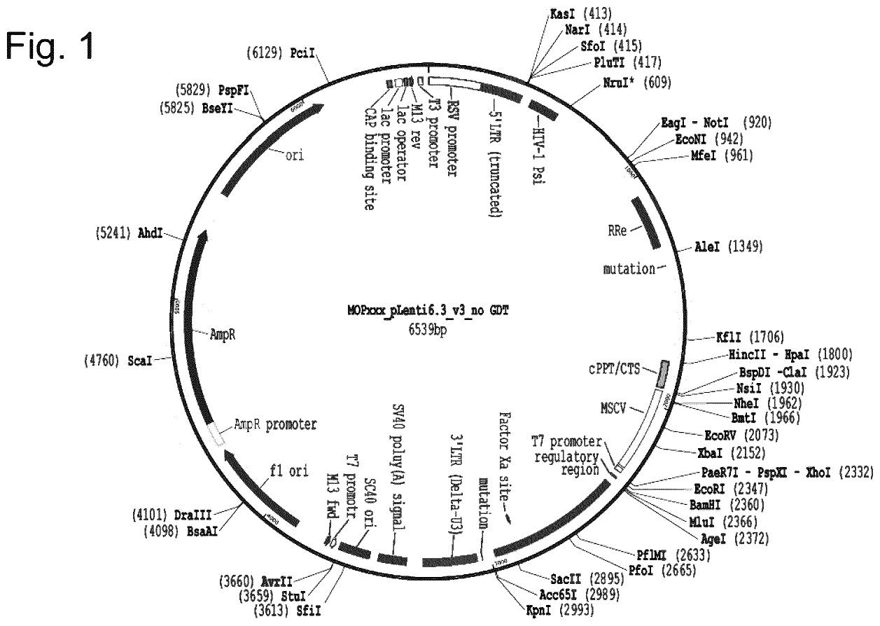

### SUMMARY OF THE INVENTION

In a first aspect, the invention relates a δT-cell receptor chain or a part thereof comprising a CDR3 region, said δT-cell receptor chain or part thereof being represented by an amino acid sequence, said amino acid sequence comprising at least 70% sequence identity with amino acid sequence SEQ ID NO: 7, 9, 13, 15, 19, and/or 21.

In a second aspect, the invention relates to a γT-cell receptor chain or a part thereof comprising a CDR3 region, said γT-cell receptor chain or part thereof being represented by an amino acid sequence, said amino acid sequence comprising at least 85% sequence identity with amino acid sequence SEQ ID NO: 10, 12, 16, 18, 22, and/or 24.

In a third aspect, the invention relates to a nucleic acid molecule encoding an amino acid sequence as defined in the first aspect, wherein said nucleic acid molecule is represented by a nucleotide sequence comprising a nucleotide sequence that has at least 60% sequence identity with SEQ ID NO: 8, 14, 20, 28, 30, and/or 32 and/or a nucleotide sequence that encodes an amino acid sequence that has at least 60% amino acid identity with an amino acid sequence encoded by a nucleotide sequence SEQ ID NO: 8, 14, 20, 28, 30, and/or 32.

In a fourth aspect, the invention relates to a nucleic acid molecule encoding an amino acid sequence as defined in the second aspect, wherein said nucleic acid molecule is represented by a nucleotide sequence comprising a nucleotide sequence that has at least 80% sequence identity with SEQ ID NO: 11, 17, 23, 29, 31, and/or 33 and/or a nucleotide sequence that encodes an amino acid sequence that has at least 80% amino acid identity with an amino acid sequence encoded by a nucleotide sequence SEQ ID NO: 11, 17, 23, 29, 31, and/or 33.

In a fifth aspect, the invention relates to a γδTCR or part thereof comprising a CDR3 region comprising:
a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 7, 9, 13, 15, 19, and/or 21, and/or
a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 10, 12, 16, 18, 22, and/or 24.

In some embodiments, the γδTCR or part thereof of the fifth aspect comprises A or B or C:
A:
   a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 7 and/or 9 and/or
   a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 10 and/or 12,
B:
   a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 13 and/or 15 and/or
   a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 16 and/or 18,
C:
   a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 19 and/or 21 and/or
   a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 22 and/or 24.

In a sixth aspect, the invention relates to a nucleic acid molecule encoding a γδTCR or a part thereof according to the fifth aspect, said nucleic acid molecule being represented by a nucleotide sequence comprising:
a nucleotide sequence that has at least 60% sequence identity with SEQ ID NO: 8, 14 20, 28, 30, and/or 32 and/or a nucleotide sequence that encodes an amino acid sequence that has at least 60% amino acid identity with an amino acid sequence encoded by a nucleotide sequence SEQ ID NO: 8, 14, 20, 28, 30, and/or 32 and/or
a nucleotide sequence that has at least 80% sequence identity with SEQ ID NO: 11, 17, 23, 29, 31, and/or 33 and/or a nucleotide sequence that encodes an amino acid sequence that has at least 80% amino acid identity with an amino acid sequence encoded by a nucleotide sequence SEQ ID NO: 11, 17, 23, 29, 31, and/or 33.

In some embodiments of the nucleic acid molecule encoding a γδTCR or part thereof of the sixth aspect, said nucleic acid molecule being represented by a nucleotide sequence comprising A1, B1 or C1:
A1:
   a nucleotide sequence that has at least 60% sequence identity with SEQ ID NO: 8 and/or 28 and/or a nucleotide sequence that encodes an amino acid sequence that has at least 60% amino acid identity with an amino acid sequence encoded by a nucleotide sequence SEQ ID NO: 8 and/or 28, and/or
   a nucleotide sequence that has at least 80% sequence identity with SEQ ID NO: 11 and/or 29 and/or a nucleotide sequence that encodes an amino acid sequence that has at least 80% amino acid identity with an amino acid sequence encoded by a nucleotide sequence SEQ ID NO: 11 and/or 29,
B1:
   a nucleotide sequence that has at least 60% sequence identity with SEQ ID NO: 14 and/or 30 and/or a nucleotide sequence that encodes an amino acid sequence that has at least 60% amino acid identity with an amino acid sequence encoded by a nucleotide sequence SEQ ID NO: 14 and/or 30, and/or
   a nucleotide sequence that has at least 80% sequence identity with SEQ ID NO: 17 and/or 31 and/or a nucleotide sequence that encodes an amino acid sequence that has at least 80% amino acid identity with an amino acid sequence encoded by a nucleotide sequence SEQ ID NO: 17 and/or 31,
C1:
   a nucleotide sequence that has at least 60% sequence identity with SEQ ID NO: 20 and/or 32 and/or a nucleotide sequence that encodes an amino acid sequence that has at least 60% amino acid identity with an amino acid sequence encoded by a nucleotide sequence SEQ ID NO: 20 and/or 32, and/or
   a nucleotide sequence that has at least 80% sequence identity with SEQ ID NO: 23 and/or 33 and/or a nucleotide sequence that encodes an amino acid sequence that has at least 80% amino acid identity with an amino acid sequence encoded by a nucleotide sequence SEQ ID NO: 23 and/or 33.

In some embodiments of the first, second, and fifth aspects, the δT-cell receptor chain or a part thereof, γT-cell receptor chain or a part thereof, or γδTCR or part thereof mediates an anti-tumour or an anti-infective response.

In some embodiments of the first, second, and fifth aspects, the δT-cell receptor chain or a part thereof, γT-cell receptor chain or a part thereof, or γδTCR or part thereof is a soluble polypeptide, preferably comprising a T-cell- and/or NK-cell-binding domain.

In a seventh aspect, the invention relates to a conjugate comprising a δT-cell receptor chain or a part thereof of the first aspect, a γT-cell receptor chain or a part thereof of the second aspect, or a γδTCR or a part thereof of the fifth aspect, linked to an agent.

In some embodiments of the seventh aspect, the invention relates to a conjugate comprising a part of the δT-cell receptor chain as defined in the first aspect or comprising a part of the γT-cell receptor chain of the second aspect, linked to an agent.

In some embodiments of the seventh aspect, the agent is selected from the group consisting of a diagnostic agent, a therapeutic agent, an anti-cancer agent, a chemical, a nanoparticle, a chemotherapeutic agent a fluorescent protein or an enzyme whose catalytic activity could be detected.

In an eighth aspect, the invention relates to a nucleic acid construct comprising a nucleic acid molecule encoding the amino acid sequence as identified earlier herein (first, second, fifth aspects) and/or wherein said nucleic acid molecule is as identified earlier herein (third, fourth, sixth aspects).

In some embodiments of the eighth aspect, the nucleic acid construct is a vector, preferably a viral vector, more preferably a retroviral vector and most preferably a lentiviral vector.

In a ninth aspect, the invention relates to a cell comprising the nucleic acid construct or the vector of the eight aspect.

In some embodiments of the ninth aspect, the cell is a T-cell comprising a nucleic acid molecule encoding the amino acid sequence as identified earlier herein (first, second, fifth aspects) and/or expressing the amino acid sequence as identified earlier herein (first, second, fifth aspects) and/or comprising a nucleic acid molecule as identified earlier herein (third, fourth, sixth aspects).

In some embodiments of the ninth aspect, the cell is a T-cell expressing a γδTCR comprising A or B or C:
A:
   a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 7 and/or 9 and/or
   a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 10 and/or 12,
B:
   a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 13 and/or 15 and/or
   a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 16 and/or 18,
C:
   a δT-cell receptor chain or part thereof comprising a CDR3 region, said δT-cell receptor chain or part thereof being represented by an amino acid sequence, said amino acid sequence comprising at least 70% sequence identity with amino acid sequence SEQ ID NO: 19 and/or 21 and/or
   a γT-cell receptor chain or part thereof comprising a CDR3 region, said γT-cell receptor chain or part thereof being represented by an amino acid sequence, said amino acid sequence comprising at least 85% sequence identity with amino acid sequence SEQ ID NO: 22 and/or 24.

In some embodiments of the ninth aspect, the cell is a T-cell further comprising a polynucleotide encoding a chimeric bidirectional signaling transmembrane protein able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner.

In some embodiments, the extracellular ligand domain comprises an amino acid sequence from 41BBL, OX40L, CD86, RANK, or CD70, and the heterologous intracellular signaling domain comprises an amino acid sequence from OX40, 41BB, NKp80, IL18RAP, or IL2RB.

In some embodiments, the chimeric bidirectional signaling transmembrane protein is represented by an amino acid sequence having at least 80% identity with SEQ ID NO: 83.

In some embodiments of the ninth aspect, the T-cell is an αβT-cell.

In a tenth aspect, the invention relates to a population of cells comprising the cell of the ninth aspect.

In an eleventh aspect, the invention relates to a composition, preferably a pharmaceutical composition, comprising a δT-cell receptor chain or a part thereof of the first aspect, a γT-cell receptor chain or a part thereof of the second aspect, a γδTCR or a part thereof of the fifth aspect, a nucleic acid molecule of the third, fourth, or sixth aspects, a conjugate of the seventh aspect, a nucleic acid construct or a vector of the eighth aspect, a cell of the ninth aspect, or a population of cells of the tenth aspect.

In a further aspect, a δT-cell receptor chain or a part thereof of the first aspect, a γT-cell receptor chain or a part thereof of the second aspect, a γδTCR or a part thereof of the fifth aspect, a nucleic acid molecule of the third, fourth or sixth aspects, a conjugate of the seventh aspect, a nucleic acid construct or a vector of the eight aspect, a cell of the ninth aspect, a population of cells of the tenth aspect, or a composition of the eleventh aspect is for use as a medicament.

In some embodiments, the medicament is for preventing, treating, regressing, curing and/or delaying a cancer or an infection.

In a further aspect, the invention relates to a method for improving the anti-tumour or anti-infective response mediated by a γδT-cell receptor or a part thereof comprising a CDR3 region, wherein said receptor or part thereof comprises a Cγ2 constant region or a part thereof, said method comprising the step of replacing said Cγ2 constant region or part thereof by a Cγ1 constant region or a part thereof.

In a further aspect, the invention relates to a method for identifying a γδT-cell receptor or a part thereof comprising a CDR3 region that mediates an improved anti-tumour or anti-infective response comprising the steps of:
a) providing a γδT-cell receptor or a part thereof comprising a CDR3 region, wherein said receptor or part thereof comprises a Cγ2 constant region or a part thereof;
b) replacing said Cγ2 constant region or part thereof by a Cγ1 constant region or a part thereof;
c) expressing the γδT-cell receptor or a part thereof obtained in step b) in an engineered T-cell, preferably an αβT-cell;
d) determining the anti-tumour or anti-infective response of the engineered T-cell of step c);
e) identifying the γδT-cell receptor or part thereof that mediates the improved anti-tumour or anti-infective response.

In a further aspect, the invention relates to a method for identifying a soluble γδT-cell receptor or a part thereof comprising a CDR3 region that mediates an improved anti-tumour or anti-infective response comprising the steps of:
a) providing a soluble γδT-cell receptor or a part thereof comprising a CDR3 region, wherein said receptor or part thereof comprises a Cγ2 constant region or a part thereof and a T-cell- and/or NK-cell-binding domain;
b) replacing said Cγ2 constant region or part thereof by a Cγ1 constant region or a part thereof;
c) expressing the soluble γδT-cell receptor or a part thereof obtained in step b) in a host cell, preferably a human cell;
d) obtaining the soluble γδT-cell receptors or parts thereof expressed by the cells of step c);
e) contacting the soluble γδT-cell receptors or parts thereof obtained in step d) with a T-cell, preferably an αβT-cell, and a target cell;
f) determining the anti-tumour or anti-infective response of the T-cell of step e);
g) identifying the soluble γδT-cell receptor or part thereof that mediates the improved anti-tumour or anti-infective response.

In some embodiments, the T-cell-binding domain is a CD3-binding domain. In some embodiments, the CD3-binding domain is an scFv, preferably represented by an amino acid sequence comprising or consisting of SEQ ID NO: 146 or a variant thereof. In some embodiments, in step d) the soluble γδT-cell receptors or parts thereof are isolated and/or purified.

In some embodiments of the methods of the aspects relating to replacement of a Cγ2 constant region or part thereof by a Cγ1 constant region or part thereof, the Cγ2 constant region or part thereof is represented by an amino acid sequence comprising at least 95% sequence identity or similarity with SEQ ID NO: 161 or SEQ ID NO: 164, preferably with SEQ ID NO: 161.

In some embodiments of the methods of the aspects relating to replacement of a Cγ2 constant region or part thereof by a Cγ1 constant region or part thereof, the Cγ1 constant region or part thereof is represented by an amino acid sequence comprising at least 95% sequence identity or similarity with SEQ ID NO: 152.

In some embodiments of the methods of the aspects relating to replacement of a Cγ2 constant region or part thereof by a Cγ1 constant region or part thereof, the Cγ1 constant region or part thereof is encoded by a nucleic acid molecule represented by a nucleotide sequence comprising at least 95% sequence identity with SEQ ID NO: 151.

In some embodiments of the methods of the aspects relating to replacement of a Cγ2 constant region or part thereof by a Cγ1 constant region or part thereof, the Cγ1 constant region or part thereof is represented by an amino acid sequence that does not comprise SEQ ID NO: 158 or part thereof.

In a further aspect, there is provided a γδT-cell receptor or part thereof obtained by or obtainable by the methods of the aspects relating to replacement of a Cγ2 constant region or part thereof by a Cγ1 constant region or part thereof.

### DESCRIPTION OF THE INVENTION

### δT-Cell and γT-Cell Receptor Chain or a Part Thereof

Provided in certain aspects described herein are polypeptides comprising a δT-cell receptor chain or a variant or part or fragment thereof. In a further aspect the invention provides a δT-cell receptor chain or a part thereof, comprising a CDR3 region, and which δT-cell receptor chain or part thereof is represented by an amino acid sequence as defined herein. Each of these δT-cell receptor chains or parts thereof may be represented by an amino acid sequence that could be identified using a SEQ ID NO. In an embodiment, a δT-cell receptor chain is a δ1T-cell receptor chain or a δ3T-cell receptor chain.

Provided in certain aspects described herein are polypeptides comprising a γT-cell receptor chain or a variant or part or fragment thereof. In a further aspect the invention provides a γT-cell receptor chain or a part thereof, comprising a CDR3 region, and which γT-cell receptor chain or part thereof is represented by an amino acid sequence as defined herein. Each of these γT-cell receptor chains or parts thereof may be represented by an amino acid sequence that could be identified using a SEQ ID NO. In an embodiment, a γT-cell receptor chain is a γ9T-cell receptor chain or a γ3T-cell receptor chain or a γ4T-cell receptor chain.

Provided in certain aspects described herein are polypeptides comprising a γδT-cell receptor (also referred to herein as γδTCR) or a variant or part or fragment thereof. In a further aspect, the invention provides a γδT-cell receptor or a part thereof, comprising a CDR3 region, and which γδT-cell receptor or part thereof comprises a δT-cell receptor chain or a part thereof, comprising a CDR3 region, and a γT-cell receptor chain or a part thereof, comprising a CDR3 region. Each of the δT-cell receptor chain or part thereof and γT-cell receptor chain or part thereof may be represented by an amino acid sequence that could be identified using a SEQ ID NO.

A “variant” polypeptide as used herein refers to a polypeptide comprising an amino acid modification as compared to the amino sequence of the polypeptide it is derived from. An “amino acid modification” as described herein may refer to a modification resulting in an amino acid sequence being modified (altered). Such a modification may, for example, be an amino acid substitution, insertion and/or deletion. An amino acid substitution refers to a sequence modification that replaces an amino acid residue in a parent (reference) amino acid sequence (or a nucleotide in a nucleotide sequence comprised by a nucleic acid encoding the amino acid sequence) which results in a variant (derivative) sequence that has the same number of amino acids. An amino acid substitution may correspond to a substitution by any other amino acid. An amino acid substitution may correspond to a substitution of an L-amino acid by a D-amino acid.

An amino acid substitution may correspond to a substitution by a non-natural amino acid. An amino acid substitution may be conservative. A definition of “conservative” amino acid substitutions is provided later herein. In embodiments wherein multiple amino acids are substituted, they may correspond to consecutive positions, to positions that are not consecutive, or to positions that are spatially apart in the amino acid sequence. The skilled person undestands that amino acid modifications in the context of the disclosure may be combined, e.g., an amino acid sequence may comprise an amino acid substitution and an amino acid insertion and/or deletion relative to an amino acid sequence having a SEQ ID NO as described herein.

A “variant” polypeptide in the context of a γT-cell receptor chain or a part thereof (or γδT-cell receptors or parts thereof comprising them) may also refer to a γT-cell receptor chain or part thereof (or γδT-cell receptors or parts thereof comprising them) naturally or natively comprising a Cγ2 constant region or part thereof in which the Cγ2 constant region or part thereof has been replaced by a Cγ1 constant region or part thereof using the methods of the invention described later herein.

“Part thereof” and “fragment thereof” with respect to the polypeptides of the invention are used herein interchangeably. Part or fragment thereof may correspond to at least 1%, at least 2%, at least 3%, at least 4%, at least 5%, at least 10%, at least 20%, at least 30%, at least 40% of the length of a polypeptide, such as (for example) represented by an amino acid sequence with a specific SEQ ID NO, or it may correspond to at least 50% of the length of the SEQ ID NO, or at least 60%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%. A part or fragment of a polypeptide may correspond to an extracellular domain of a polypeptide, such as of a γT-cell receptor chain, a δT-cell receptor chain, or a γδT-cell receptor, or part of said extracellular domain, as discussed later herein. A part or fragment of a polypeptide may correspond to a complete variable region and/or a fragment or part of a constant region of a γT-cell receptor chain, a δT-cell receptor chain, or a γδT-cell receptor, for example a Cγ2 or Cγ1 region or part thereof. A part or fragment of a polypeptide may correspond to a part or fragment of a variable region and/or a fragment or part of a constant region of a γT-cell receptor chain, a δT-cell receptor chain, or a γδT-cell receptor, for example a part of a Cγ2 or Cγ1 constant region. A part or fragment of a polypeptide may correspond to a CDR3 region of a γT-cell receptor chain, a δT-cell receptor chain, or a γδT-cell receptor. A part or fragment of a polypeptide may correspond to a soluble polypeptide, such as a soluble γT-cell receptor chain, a soluble δT-cell receptor chain, or a soluble γδT-cell receptor, as described later herein. A part or fragment of a polypeptide is preferably a functional part or fragment thereof. It may mean that this part or fragment exhibits a similar activity as the original polypeptide it derives from. In the context of the invention, an activity may be an anti-tumour response as explained later herein. In the context of the invention, an activity may be an anti-infective response as explained later herein. A similar anti-tumour or anti-infective response may mean that the part or fragment of the polypeptide mediates at least 50% of said anti-tumour or anti-infective response, or at least 60%, or at least 70%, or at least 80%, or at least 90%, or at least 100% or at least 110% or at least 120% or more, as compared to the original polypeptide it is derived from. In some embodiments, a part or fragment of a γT-cell receptor chain, δT-cell receptor chain, or γδT-cell receptor corresponds to an extracellular domain or part or fragment thereof, as described later herein.

Each δT-cell receptor chain or part thereof comprising a CDR3 region identified herein may also be represented by its coding nucleic acid sequence instead of its amino acid sequence. Therefore, the invention also relates to a nucleic acid molecule encoding said receptor chain or part thereof. The same holds for each of the γT-cell receptor chain or part thereof comprising a CDR3 region identified herein. The same also holds for the γδTCR identified herein: it can be identified by the receptor chains it comprises or by the nucleic acid molecules encoding the chains it comprises. The same also holds for the cell, such as the T-cell, expressing said γδTCR identified later herein: the cell, such as T-cell, can be defined by reference to the receptor chains or parts thereof it expresses or by the nucleic acid molecules encoding these chains or parts thereof it comprises.

Preferably, each δT-cell receptor chain, γT-cell receptor chain, γδT-cell receptor, variant, or part thereof is a mammalian, preferably human, δT-cell receptor chain, γT-cell receptor chain, γδT-cell receptor, variant, or part thereof.

Each δT-cell receptor chain, γT-cell receptor chain, γδT-cell receptor, variant, or part thereof, may be an isolated polypeptide. Each δT-cell receptor chain, γT-cell receptor chain, γδT-cell receptor, variant, or part thereof, may be synthetically made. Each δT-cell receptor chain, γT-cell receptor chain, γδT-cell receptor, variant, or part thereof, may be comprised, preferably expressed, by a cell as described later herein, for example in a cellular membrane (surface expression). A cell may alternatively express the δT-cell receptor chain, γT-cell receptor chain, γδT-cell receptor, variant, or part thereof as a soluble polypeptide, as described later herein.

In embodiments wherein the δT-cell receptor chain, γT-cell receptor chain, γδT-cell receptor, variant, or part thereof is comprised, preferably expressed, by a cell, said δT-cell receptor chain, γT-cell receptor chain, γδT-cell receptor, variant, or part thereof is preferably exogenous to said cell. Exogenous in this context refers to the corresponding polypeptide being introduced to said cell, for example using one of the methods as described later herein. In some embodiments, an exogenous γT-cell receptor chain, γδT-cell receptor, variant, or part thereof is not naturally present in the cell it is introduced in. For example, an αβT-cell may express an exogenous γδT-cell receptor or a part thereof. As an additional example, a γδT-cell may express an exogenous γδT-cell receptor or a part thereof.

In a first aspect, there is provided a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7, 9, 13, 15, 19, and/or 21, or with amino acid sequence SEQ ID NO: 7, 9, 13, 15, 19, 21, 142, and/or 153.

Preferably, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In some embodiments, there is provided a δT-cell receptor chain or a part thereof comprising a CDR3 region, said δT-cell receptor chain or part thereof being represented by an amino acid sequence, said amino acid sequence comprising at least 60%, at least 70%, at least 80%, at least 85%, at least 90%, or 100%, preferably at least 70%, sequence identity with amino acid sequence SEQ ID NO: 7, 13, and/or 19.

In an embodiment, there is provided a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7 and/or 9, preferably at least 70% sequence identity with amino acid sequence SEQ ID NO: 7.

In another embodiment, there is provided a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 13 and/or 15, or with amino acid sequence SEQ ID NO: 13, 15, 142, and/or 153, preferably at least 70% sequence identity with amino acid sequence SEQ ID NO: 13.

In an embodiment, there is provided a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 19 and/or 21, preferably at least 70% sequence identity with amino acid sequence SEQ ID NO: 19.

Preferably, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a second aspect, there is provided a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10, 12, 16, 18, 22, and/or 24, or with amino acid sequence SEQ ID NO: 10, 12, 16, 18, 22, 24, 131, 133, 135, 143, 144, 154, 155, and/or 162.

Preferably, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In some embodiments, there is provided a γT-cell receptor chain or a part thereof comprising a CDR3 region, said γT-cell receptor chain or part thereof being represented by an amino acid sequence, said amino acid sequence comprising at least 80%, at least 85%, at least 90%, or 100%, preferably at least 85%, sequence identity with amino acid sequence SEQ ID NO: 10, 16, and/or 22.

In an embodiment, there is provided a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10 and/or 12, or with amino acid sequence SEQ ID NO: 10, 12. and/or 131, preferably at least 85% sequence identity with amino acid sequence SEQ ID NO: 10.

In an embodiment, there is provided a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 16 and/or 18, or with amino acid sequence SEQ ID NO: 16, 18, 133, 143, 144, 154, 155, and/or 162, preferably at least 85% sequence identity with amino acid sequence SEQ ID NO: 16.

In an embodiment, there is provided a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 22 and/or 24, or with amino acid sequence SEQ ID NO: 22, 24, and/or 135, preferably at least 85% sequence identity with amino acid sequence SEQ ID NO: 22. Preferably, the identity or similarity is of at least 81%, 82%, 83%, 84%, 85%, 86%, 87%, 88%, 89%, 90%, 91%, 92%, 93%, 94%, 95%, 96%, 97%, 98%, 99% or 100%.

In a third aspect, there is provided a nucleic acid molecule represented by a nucleotide sequence comprising a nucleotide sequence that has at least 60% sequence identity with SEQ ID NO: 8, 14, 20, 28, 30, and/or 32 and/or a nucleotide sequence that encodes an amino acid sequence that has at least 60% amino acid identity or similarity with an amino acid sequence encoded by a nucleotide sequence SEQ ID NO: 8, 14, 20, 28, 30, and/or 32. In an embodiment, the nucleic acid molecule of the third aspect encodes a δT-cell receptor chain of the first aspect as identified earlier herein.

Preferably, the identity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a fourth aspect, there is provided a nucleic acid molecule represented by a nucleotide sequence comprising a nucleotide sequence that has at least 80% sequence identity with SEQ ID NO: 11, 17, 23, 29, 31, and/or 33, or with SEQ ID NO: 11, 17, 23, 29, 31, 33, 130, 132, and/or 134 and/or a nucleotide sequence that encodes an amino acid sequence that has at least 80% amino acid identity or similarity with an amino acid sequence encoded by a nucleotide sequence SEQ ID NO: 11, 17, 23, 29, 31, and/or 33, or SEQ ID NO: 11, 17, 23, 29, 31, 33, 130, 132, and/or 134. In an embodiment, the nucleic acid molecule of the fourth aspect encodes a γT-cell receptor chain of the second aspect as identified earlier herein.

Preferably, the identity is of at least 81%, 82%, 83%, 84%, 85%, 86%, 87%, 88%, 89%, 90%, 91%, 92%, 93%, 94%, 95%, 96%, 97%, 98%, 99% or 100%.

Each of the preferred δT-cell or γT-cell receptor chains or parts thereof defined above by sequence identity (defined by reference to an amino acid sequence or by reference to a nucleic acid molecule encoding them) and as encompassed by the invention are preferably considered to be able to exhibit and/or mediate an anti-tumour activity/response or an anti-infective activity/response as explained later herein.

The same holds for the γδTCRs or parts thereof comprising a CDR3 region described later herein Accordingly, in some embodiments, a δT-cell receptor chain or a part thereof, a γT-cell receptor chain or a part thereof, or a γδTCR or a part thereof, as described herein, mediates an anti-tumour response. In some embodiments, a δT-cell receptor chain or a part thereof, a γT-cell receptor chain or a part thereof, or a γδTCR or a part thereof, as described herein, mediates an anti-infective response.

In some embodiments, a polypeptide described herein, such as a polypeptide comprising a δT-cell receptor chain or a part thereof, a γT-cell receptor chain or a part thereof, or a γδTCR or a part thereof, is a soluble polypeptide. A “soluble” polypeptide as used herein refers to a polypeptide that may be in solution, i.e., a polypeptide that is not embedded in a cellular membrane. In some embodiments, a soluble polypeptide comprises or consists of the extracellular domain of a γT-cell receptor chain, a δT-cell receptor chain, a γδT-cell receptor, or a part thereof, optionally fused to additional domains, as described herein.

The skilled person is able to obtain an extracellular domain of a γT-cell receptor chain, a δT-cell receptor chain, a γδT-cell receptor, or a part thereof described herein using standardized nomenclature such as IMGT to pinpoint the exact amino acids corresponding to the domain. IMGT nomenclature is described in Lefranc et al., 2005 (Nucl Acids Res 33: D593-D597) and Lefranc et al., 2014 (Front Immunol 5:22), both of which are incorporated herein in their entireties, and is further described in the public database available at imgt.org. Further, the transmembrane domains of human γT- and δT-cell receptor chains are generally conserved and their sequences are available to the skilled person; see Uniprot Ref: POCF51 for TRGC1 chains, Uniprot Ref: P03986 for TRGC2 chains, and Uniprot Ref: B7Z8K6 for TRDC chains. Using this information, the skilled person may easily arrive at γT-cell receptor chains, δT-cell receptor chains, γδT-cell receptors, or parts thereof, which do not comprise a transmembrane domain. Accordingly, in some embodiments, a soluble polypeptide does not comprise a transmembrane domain of a γT-cell receptor chain, a δT-cell receptor chain, or a γδT-cell receptor, or a part thereof. In some embodiments, a soluble polypeptide does not comprise a cytoplasmic domain of a γT-cell receptor chain, a δT-cell receptor chain, or a γδT-cell receptor, or a part thereof. In some embodiments, a soluble polypeptide does not comprise SEQ ID NO: 158 or a part thereof. In some embodiments, a soluble polypeptide does not comprise SEQ ID NO: 159 or a part thereof. In some embodiments, a soluble polypeptide does not comprise SEQ ID NO: 160 or a part thereof.

In some embodiments, a polypeptide, preferably a soluble polypeptide, comprises a δT-cell receptor chain or a part thereof, comprising a CDR3 region, wherein said δT-cell receptor chain or part thereof comprises an amino acid sequence, said amino acid sequence comprising at least 60%, at least 70%, at least 80%, at least 85%, at least 90%, at least 95%, or 100%, preferably at least 70%, sequence identity or similarity with the amino acid sequence SEQ ID NO: 7.

In some embodiments, a polypeptide, preferably a soluble polypeptide, comprises a γT-cell receptor chain or a part thereof, comprising a CDR3 region, wherein said γT-cell receptor chain or part thereof comprises an amino acid sequence, said amino acid sequence comprising at least 60%, at least 70%, at least 80%, at least 85%, at least 90%, at least 95%, or 100%, preferably at least 85%, sequence identity or similarity with the amino acid sequence SEQ ID NO: 10.

In some embodiments, a polypeptide, preferably a soluble polypeptide, comprises:
a δT-cell receptor chain or a part thereof, comprising a CDR3 region, wherein said δT-cell receptor chain or part thereof comprises an amino acid sequence, said amino acid sequence comprising at least 60%, at least 70%, at least 80%, at least 85%, at least 90%, at least 95%, or 100%, preferably at least 70%, sequence identity or similarity with the amino acid sequence SEQ ID NO: 7, and/or;
a γT-cell receptor chain or a part thereof, comprising a CDR3 region, wherein said γT-cell receptor chain or part thereof comprises an amino acid sequence, said amino acid sequence comprising at least 60%, at least 70%, at least 80%, at least 85%, at least 90%, at least 95%, or 100%, preferably at least 85%, sequence identity or similarity with the amino acid sequence of SEQ ID NO: 10.

In some embodiments, the polypeptide does not comprise SEQ ID NO: 158 or a part thereof. In some embodiments, the polypeptide does not comprise SEQ ID NO: 159 or a part thereof. In some embodiments, the polypeptide does not comprise SEQ ID NO: 160 or a part thereof.

In some embodiments, a polypeptide, preferably a soluble polypeptide, comprises a δT-cell receptor chain or a part thereof, comprising a CDR3 region, wherein said δT-cell receptor chain or part thereof comprises an amino acid sequence, said amino acid sequence comprising at least 60%, at least 70%, at least 80%, at least 85%, at least 90%, at least 95%, or 100%, preferably at least 70%, sequence identity or similarity with the amino acid sequence SEQ ID NO: 13, 142, and/or 153.

In some embodiments, a polypeptide, preferably a soluble polypeptide, comprises a γT-cell receptor chain or a part thereof, comprising a CDR3 region, wherein said γT-cell receptor chain or part thereof comprises an amino acid sequence, said amino acid sequence comprising at least 60%, at least 70%, at least 80%, at least 85%, at least 90%, at least 95%, or 100%, preferably at least 85%, sequence identity or similarity with the amino acid sequence SEQ ID NO: 16, 143, 144, 154, and/or 155, preferably with the amino acid sequence SEQ ID NO: 16, 143, and/or 154. In some embodiments, a polypeptide, preferably a soluble polypeptide, comprises:
a δT-cell receptor chain or a part thereof, comprising a CDR3 region, wherein said δT-cell receptor chain or part thereof comprises an amino acid sequence, said amino acid sequence comprising at least 60%, at least 70%, at least 80%, at least 85%, at least 90%, at least 95%, or 100%, preferably at least 70%, sequence identity or similarity with the amino acid sequence SEQ ID NO: 13, 142, and/or 153;
a γT-cell receptor chain or a part thereof, comprising a CDR3 region, wherein said γT-cell receptor chain or part thereof comprises an amino acid sequence, said amino acid sequence comprising at least 60%, at least 70%, at least 80%, at least 85%, at least 90%, at least 95%, or 100%, preferably at least 85%, sequence identity or similarity with the amino acid sequence of SEQ ID NO: 16, 143, 144, 154, and/or 155, preferably with the amino acid sequence SEQ ID NO: 16, 143, and/or 154.

In some embodiments, the polypeptide does not comprise SEQ ID NO: 158 or a part thereof. In some embodiments, the polypeptide does not comprise SEQ ID NO: 159 or a part thereof. In some embodiments, the polypeptide does not comprise SEQ ID NO: 160 or a part thereof. In some embodiments, a polypeptide, preferably a soluble polypeptide, comprises a δT-cell receptor chain or a part thereof, comprising a CDR3 region, wherein said δT-cell receptor chain or part thereof comprises an amino acid sequence, said amino acid sequence comprising at least 60%, at least 70%, at least 80%, at least 85%, at least 90%, at least 95%, or 100%, preferably at least 70%, sequence identity or similarity with the amino acid sequence SEQ ID NO: 19. In some embodiments, a polypeptide, preferably a soluble polypeptide, comprises a γT-cell receptor chain or a part thereof, comprising a CDR3 region, wherein said γT-cell receptor chain or part thereof comprises an amino acid sequence, said amino acid sequence comprising at least 60%, at least 70%, at least 80%, at least 85%, at least 90%, at least 95%, or 100%, preferably at least 85%, sequence identity or similarity with the amino acid sequence SEQ ID NO: 22. In some embodiments, a polypeptide, preferably a soluble polypeptide, comprises:
a δT-cell receptor chain or a part thereof, comprising a CDR3 region, wherein said δT-cell receptor chain or part thereof comprises an amino acid sequence, said amino acid sequence comprising at least 60%, at least 70%, at least 80%, at least 85%, at least 90%, at least 95%, or 100%, preferably at least 70%, sequence identity or similarity with the amino acid sequence SEQ ID NO: 19, and/or;
a γT-cell receptor chain or a part thereof, comprising a CDR3 region, wherein said γT-cell receptor chain or part thereof comprises an amino acid sequence, said amino acid sequence comprising at least 60%, at least 70%, at least 80%, at least 85%, at least 90%, at least 95%, or 100%, preferably at least 85%, sequence identity or similarity with the amino acid sequence of SEQ ID NO: 22.

In some embodiments, the polypeptide does not comprise SEQ ID NO: 158 or a part thereof. In some embodiments, the polypeptide does not comprise SEQ ID NO: 159 or a part thereof. In some embodiments, the polypeptide does not comprise SEQ ID NO: 160 or a part thereof.

Preferably, the identity or similarity is at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%.

In an embodiment, a variant or part of a δT-cell (or γT-cell) receptor chain, or a variant or part of a γδTCR described herein is a soluble polypeptide. Such a soluble polypeptide may also be called a binding unit. Such a soluble polypeptide can include various forms to binding entities such as, but not limited to, a γT-cell receptor chain, a δT-cell receptor chain, a γδTCR, antibody, scFv, BCR, VHH, or any combination thereof. In some cases, at least a portion or fragment or part of a TCR, such as a Vγ3Vδ1 or Vγ4Vδ3 or Vγ9Vδ1 can be generated and utilized in a composition (preferably a pharmaceutical compositions) as described herein. For example, TCR-antibody chimeras can be generated and tested before arriving at a desired chimera. For example, γδ-variable domains can replace heavy and light chain variable domains of an antibody. In addition to enhanced binding, an Fc domain of an antibody can mediate cytotoxicity through Fcγ-receptor positive immune cells and/or a complementary system.

Accordingly, a soluble polypeptide described herein may in some embodiments be a chimeric polypeptide, i.e., a polypeptide that comprises various forms or parts of binding entities such as, but not limited to, a γT-cell receptor chain, a δT-cell receptor chain, a γδT-cell receptor (or parts thereof such as extracellular domains or parts thereof), an antibody, an scFv, a B-cell receptor, a VHH, or any combination thereof.

In some embodiments, the soluble polypeptide comprises a γT-cell receptor chain, a δT-cell receptor chain, a γδT-cell receptor, or a part thereof (such as e.g., an extracellular domain or part thereof), that is fused with a T-cell- and/or NK-cell-binding domain. Such a soluble polypeptide may be called a bispecific polypeptide. A γδT-cell receptor or part thereof fused with a T-cell- and/or NK-cell-binding domain may alternatively be called a γδTCR bispecific engager.

Such a bispecific polypeptide or γδTCR bispecific engager may be advantageous, as it may first bind to a T- and/or NK-cell and then recruit the cell to a tumour cell, or to an infection site, thus mediating an anti-tumour or an anti-infective response without the requirement for its expression in a cellular membrane of an engineered T- and/or NK-cell.

A T-cell- and/or NK-cell-binding domain is to be understood as a domain that specifically binds to a T-cell and/or NK-cell, for example via binding to an antigen that is present on or displayed by the T-cell and/or NK-cell. In some embodiments, the T-cell and/or NK-cell is a mammalian cell, preferably a human cell. Preferably, binding of a T-cell- or NK-cell-binding domain to the respective T-cell or NK-cell results in the activation of the T-cell or NK-cell.

In some embodiments, the T-cell- and/or NK-cell-binding domain is derived from, or is, an antibody, a single heavγ chain variable domain antibody (such as for example a camelid VHH), a shark immunoglobulin-derived variable new antigen receptor, an scFv, a tandem scFv, a Fab, an Fc domain of an antibody, an scFab, an antibody mimetic (such as for example a designed ankyrin repeat protein), a binding protein based on a Z domain of protein A, a binding protein based on a fibronectin type III domain, a lipocalin, or combinations thereof.

In some embodiments, the T-cell- and/or NK-cell-binding domain is of mammalian origin, preferably of human origin.

In some embodiments, the T-cell- and/or NK-cell-binding domain is selected from the group of CD3-, CD4-, CD8-, CD16-, CD56-, CD103-, CD154-, CD314-binding domains, and combinations thereof. In some embodiments, a T-cell-binding domain is a CD3-binding domain, also referred to herein as an “anti-CD3” binding domain. A γδTCR bispecific engager comprising a CD3-binding domain may be called a γδTCR-CD3 bispecific engager.

Such binding domains are known to the skilled person, and are further described in e.g., WO2007/062245, Liao et al., 2000 (Gene Ther 7: 339-47), WO2001/051644, Arakawa et al., 1996 (J Biochem 120: 657-62), Adair et al., 1994 (Human Antibodies 5: 41-47), Kipriyanov et al., 1997 (Protein Engin Design Selection 10:445), van Diest et al., 2021 (J Immunother Cancer 2021; 9:e003850), and WO2019/156566, all of which are incorporated herein by reference in their entireties. For example, such binding domains include commercially available binding domains such as the ones offered by Creative Biolabs (Shirley, NY, USA). An additional example of such a domain is represented by SEQ ID NO: 146.

In some embodiments, a soluble polypeptide is a chimeric polypeptide comprising a γT-cell receptor chain, a δT-cell receptor chain, a γδT-cell receptor, or a part thereof (such as e.g., an extracellular domain or part thereof), and an scFv domain, preferably an anti-CD3 scFv domain. In some embodiments, the anti-CD3 scFv domain is represented by SEQ ID NO: 146 or a variant thereof.

In some embodiments, a soluble polypeptide is a chimeric polypeptide comprising a γT-cell receptor chain, a δT-cell receptor chain, a γδT-cell receptor, or a part thereof (such as e.g., an extracellular domain or part thereof), and an Fc domain of an antibody.

In some embodiments, the γT-cell receptor chain, δT-cell receptor chain, γδT-cell receptor, or part thereof (such as e.g., extracellular domain or part thereof) comprised in the soluble, such as chimeric, polypeptides described herein are fused to an extracellular domain of an immune checkpoint-related molecule (or part thereof), such as for example an immune checkpoint inhibitor. The term “immune checkpoint inhibitor” as used herein refers to polypeptides, such as, but not limited to, inhibitory receptors, expressed by T- and/or NK-cells.

Accordingly, in some embodiments, a soluble polypeptide is a chimeric polypeptide comprising a γT-cell receptor chain, a δT-cell receptor chain, a γδT-cell receptor, or a part thereof (such as e.g., an extracellular domain or part thereof), an extracellular immune checkpoint inhibitor domain, and a T-cell- and/or NK-cell-binding domain, preferably an anti-CD3 scFv or Fc domain.

In some embodiments, the anti-CD3 scFv domain is represented by SEQ ID NO: 146 or a variant thereof. Such a soluble polypeptide may be called a trispecific polypeptide. Suitable extracellular immune checkpoint inhibitor domains may be derived from, but are not limited to, the group consisting of the adenosine A2A receptor, programmed death 1 (PD1) receptor, T-cell immunoglobulin domain, mucin domain 3, and V-domain Ig suppressor of T-cell activation (TIGIT). Among the suitable immune checkpoint inhibitor domains, the extracellular domain of PD1 (or part thereof) is preferred. Such trispecific polypeptides may be advantageous in mediating an enhanced anti-tumour or anti-infective response. As a non-limiting example, the presence of the extracellular PD1 domain (or part thereof) in a trispecific polypeptide may interact with the PD-L1 ligand in a tumour cell, thereby enhancing the anti-tumour response of the T- and/or NK-cell that is recruited to the tumour cell via the binding domain of the polypeptide. Accordingly, in some embodiments, a soluble polypeptide is a chimeric polypeptide comprising a γT-cell receptor chain, a δT-cell receptor chain, a γδT-cell receptor, or a part thereof (such as e.g., an extracellular domain or part thereof), an extracellular domain of PD1, and a T-cell- and/or NK-cell-binding domain, preferably an anti-CD3 scFv or Fc domain. In some embodiments, the anti-CD3 scFv domain is represented by SEQ ID NO: 146 or a variant thereof.

In some embodiments, a soluble, such as a chimeric, polypeptide described herein may optionally further comprise a linker between the domains which provides conformational flexibility to the chimeric polypeptide. Suitable linkers are known to the skilled person, and may for example be selected from polypeptides comprising from 1 to 60 amino acid residues, from 5 to 40 amino acid residues, or from 10 to 20 amino acid residues. Examples of suitable linkers are described in e.g., WO1999/42077, WO2006/040153, WO2006/122825, WO2011/001152A1, and WO2019/156566, all of which are incorporated herein by reference in their entireties. Additional examples of suitable linkers are Gly-Ser linkers, such as, but not limited to, (Gly4Ser)3, (Gly4Ser)7, or (Gly3Ser2)3. Additional examples of suitable linkers are provided in Table 4 later herein. Preferred linkers are represented by SEQ ID NO: 145 and SEQ ID NO: 147.

A soluble, such as a chimeric, polypeptide as described herein may optionally comprise additional domains, for example a domain facilitating polypeptide excretion (in embodiments wherein the soluble polypeptide is produced by a cell, i.e., a signal peptide), and/or polypeptide isolation and/or purification and/or stability. Such domains and their applications are known in the art and are further described in standard handbooks such as Sambrook and Green, Molecular Cloning. A Laboratory Manual, 4th Edition, Cold Spring Harbor Laboratory Press (2012); Ausubel et al., Current Protocols in Molecular Biology, 3rd edition, John Wiley & Sons Inc (2003), both of which are incorporated herein by reference in their entireties. Additional examples of signal peptides are represented by SEQ ID NO: 156 (for a γ chain) and SEQ ID NO: 157 (for a δ chain). Examples of suitable domains facilitating polypeptide isolation and/or purification, and/or stability, may be derived from a His-tag, AVI-tag, c-myc domain, hemagglutinin tag, glutathione-S-transferase, maltose-binding protein, FLAG tag peptide, biotin acceptor peptide, streptravidin-binding peptide, calmodulin-binding peptide, bovine serum albumin, and others. Additional examples are represented by SEQ ID NO: 148 (AVI-tag) and SEQ ID NO: 149 (His-tag).

In soluble, such as chimeric, polypeptides described herein, a T-cell- and/or NK-cell-binding domain, an immune checkpoint inhibitor domain, and/or an additional domain may be fused to a γT-cell receptor chain, a δT-cell receptor chain, a γδT-cell receptor, or a part thereof (such as e.g., an extracellular domain or part thereof) at the N-terminus or C-terminus of the receptor chain, receptor, or part thereof. In cases where multiple domains are comprised in a chimeric polypeptide, the skilled person understands that each additional domain may also be fused at the N-terminus or C-terminus of the domain it is fused to. Optionally, linkers may be comprised between the domains as described earlier herein.

In some embodiments, a soluble, such as chimeric, polypeptide as described herein is a dimer, or a higher multimer such as a trimer. In some embodiments, dimerization or multimerization is facilitated by the inclusion of a dimerization or multimerization domain in the polypepide, for example a leucine zipper, a jun-fos interaction domain (such as for example described in Pack and Pluckthun, 1992, Biochemistry 31, 1579-1584; de Kruif and Logtenberg, 1996. JBC 271: 7630-7634, incorporated herein by reference in their entireties), or any other suitable such domain known to the skilled person. Alternatively, a bivalent or multivalent polypeptide may be generated via chemical cross-linking using standard methods, also described in standard handbooks such as Wong S. S, Chemistry of Protein Conjugation and Cross-Linking, 1st edition, CRC Press (1991), incorporated herein by reference in its entirety.

The skilled person understands that a dimer or a higher multimer as described herein may be a dimer or multimer of polypeptides comprising the same or different γT-cell receptor chains, δT-cell receptor chains, γδT-cell receptors, or parts thereof (such as e.g., extracellular domains or parts thereof), and/or T-cell and/or NK-cell-binding domains, said polypeptides and/or domains optionally having different targets.

The skilled person may arrive at the soluble, such as chimeric, polypeptides described herein utilizing standard molecular toolbox techniques, for example as described in standard handbooks such as Sambrook and Green (2012, supra) and Ausubel et al. (2003, supra). A soluble, such as chimeric, polypeptide described herein may be synthetic or may be produced by an engineered (host) cell, as described later herein. Optionally, the polypeptide may be isolated and/or purified after its production, for example via the use of a His-tag or AVI-tag which is comprised in the polypeptide (in combination with chromatography) or via another suitable method. Suitable downstream processing methods for isolation and/or purification of polypeptides from cell cultures are well-known in the art and are described in standard handbooks such as Wesselingh, J. A and Krijgsman, J., 1st edition, Downstream Processing in Biotechnology, Delft Academic Press (2013), incorporated herein by reference in its entirety. Examples of suitable isolation and/or purification techniques are chromatographic methods such as high performance liquid chromatography, size exclusion chromatography, ion exchange chromatography, affinity chromatography (such as for example utilizing His-tags or AVI-tags), immunoaffinity chromatography, immunoprecipitation, and the like. As a non-limiting example, a chimeric polypeptide comprising a γT-cell receptor chain, a δT-cell receptor chain, a γδT-cell receptor, or a part thereof (such as e.g., an extracellular domain or part thereof), and a T- and/or NK-cell-binding domain (for example an anti-CD3 scFv or Fc domain), and a His-tag may be produced by HEK293F cells and subsequently purified using a Histrap column (Sigma-Aldrich, MO, USA). Purity may be assessed via standard SDS-Page gel electrophoresis and/or Coomassie staining. An additional example of production and purification of a soluble polypeptide described herein is given in the experimental section.

Folding of the chimeric polypeptide can be probed using conformational-specific antibodies that can target γ and δ variable domains. Chimeric polypeptides may then be used in antibody dependent cell mediated cytotoxicity (ADCC) and complement dependent cytotoxicity (CDC) assays to determine their mediation of an anti-tumour or anti-infective response. After performing in vitro assays, the activity of chimeric polypeptides can be tested in vitro and/or in vivo. Alternatively, any of the assays described later herein may be used.

In a further aspect, the invention also relates to a conjugate comprising the δT-cell receptor chain or a part thereof as defined herein which is linked to an agent. The invention also relates to a γT-cell receptor chain or a part thereof as defined herein which is linked to an agent. The invention also relates to a γδTCR or a part thereof as defined herein which is linked to an agent. The type of agent used depends on the type of applications envisaged. Such conjugates may be linked to substrates (e.g. chemicals, nanoparticles) and may be used e.g. to deliver chemotherapy to a target of interest. In addition, in diagnostics expression of defined ligands may be tested by taking advantage of the soluble TCRs linked to fluorochromes which are then used as staining tool or for the biochemical isolation of the ligand. In an embodiment, the agent is selected from the group consisting of a diagnostic agent, a therapeutic agent, an anti-cancer agent, a chemical, a nanoparticle, a chemotherapeutic agent, a fluorescent protein, or an enzyme whose catalytic activity could be detected.

In one embodiment, the fluorescent protein can be selected from the group consisting of: green fluorescent protein (GFP), yellow fluorescent protein (YFP), red fluorescent protein (RFP), Blue fluorescent protein (BFP, Heim R., et al. (1994), Proc. Natl. Acad. Sci., 20; 91 (26): 12501-12504, and Heim R., et al (1996) Curr. Biol., 1; 6(2):178-182), a cyan fluorescent variant known as CFP (Heim R., et al. (1996) supra; Tsien R., et al, (1998) Annu. Rev. Biochem., 67: 509-544); a yellow fluorescent variant known as YFP (Ormo M., et al. (1996), Science, 6; 273(5280): 1392-1395; Wachter R. M., et al. (1998), Structure. 1998 Oct. 15; 6(10):1267-77. doi: 10.1016/s0969-2126(98)00127-0. PMID: 9782051); a violet-excitable green fluorescent variant known as Sapphire (Tsien 1998; Zapata-Hommer et al. (2003), BMC Biotechnol. 2003 May 22; 3:5. doi: 10.1186/1472-6750-3-5. Epub 2003 May 22. PMID: 12769828; PMCID: PMC161811. ); Td Tomato (Shaner N.C., et al. (2004) Nat Biotechnol. 2004 December; 22(12):1567-72. doi: 10.1038/nbt1037. Epub 2004 Nov. 21. PMID: 15558047); a cyan-excitable green fluorescing variant known as enhanced green fluorescent protein (eGFP) (Yang Te-Tuan, et al. (1996), Nucleic Acids Research, Volume 24, Issue 22, 1 Nov. 1996, Pages 4592-4593, https://doi.org/10.1093/nar/24.22.4592). The presence of a fluorescent protein can be assessed by live cell imaging, flow cytometry, and/or fluorescent spectrophotometry. Fluorescent reporters can be detected using various means including but not limited to microscopy, visual observation, flow cytometry, Luminex, and the like. In an aspect, a fluorescent reporter is detected using flow cytometry.

In one embodiment, the enzyme whose activity could be detected may be luciferase, beta galactosidase, beta lactamase, catalase, alkaline phosphatase, and the like. As a non limiting example, luciferase activity can be detected by commercially available assays, e.g., by the Luciferase 1000 Assay System, Nano-Glo or the Bio-Glo (Promega). The Luciferase 1000 Assay System contains coenzyme A (CoA) besides luciferin as a substrate, resulting in a strong light intensity lasting for at least one minute. Alternatively, D-luciferin can also be utilized. In some cases, for an intracellular luciferase assay it may be helpful to lyse the cells prior to detection. In another embodiment a Luciferase assay is used wherein the luciferase is secreted from the cells. Hence the assay can be performed without lysis of the cells.

In a further aspect, the invention relates to a nucleic acid construct comprising a nucleic acid molecule encoding the δT-cell (and/or γT-cell) receptor chain or a part thereof, or the γδTCR or part thereof, represented by an amino acid sequence as identified herein. The nucleic acid construct may be comprised in a vector as described later herein. Preferred vectors are viral vectors, with retroviral and lentiviral vectors being more preferred and lentiviral vectors being most preferred.

In a further aspect, there is provided a γδTCR or a part thereof comprising a CDR3 region comprising:
a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7, 9, 13, 15, 19, and/or 21, or with amino acid sequence SEQ ID NO: 7, 9, 13, 15, 19, 21, 142, and/or 153, and/or
a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10, 12, 16, 18, 22, and/or 24, or with amino acid sequence SEQ ID NO: 10, 12, 16, 18, 22, 24, 131, 133, 135, 143, 144, 154, 155, and/or 162.

In some embodiments, there is provided a γδTCR or a part thereof comprising a CDR3 region comprising:
a δT-cell receptor chain or part thereof comprising a CDR3 region, said δT-cell receptor chain or part thereof being represented by an amino acid sequence, said amino acid sequence comprising at least 70%, at least 80%, at least 85%, at least 90%, or 100%, preferably at least 70%, sequence identity with amino acid sequence SEQ ID NO: 7, 13, and/or 19, and/or
a γT-cell receptor chain or part thereof comprising a CDR3 region, said γT-cell receptor chain or part thereof being represented by an amino acid sequence, said amino acid sequence comprising at least 80%, at least 85%, at least 90%, or 100%, preferably at least 85%, sequence identity with amino acid sequence SEQ ID NO: 10, 16, and/or 22.

Preferably, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a γδTCR or a part thereof comprises A or B or C:
A:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7 and/or 9, preferably at least 70% sequence identity with amino acid sequence SEQ ID NO: 7, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10 and/or 12, or with amino acid sequence SEQ ID NO: 10, 12. and/or 131, preferably at least 85% sequence identity with amino acid sequence SEQ ID NO: 10,
B:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 13 and/or 15, or with amino acid sequence SEQ ID NO: 13, 15, 142, and/or 153, preferably at least 70% sequence identity with amino acid sequence SEQ ID NO: 13, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 16 and/or 18, or with amino acid sequence SEQ ID NO: 16, 18, 133, 143, 144, 154, 155, and/or 162, preferably at least 85% sequence identity with amino acid sequence SEQ ID NO: 16,
C:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 19 and/or 21, preferably at least 70% sequence identity with amino acid sequence SEQ ID NO: 19, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 22 and/or 24, or with amino acid sequence SEQ ID NO: 22, 24, and/or 135, preferably at least 85% sequence identity with amino acid sequence SEQ ID NO: 22.

Preferably, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

The γδTCRs or parts thereof described above may be comprised in a polypeptide, such as a soluble polypeptide, as described earlier herein.

In a further aspect, there is provided a nucleic acid molecule encoding a γδTCR or a part thereof as defined earlier herein, said nucleic acid molecule being represented by a nucleotide sequence comprising:
a nucleotide sequence (encoding a δT-cell receptor chain or part thereof comprising a CDR3 region) that has at least 60% sequence identity with SEQ ID NO: 8, 14, 20, 28, 30, and/or 32 and/or a nucleotide sequence that encodes an amino acid sequence that has at least 60% amino acid identity or similarity with an amino acid sequence encoded by a nucleotide sequence SEQ ID NO: 8, 14, 20, 28, 30, and/or 32, and/or
a nucleotide sequence (encoding a γT-cell receptor chain or part thereof comprising a CDR3 region) that has at least 80% sequence identity with SEQ ID NO: 11, 17, 23, 29, 31, and/or 33, or with SEQ ID NO: 11, 17, 23, 29, 31, 33, 130, 132, and/or 134, and/or a nucleotide sequence that encodes an amino acid sequence that has at least 80% amino acid identity or similarity with an amino acid sequence encoded by a nucleotide sequence SEQ ID NO: 11, 17, 23, 29, 31, and/or 33, or SEQ ID NO: 11, 17, 23, 29, 31, 33, 130, 132, and/or 134.

In an embodiment, there is provided a nucleic acid molecule encoding a γδTCR or a part thereof, said nucleic acid molecule being represented by a nucleotide sequence comprising A1, B1 or C1:
A1:
   a nucleotide sequence that has at least 60% sequence identity with SEQ ID NO: 8 and/or 28 and/or a nucleotide sequence that encodes an amino acid sequence that has at least 60% amino acid identity or similarity with an amino acid sequence encoded by a nucleotide sequence SEQ ID NO: 8 and/or 28, and/or
   a nucleotide sequence that has at least 80% sequence identity with SEQ ID NO: 11 and/or 29, or with SEQ ID NO: 11, 29, and/or 130, and/or a nucleotide sequence that encodes an amino acid sequence that has at least 80% amino acid identity or similarity with an amino acid sequence encoded by a nucleotide sequence SEQ ID NO: 11 and/or 29, or SEQ ID NO: 11, 29, and/or 130,
B1:
   a nucleotide sequence that has at least 60% sequence identity with SEQ ID NO: 14 and/or 30 and/or a nucleotide sequence that encodes an amino acid sequence that has at least 60% amino acid identity or similarity with an amino acid sequence encoded by a nucleotide sequence SEQ ID NO: 14 and/or 30, and/or
   a nucleotide sequence that has at least 80% sequence identity with SEQ ID NO: 17 and/or 31, or with SEQ ID NO: 17, 31, and/or 132, and/or a nucleotide sequence that encodes an amino acid sequence that has at least 80% amino acid identity or similarity with an amino acid sequence encoded by a nucleotide sequence SEQ ID NO: 17 and/or 31, or SEQ ID NO: 17, 31, and/or 132,
C1:
   a nucleotide sequence that has at least 60% sequence identity with SEQ ID NO: 20 and/or 32 and/or a nucleotide sequence that encodes an amino acid sequence that has at least 60% amino acid identity or similarity with an amino acid sequence encoded by a nucleotide sequence SEQ ID NO: 20 and/or 32, and/or
   a nucleotide sequence that has at least 80% sequence identity with SEQ ID NO: 23 and/or 33, or SEQ ID NO: 23, 33, and/or 134, and/or a nucleotide sequence that encodes an amino acid sequence that has at least 80% amino acid identity or similarity with an amino acid sequence encoded by a nucleotide sequence SEQ ID NO: 23 and/or 33, or 23, 33, and/or 134.

Preferably, the identity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the nucleic acid molecule represented by the nucleic acid sequence A1 encodes the amino acid sequence A.

In an embodiment, the nucleic acid molecule represented by the nucleic acid sequence B1 encodes the amino acid sequence B.

In an embodiment, the nucleic acid molecule represented by the nucleic acid sequence C1 encodes the amino acid sequence C.

A nucleic acid molecule described herein may in some cases be a synthetic nucleic acid molecule or be part of a synthetic construct. A nucleic acid molecule described herein may in some cases be a codon optimized molecule, preferably for expression in a mammalian cell, more preferably in a human cell. A definition of codon optimization is provided later herein.

In some embodiments, there is provided a nucleic acid molecule encoding a soluble polypeptide as described herein.

The general part of the description dedicated to the definitions provides detailed explanation as to nucleic acid molecules and polypeptide encompassed by the invention, nucleic construct, viral vector and cells comprising said construct or vector.

Each δT-cell (or γT-cell) receptor chain and γδTCR or part thereof defined by reference to their amino acid or encoding nucleic acid sequence is expected to be biologically relevant for designing a medicament for preventing, treating, regressing, curing and/or delaying a cancer or an infection, since each of these chains and γδTCR or part thereof exhibits and/or mediates an anti-tumour activity/response or an anti-infective response.

In a further aspect, there is provided, a nucleic acid construct comprising a nucleic acid molecule encoding the amino acid sequence as identified earlier herein and/or wherein said nucleic acid molecule is as identified earlier herein. In some embodiments, the nucleic acid construct is comprised in a vector. Preferred vectors are viral vectors, among which retroviral and lentiviral vectors are more preferred, with lentiviral vectors being most preferred. Further explanation of nucleic acid constructs and vectors according to the invention are provided later herein.

### Cells

In a further aspect, there is provided a cell comprising the nucleic acid construct or the vector as earlier defined herein. Preferably, the cell expresses the polypeptide as earlier defined herein. In an embodiment, the cell is a mammalian cell, preferably a human cell. In an embodiment, the cell is an immune cell such as a T-cell, an alpha-beta T-cell, a gamma-delta T-cell, CD4+ T-cell, CD8+ T-cell, a T effector cell, a lymphocyte, a B-cell, an NK-cell, an NKT-cell, a myeloid cell, a monocyte, a macrophage, or a neutrophil. In an embodiment, the cell is a T-cell. In a preferred embodiment, the T-cell is an αβT-cell. In an embodiment, the cell is from a human cell line, for example it is a HEK293 or a HEK293F or a derivative thereof.

In an embodiment, a cell is a T-cell comprising a nucleic acid molecule encoding the amino acid sequence as identified earlier herein and/or expressing the amino acid sequence as identified earlier herein and/or comprising a nucleic acid molecule as identified earlier herein.

In an embodiment, a cell is a T-cell, preferably an αβT-cell, expressing a γδTCR or part thereof comprising a CDR3 region comprising A or B or C:
A:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7 and/or 9, preferably at least 70% sequence identity with amino acid sequence SEQ ID NO: 7, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10 and/or 12, or with SEQ ID NO: 10, 12, and/or 131, preferably at least 85% sequence identity with amino acid sequence SEQ ID NO: 10,
B:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 13 and/or 15, or with SEQ ID NO: 13, 15, 142, and/or 153, preferably at least 70% sequence identity with amino acid sequence SEQ ID NO: 13, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 16 and/or 18, or with SEQ ID NO: 16, 18, 133, 143, 144, 154, 155, and/or 162, preferably at least 85% sequence identity with amino acid sequence SEQ ID NO: 16,
C:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 19 and/or 21, preferably at least 70% sequence identity with amino acid sequence SEQ ID NO: 19, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 22 and/or 24, or with SEQ ID NO: 22, 24, and/or 135, preferably at least 85% sequence identity with amino acid sequence SEQ ID NO: 22.

Preferably, the identity or similarity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a cell is a T-cell, preferably an αβT-cell, comprising a nucleic acid molecule encoding a γδTCR or part thereof corresponding to A, B or C as defined above and said nucleic acid molecule is represented by a nucleotide sequence comprising A1, B1 or C1:
A1:
   a nucleotide sequence that has at least 60% sequence identity with SEQ ID NO: 8 and/or 28 and/or a nucleotide sequence that encodes an amino acid sequence that has at least 60% amino acid identity or similarity with an amino acid sequence encoded by a nucleotide sequence SEQ ID NO: 8 and/or 28, and/or
   a nucleotide sequence that has at least 80% sequence identity with SEQ ID NO: 11 and/or 29, or with SEQ ID NO: 11, 29, and/or 130, and/or a nucleotide sequence that encodes an amino acid sequence that has at least 80% amino acid identity or similarity with an amino acid sequence encoded by a nucleotide sequence SEQ ID NO: 11 and/or 29, or SEQ ID NO: 11, 29, and/or 130,
B1:
   a nucleotide sequence that has at least 60% sequence identity with SEQ ID NO: 14 and/or 30 and/or a nucleotide sequence that encodes an amino acid sequence that has at least 60% amino acid identity or similarity with an amino acid sequence encoded by a nucleotide sequence SEQ ID NO: 14 and/or 30, and/or
   a nucleotide sequence that has at least 80% sequence identity with SEQ ID NO: 17 and/or 31, or with SEQ ID NO: 17, 31, and/or 132, and/or a nucleotide sequence that encodes an amino acid sequence that has at least 80% amino acid identity or similarity with an amino acid sequence encoded by a nucleotide sequence SEQ ID NO: 17 and/or 31, or SEQ ID NO: 17, 31, and/or 132,
C1:
   a nucleotide sequence that has at least 60% sequence identity with SEQ ID NO: 20 and/or 32 and/or a nucleotide sequence that encodes an amino acid sequence that has at least 60% amino acid identity or similarity with an amino acid sequence encoded by a nucleotide sequence SEQ ID NO: 20 and/or 32, and/or
   a nucleotide sequence that has at least 80% sequence identity with SEQ ID NO: 23 and/or 33, or SEQ ID NO: 23, 33, and/or 134, and/or a nucleotide sequence that encodes an amino acid sequence that has at least 80% amino acid identity or similarity with an amino acid sequence encoded by a nucleotide sequence SEQ ID NO: 23 and/or 33, or SEQ ID NO: 23, 33, and/or 134.

Preferably, the identity is of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the nucleic acid molecule represented by the nucleic acid sequence A1 encodes the amino acid sequence A.

In an embodiment, the nucleic acid molecule represented by the nucleic acid sequence B1 encodes the amino acid sequence B.

In an embodiment, the nucleic acid molecule represented by the nucleic acid sequence C1 encodes the amino acid sequence C.

Below we provide more explanation as to the T-cells encompassed by the invention and how to obtain them.

### T-cells

T-cells, or T lymphocytes, belong to a group of white blood cells named lymphocytes, which play a role in cell-mediated immunity. T-cells originate from hematopoietic stem cells in the bone marrow, mature in the thymus (that is where the T is derived from), and gain their full function in peripheral lymphoid tissues. During T-cell development, CD4-CD8-T-cells (negative for both the CD4 and CD8 co-receptor) are committed either to an as or γδ fate as a result of an initial βTCR or δTCR gene rearrangement. Cells that undergo early β chain rearrangement express a pre-TCR structure composed of a complete β-chain and a pre-TCRα-chain on the cell surface. Such cells switch to a CD4⁺CD8⁺ state, rearrange the TCRα-chain locus, and express a mature αβTCR on the surface. CD4⁻CD8⁻ T-cells that successfully complete the γ gene rearrangement before the β-gene rearrangement express a functional γδTCR and remain CD4⁻CD8⁻ (Claudio Tripodo et al. Gamma delta T-cell lymphomas Nature Reviews Clinical Oncology 6, 707-717, December 2009). The T-cell receptor associates with the CD3 protein complex. Mature T-cells, i.e. expressing an αβTCR or a γδTCR, express the T-cell receptor complex on the cell surface. The γδT-cells, which constitute about 1-5% of the total population of T-cells, can be divided in further subpopulations which, in humans, is based on TCRδ-chain expression. Within the extracellular domain of a T-cell receptor three complementarity determining regions (CDR1, CDR2, CDR3) are located. These regions are in general the most variable domains and contribute significantly to the diversity among TCRs. CDR regions are composed during the development of a T-cell where so-called Variable-(V), Diverse-(D), and Joining-(J)-gene segments are randomly combined to generate diverse TCRs. Of the three CDR regions CDR3, for both αβT-cells and γδT-cells, is the most variable one, and is therefore the key player in antigen/ligand recognition.

### αβT-cells

αβT-cells may be defined with respect to function as T lymphocytes that express an αβTCR, which recognize peptides bound to MHC molecules (major histocompatibility complex), which are expressed on the surface of various cells. MHC molecules present peptides derived from the proteins of a cell. When for example a cell is infected with a virus, the MHC will present viral peptides, and the interaction between the αβTCR on the T-cell and the MHC-complex on the target cell (i.e. the virus infected cell) activates specific types of T-cells which initiate and immune responses to eliminate the infected cell. Hence, αβT-cells may be functionally defined as being cells capable of recognizing peptides bound to MHC molecules. αβT-cells may be selected from peripheral blood for example via the CD3 antigen as described below and, in the examples, as the large majority of T-cells have the αβTCR. αβT-cells may also be selected with an antibody specific for the αβTCR, such as described below. From such selected cells, the nucleic acid (or amino acid) sequence corresponding to the αT-cell receptor chain and the PT-cell receptor chain may be determined by sequencing. Hence, αβT-cells may also be defined as being cells comprising a nucleic acid (or amino acid) sequence corresponding to the αT-cell receptor chain and/or the PT-cell receptor chain. In an embodiment, αβT-cells express an αβTCR, preferably an endogenous αβTCR.

Optionally, αβT-cells described herein may have decreased or no expression, preferably surface expression, of an endogenous αβTCR. Decreased expression may correspond to at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%, decreased expression of an αβTCR relative to an otherwise comparable αβT-cell not comprising a genomic modification or not having been subjected to selective expansion.

Optionally, an αβT-cell may comprise a higher ratio of an exogenous γδTCR as compared to an endogenous (naturally expressed) αβTCR. In certain cases, decreased expression, preferably surface expression, of an endogenous αβTCR and/or a higher ratio of an exogenous γδTCR to an endogenous αβTCR may be achieved by way of preferential expansion of said T-cells, benefitting the growth and survival of said T-cells. As non-limiting examples, T-cells comprising an exogenous γδTCR may be stimulated by anti-CD3/CD28 antibodies, by contact with antigens that are specific for the exogenous γδTCR, or with cells expressing such antigens, optionally the stimulation being serial stimulation. The preferential expansion may result in a population of said T-cells with limited or absenT-cell surface expression of the endogenous αβTCR, while expressing sufficient amounts of the exogenous γδTCR (referred to as a population enriched for a single positive phenotype). Such cells may have reduced alloreactivity (e.g., graft versus host disease) as compared to cells having surface expression of the endogenous αβTCR. Reduced alloreactivity may result in improved therapeutic applications with decreased side-effects.

In certain cases, decreased expression, preferably surface expression, of an endogenous αβTCR and/or a higher ratio of an exogenous γδTCR to an endogenous αβTCR may be achieved by way of preferential expansion of αβT-cells also expressing a chimeric bidirectional signaling protein (as described herein), benefitting the growth and survival of said T-cells. Accordingly, the skilled person understands that, in some cases, decreased expression, preferably surface expression, of an endogenous αβTCR and/or a higher ratio of an exogenous γδTCR to an endogenous αβTCR may be achieved by preferential expansion of αβT-cells expressing a chimeric bidirectional signaling protein combined with stimulation as discussed above, optionally the stimulation being serial stimulation, benefitting the growth and survival of said T-cells.

In other cases, decreased expression, preferably surface expression, of an endogenous αβTCR and/or a higher ratio of an exogenous γδTCR to an endogenous αβTCR, can be achieved by positively or negatively selecting for cells that express the exogenous γδTCR and have reduced surface expression of the endogenous receptor or lack the endogenous receptor.

In other cases, decreased expression, preferably surface expression, of an endogenous αβTCR and/or a higher ratio of an exogenous γδTCR to an endogenous αβTCR can be achieved via genomic modification, for example a genomic modification which results in the reduction or elimination of surface expression of the endogenous αβTCR. A genomic modification may be combined with preferential expansion and/or selection as discussed above. Genomic modification techniques are discussed later herein.

Optionally, an αβT-cell of the invention may comprise a ratio of an exogenous γδTCR to an endogenous αβTCR that is at least 0.5:1, at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1 at least 15:1, at least 20:1, at least 30:1, at least 40:1, at least 50:1, at least 60:1, at least 70:1, at least 80:1, at least 90:1, at least 100:1, at least 150:1, at least 200:1, at least 250:1, or at least 300:1.

Surface expression of endogenous αβTCRs and exogenous γδTCRs may be assessed with flow cytometric methods as described later herein. An example is further provided in the experimental section herein.

### γδT-cells

γδT-cells may be functionally defined in that they are specifically and rapidly activated by e.g. a set of non-peptidic phosphorylated isoprenoid precursors, collectively named phosphoantigens or stress signals medicated by non-classical HLA molecules like CD1 (this is the case for the Vγ9Vδ2 T-cell subset). Phosphoantigens are produced by virtually all living cells, though the levels are usually very low in healthy cells, and increased in transformed/malignanT-cells or cells infected with e.g. Mycobacterium tuberculosis, which deliver a derivate of phosphoantigens. Activation of γδT-cells comprises clonal expansion, cytotoxic activity and expression and release of cytokines. γδT-cells are also defined by expression of the γδT cell receptor. For example, cells may be selected using an antibody specific for the γδT cell receptor such as described below. From such selected cells, the nucleic acid (or amino acid sequence) sequence corresponding to the γT-cell receptor chain and/or the δT-cell receptor chain may be determined by sequencing. Hence, γδT-cells may also be defined as being cells naturally comprising a nucleic acid (or amino acid) sequence corresponding to a γT-cell receptor chain and/or a δT-cell receptor chain. In an embodiment, γδT-cells express a γδTCR. In an embodiment, a γδT-cell expresses an exogenous γδTCR or a part thereof, which may be any of the γδTCRs or parts thereof described herein. In an embodiment, a γδT-cell expresses an exogenous γδTCR or a part thereof and does not express an endogenous γδTCR or a part thereof. The skilled person may arrive at γδT-cells not expressing an endogenous γδTCR or part thereof using any of the suitable methods discussed herein, for example via genomic modification (for example via a deletion of the endogenous γδTCR-encoding nucleotide sequence or via another method).

The person skilled in the art is well capable of selecting and/or identifying cell populations characterized by expression of an antigen or receptor on the surface of the cell such as described throughout herein. It is understood that with regard to expression on the surface of cells, such as CD3, CD4, CD8, αβTCR, γδTCR, or parts thereof, this is typically done in a population of cells of which a portion of cells have a much higher level of expression of the antigen or respective polypeptide when compared to cells having a lower level of expression. Hence, the terms positive or negative are to be understood as being relative, i.e. positive cells have a much higher expression level as compared to cells being negative. Cells being negative in this sense may thus still have an expression level which may be detected.

Expression on the surface of cells may be analyzed using Fluorescence Activated Cell Sorting (FACS), and many specific antibodies are commercially available, e.g. such as for CD3, CD4, CD8, αβTCR, γδTCR, δ1TCR and 62TCR that are suitable for such FACS analysis, such as described in the examples and as available. As an example, αβT-cells can also be defined and selected as being positive for αβTCR in FACS. The same holds for γδT-cells and γδTCR expression. Antibodies suitable for FACS or similar separation techniques (such as e.g. antibodies conjugated to magnetic beads) are widely available. Conditions are selected, such as provided by the antibody manufacturer that allows the selection of negative and/or positive cells. Examples of antibodies that may be suitable for selection of γδT-cells, or engineered γδT-cells are commercially available, such as available from BD Pharmingen (BD, Franklin Lakes, NJ USA) is Vδ2-FITC (clone B6, #555738), or such as from Thermofisher Scientific (Waltham, MA USA) is Vγ1-PE-Cγ7 (clone TS8.2, #25-5679-42), or such as available from Biolegend (San Diego, CA, USA) is αβTCR-BV785 (clone IP26, #306742) or such as available from Beckman Coulter (Brea, CA, USA) is pan-γδTCR-PE (clone IMMU510, #IM1418U), or such as available from Miltenyi Biotec (Bergisch Gladbach, Germany) is CD3-VioGreen (clone REA613, #130-113-142). Similarly, suitable antibodies for αβT-cell depletion/selection, such as anti-Biotin αβTCR (clone IP26, Biolegend, San Diego, CA, USA, #306704) and many others are commercially available.

Accordingly, in the invention, T-cells are provided. The T-cells may be primary cells, for example from a subject, such as described in the examples for a human subject. The T-cells may be αβT- or γδT-cells derived from a human subject. Alternatively, the T-cells may be T-cell lines, such as SupT-1 or JurkaT-cells or any other widely available cell line. Any cell type, being a primary cell or any cell line will suffice, as long as the cell population, or a substantial part thereof, expresses a T-cell receptor, i.e. such as being positive for the αβTCR or the γδTCR in a FACS sorting or the like as described above, such a cell population may be contemplated. Also, any cell or cell population may be contemplated that, when provided with a γδTCR according to the invention is capable of forming a functional TCR complex and exerting e.g. a functional cytotoxic response and/or cytokine production as later defined herein. The cell that is provided may also be a progenitor cell, preferably a blood progenitor cell such as a thymocyte or a blood stem cell, which after it has been provided with the right stimuli can develop into T-cells.

Preferably, T-cells provided express or are able to express a γδTCR. T-cells may have been transduced to express a γδTCR (e.g., with a nucleic acid molecule, construct, or vector as described herein) or already express a γTCR and have been transduced to express a δTCR (or respectively already express a δTCR and have been transduced to express a γTCR), comprising the nucleic acid sequences encoding the sequence as earlier identified herein). All theoretical combinations of a γ- with a δ-chain of the TCR are encompassed. Preferred γ-chains comprise a Cγ1 constant region or part thereof, further information on which is provided later herein.

A preferred δTCR chain is a δ1TCR chain. Another preferred δTCR chain is a δ3TCR chain. A preferred γTCR chain is a γ3TCR chain. Another preferred γTCR chain is a γ4TCR chain. Another preferred γTCR chain is a γ9TCR chain.

In an embodiment, a γδTCR is a γ3δ1TCR. In another embodiment the γδTCR is a γ9δ1TCR. In another embodiment the γδTCR is a γ4δ3TCR.

The cells are useful for therapy, for example prevention, suppression, treatment of a disease. A disease in this context may be a cancer or an infection. A cancer may be a liquid cancer such as Acute myeloid leukemia (AML) and Multiple Myeloma (MM). A cancer may be a solid cancer such as ovarian cancer, breast cancer or colon cancer. A cancer may be a kidney cancer. A cancer may be a renal cancer. A cancer may be a skin cancer, for example melanoma. A cancer may be a lung cancer.

Such amino acid sequences are preferably defined by InMunoGeneTics information system (http://www.imgt.org/IMGTScientificChart/Nomenclature/IMGT-FRCDRdefinition.html; LeFranc et al., IMGT®, the international ImMunoGeneTics information system® 25 years on. Nucleic Acids Res. 2015 January; 43 (Database issue):D413-D422). It is also encompassed that the parts that are similar comprise conservative substitutions of a given amino acid. A list of amino acids that are considered to be a conservative substitution of another amino acid is provided in the general part of the description dedicated to the definitions under identity/similarity. In an embodiment said conserved part is comprised within a CDR3 region of a δT-cell (or γT-cell) receptor chain or part thereof or comprises a CDR3 region of a δT-cell (or γT-cell) receptor chain or consists of a δT-cell (or γT-cell) receptor chain. More preferably said conserved part is comprised within a CDR3 region of a δT-cell (or γT-cell) receptor chain and is from 3 to 53 amino acids.

A conserved part may be identical or have a relatively high identity percentage or may be similar or have a relative high similarity identity percentage with a given sequence. In the context of the application, “high” identity or similarity in relation with a δT-cell receptor chain or part thereof may mean an identity or a similarity of at least 60% or of at least 70% or more.

In the context of the application, “high” identity or similarity in relation with a γT-cell receptor chain or part thereof may mean an identity or a similarity of at least 80% or of at least 85% or more.

In order to validate the biological relevance of the γT-cell and/or δT-cell receptor chain and/or the γδTCR and/or parts thereof, the anti-tumour or anti-infective activity/response of a T-cell expressing a defined nucleic acid molecule encoding an amino acid sequence as defined herein is determined. The T-cell may already express a δT- cell (or γT-cell) receptor chain or a part thereof identified herein. It is clear that the biological relevance of a δT-cell (or a γT-cell respectively) receptor chain or part thereof may only be assessed when a T-cell is transduced with (or expresses) a δT-cell (or a γT-cell respectively) receptor chain. In an embodiment, an anti-tumour or anti-infective activity/response of such sequence is assessed in a T-cell that does not endogenously express a gamma or delta chain of the TCR on their cell surface. Such a cell may be an αβT-cell or a NK cell.

The nucleic acid sequences encoding the δT-cell receptor chain or part thereof, preferably the δ1 or the δ3-T-cell receptor chain or part thereof, may be introduced into T-cells to provide an engineered T-cell as explained in the general part of the description dedicated to the definitions.

Alternatively, a nucleic acid sequence encoding a γT-cell receptor chain or part thereof, preferably the γ3T- or γ4T- or γ9T-cell receptor chain or part thereof, may be introduced into T-cells to provide an engineered T-cell as explained in the general part of the description dedicated to the definitions.

It is clear to a skilled person that the T-cells used should also express a γT-cell receptor chain in order to assess the biological relevance of a δT-cell receptor chain. In other words, a γδTCR is preferably expressed in said T-cells, the δTCR being the one identified herein.

It is also clear to a skilled person that the T-cells used should express a δT-cell receptor chain in order to assess the biological relevance of a γT-cell receptor chain. In other words, a γδTCR is preferably expressed in said T-cells, the γTCR being the one identified herein.

In a preferred embodiment, the nucleic acid molecule encoding the δT-cell (or γT-cell) receptor chain or part thereof is provided in an expression vector or in a retroviral or lentiviral vector in a T-cell. This has been extensively explained in the general part of the description dedicated to the definitions.

T-cells may optionally be expanded before or after the transfer of the nucleic acids encoding the δT- and/or γT-cell receptor chain. Preferably, the expansion is after the transfer such that the amount of nucleic acids that needs to be transferred is as low as possible. This expansion of T-cells may be performed by stimulation with anti-CD3/CD28 polymeric nanomatrix beads, in the presence of IL-7 and IL-15. Expansion may be performed using commercially available kits, such as T-cell TransAct™ (Miltenyi Biotec, Bergisch Gladbach, Germany). A further example is provided in the experimental section herein.

The anti-tumour or anti-infective activity/response of the provided T-cell expressing a δT-cell (and/or γT-cell) receptor chain may be assessed using any technique known to the skilled person. A δT-cell receptor chain may preferably be a δ1 or a δ3-T-cell receptor chain. A γT-cell receptor chain may preferably be a γ3, γ4 or a γ9T-cell receptor chain.

As soon as an effect can be seen in the assays described herein, one can conclude that the δT-cell and/or γT-cell receptor chain and/or γδTCR exhibits an anti-tumour or anti-infective response or anti-tumour or anti-infective activity. Therefore, as soon as an effect had been seen/determined/assessed on tumour or infected cell division rate, tumour or infected cell death, tumour or infected cell cytolysis/cytotoxicity, binding to the tumour or infected cell, induction of the production of a cytokine such as IFN-γ, IL-2 or TNFα, the δT-cell and/or γT-cell receptor chain and/or γδTCR would be considered to exhibit an anti-tumour or an anti-infective response. In these assays, a negative control may be T-cells that are untransduced or that are transduced by an empty viral vector or that are transduced by a control δT-cell and/or γT-cell receptor chain.

In one embodiment, determining an anti-tumour or anti-infective response or reactivity or activity comprises contacting the T-cells with tumour cells or tumour cell lines or infected cells. Determining an anti-tumour or anti-infective activity may include any assay in which an anti-tumour or anti-infective effect may be determined, such as having an effect on tumour or infected cell division rate, i.e. the speed with which the tumour or infected cells divide, tumour or infected cell death, cytolysis/cytotoxicity of the tumour or infected cell, binding to the tumour or infected cells, induction of the production of a cytokine such as IFN-γ, IL-2 or TNFα.

Tumour cells may be any kind of tumour cells. For example, primary tumour cells from a patient. The tumour cells may be tumour cells from cell lines, such as the cell lines listed hereafter: Caki-2, HT29, SK-OV-3, 769-P, 786-0, COV504, MDA-MB-231, BLM, Hs895.T, SW480, RKO, IgR39D, HAP-1, OVCAR-3, MZ1851RC, NCI-226, or others, which are well known in the art. Tumour cell lines may easily be obtained from the American Type Culture Collection (ATCC, Manassas, Virginia) and the like.

Infected cells may, for example, be cells that have been infected by a bacterium or a virus. The infection may result in the infected cell displaying an antigen or epitope that is a target of a γT-cell receptor chain, a δT-cell receptor chain, a γδT-cell receptor, or a part thereof as described herein. Non-limiting examples are Plasmodium falciparum, Mycobacterium (M.) tuberculosis and M. leprae. Infectious agents may, for example be, bacteria or fungal cells.

In a preferred embodiment, determining the anti-tumour or anti-infective response includes contacting the T-cell expressing a defined nucleic acid molecule encoding an amino acid comprising a δT-cell and/or γT-cell receptor chain and/or γδTCR and/or part thereof identified herein and measuring its ability to lyse the tumour or infected cell and/or induce the production of a cytokine such as IFN-γ, IL-2 or TNFα. This contacting step may, for example, have a duration from 10 hours to 1, 2, 3, 4, 5 days. Measuring the ability to lyse the tumour or infected cells may include providing a fixed amount of tumour or infected cells with which T-cell expressing a defined nucleic acid molecule encoding an amino acid comprising a δT-cell and/or γT-cell receptor chain and/or γδTCR or part thereof identified herein is contacted and after an incubation period the number of viable tumour or infected cells is counted.

An anti-tumour or anti-infective response may have been identified or determined when the number of viable tumour or infected cells at the end of the incubation step is less than 95%, less than 90%, less than 80%, less than 70%, less than 60%, less than 50%, less than 40%, less than 30%, less than 20%, less than 10% of the number of initial tumour or infected cells at the onset of the incubation step.

Alternatively, an anti-tumour or anti-infective response may have been identified or determined when the number of viable tumour or infected cells at the end of the incubation step with the T-cells is lower than the number of tumour or infected cells at the end of a similar incubation/contacting step with control T-cells not comprising sequences of the invention. Lower in this context may mean at least 5% lower, at least 10% lower, at least 20% lower, at least 30% lower, at least 40% lower, at least 50% lower, at least 60% lower, at least 70% lower, at least 80% lower, at least 90% lower.

In addition, or as alternative to the counting of the number of viable tumour or infected cells at the end of the incubation/contacting step, one may also perform a ⁵¹Chromium-release assay which is known to the skilled person. The amount of ⁵¹Chromium release is a measure of the number of cells that have been lysed.

In an embodiment, one may assess the cytotoxicity of the δT-cell and/or γT-cell receptor chain and/or of the γδTCR and/or part thereof (or the cytotoxicity or T-cells expressing them) by incubating T-cells expressing them with tumour or infected cell lines at E:T (effector:target) ratio 1:1. Other suitable E:T ratios may be 1:2, 1:3, 1:4, 1:5, and the like. Suitable tumour and infected cell lines have been described earlier herein. The incubation may, for example, have a duration of 1, 2, 3, 4 days. In an embodiment, the duration is 2 days. Control T-cells as described herein may also be used. Cytotoxicity may be measured by xCELLigence and plotted as percentage of cytolysis relative to maximum cytolysis induced by treatment of the target cells with the detergent Triton-X-100. Cytotoxicity may also be measured by a luciferase-based cytotoxicity assay, in which the target cells are pre-transduced with luciferase and cytotoxicity is measured by measuring decreased luciferase activity relative to target cells cultured alone or with control T-cells as described herein. These assays are known to the skilled person and examples are provided in the experimental section herein.

In an embodiment, when the percentage of cytolysis after contacting of tumour or infected cells with T-cells expressing δT-cell and/or γT-cell receptor chain and/or of γδTCR and/or part thereof assessed at the end of the incubation step is higher (preferably at least 5%, at least 10%, at least 20%, at least 30%, at least 40%, at least 50%, at least 60%, at least 70%, at least 80%, at least 90%, at least 100% or more) than the percentage of cytolysis assessed when the same tumour or infected cells are contacted with control T-cells, the δT-cell and/or γT-cell receptor chain and/or γδTCR and/or part thereof and/or T-cells expressing them are said to exhibit an anti-tumour or anti-infective response.

In an embodiment, when the percentage of cytolysis after contacting of tumour or infected cells with T-cells expressing the δT-cell and/or γT-cell receptor chain and/or γδTCR and/or part thereof assessed at the end of the incubation step is at least 5%, at least 10%, at least 20%, at least 30%, at least 40%, at least 50%, at least 60%, at least 70%, at least 80%, at least 90%, at least 100%, the δT-cell (and/or γT-cell) receptor chain and/or γδTCR and/or part thereof and/or T-cells expressing them is said to exhibit an anti-tumour or anti-infective response.

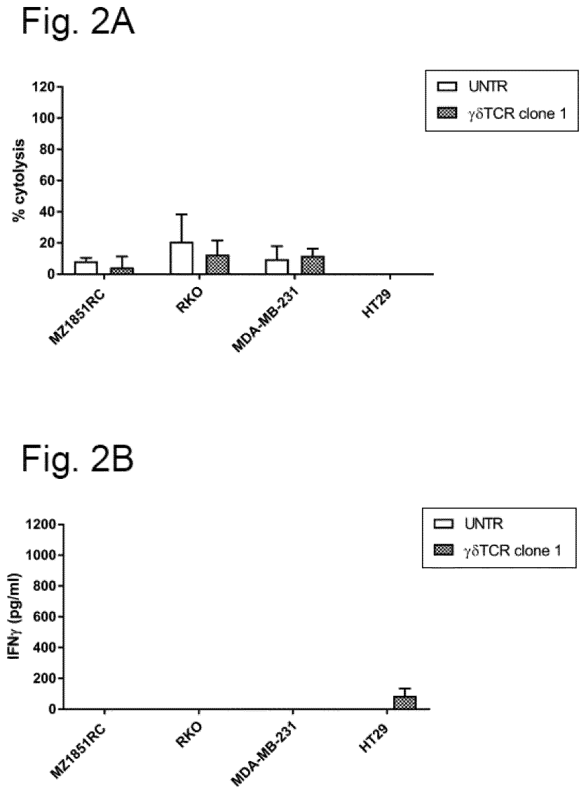

FIGS. 2, 3 and 4 nicely demonstrate the anti-tumour response of γδTCR of clones 2, 3 and 4.

Similarly, the production of a cytokine such as IFN-γ, IL-2 or TNFα or the secretion or the expression of activation markers may also be determined, e.g. via antibody staining, ELISA and/or quantitative PCR for the expressed mRNA. Assays for determining the production of a cytokine such as IFN-γ, IL-2 or TNFα are commercially widely available. When the production of a cytokine such as IL-2, TNFα or IFN-γ is detected at the end of the contacting step, the T-cell expressing a δT-cell and/or γT-cell receptor chain and/or γδTCR and/or part thereof identified herein is said to exhibit an anti-tumour or anti-infective response. Alternatively and preferably, when the amount of IFN-γ, IL-2 or TNFα produced at the end of the contacting step with said T-cells is higher (preferably at least 5%, at least 10%, at least 20%, at least 30%, at least 40%, at least 50%, at least 60%, at least 70%, at least 80%, at least 90%, at least 100% or more) than the amount of IFN-γ, IL-2 or TNFα produced when tumour or infected cells are contacted with control T-cells, the T-cells is said to exhibit an anti-tumour or anti-infective response.

An anti-tumour or anti-infective response may also be determined by assessing the binding of the T-cells expressing a δT-cell and/or γT-cell receptor chain and/or a γδTCR and/or part thereof identified herein to the tumour or infected cell after contacting both cells together. Such a contacting step may, for example, have a duration from 10 hours to 1, 2, 3, 4, 5 days. When binding of said T-cell to the tumour or infected cell is detected at the end of the contacting step, said T-cell is said to exhibit an anti-tumour or anti-infective response. Alternatively and preferably, when the binding of said T-cell to said tumour or infected cell at the end of the contacting step is higher (preferably at least 5%, at least 10%, at least 20%, at least 30%, at least 40%, at least 50%, at least 60%, at least 70%, at least 80%, at least 90%, at least 100% or more) than the binding of control T-cells (see earlier definition) to the same tumour or infected cell, the T-cell is said to exhibit an anti-tumour or anti-infective response.

Optionally, any of the assays described above may be performed multiple times, for example by collecting the T-cells at the end of the contacting/incubation steps and re-exposing them to the same tumour or infected cells (serial stimulation). T-cells expressing a δT-cell and/or γT-cell receptor chain and/or a γδTCR and/or part thereof described herein may exhibit an improved anti-tumour or anti-infective response compared to control T-cells not comprising sequences of the invention after a subsequent stimulation. This improved response may be at least 5%, at least 10%, at least 20%, at least 30%, at least 40%, at least 50%, at least 60%, at least 70%, at least 80%, at least 90%, at least 100% higher, or more compared to the control T-cells.

The ability of a δT-cell and/or γT-cell receptor chain and/or a γδTCR and/or part thereof to mediate an anti-tumor or anti-infective response may in some cases also be assessed by assessing their surface expression on a T-cell, preferably a γδT-cell or an αβT-cell, more preferably an αβT-cell. As shown in the experimental section herein, improved surface expression of an exogenous δT-cell and/or γT-cell receptor chain and/or a γδTCR and/or part thereof by a T-cell may in some cases correlate with an improved anti-tumour or anti-infective response. Surface expression may be determined by flow cytometric methods known to the skilled person, for example in combination with using antibodies specific to αβTCRs (e.g., clone IP26 as described above) and γδTCRs (e.g., clone IMMU510 as described above). An additional example of determination of γδTCR surface expression is given in the experimental section herein.

The skilled person understands that the assays described above may also be applicable to the soluble polypeptides described herein. As a non-limiting example, a chimeric polypeptide comprising a γT-cell receptor chain, a δT-cell receptor chain, a γδT-cell receptor, or a part thereof (such as e.g., an extracellular domain or part thereof) and a T-cell- and/or NK-cell-binding domain (and optionally any other suitable domain discussed herein) may be provided together with T-cells in the assays described herein, and the anti-tumour or anti-infective response of the T-cells as mediated by the chimeric polypeptide may then be assessed using any of the assays described above. Control T-cells as described above, such as T-cells that have not been provided together with the polypeptides of the invention, may similarly be used for the comparisons. Optionally, different amounts of soluble polypeptides and/or ratios of soluble polypeptides to T-cells may be tested, for example 1 μg, 3 μg, or 10 μg, per 200 μl of asay mixture at an effector to target ratio of 1:1 or any other ratio discussed herein. An example of determination of T-cell cytoxicity and IFN-γ production mediated by soluble polypeptides of the invention is provided in the experimental section herein.

### Population of Cells

In a further aspect, there is provided a population of cells comprising the cell as defined earlier herein. In an embodiment, such a cell comprises a nucleic acid molecule A1, B1 or C1 or expresses a γδTCR A, B or C as earlier defined herein.

In an embodiment, it means that at least 10%, at least 20%, at least 30%, at least 40%, at least 50%, at least 60%, at least 70%, at least 80%, at least 90%, at least 95%, or 100% of the cells within said population are cells comprising a nucleic acid molecule A1, B1 or C1 or expressing a γδTCR A, B or C as earlier defined herein. Preferably, the cells are T-cells, more preferably γδT-cells, αβT-cells or NK-cells, most preferably αβT-cells.

The person skilled in the art is well capable of selecting and/or identifying cell populations characterized by expression of such δT-cell or γT-cell receptor chain or a part thereof or a γδTCR or a part thereof using FACS as explained earlier herein.

### Expression of a Chimeric Bidirectional Signaling Transmembrane Protein (Also Called Chimeric Protein)

In an embodiment, a T-cell, preferably a γδT-cell or an αβT-cell, more preferably an αβT-cell:
a) comprises a nucleic acid molecule encoding the amino acid sequence as identified earlier herein and/or expresses the amino acid sequence as identified earlier herein and/or comprises a nucleic acid molecule as identified earlier herein and
b) further comprises a polynucleotide encoding a chimeric bidirectional signaling transmembrane protein able to transduce at least two intracellular signals, said protein comprising:
   an extracellular ligand domain, able to interact with the extracellular domain of its interaction partner
   a transmembrane domain, and
   a heterologous intracellular signaling domain transducing a first signal after binding of the extracellular ligand domain to its interaction partner.

In an embodiment, said T-cell expresses a γδTCR or part thereof comprising a CDR3 region, comprising A or B or C:
A:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7 and/or 9, preferably at least 70% sequence identity with SEQ ID NO: 7, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10 and/or 12, or with SEQ ID NO: 10, 12, and/or 131, preferably at least 85% sequence identity with SEQ ID NO: 10,
B:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 13 and/or 15, or with amino acid sequence SEQ ID NO: 13, 15, 142, and/or 153, preferably at least 70% sequence identity with SEQ ID NO: 13, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 16 and/or 18, or with SEQ ID NO: 16, 18, 133, 143, 144, 154, 155, and/or 162, preferably at least 85% sequence identity with SEQ ID NO: 16,
C:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 19 and/or 21, preferably at least 70% sequence identity with SEQ ID NO: 19, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 22 and/or 24, or with amino acid sequence SEQ ID NO: 22, 24, and/or 135, preferably at least 85% sequence identity with SEQ ID NO: 22,
      and further comprises a polynucleotide encoding a chimeric bidirectional signaling transmembrane protein able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner.

Each of the γδTCR or part thereof comprising A or B or C has been earlier defined herein.

Throughout the application, the expression “chimeric bidirectional signaling transmembrane protein” may be replaced by the expression “chimeric protein”.

Below a few definitions are provided relating to the chimeric protein.

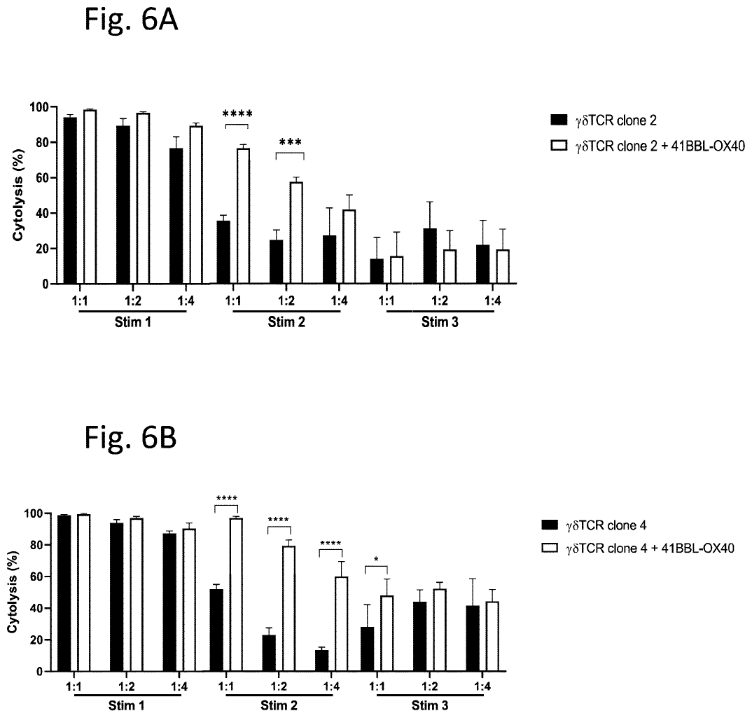

Discovered herein is that multi-directional signal transducer proteins or chimeric proteins can be used as a strategy to overcome limitations that hamper the production and use of T-cells for example, difficulties in generating sufficient numbers of the desired cells, limited proliferative ability or lifespan of the cells, limited induction of effector function upon cell recognition of antigen, and cell exhaustion. These proteins may be co-expressed in conjunction with the γT-cell receptor chains, δT-cell receptor chains, γδT-cell receptors, or parts thereof described herein, in T-cells, preferably γδT-cells or αβT-cells, more preferably αβT-cells. Therefore, these chimeric proteins can be used to improve the production and use of T-cells as identified herein, indeed, the expression of such chimeric protein has a positive effect on some properties exhibited by these T-cells, such as their immune effector function as demonstrated in FIG. 6 (intensity and duration).

These chimeric proteins are engineered fusion proteins that contain an extracellular ligand domain that binds to an interaction partner, a transmembrane domain, and a heterologous intracellular signaling domain (from or derived from a different protein than the extracellular ligand domain). When the extracellular ligand binds to its interaction partner, multi-directional signaling is induced that comprises at least one signal mediated by the heterologous intracellular signaling domain of said chimeric protein, and at least one signal mediated by an intracellular signaling domain of the interaction partner.

An extracellular ligand domain can be selected based on its ability to induce signaling mediated by a desired interaction partner. In some cases, an extracellular ligand domain can be selected based on its ability to elicit signaling mediated by the heterologous intracellular signaling domain of the chimeric protein upon binding to the interaction partner. The “at least two intracellular signals” are inducible. It means that the chimeric bidirectional signaling transmembrane protein may be considered as having two configurations: one wherein no signal is induced and one wherein “at least two intracellular signals” are induced upon interaction of the extracellular ligand domain of the chimeric protein with the extracellular ligand domain of its interaction partner. These “at least two intracellular signals” may occur simultaneously or sequentially. The inducibility of these “at least two intracellular signals” is attractive as the chimeric protein is controllable by the interaction partner and vice versa. This inducibility may be assessed using techniques known to the skilled person and depending on the identity of the heterologous intracellular signaling domain of the chimeric protein and of the intracellular domain of the interaction partner. In addition, one of these “at least two intracellular signals” may depend on the activation of additional receptor, for example but not limited to the γδTCR.

An extracellular ligand domain can comprise an amino acid sequence that is from or derived from a protein that is expressed on a cell surface. In some embodiments the protein expressed on a cell surface has agonist activity on a cognate receptor.

The extracellular ligand domain can comprise an amino acid sequence that is from or derived from a type I transmembrane protein. In some embodiments, the extracellular ligand domain comprises an amino acid sequence that is from or derived from a type II transmembrane protein. The extracellular ligand domain can comprise an amino acid sequence that is from or derived from a tumour necrosis factor superfamily member. In some cases, the extracellular ligand domain comprises an amino acid sequence that is from or derived from an immune co-receptor ligand, for example, an immune co-stimulatory ligand. In some embodiments, the extracellular ligand domain comprises an amino acid sequence that is from or derived from an immunoglobulin superfamily member. The extracellular ligand domain can comprise an amino acid sequence that is from or derived from 41BBL, OX40L, CD86, or RANK. The extracellular ligand domain can comprise an amino acid sequence that is from or derived from 41 BBL, OX40L, CD86, RANK, or CD70. In some embodiments, the extracellular ligand domain comprises an amino acid sequence that is from or derived from 41 BBL. In an embodiment, the extracellular ligand domain is from or derived from 41 BBL which is a type II transmembrane protein. In some embodiments, the extracellular ligand domain comprises an amino acid sequence that is from or derived from OX40L. In some embodiments, the extracellular ligand domain comprises an amino acid sequence that is from or derived from CD86. In some embodiments, the extracellular ligand domain comprises an amino acid sequence that is from or derived from RANK. In some embodiments, the extracellular ligand domain comprises an amino acid sequence that is from or derived from CD70.

The extracellular ligand domain can comprise an amino acid sequence that is from or derived from a receptor, for example, an ion channel, GPCR, or receptor tyrosine kinase. In some embodiments, the extracellular ligand domain comprises an amino acid sequence that is from or derived from a tumour necrosis factor receptor superfamily member. In some embodiments, the extracellular ligand domain comprises an amino acid sequence that is from or derived from an immune co-receptor.

The extracellular ligand domain can comprise an amino acid sequence that is from or derived from a cytokine. The extracellular ligand domain can comprise an amino acid sequence that is from or derived from a C-type lectin. The extracellular ligand domain can comprise an amino acid sequence that is from or derived from a soluble protein, for example, a secreted or cytoplasmic protein.

An extracellular ligand domain can comprise a peptide ligand of an interaction partner, for example, a naturally-occurring or a synthetic peptide ligand.

An extracellular ligand domain can comprise an amino acid sequence that is from or derived from an antigen-binding protein. Non-limiting examples of antigen-binding proteins include antibodies, variable regions (e.g., variable chain heavy region (VH) and/or variable chain light region (VL)), short chain variable fragments (scFv), single domain antibodies, Fab, Fab′, F(ab′)₂, dimers and trimers of Fab conjugates, Fv, minibodies, diabodies, triabodies, tetrabodies, affibodies, ankyrin proteins, ankyrin repeats, DARPins, monobodies, nanobodies, avimers, adnectins, anticalins, Fynomers, Kunitz domains, knottins, or β-hairpin mimetics. In some embodiments, an extracellular ligand domain comprises one or more single-chain variable fragments (scFvs). A scFv (single-chain variable fragment) is a fusion protein that can comprise VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody). Minibodies are scFv-CH3fusion proteins that assemble into bivalent dimers. In some embodiments, an extracellular ligand domain comprises one or more DARPins. In some embodiments, an extracellular ligand domain comprises one or more complementarity determining regions (CDRs) from an antibody or T-cell receptor, for example, one, three or six CDRs. Antigen-binding fragments derived from monoclonal antibodies can be, for example, chimeric, humanized or fully human.

An extracellular ligand domain can be selected based on its binding affinity for a desired interaction partner. In some embodiments, an extracellular ligand domain binds to an interaction partner with a KD of, for example, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 900 μM, less than about 800 μM, less than about 700 μM, less than about 600 μM, less than about 500 μM, less than about 400 μM, less than about 300 μM, less than about 200 μM, less than about 100 μM, less than about 50 μM, less than about 10 μM, less than about 1 μM, less than about 500 fM, or less than about 100 fM.

An extracellular ligand domain can comprise an amino acid sequence that is from or derived from a wild type protein amino acid sequence. A wild type protein amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide.

An extracellular ligand domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, affinity for an interaction partner, or level of signaling mediated by an interaction partner. An extracellular ligand domain can comprise an amino acid sequence that is modified compared to a wild type protein amino acid sequence or an amino acid sequence disclosed herein, for example, to promote folding of the chimeric protein into a biologically active conformation. In some embodiments, part or all of an extracellular ligand domain comprises an amino acid sequence that is inverted compared to a wild type amino acid sequence (i.e. expressed as a retro-protein).

An extracellular ligand domain can comprise, consist essentially of, or consist of an amino acid sequence with at least a minimal level of sequence identity compared to a wild type protein amino acid sequence or any other amino acid sequence disclosed herein. In an embodiment, such extracellular ligand domain having at least a minimal level of sequence identity compared to a given amino acid sequence is functional and therefore encompassed by the invention as long as this extracellular ligand domain is able to bind or interact with the extracellular domain of its interaction partner. The level of binding or interaction should be detectable using an assay known to the skilled person. Examples of suitable assays are western blotting or FACS, ELISA or SPR assays. Depending on the extracellular ligand domain used, the skilled person will know which assay is the most appropriate. For example, for OX40, NFKB signaling will be assessed, for 41 BBL the binding of 41 BB will be assessed. In an embodiment, the activity of the extracellular ligand domain is assessed when said extracellular ligand domain is still comprised within the full length transmembrane molecule it originates from. For example, an extracellular ligand domain can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to a wild type protein amino acid sequence or any other amino acid sequence disclosed herein, for example, any one of SEQ ID NOs: 39-44 or 121 (see table 1). In cases where part or all of an extracellular ligand domain comprises an amino acid sequence that is inverted compared to a wild type amino acid sequence (i.e. expressed as a retro-protein), the wild type protein amino acid sequence can be inverted prior to calculating sequence identity.

In some embodiments, an extracellular ligand domain can comprise, consist essentially of, or consist of an amino acid sequence that is a wild type protein amino acid sequence or any other amino acid sequence disclosed herein.

Table 1 provides non-limiting examples of amino acid sequences that extracellular domains of the disclosure can comprise, consist of, consist essentially of, or be derived from. EC: extracellular.

TABLE 1

| SEQ ID | | | |
|---|---|---|---|
| NO: | Name | Description | Sequence |
| 39 | 41BBL-EC | WT 41BBL | ACPWAVSGARASPGSAASPRLREGPELSPDD |
| | | EC domain | PAGLLDLRQGMFAQLVAQNVLLIDGPLSWYSD |
| | | | PGLAGVSLTGGLSYKEDTKELVVAKAGVYYVF |
| | | | FQLELRRVVAGEGSGSVSLALHLQPLRSAAGA |
| | | | AALALTVDLPPASSEARNSAFGFQGRLLHLSA |
| | | | GQRLGVHLHTEARARHAWQLTQGATVLGLFR |
| | | | VTPEIPAGLPSPRSE |
| 40 | OX40L | WT OX40L | QVSHRYPRIQSIKVQFTEYKKEKGFILTSQKED |
| | | EC domain | EIMKVQNNSVIINCDGFYLISLKGYFSQEVNISL |
| | | | HYQKDEEPLFQLKKVRSVNSLMVASLTYKDKV |
| | | | YLNVTTDNTSLDDFHVNGGELILIHQNPGEFCV |
| | | | L |
| 41 | CD86 | WT CD86 EC | MDPQCTMGLSNILFVMAFLLSGAAPLKIQAYFN |
| | | domain | ETADLPCQFANSQNQSLSELVVFWQDQENLV |
| | | | LNEVYLGKEKFDSVHSKYMGRTSFDSDSWTL |
| | | | RLHNLQIKDKGLYQCIIHHKKPTGMIRIHQMNS |
| | | | ELSVLANFSQPEIVPISNITENVYINLTCSSIHGY |
| | | | PEPKKMSVLLRTKNSTIEYDGVMQKSQDNVTE |
| | | | LYDVSISLSVSFPDVTSNMTIFCILETDKTRLLS |
| | | | SPFSIELEDPQPPPDHIP |
| 42 | RANK | WT RANK EC | MAPRARRRRPLFALLLLCALLARLQVALQIAPP |
| | | domain | CTSEKHYEHLGRCCNKCEPGKYMSSKCTTTS |
| | | | DSVCLPCGPDEYLDSWNEEDKCLLHKVCDTG |
| | | | KALVAVVAGNSTTPRRCACTAGYHWSQDCEC |
| | | | CRRNTECAPGLGAQHPLQLNKDTVCKPCLAG |
| | | | YFSDAFSSTDKCRPWTNCTFLGKRVEHHGTE |
| | | | KSDAVCSSSLPARKPPNEPHVYLP |
| 43 | Reverse | 41BBL core | MLLLVTSLLLCELPHPAFLLIPDQGMFAQLVAQ |
| | 41BBL | protein in a | NVLLIDGPLSWYSDPGLAGVSLTGGLSYKEDT |
| | | type I | KELVVAKAGVYYVFFQLELRRVVAGEGSGSVS |
| | | orientated | LALHLQPLRSAAGAAALALTVDLPPASSEARN |
| | | way with IgK | SAFGFQGRLLHLSAGQRLGVHLHTEARARHA |
| | | leader | WQLTQGATVLGLFRVTPEIPAGLPSPRSERLD |
| | | sequence and | LLGAPDDPSLEPGERLRPSAASGPSARA |
| | | inverted | |
| | | extracellular | |
| | | N-terminal | |
| | | part | |
| 44 | 41BBL - | WT 41BBL | ACPWAVSGARASPGSAASPRLREGPELSPDD |
| | CD86 IgV | EC domain | PAGLLDLRQGMFAQLVAQNVLLIDGPLSWYSD |
| | domain | with CD86 IgV | PGLAGVSLTGGLSYKEDTKELVVAKAGVYYVF |
| | | domain linked | FQLELRRVVAGEGSGSVSLALHLQPLRSAAGA |
| | | to it with | AALALTVDLPPASSEARNSAFGFQGRLLHLSA |
| | | GGGS linker | GQRLGVHLHTEARARHAWQLTQGATVLGLFR |
| | | | VTPEIPAGLPSPRSESLNGGGGSGGGGSGGG |
| | | | GSGGGGSGGGGSTSAPLKIQAYFNETADLPC |
| | | | QFANSQNQSLSELVVFWQDQENLVLNEVYLG |
| | | | KEKFDSVHSKYMGRTSFDSDSWTLRLHNLQIK |
| | | | DKGLYQCIIHHKKPTGMIRIHQMNSELSVLAN |
| 121 | CD70 | WT CD70 EC | QRFAQAQQQLPLESLGWDVAELQLNHTGPQQ |
| | | domain | DPRLYWQGGPALGRSFLHGPELDKGQLRIHR |
| | | | DGIYMVHIQVTLAICSSTTASRHHPTTLAVGICS |
| | | | PASRSISLLRLSFHQGCTIASQRLTPLARGDTL |
| | | | CTNLTGTLLPSRNTDETFFGVQWVRP |

An extracellular ligand domain can comprise an amino acid sequence with one or more amino acid insertions, deletions, or substitutions compared to a wild type protein amino acid sequence or any other amino acid sequence disclosed herein.

For example, an extracellular ligand domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a wild type protein amino acid sequence or any other amino acid sequence disclosed herein, for example, any one of SEQ ID NOs: 39-44 or 121.

In some embodiments, an extracellular ligand domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a wild type protein amino acid sequence or any other amino acid sequence disclosed herein, for example, any one of SEQ ID NOs: 39-44 or 121. In some embodiments, an extracellular ligand domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to a wild type protein amino acid sequence or any other amino acid sequence disclosed herein, for example, any one of SEQ ID NOs: 39-44 or 121.

The one or more insertions can be at the N-terminus, C-terminus, within the amino acid sequence, or a combination thereof. The one or more insertions can be contiguous, non-contiguous, or a combination thereof.

In some embodiments, an extracellular ligand domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to a wild type protein amino acid sequence or any other amino acid sequence disclosed herein, for example, any one of SEQ ID NOs: 39-44 or 121.

In some embodiments, an extracellular ligand domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to a wild type protein amino acid sequence or any other amino acid sequence disclosed herein, for example, any one of SEQ ID NOs: 39-44 or 121. In some embodiments, an extracellular ligand domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to a wild type protein amino acid sequence or any other amino acid sequence disclosed herein, for example, any one of SEQ ID NOs: 39-44 or 121.

The one or more deletions can be at the N-terminus, C-terminus, within the amino acid sequence, or a combination thereof. The one or more deletions can be contiguous, non-contiguous, or a combination thereof.

In some embodiments, an extracellular ligand domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a wild type protein amino acid sequence or any other amino acid sequence disclosed herein, for example, any one of SEQ ID NOs: 39-44 or 121.

In some embodiments, an extracellular ligand domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a wild type protein amino acid sequence or any other amino acid sequence disclosed herein, for example, any one of SEQ ID NOs: 39-44 or 121.

In some embodiments, an extracellular ligand domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to a wild type protein amino acid sequence or any other amino acid sequence disclosed herein, for example, any one of SEQ ID NOs: 39-44 or 121.

The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide.

A chimeric protein can have any suitable number of extracellular ligand domains. In some embodiments a chimeric protein has one extracellular ligand domain. In some embodiments, a chimeric protein has two extracellular ligand domains. In some embodiments, a chimeric protein has 1, 2, 3, 4, 5, 6, 7, 8, 9, or 10 extracellular ligand domain(s). In some embodiments, a chimeric protein has at least 1, at least 2, at least 3, at least 4, at least 5, at least 6, at least 7, at least 8, at least 9, or at least 10 extracellular ligand domain(s). In some embodiments, a chimeric protein has at most 1, at most 2, at most 3, at most 4, at most 5, at most 6, at most 7, at most 8, at most 9, or at most 10 extracellular ligand domain(s).

An interaction partner of an extracellular ligand domain is present on the surface of a cell and upon binding of the extracellular ligand domain to the interaction partner, signaling via an intracellular domain of the interaction partner is induced. Induction of the signaling pathway can contribute to a range of target biological outcomes and biological functions disclosed herein, for example, enhanced cellular proliferation, survival, and greater magnitude and duration of immune effector functions.

An interaction partner may be a co-immune receptor.

In an embodiment, a T-cell comprises, preferably expresses the chimeric bidirectional signaling transmembrane protein and the interaction partner, each as a transmembrane protein. In an embodiment, there is no cell comprising or expressing the interaction partner and that will not comprise or will not express the signal bidirectional signaling transmembrane protein. The interaction partner may be endogenously expressed on a cell and said cell may be transduced or transform with the chimeric bidirectional signaling transmembrane protein. Alternatively, both the interaction partner and the chimeric bidirectional signaling transmembrane protein may be transduced into the same cell.

In an embodiment, the interaction partner of the chimeric bidirectional signaling transmembrane protein comprises:
an extracellular domain able to interact with the extracellular ligand domain of the chimeric protein,
a transmembrane domain, and
an intracellular domain transducing a second signal after binding of the extracellular domain of the interaction partner to the extracellular ligand domain of the chimeric protein.

In some embodiments, binding of the extracellular ligand domain to the interaction partner modulates a second signaling pathway, for example, induces, or increases or decreases activity of the second signaling pathway. In some embodiments, the interaction partner is present in a signaling complex and upon binding of the extracellular ligand domain of the chimeric protein to the interaction partner, signaling mediated by the interaction partner is modulated, e.g., signaling mediated by the signaling complex is increased or decreased. In some embodiments, upon binding of the extracellular ligand domain to the interaction partner, activity of a first signaling pathway is reduced and a different signaling pathway is induced. An interaction partner can be selected based on its ability to modulate (e.g., induce) a signaling pathway that is associated with a desired biological outcome or biological function.

In some embodiments, the chimeric protein binds to the interaction partner as a monomer. In some embodiments, the chimeric protein forms a dimer when bound to the interaction partner. In some embodiments, the chimeric protein forms a trimer when bound to the interaction partner. In some embodiments, the chimeric protein binds to the interaction partner as a tetramer, a pentamer, a hexamer, or a multimer. When bound as a multimer (e.g., a dimer, trimer, tetramer, pentamer, hexamer, or higher order multimer), the chimeric protein can form a homo-multimer (e.g., homodimer, homotrimer, homotetramer, homopentamer, homohexamer, or higher order homomultimer). In some cases, the chimeric protein binds to the interaction partner as a hetero-multimer (e.g., a heterodimer, heterotrimer, heterotetramer, heteropentamer, heterohexamer, or higher order heteromultimer).

In some embodiments, the interaction partner that binds to the extracellular ligand domain is expressed by an immune cell. In some embodiments, the interaction partner is expressed by a leukocyte, such as a lymphocyte, e.g., a T-cell. In some embodiments, the interaction partner is expressed by a cancer cell. In some embodiments, the interaction partner is expressed by a mammalian cell. In some embodiments, the interaction partner is expressed by a human cell. In some embodiments, the interaction partner is expressed by an alpha-beta T-cell, a gamma delta T-cell, CD4+ T-cell, CD8+ T-cell, a T effector cell, a lymphocyte, a B cell, an NK cell, an NKT-cell, a myeloid cell, a monocyte, a macrophage, a neutrophil, a basophil, a dendritic cell, an eosinophil, a granulocyte, a helper T-cell, a memory T-cell, a Langerhans cell, a lymphoid cell, an innate lymphoid cell (ILC), a masT-cell, a megakaryocyte, a plasma cell, a regulatory T-cell, a thymocyte, a fibroblast, a keratinocyte, a mesenchymal stem cell, an endothelial cell, a stromal cell, or any mixture or combination of cells thereof. In some embodiments, the interaction partner is expressed by a primary cell.

In some embodiments, the interaction partner is expressed by a cell that is the same cell type as the cell that expresses the chimeric protein (that is the T-cell as earlier defined herein). In some embodiments, the chimeric protein and the interaction partner are both expressed by the same cell (that is the T-cell as earlier defined herein).

An interaction partner can be a receptor, for example, for example a tumour necrosis factor receptor superfamily member. The interaction partner can be, for example, 41 BB, OX40, RANKL, or IL18RAP (IL18RB). The interaction partner can be, for example, 41BB, OX40, RANKL, IL18RAP (IL18RB), or CD27. In some embodiments, the interaction partner is 41BB. In some embodiments, the interaction partner is OX40. In some embodiments, the interaction partner is RANKL. In some embodiments, the interaction partner is IL18RAP. In some embodiments, the interaction partner is CD27.

In some embodiments, an interaction partner is an immunoglobulin superfamily member, or an immune co-receptor, for example an activating immune co-receptor, such as CD86. In some embodiments, an interaction partner is a cytokine receptor. In some embodiments, an interaction partner is a C-type lectin receptor. In some embodiments, the interaction partner is an ion channel, GPCR, serine peptidase, integrin, tetraspanin, or receptor tyrosine kinase. In some embodiments, an interaction partner is a tumour necrosis factor superfamily member that comprises an intracellular domain that can mediate signaling. In some embodiments, the interaction partner is 41 BBL or OX40L.

In an embodiment, the at least two inducible intracellular signals transduced by the chimeric bidirectional signaling transmembrane protein contribute to an improvement of a biological parameter and/or function of the T-cell as earlier defined herein expressing the chimeric protein and the γδTCR and/or an improvement of a biological parameter and/or function induced by such a cell. In an embodiment the function is an anti-tumour or anti-infective response or activity as earlier defined herein. In an embodiment, the anti-tumour or anti-infective response or activity is stronger and more durable as demonstrated in FIG. 6. The assessment of an anti-tumour or anti-infective response or reactivity or activity has been already explained herein.

In some embodiments, upon binding of the extracellular ligand domain to the interaction partner, at least one, at least two, at least three, at least four, at least five, or at least six signaling pathways are induced that are mediated by the intracellular domain of the interaction partner. In some embodiments, upon binding of the extracellular ligand domain to the interaction partner, one, two, three, four, five, or six signaling pathways are induced that are mediated by the intracellular domain of the interaction partner. In some embodiments, upon binding of the extracellular ligand domain to the interaction partner, one signaling pathway is induced that is mediated by the intracellular domain of the interaction partner.

The extracellular part of the chimeric protein can comprise one or more additional extracellular domains as well as the one or more extracellular ligand domains.

In some embodiments, a chimeric protein comprises one or more additional extracellular domains from the same protein as the extracellular ligand domain, e.g., stretches of amino acids that do not participate in binding to an interaction partner, or do not induce signaling mediated by an interaction partner that binds to the extracellular ligand domain. In some embodiments, an additional extracellular domain does not participate in binding to the interaction partner but, increases or decreases a level of signaling mediated by the interaction partner.

In some embodiments, a chimeric protein comprises an additional extracellular domain that is from or derived from the same protein as the transmembrane domain, e.g., the same protein or a different protein than the heterologous intracellular signaling domain. In some embodiments, such an additional extracellular domain does not induce signaling mediated by an interaction partner.

In some embodiments, a chimeric protein comprises an additional extracellular domain that is from or derived from the same protein as the heterologous intracellular signaling domain. In some embodiments, such an additional extracellular domain does not induce signaling mediated by an interaction partner. In some cases, an additional extracellular domain can be selected based on its ability to elicit signaling in mediated by the heterologous intracellular signaling domain of the chimeric protein upon binding of the extracellular ligand domain to the interaction partner.

An additional extracellular domain can be or can comprise a cleavage site, for example, an ADAM family cleavage site or a metalloprotease family cleavage site. An additional extracellular domain can be or can comprise a multimerization domain (e.g., a domain that facilitates formation of a homo- or hetero-dimer, trimer, tetramer, pentamer, hexamer, or higher order multimer, such as a tenascin-C oligomerization domain, a thrombospondin oligomerization domain, or a GCN4 oligomerization domain). An additional extracellular domain can be or can comprise a cellular localization motif, e.g., a lipid raft localization motif or a nuclear localization motif. An additional extracellular domain can be or can comprise a target peptide, e.g., a signal peptide. An additional extracellular domain can comprise a linker.

An additional extracellular domain can comprise an amino acid sequence that is from or derived from a wild type protein amino acid sequence. An additional extracellular domain can comprise an amino acid sequence that is from or derived from any protein or type of protein disclosed elsewhere herein. An additional extracellular domain can comprise an amino acid sequence that is modified compared to a wild type protein amino acid sequence or any other amino acid sequence disclosed herein, for example, to achieve a desirable level of expression, surface expression, stability, resistance to aggregation, resistance to shedding, or resistance to degradation. An additional extracellular domain can comprise an amino acid sequence that is modified compared to a wild type protein amino acid sequence or an amino acid sequence disclosed herein, for example, to promote folding of the chimeric protein into a biologically active conformation. In some embodiments, part or all of an additional extracellular domain comprises an amino acid sequence that is inverted compared to a wild type amino acid sequence (i.e. expressed as a retro-protein).

An additional extracellular domain can comprise an amino acid sequence with one or more amino acid insertions, deletions, or substitutions compared to a wild type protein amino acid sequence or any other amino acid sequence as disclosed elsewhere herein. An additional extracellular domain can comprise at least a minimal level of sequence identity compared to a wild type protein amino acid sequence or any other amino acid sequence as disclosed elsewhere herein.

Chimeric proteins comprise at least one heterologous intracellular signaling domain. “Heterologous” refers to the fact that the intracellular signaling domain is from or is derived from a different protein than the extracellular ligand domain. A signaling pathway mediated by the heterologous intracellular signaling domain is induced upon binding of the extracellular ligand domain to an interaction partner. The induction of the signaling pathway can contribute to a range of target biological outcomes and biological functions disclosed herein, for example, enhanced cellular proliferation, survival, and greater magnitude and duration of immune effector functions such as anti-tumour or anti-infective activity.

A heterologous intracellular signaling domain can be selected based on its ability to induce a signaling pathway that is associated with a desired biological outcome or biological function. A heterologous intracellular signaling domain can comprise an amino acid sequence that is from or derived from a transmembrane protein, for example, a protein that is expressed on a cell surface. The heterologous intracellular signaling domain can comprise an amino acid sequence that is from or derived from a type I transmembrane protein. In some embodiments, the heterologous intracellular signaling domain comprises an amino acid sequence that is from or derived from a type II transmembrane protein.

The heterologous intracellular signaling domain can comprise an amino acid sequence that is from or derived from a tumour necrosis factor receptor superfamily member. The heterologous intracellular signaling domain can comprise an amino acid sequence that is from or derived from an immunoglobulin superfamily member. The heterologous intracellular signaling domain can comprise an amino acid sequence that is from or derived from a cytokine receptor. The heterologous intracellular signaling domain can comprise an amino acid sequence that is from or derived from a C-lectin family member. The heterologous intracellular signaling domain can comprise an amino acid sequence that is from or derived from 41 BB, OX40, NKp80, or IL18RAP. The heterologous intracellular signaling domain can comprise an amino acid sequence that is from or derived from 41BB, OX40, NKp80, IL18RAP, or IL2RB. In some embodiments, the heterologous intracellular signaling domain comprises an amino acid sequence that is from or derived from 41BB. In some embodiments, the heterologous intracellular signaling domain comprises an amino acid sequence that is from or derived from OX40. In some embodiment, the heterologous intracellular signaling domain comprises an amino acid sequence that is from or derived from OX40 and is from or derived from a type I transmembrane OX40 protein. In some embodiments, the heterologous intracellular signaling domain comprises an amino acid sequence that is from or derived from NKp80. In some embodiments, the heterologous intracellular signaling domain comprises an amino acid sequence that is from or derived from IL18RAP. In some embodiments, the heterologous intracellular signaling domain comprises an amino acid sequence that is from or derived from IL2RB.

The heterologous intracellular signaling domain can comprise an amino acid sequence that is from or derived from a receptor, for example, an ion channel, GPCR, serine protease, an immunoglobulin superfamily member, complement receptor, TIR domain containing receptor, or receptor tyrosine kinase. The heterologous intracellular signaling domain can comprise an amino acid sequence that is from or derived from a cytokine receptor. The heterologous intracellular signaling domain can comprise an amino acid sequence that is from or derived from a C-type lectin receptor. The heterologous intracellular signaling domain can comprise an amino acid sequence that is from or derived a cytoplasmic protein that participates in a signaling pathway. The heterologous intracellular signaling domain can comprise an amino acid sequence that is from or derived a nuclear protein that participates in a signaling pathway.

In some embodiments, the heterologous intracellular signaling domain comprises an amino acid sequence that is from or derived from an intracellular domain of a tumour necrosis factor superfamily member. In some embodiments, the heterologous intracellular signaling domain comprises an amino acid sequence that is from or derived from an intracellular domain of an immune co-receptor. In some cases, the heterologous intracellular signaling domain comprises an amino acid sequence that is from or derived from an intracellular domain of an immune co-receptor ligand that contains a signaling domain, for example, an intracellular signaling domain of an immune co-stimulatory ligand. In many cases it is not necessary to use the entire chain, for example, a truncated portion of the signaling domain can be used in the heterologous intracellular signaling domain.

The heterologous intracellular signaling domain can be structurally distinct from intracellular domains found in chimeric antigen receptors and similar chimeric proteins. For example, the heterologous intracellular signaling domain can lack one or more components associated with TCR complex signaling. In some embodiments, the heterologous intracellular signaling domain does not contain an ITAM. In some embodiments, the heterologous intracellular signaling domain contains a hemITAM but does not contain an ITAM. In some embodiments, the heterologous intracellular signaling domain is not phosphorylated upon binding of the chimeric protein to the interaction partner. In some embodiments, the heterologous intracellular signaling domain does not contain an intracellular domain from a CD3 chain, for example does not contain an intracellular domain of a CD3 zeta chain. In some embodiments, the heterologous intracellular signaling domain does not contain an intracellular domain from a TCR signaling complex. In some embodiments, the heterologous intracellular signaling domain is phosphorylated upon binding of the chimeric protein to the interaction partner.

A heterologous intracellular signaling domain can comprise an amino acid sequence that is from or derived from a wild type protein amino acid sequence. A heterologous intracellular signaling domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, signaling strength, or affinity for a protein that participates in downstream signaling, e.g., an adapter protein. A heterologous intracellular signaling domain can comprise an amino acid sequence that is modified compared to a wild type protein amino acid sequence or an amino acid sequence disclosed herein, for example, to promote folding of the chimeric protein into a biologically active conformation. In some embodiments, part or all of a heterologous intracellular signaling domain comprises an amino acid sequence that is inverted compared to a wild type amino acid sequence (i.e. expressed as a retro-protein).

A heterologous intracellular signaling domain can comprise, consist essentially of, or consist of an amino acid sequence with at least a minimal level of sequence identity compared to a wild type protein amino acid sequence or any other amino acid sequence disclosed herein. For example, a heterologous intracellular signaling domain can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to a wild type protein amino acid sequence or any other amino acid sequence disclosed herein, for example, any one of SEQ ID NOs: 45-57 or 122 (see table 2). In an embodiment, such heterologous intracellular signaling domain having at least a minimal level of sequence identity compared to a given amino acid sequence is functional and therefore encompassed by the invention as long as this intracellular signaling domain is able to transduce a first signal after binding of the extracellular ligand domain to its interaction partner. The first signal should be detectable using an assay known to the skilled person. Examples of suitable assays are western blotting or FACS, luminescence assays.

Depending on the identity of the heterologous intracellular signaling domain used, the skilled person will know which assay is appropriate to use. A NfκB reporter assay may be used to assess the activity of said heterologous intracellular domain. In an embodiment, the activity of the heterologous intracellular signaling domain is assessed when said intracellular signaling domain is still comprised within the full length transmembrane molecule it originates from.

In cases where part or all of a heterologous intracellular signaling domain comprises an amino acid sequence that is inverted compared to a wild type amino acid sequence (i.e. expressed as a retro-protein), the wild type protein amino acid sequence can be inverted prior to calculating sequence identity. In some embodiments, a heterologous intracellular signaling domain can comprise, consist essentially of, or consist of an amino acid sequence that is a wild type protein amino acid sequence or any other amino acid sequence disclosed herein, for example, any one of SEQ ID NOs: 45-57 or 122.

Table 2 provides non-limiting examples of amino acid sequences that intracellular domains and heterologous intracellular signaling domain of the disclosure can comprise, consist of, consist essentially of, or be derived from. ICD: intracellular domain

TABLE 2

| SEQ | | | |
|---|---|---|---|
| ID | | | |
| NO: | Name | Description | Sequence |
| 45 | OX40-ICD | From WT | RDQRLPPDAHKPPGGGSFRTPIQEEQADAHSTL |
| | | OX40 | AKI |
| 46 | OX40-ICD- | Inverted | IKALTSHADAQEEQIPTRFSGGGPPKHADPPLRQ |
| | REV | OX40-ICD | DR |
| 47 | OX40-ICD- | From WT | VAAILGLGLVLGLLGPLAILLALYLLRRDQRLPPD |
| | TM | OX40, | AHKPPGGGSFRTPIQEEQADAHSTLAKI |
| | | includes | |
| | | OX40 TM | |
| | | domain | |
| 48 | 41BB-ICD | From WT | KRGRKKLLYIFKQPFMRPVQTTQEEDGCSCRFP |
| | | 41BB | EEEEGGCEL |
| 49 | 41BB-ICD- | Inverted | MLGKRGRKKLLYIFKQPFMRPVQTTQEEDGCSC |
| | REV | 41BB-ICD | RFPEEEEGGCEL |
| 50 | NKp80-ICD | NKp80-ICD | MQDEDGYMTLNVQSKKRSSAQTSQLTFKDYSV |
| | | | TLHW |
| 51 | IL18RAP- | From WT | AASALLYRHWIEIVLLYRTYQSKDQTLGDKKDFD |
| | ICD | IL18RAP | AFVSYAKWSSFPSEATSSLSEEHLALSLFPDVLE |
| | | | NKYGYSLCLLERDVAPGGVYAEDIVSIIKRSRRGI |
| | | | FILSPNYVNGPSIFELQAAVNLALDDQTLKLILIKF |
| | | | CYFQEPESLPHLVKKALRVLPTVTWRGLKSVPP |
| | | | NSRFWAKMRYHMPVKNSQGFTWNQLRITSRIF |
| | | | QWKGLSRTETTGRSSQPKEW |
| 52 | 41BB-ICD- | From WT | IISFFLALTSTALLFLLFFLTLRFSVVKRGRKKLLYI |
| | TM | 41BB, | FKQPFMRPVQTTQEEDGCSCRFPEEEEGGCEL |
| | | includes | |
| | | 41BB TM | |
| | | domain | |
| 53 | OX40-ICD- | From WT | IKALTSHADAQEEQIPTRFSGGGPPKHADPPLRQ |
| | ALYLLR | OX40 | DRRLLYLA |
| 54 | OX40-ICD- | Inverted | ALYLLRRDQRLPPDAHKPPGGGSFRTPIQEEQA |
| | ALYLLR- | OX40-ICD- | DAHSTLAKI |
| | REV | AYLLR | |
| 55 | TRAF | TRAF | PIQEEQ |
| | domain OX40 | domain OX40 | |
| 56 | YMTLN motif | YMTLN motif | YMTLN |
| 57 | TRAF | TRAF | QTTQEEDGCSCRFPEEEE |
| | domain 41BB | domain 41BB | |
| 122 | IL2RB ICD | IL2RB | VLHTPDQGQLEQLSLYADTNLPLLQTVKDRELVE |
| | | truncated | LPSIEPALGGPSFSSSPFPSSLWKQVDGGHESS |
| | | signaling | LQSFFKSPDPTNCKLVKKLWPGTNRCN |
| | | domain | |

A heterologous intracellular signaling domain can comprise an amino acid sequence with one or more amino acid insertions, deletions, or substitutions compared to a wild type protein amino acid sequence or any other amino acid sequence disclosed herein.

For example, a heterologous intracellular signaling domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a wild type protein amino acid sequence or any other amino acid sequence disclosed herein, for example, any one of SEQ ID NOs: 45-57 or 122.

In some embodiments, a heterologous intracellular signaling domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a wild type protein amino acid sequence or any other amino acid sequence disclosed herein, for example, any one of SEQ ID NOs: 45-57 or 122.

In some embodiments, a heterologous intracellular signaling domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to a wild type protein amino acid sequence or any other amino acid sequence disclosed herein, for example, any one of SEQ ID NOs: 45-57 or 122.

The one or more insertions can be at the N-terminus, C-terminus, within the amino acid sequence, or a combination thereof. The one or more insertions can be contiguous, non-contiguous, or a combination thereof.

In some embodiments, a heterologous intracellular signaling domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to a wild type protein amino acid sequence or any other amino acid sequence disclosed herein, for example, any one of SEQ ID NOs: 45-57 or 122.

In some embodiments, a heterologous intracellular signaling domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to a wild type protein amino acid sequence or any other amino acid sequence disclosed herein, for example, any one of SEQ ID NOs: 45-57 or 122.

In some embodiments, a heterologous intracellular signaling domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to a wild type protein amino acid sequence or any other amino acid sequence disclosed herein, for example, any one of SEQ ID NOs: 45-57 or 122. The one or more deletions can be at the N-terminus, C-terminus, within the amino acid sequence, or a combination thereof. The one or more deletions can be contiguous, non-contiguous, or a combination thereof.

In some embodiments, a heterologous intracellular signaling domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a wild type protein amino acid sequence or any other amino acid sequence disclosed herein, for example, any one of SEQ ID NOs: 45-57 or 122.

In some embodiments, a heterologous intracellular signaling domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a wild type protein amino acid sequence or any other amino acid sequence disclosed herein, for example, any one of SEQ ID NOs: 45-57 or 122.

In some embodiments, a heterologous intracellular signaling domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to a wild type protein amino acid sequence or any other amino acid sequence disclosed herein, for example, any one of SEQ ID NOs: 45-57 or 122. The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

In some embodiments, the heterologous intracellular signaling domain signals as a monomer. In some embodiments, the heterologous intracellular signaling domain signals as a dimer. In some embodiments, the heterologous intracellular signaling domain signals as a trimer. In some embodiments, the heterologous intracellular signaling domain signals as a tetramer, a pentamer, a hexamer, or a multimer. When signaling as a multimer (e.g., a dimer, trimer, tetramer, pentamer, hexamer, or higher order multimer), the heterologous intracellular signaling domain can signal as a homo-multimer (e.g., homodimer, homotrimer, homotetramer, homopentamer, homohexamer, or higher order homomultimer). In some cases, the heterologous intracellular signaling domain signals as a hetero-multimer (e.g., a heterodimer, heterotrimer, heterotetramer, heteropentamer, heterohexamer, or higher order heteromultimer). In some embodiments, the heterologous intracellular signaling domain signals in a different conformation or as a different multimer than a full length wild type protein from which the heterologous intracellular signaling domain is from or derived from.

A chimeric protein can have any suitable number of heterologous intracellular signaling domains. In some embodiments a chimeric protein has one heterologous intracellular signaling domain. In some embodiments, a chimeric protein has two heterologous intracellular signaling domains. In some embodiments, a chimeric protein has 1, 2, 3, 4, 5, 6, 7, 8, 9, or 10 heterologous intracellular signaling domain(s). In some embodiments, a chimeric protein has at least 1, at least 2, at least 3, at least 4, at least 5, at least 6, at least 7, at least 8, at least 9, or at least 10 heterologous intracellular signaling domain(s). In some embodiments, a chimeric protein has at most 1, at most 2, at most 3, at most 4, at most 5, at most 6, at most 7, at most 8, at most 9, or at most 10 heterologous intracellular signaling domain(s).

In some embodiments, upon binding of the extracellular ligand domain to the interaction partner, at least one, at least two, at least three, at least four, at least five, or at least six signaling pathways are induced that are mediated by the heterologous intracellular signaling domain. In some embodiments, upon binding of the extracellular ligand domain to the interaction partner, one, two, three, four, five, or six signaling pathways are induced that are mediated by the heterologous intracellular signaling domain. In some embodiments, upon binding of the extracellular ligand domain to the interaction partner, one signaling pathway is induced that is mediated by the heterologous intracellular signaling domain.

A chimeric protein can comprise one or more additional intracellular domains as well as the one or more heterologous intracellular signaling domains.

In some embodiments, a chimeric protein comprises one or more additional intracellular domains from or derived from the same protein as the heterologous intracellular signaling domain, e.g., stretches of amino acids that do not participate in signaling. In some embodiments, an additional intracellular domain does not directly participate in signaling (e.g., does not bind a signaling pathway component or undergo a chemical or structural change as part of a signaling pathway), but increases or decreases a level of signaling mediated by the heterologous intracellular signaling domain.

In some embodiments, a chimeric protein comprises an additional intracellular domain that is from or derived from the same protein as the transmembrane domain, which can be e.g., the same protein or a different protein than the extracellular ligand domain. Such an intracellular domain can comprise a signaling domain or can lack a signaling domain.

In some embodiments, a chimeric protein comprises an intracellular domain that is from or derived from the same protein as the extracellular ligand domain. Such an intracellular domain can lack a signaling domain or can comprise a different signaling domain to the heterologous intracellular signaling domain that is present in the chimeric protein. In some embodiments, one or more amino acids are added to achieve sequence similarity and/or structural similarity to the protein that is the source of the extracellular ligand domain. For example, in some embodiments, the amino acids MLG can be added to the intracellular N-terminus of a chimeric protein that contains a 41 BBL extracellular ligand domain.

An additional intracellular domain can be or can comprise a cleavage site, for example, an ADAM family cleavage site or a metalloprotease family cleavage site. An additional intracellular domain can be or can comprise a multimerization domain (e.g., a domain that facilitates formation of a homo- or hetero-dimer, trimer, tetramer, pentamer, hexamer, or higher order multimer, such as a tenascin-C oligomerization domain, a thrombospondin oligomerization domain, or a GCN4 oligomerization domain). An additional intracellular domain can be or can comprise a target peptide, e.g. a signal peptide. An additional intracellular domain can be or can comprise a cellular localization motif, e.g., a lipid raft localization motif or a nuclear localization motif. An additional intracellular domain can comprise a linker.

An additional intracellular domain can comprise an amino acid sequence that is from or derived from a wild type protein amino acid sequence. An additional intracellular domain can comprise an amino acid sequence that is from or derived from any protein or type of protein disclosed elsewhere herein. An additional intracellular domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, signaling strength, or affinity for a protein that participates in downstream signaling, e.g., an adapter protein. An additional intracellular domain can comprise an amino acid sequence that is modified compared to a wild type protein amino acid sequence or an amino acid sequence disclosed herein, for example, to promote folding of the chimeric protein into a biologically active conformation. In some embodiments, part or all of an additional intracellular domain comprises an amino acid sequence that is inverted compared to a wild type amino acid sequence (i.e. expressed as a retro-protein).

An additional intracellular domain can comprise an amino acid sequence with one or more amino acid insertions, deletions, or substitutions compared to a wild type protein amino acid sequence or any other amino acid sequence as disclosed elsewhere herein. An additional intracellular domain can comprise at least a minimal level of sequence identity compared to a wild type protein amino acid sequence or any other amino acid sequence as disclosed elsewhere herein.

In some embodiments, the entire intracellular part of the chimeric protein (containing the one or more heterologous intracellular signaling domain(s) and any additional intracellular domains) can be structurally distinct from intracellular domains found in chimeric antigen receptors and similar chimeric proteins. For example, the entire intracellular part of the chimeric protein can lack one or more components associated with TCR complex signaling. In some embodiments, the entire intracellular part of the chimeric protein does not contain an ITAM (e.g., contains a hemITAM but not an ITAM, or does not contain a hemITAM or an ITAM). In some embodiments, the entire intracellular part of the chimeric protein is not phosphorylated upon binding of the chimeric protein to the interaction partner. In some embodiments, an intracellular part of a chimeric protein is phosphorylated upon binding of the chimeric protein to the interaction partner. In some embodiments, the entire intracellular part of a chimeric protein does not contain an intracellular domain from a CD3 chain, for example does not contain an intracellular domain of a CD3 zeta chain, or does not contain an intracellular domain from any CD3 chain. In some embodiments, the entire intracellular part of a chimeric protein does not contain an intracellular domain from a TCR signaling complex.

The chimeric proteins comprise a transmembrane domain that connects the extracellular ligand domain to the heterologous intracellular signaling domain.

In some embodiments, part or all of the transmembrane domain is from the same protein as the extracellular ligand domain. In cases where part or all of the transmembrane domain is from the same protein as the extracellular ligand domain, the transmembrane domain and the extracellular ligand domain can be part of a contiguous amino acid sequence (e.g., that matches or corresponds to a wild type sequence), or can be separated by one or more amino acid insertions, deletions, and/or substitutions. In an embodiment, the transmembrane domain or part thereof is from or derived from the same protein as the extracellular ligand domain.

In some embodiments, part or all of the transmembrane domain is from the same protein as the heterologous intracellular signaling domain. In cases where part or all of the transmembrane domain is from the same protein as the heterologous intracellular signaling domain, the transmembrane domain and the heterologous intracellular signaling domain can be part of a contiguous amino acid sequence (e.g., that matches or corresponds to a wild type sequence), or can be separated by one or more amino acid insertions, deletions, and/or substitutions.

In some embodiments, part or all of the transmembrane domain is from or derived from a different protein than the extracellular ligand domain and the heterologous intracellular signaling domain. A transmembrane domain can comprise an amino acid sequence that is from or derived from a transmembrane protein, for example, a protein that is expressed on a cell surface. The transmembrane domain can comprise an amino acid sequence that is from or derived from a type I transmembrane protein. In some embodiments, the transmembrane domain comprises an amino acid sequence that is from or derived from a type II transmembrane protein.

The transmembrane domain can comprise an amino acid sequence that is from or derived from a tumour necrosis factor receptor superfamily member. The transmembrane domain can comprise an amino acid sequence that is from or derived from 41 BB, OX40, NKp80, RANK, or IL18RAP. The transmembrane domain can comprise an amino acid sequence that is from or derived from 41BB, OX40, NKp80, RANK, IL18RAP, or CD70. In some embodiments, the transmembrane domain comprises an amino acid sequence that is from or derived from 41 BB. In some embodiments, the transmembrane domain comprises an amino acid sequence that is from or derived from OX40. In some embodiments, the transmembrane domain comprises an amino acid sequence that is from or derived from NKp80. In some embodiments, the transmembrane domain comprises an amino acid sequence that is from or derived from RANK. In some embodiments, the transmembrane domain comprises an amino acid sequence that is from or derived from IL18RAP. In some embodiments, the transmembrane domain comprises an amino acid sequence that is from or derived from CD70.

In some embodiments, the transmembrane domain comprises an amino acid sequence that is from or derived from a tumour necrosis factor superfamily member or an immunoglobulin superfamily. The transmembrane domain can comprise an amino acid sequence that is from or derived from 41BBL, OX40L, CD86, or RANK. The transmembrane domain of a chimeric signaling protein can comprise an amino acid sequence that is from or derived from 41BBL, OX40L, CD86, RANK, or CD70. In some embodiments, the transmembrane domain comprises an amino acid sequence that is from or derived from 41BBL. In some embodiments, the transmembrane domain comprises an amino acid sequence that is from or derived from OX40L. In some embodiments, the transmembrane domain comprises an amino acid sequence that is from or derived from CD86. In some embodiments, the transmembrane domain comprises an amino acid sequence that is from or derived from RANK. In some embodiments, the transmembrane domain comprises an amino acid sequence that is from or derived from CD70. The transmembrane domain can comprise an amino acid sequence that is from or derived from a receptor, for example, an ion channel, GPCR, selectin family member, cytokine receptor, adhesion molecule, or receptor tyrosine kinase. The transmembrane domain can comprise an amino acid sequence that is from or derived from a cytokine receptor. The transmembrane domain can comprise an amino acid sequence that is from or derived from a C-type lectin or C type lectin receptor. In some embodiments, the transmembrane domain comprises an amino acid sequence that is from or derived from an immune co-receptor. In some cases, the transmembrane domain comprises an amino acid sequence that is from or derived from an immune co-receptor ligand, for example, an immune co-stimulatory ligand.

In an aspect, a transmembrane domain is from an alpha chain of a T-cell receptor (TCR), beta chain of a TCR, CD8, CD4, CD28, CD45, PD-1 and/or CD152.

A transmembrane domain can comprise an amino acid sequence that is from or derived from a wild type protein amino acid sequence. A transmembrane domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, signaling strength, localization, or multimerization of the chimeric protein. A transmembrane domain can comprise an amino acid sequence that is modified compared to a wild type protein amino acid sequence or an amino acid sequence disclosed herein, for example, to promote folding of the chimeric protein into a biologically active conformation. In some embodiments, part or all of a transmembrane domain comprises an amino acid sequence that is inverted compared to a wild type amino acid sequence (i.e. expressed as a retro-protein). A transmembrane domain can comprise an artificial hydrophobic sequence. In some embodiments, a transmembrane domain can comprise a cellular localization motif, e.g., a lipid raft localization motif or a nuclear localization motif.

In one non-limiting example, a chimeric protein can contain an extracellular ligand domain from RANK, and a transmembrane domain from IL18RAP. In some embodiments, inclusion of the transmembrane domain from IL18RAP induces formation of the chimeric protein into a dimeric state, unlike wild type RANK, which can function as a trimer. In the same way, transmembrane domains of the disclosure can induce formation of the chimeric protein into a monomeric or multimeric state that is different than the state adopted by the full length wild type version of the protein the extracellular ligand domain is from or derived from, and/or that is different than the full length wild type version of the protein the heterologous intracellular domain is from or derived from.

A transmembrane domain can comprise, consist essentially of, or consist of an amino acid sequence with at least a minimal level of sequence identity compared to a wild type protein amino acid sequence or any other amino acid sequence disclosed herein. For example, a transmembrane domain can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to a wild type protein amino acid sequence or any other amino acid sequence disclosed herein, for example, any one of SEQ ID NOs: 58-65 or 123 (see table 3). In an embodiment, such transmembrane domain having at least a minimal level of sequence identity compared to a given amino acid sequence is functional and therefore encompassed by the invention as long as this transmembrane domain is able to induce a multimerisation of the chimeric bidirectional signaling transmembrane protein comprising it upon binding of the extracellular domain of its interaction partner. The level of binding or interaction should be detectable using an assay known to the skilled person. Examples of suitable assays are western blotting or FACS, single photon microscopy assays.

In cases where part or all of a transmembrane domain comprises an amino acid sequence that is inverted compared to a wild type amino acid sequence (i.e. expressed as a retro-protein), the wild type protein amino acid sequence can be inverted prior to calculating sequence identity. In some embodiments, a transmembrane domain can comprise, consist essentially of, or consist of an amino acid sequence that is a wild type protein amino acid sequence or any other amino acid sequence disclosed herein, for example, any one of SEQ ID NOs: 58-65 or 123.

Table 3 provides non-limiting examples of amino acid sequences that a transmembrane domain of the disclosure can comprise, consist of, consist essentially of, or be derived from. TM: transmembrane

TABLE 3

| SEQ | | | |
|---|---|---|---|
| ID | | | |
| NO: | Name | Description | Sequence |
| 58 | OX40-TM | From WT OX40 | VAAILGLGLVLGLLGPLAILL |
| 59 | OX40L- | From WT | LLLVASVIQGLGLLLCFTYIC |
| | TM | OX40L | LHFSAL |
| 60 | 41BB-TM | From WT 41BB | IISFFLALTSTALLFLLFFLT |
| | | | LRFSVV |
| 61 | 41BBL- | From WT | WALVAGLLLLLLLAAACAVFL |
| | TM | 41BBL | |
| 62 | NKp80- | From WT | ILLGISGTVNGILTLTLISLI |
| | TM | NKp80 | |
| 63 | CD86-TM | From WT CD86 | WITAVLPTVIICVMVFCLILW |
| 64 | IL18RAP- | From WT | GWVLLYILLGTIGTLVAVL |
| | TM | IL18RAP | |
| 65 | RANK-TM | From WT RANK | GLIILLLFASVALVAAIIFGV |
| 123 | CD70-TM | TM CD70 | VLRAALVPLVAGLVICLVVCI |

A transmembrane domain can comprise an amino acid sequence with one or more amino acid insertions, deletions, or substitutions compared to a wild type protein amino acid sequence or any other amino acid sequence disclosed herein.

For example, a transmembrane domain can comprise an amino acid sequence with at least 1, at least 2, at least 3, at least 4, at least 5, at least 6, at least 7, at least 8, at least 9, or at least 10 amino acid insertions relative to a wild type protein amino acid sequence or any other amino acid sequence disclosed herein, for example, any one of SEQ ID NOs: 58-65 or 123. In some embodiments, a transmembrane domain comprises an amino acid sequence with at most 1, at most 2, at most 3, at most 4, at most 5, at most 6, at most 7, at most 8, at most 9, or at most 10 amino acid insertions relative to a wild type protein amino acid sequence or any other amino acid sequence disclosed herein, for example, any one of SEQ ID NOs: 58-65 or 123. In some embodiments, a transmembrane domain comprises an amino acid sequence with 1, 2, 3, 4, 5, 6, 7, 8, 9, or 10 amino acid insertions relative to a wild type protein amino acid sequence or any other amino acid sequence disclosed herein, for example, any one of SEQ ID NOs: 58-65 or 123. The one or more insertions can be at the N-terminus, C-terminus, within the amino acid sequence, or a combination thereof. The one or more insertions can be contiguous, non-contiguous, or a combination thereof.

In some embodiments, a transmembrane domain comprises an amino acid sequence with at least 1, at least 2, at least 3, at least 4, at least 5, at least 6, at least 7, at least 8, at least 9, or at least 10 amino acid deletions relative to a wild type protein amino acid sequence or any other amino acid sequence disclosed herein, for example, any one of SEQ ID NOs: 58-65 or 123. In some embodiments, a transmembrane domain comprises an amino acid sequence with at most 1, at most 2, at most 3, at most 4, at most 5, at most 6, at most 7, at most 8, at most 9, or at most 10 amino acid deletions relative to a wild type protein amino acid sequence or any other amino acid sequence disclosed herein, for example, any one of SEQ ID NOs: 58-65 or 123. In some embodiments, a transmembrane domain comprises an amino acid sequence with 1, 2, 3, 4, 5, 6, 7, 8, 9, or 10 amino acid deletions relative to a wild type protein amino acid sequence or any other amino acid sequence disclosed herein, for example, any one of SEQ ID NOs: 58-65 or 123. The one or more deletions can be at the N-terminus, C-terminus, within the amino acid sequence, or a combination thereof. The one or more deletions can be contiguous, non-contiguous, or a combination thereof.

In some embodiments, a transmembrane domain comprises an amino acid sequence with at least 1, at least 2, at least 3, at least 4, at least 5, at least 6, at least 7, at least 8, at least 9, or at least 10, amino acid substitutions relative to a wild type protein amino acid sequence or any other amino acid sequence disclosed herein, for example, any one of SEQ ID NOs: 58-65 or 123. In some embodiments, a transmembrane domain comprises an amino acid sequence with at most 1, at most 2, at most 3, at most 4, at most 5, at most 6, at most 7, at most 8, at most 9, or at most 10 amino acid substitutions relative to a wild type protein amino acid sequence or any other amino acid sequence disclosed herein, for example, any one of SEQ ID NOs: 58-65 or 123. In some embodiments, a transmembrane domain comprises an amino acid sequence with 1, 2, 3, 4, 5, 6, 7, 8, 9, or 10 amino acid sequence or any other amino acid sequence disclosed herein, for example, any one of SEQ ID NOs: 58-65 or 123. The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

Chimeric proteins can comprise one or more linkers that connect amino acid sequences, for example, amino acid sequences from or derived from different proteins. A linker can connect, for example, an extracellular ligand domain to a transmembrane domain, a heterologous intracellular signaling domain to a transmembrane domain, one extracellular ligand domain to a second extracellular ligand domain or an additional extracellular domain, one heterologous intracellular signaling domain to another heterologous intracellular signaling domain or an additional intracellular domain, or any domain disclosed herein to another amino acid sequence.

A linker or can allow for separation and flexibility of the domains it separates, for example, a transmembrane domain and an extracellular ligand domain. The length of a linker can be adjusted to alter the ability of a domain to bind to, for example, an interaction partner (for the extracellular ligand domain), or a factor that participates in a signaling pathway (e.g., for the heterologous intracellular signaling domain).

A linker sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. In some embodiments, a linker is at least 1, at least 3, at least 5, at least 7, at least 9, at least 11, or at least 15 amino acids in length. In some embodiments, a linker is at most 5, at most 7, at most 9, at most 11, at most 15, at most 20, at most 25, or at most 50 amino acids in length.

A flexible linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains.

A linker can comprise any of the sequences in Table 4, or repeats thereof (e.g., 2, 3, 4, 5, 6, 7, 8, 9, or 10 repeats of any of SEQ ID NOs: 66-82).

Table 4 provides non-limiting examples of amino acid linkers that can be used.

TABLE 4

| SEQ | | |
|---|---|---|
| ID | | |
| NO: | Description | Sequence |
| 66 | Flexible | TSGS |
| | linker | |
| 67 | Flexible | GGGGS |
| | linker | |
| 68 | Flexible | GGGS |
| | linker | |
| 69 | Flexible | GG |
| | linker | |
| 70 | Flexible | KESGSVSSEQLAQFRSLD |
| | linker | |
| 71 | Flexible | EGKSSGSGSESKST |
| | linker | |
| 72 | Flexible | GSAGSAAGSGEF |
| | linker | |
| 73 | Rigid linker | EAAAK |
| 74 | Rigid linker | EAAAR |
| 75 | Rigid linker | PAPAP |
| 76 | Rigid linker | AEAAAKEAAAKA |
| 77 | Rigid linker | ILTHDSSIRYLQEIYNSNNQKIVNLKEKVA |
| | | QLEAQCQEPCKDTVQIHDITG |
| 78 | Flexible | GGS |
| | linker | |
| 79 | Flexible | SLNGGGGSGGGGGGGGSGGGGSGGGGSTS |
| | linker | |
| 80 | Flexible | SGGSGGGGSGGGSGGGGSLQ |
| | linker | |
| 81 | Flexible | SGGGSGGGGSGGGGSGGGGSGGGSLQ |
| | linker | |
| 82 | Flexible | GGGGSGGGGSGGGGS |
| | linker | |
| 145 | linker | RTGGGGSGGGGSGGGGS |
| 147 | linker | GSG |

In some embodiments, a chimeric protein comprises a linker with at least 1, at least 2, at least 3, at least 4, or at least 5 amino acid insertions, deletions, or substitutions relative to any of SEQ ID NOs: 66-82. The insertions, deletions, or substitutions can be at the N-terminus, the C-terminus, within the sequence, or a combination thereof. The insertions, deletions, or substitutions can be contiguous or non-contiguous. In some cases, the substitutions are conservative. In some cases, the substitutions are non-conservative.

In some embodiments, a chimeric protein does not contain any linkers, for example, the chimeric protein is a direct fusion of amino acid sequences from other proteins with no intervening amino acid sequence.

All the linkers described above as suitable for the chimeric bidirectional signaling transmembrane protein may also be suitable for the soluble polypeptides comprising a γT-cell receptor chain, a δT-cell receptor chain, a γδT-cell receptor, or parts thereof (such as e.g., an extracellular domain thereof) described earlier herein.

In an embodiment, the chimeric bidirectional signaling transmembrane protein able to transduce at least two inducible intracellular signals, comprises:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner.

In an embodiment, the chimeric bidirectional signaling transmembrane protein able to transduce at least two inducible intracellular signals, comprises:
an extracellular ligand domain, able to interact with the extracellular domain of its interaction partner wherein the extracellular ligand domain is represented by a sequence having at least 80% identity with one of SEQ ID NO: 39-44 or 121 as identified in table 1,
a transmembrane domain represented by a sequence having at least 80% identity with one of SEQ ID NO: 58-65 or 123 as identified in table 3, and
a heterologous intracellular signaling domain transducing a first signal after binding of the extracellular ligand domain to its interaction partner, wherein the heterologous intracellular signaling domain is represented by a sequence having at least 80% identity with one of SEQ ID NO: 45-57 or 122 as identified in table 2,
   wherein the second intracellular signal is transduced via the intracellular domain of the interaction partner. In this embodiment, the sequence identity may be at least 80%, 81%, 82%, 83%, 84%, 85%, 86%, 87%, 88%, 89%, 90%, 91%, 92%, 93%, 94%, 95%, 96%, 97%, 98%, 99% or 100%.

In one embodiment, the transmembrane domain and the extracellular ligand domain are from the same proteins. Examples are CD86-OX40, 41 BBL-OX40, OX40L-41 BB.
(a) In an embodiment, the chimeric bidirectional signaling transmembrane protein able to transduce at least two inducible intracellular signals comprises: an extracellular ligand domain which is from or derived from a type I transmembrane protein and a heterologous intracellular signaling domain which is from or derived from a type II transmembrane protein or
(b) an extracellular ligand domain which is from or derived from a type II transmembrane protein and a heterologous intracellular signaling domain which is from or derived from a type I transmembrane protein.

Such chimeric proteins comprising part of a type I and part of a type II transmembrane protein exhibit surprising and unexpected effects, as type I and type II transmembrane proteins cannot be readily combined into a functional protein. For example, many attempts to fuse an amino acid sequence from a type I transmembrane protein to an amino acid sequence from type II transmembrane protein fail to yield a functional protein, for example, due to an altered N-terminal or C-terminal location of one of the amino acid sequences, inability of the resulting protein to adopt a functional conformation, tertiary structure, transmembrane orientation, or a combination thereof. Surprisingly some of these chimeric proteins have been successfully generated in the experimental part and have been found active.

In an embodiment, the chimeric bidirectional signaling transmembrane protein comprises:
an extracellular ligand domain comprising an amino acid sequence from a tumour necrosis factor superfamily member, a cytokine, a C-type lectin, an immunoglobulin superfamily member, or an antibody or antigen-binding fragment thereof; and
a heterologous intracellular signaling domain comprising an amino acid sequence from a tumour necrosis factor receptor superfamily member, a cytokine receptor, or a C-type lectin receptor.

In an embodiment, the chimeric bidirectional signaling transmembrane protein comprises:
an extracellular ligand domain comprising an amino acid sequence from 41BBL, OX40L, CD86, or RANK, and
a heterologous intracellular signaling domain comprising an amino acid sequence from OX40, 41BB, NKp80, or IL18RAP.

In an embodiment, the chimeric bidirectional signaling transmembrane protein comprises:
an extracellular ligand domain comprising an amino acid sequence from 41BBL, OX40L, CD86, RANK, or CD70, and
a heterologous intracellular signaling domain comprising an amino acid sequence from OX40, 41BB, NKp80, IL18RAP, or IL2RB.

In an embodiment, the chimeric bidirectional signaling transmembrane protein comprises:
(a) the extracellular ligand domain comprises an amino acid sequence from 41BBL and the heterologous intracellular signaling domain comprises an amino acid sequence from OX40, preferably wherein the extracellular ligand domain is from or is derived from a type II transmembrane protein 41 BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40,
(c) the extracellular ligand domain comprises an amino acid sequence from 41BBL and the heterologous intracellular signaling domain comprises an amino acid sequence from NKp80,
(d) the extracellular ligand domain comprises an amino acid sequence from RANK and the heterologous intracellular signaling domain comprises an amino acid sequence from IL18RAP,
(e) the extracellular ligand domain comprises an amino acid sequence from RANK and the heterologous intracellular signaling domain comprises an amino acid sequence from OX40,
(f) the extracellular ligand domain comprises an amino acid sequence from RANK and the heterologous intracellular signaling domain comprises an amino acid sequence from 41 BB,
(g) the extracellular ligand domain comprises an amino acid sequence from OX40L and the heterologous intracellular signaling domain comprises an amino acid sequence from 41 BB, or
(h) the extracellular ligand domain comprises an amino acid sequence from CD86 and the heterologous intracellular signaling domain comprises an amino acid sequence from IL18RAP,
(i) the extracellular ligand domain comprises an amino acid sequence from CD70 and the heterologous intracellular signaling domain comprises an amino acid sequence from OX40, or
(j) the extracellular ligand domain comprises an amino acid sequence from 41BBL and the heterologous intracellular signaling domain comprises an amino acid sequence from OX40 and an amino acid sequence from IL2RB.

In an embodiment, the chimeric bidirectional signaling transmembrane protein identified under a) is represented by an amino acid sequence having at least 80% identity with SEQ ID NO: 38, 83, 84, 95, 96, 97, 98, 99, 100, 101, 102, 124, or 125 as identified in table 5.

In an embodiment, the chimeric bidirectional signaling transmembrane protein identified under b) is represented by an amino acid sequence having at least 80% identity with SEQ ID NO 90, 91 or 111 as identified in table 5.

In an embodiment, the chimeric bidirectional signaling transmembrane protein identified under c) is represented by an amino acid sequence having at least 80% identity with SEQ ID NO: 85 or 86 as identified in table 5.

In an embodiment, the chimeric bidirectional signaling transmembrane protein identified under d) is represented by an amino acid sequence having at least 80% identity with SEQ ID NO: 116 as identified in table 5.

In an embodiment, the chimeric bidirectional signaling transmembrane protein identified under e) is represented by an amino acid sequence having at least 80% identity with SEQ ID NO: 114 as identified in table 5.

In an embodiment, the chimeric bidirectional signaling transmembrane protein identified under f) is represented by an amino acid sequence having at least 80% identity with SEQ ID NO: 115 as identified in table 5.

In an embodiment, the chimeric bidirectional signaling transmembrane protein identified under g) is represented by an amino acid sequence having at least 80% identity with SEQ ID NO: 87, 88, 89, or 103 as identified in table 5.

In an embodiment, the chimeric bidirectional signaling transmembrane protein identified under h) is represented by an amino acid sequence having at least 80% identity with SEQ ID NO: 109 or 110 as identified in table 5.

In an embodiment, the chimeric bidirectional signaling transmembrane protein identified under i) is represented by an amino acid sequence having at least 80% identity or similarity with SEQ ID NO: 128 or 129 as identified in table 5.

In an embodiment, the chimeric bidirectional signaling transmembrane protein identified under j) is represented by an amino acid sequence having at least 80% identity or similarity with SEQ ID NO: 125 as identified in table 5.

In these embodiments, the sequence identity may be at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the chimeric bidirectional signaling transmembrane protein does not contain an ITAM or an intracellular domain from a TCR signaling complex. In this context in an embodiment, an ITAM motif is “YxxL/1-x6-8-YxxL/I” wherein x stands for any amino acid. X6-8 means any stretch of 6, 7 or 8 amino acids, Y is Tyrosine, L is Leucine, I is Isoleucine (PFAM source https://pfam.xfam.org/family/ITAM or https://www.sciencedirect.com/science/article/abs/pii/S0962892406001498, corresponding to Lanier 2006, Trends Biotechnol 16(8): 388-390, incorporated herein by reference in its entirety).

Non-limiting examples of the chimeric protein sequences, and sequences that can be included in the chimeric proteins, are provided in Table 5.

TABLE 5

| SEQ ID | | | |
|---|---|---|---|
| NO: | Name | Description | Sequence |
| 38 | 41BBL- | WT 41BBL with | MLGRDQRLPPDAHKPPGGGSFRTPIQEEQADAHST |
| | OX40_GSG | 41BBL TM and | LAKITSGSYASDASLDPEAPWPPAPRARACRVLPW |
| | | OX40 | ALVAGLLLLLLLAAACAVFLACPWAVSGARASPGSA |
| | | intracellular | ASPRLREGPELSPDDPAGLLDLRQGMFAQLVAQNV |
| | | signaling | LLIDGPLSWYSDPGLAGVSLTGGLSYKEDTKELVVA |
| | | domain with | KAGVYYVFFQLELRRVVAGEGSGSVSLALHLQPLR |
| | | GSG linker | SAAGAAALALTVDLPPASSEARNSAFGFQGRLLHLS |
| | | | AGQRLGVHLHTEARARHAWQLTQGATVLGLFRVTP |
| | | | EIPAGLPSPRSEGSG |
| 83 | 41BBL- | WT 41BBL with | MLGRDQRLPPDAHKPPGGGSFRTPIQEEQADAHST |
| | OX40 | 41BBL TM and | LAKITSGSYASDASLDPEAPWPPAPRARACRVLPW |
| | | OX40 | ALVAGLLLLLLLAAACAVFLACPWAVSGARASPGSA |
| | | intracellular | ASPRLREGPELSPDDPAGLLDLRQGMFAQLVAQNV |
| | | signaling | LLIDGPLSWYSDPGLAGVSLTGGLSYKEDTKELVVA |
| | | domain | KAGVYYVFFQLELRRVVAGEGSGSVSLALHLQPLR |
| | | | SAAGAAALALTVDLPPASSEARNSAFGFQGRLLHLS |
| | | | AGQRLGVHLHTEARARHAWQLTQGATVLGLFRVTP |
| | | | EIPAGLPSPRSE |
| 84 | 41BBL13W- | 41BBL ligand | MLGIKALTSHADAQEEQIPTRFSGGGPPKHADPPLR |
| | OX40rev | binding domain, | QDRTSGSWPPAPRARACRVLPWALVAGLLLLLLLA |
| | | TM domain, | AACAVFLACPWAVSGARASPGSAASPRLREGPELS |
| | | and partial | PDDPAGLLDLRQGMFAQLVAQNVLLIDGPLSWYSD |
| | | intracellular | PGLAGVSLTGGLSYKEDTKELVVAKAGVYYVFFQLE |
| | | domain with | LRRVVAGEGSGSVSLALHLQPLRSAAGAAALALTVD |
| | | deletion of the | LPPASSEARNSAFGFQGRLLHLSAGQRLGVHLHTE |
| | | first 12 amino | ARARHAWQLTQGATVLGLFRVTPEIPAGLPSPRSE |
| | | acids | |
| | | containing a | |
| | | putative casein | |
| | | kinase I motif; | |
| | | and an OX40 | |
| | | intracellular | |
| | | signaling | |
| | | domain that is | |
| | | inverted | |
| 85 | 41BBL- | 41BBL WT with | MQDEDGYMTLNVQSKKRSSAQTSQLTFKDYSVTLH |
| | NKp80 | 41BBL TM | WYASDASLDPEAPWPPAPRARACRVLPWALVAGL |
| | | without first 2 | LLLLLLAAACAVFLACPWAVSGARASPGSAASPRLR |
| | | amino acids | EGPELSPDDPAGLLDLRQGMFAQLVAQNVLLIDGPL |
| | | with NKp80 | SWYSDPGLAGVSLTGGLSYKEDTKELVVAKAGVYY |
| | | intracellular | VFFQLELRRVVAGEGSGSVSLALHLQPLRSAAGAA |
| | | signaling | ALALTVDLPPASSEARNSAFGFQGRLLHLSAGQRL |
| | | domain | GVHLHTEARARHAWQLTQGATVLGLFRVTPEIPAG |
| | | | LPSPRSE |
| 86 | 41BBLmincyto- | 41BBL | MQDEDGYMTLNVQSKKRSSAQTSQLTFKDYSVTLH |
| | NKp80 | ligand | WYKWALVAGLLLLLLLAAACAVFLACPWAVSGARA |
| | | domain and TM | SPGSAASPRLREGPELSPDDPAGLLDLRQGMFAQL |
| | | linked to an | VAQNVLLIDGPLSWYSDPGLAGVSLTGGLSYKEDTK |
| | | NKp80 | ELVVAKAGVYYVFFQLELRRVVAGEGSGSVSLALHL |
| | | intracellular | QPLRSAAGAAALALTVDLPPASSEARNSAFGFQGR |
| | | signaling | LLHLSAGQRLGVHLHTEARARHAWQLTQGATVLGL |
| | | domain, lacking | FRVTPEIPAGLPSPRSE |
| | | the intracellular | |
| | | sequence of | |
| | | 41BBL | |
| 87 | OX40L- | WT OX40L | MLGKRGRKKLLYIFKQPFMRPVQTTQEEDGCSCRF |
| | 41BB_1 | including TM | PEEEEGGCELTSGSERVQPLEENVGNAARPRFERN |
| | | and IC domain | KLLLVASVIQGLGLLLCFTYICLHFSALQVSHRYPRIQ |
| | | and 41BB | SIKVQFTEYKKEKGFILTSQKEDEIMKVQNNSVIINCD |
| | | intracellular | GFYLISLKGYFSQEVNISLHYQKDEEPLFQLKKVRSV |
| | | signaling | NSLMVASLTYKDKVYLNVTTDNTSLDDFHVNGGELI |
| | | domain flipped | LIHQNPGEFCVL |
| | | in structural | |
| | | orientation | |
| 88 | OX40L- | OX40L WT | MLGKRGRKKLLYIFKQPFMRPVQTTQEEDGCSCRF |
| | 41BB_2 | including TM | PEEEEGGCELGGSAERVQPLEENVGNAARPRFER |
| | | and IC domain | NKLLLVASVIQGLGLLLCFTYICLHFSALQVSHRYPRI |
| | | with 41BB | QSIKVQFTEYKKEKGFILTSQKEDEIMKVQNNSVIIN |
| | | intracellular | CDGFYLISLKGYFSQEVNISLHYQKDEEPLFQLKKV |
| | | signaling | RSVNSLMVASLTYKDKVYLNVTTDNTSLDDFHVNG |
| | | domain | GELILIHQNPGEFCVL |
| | | separated by | |
| | | GGSA linker | |
| 89 | OX40L- | OX40L ligand | MLGLECGGEEEEPFRCSCGDEEQTTQVPRMFPQK |
| | 41BBrev | binding and TM | FIYLLKKRGRKLGGSAERVQPLEENVGNAARPRFE |
| | | domain with | RNKLLLVASVIQGLGLLLCFTYICLHFSALQVSHRYP |
| | | 41BB | RIQSIKVQFTEYKKEKGFILTSQKEDEIMKVQNNSVII |
| | | intracellular | NCDGFYLISLKGYFSQEVNISLHYQKDEEPLFQLKK |
| | | signaling | VRSVNSLMVASLTYKDKVYLNVTTDNTSLDDFHVN |
| | | domain that is | GGELILIHQNPGEFCVL |
| | | inverted | |
| 90 | CD86- | CD86 WT | MDPQCTMGLSNILFVMAFLLSGAAPLKIQAYFNETA |
| | OX40 | including TM | DLPCQFANSQNQSLSELVVFWQDQENLVLNEVYLG |
| | | and IC domain | KEKFDSVHSKYMGRTSFDSDSWTLRLHNLQIKDKG |
| | | linked to OX40 | LYQCIIHHKKPTGMIRIHQMNSELSVLANFSQPEIVPI |
| | | intracellular | SNITENVYINLTCSSIHGYPEPKKMSVLLRTKNSTIEY |
| | | signaling | DGVMQKSQDNVTELYDVSISLSVSFPDVTSNMTIFC |
| | | domain | ILETDKTRLLSSPFSIELEDPQPPPDHIPWITAVLPTVI |
| | | | ICVMVFCLILWKWKKKKRPRNSYKCGTNTMEREES |
| | | | EQTKKREKIHIPERSDEAQRVFKSSKTSSCDKSDTC |
| | | | FGSGRDQRLPPDAHKPPGGGSFRTPIQEEQADAHS |
| | | | TLAKI |
| 91 | CD86del | CD86 ligand | MDPQCTMGLSNILFVMAFLLSGAAPLKIQAYFNETA |
| | P276- | domain and TM | DLPCQFANSQNQSLSELVVFWQDQENLVLNEVYLG |
| | OX40 | domain, with a | KEKFDSVHSKYMGRTSFDSDSWTLRLHNLQIKDKG |
| | | deletion of | LYQCIIHHKKPTGMIRIHQMNSELSVLANFSQPEIVPI |
| | | amino acids | SNITENVYINLTCSSIHGYPEPKKMSVLLRTKNSTIEY |
| | | 277-329 of | DGVMQKSQDNVTELYDVSISLSVSFPDVTSNMTIFC |
| | | CD86, and an | ILETDKTRLLSSPFSIELEDPQPPPDHIPWITAVLPTVI |
| | | OX40 | ICVMVFCLILWKWKKKKRPGRDQRLPPDAHKPPGG |
| | | intracellular | GSFRTPIQEEQADAHSTLAKI |
| | | signaling | |
| | | domain | |
| 92 | 41BBL | WT 41BBL | YASDASLDPEAPWPPAPRARACRVLPWALVAGLLL |
| | | | LLLLAAACAVFLACPWAVSGARASPGSAASPRLRE |
| | | | GPELSPDDPAGLLDLRQGMFAQLVAQNVLLIDGPLS |
| | | | WYSDPGLAGVSLTGGLSYKEDTKELVVAKAGVYYV |
| | | | FFQLELRRVVAGEGSGSVSLALHLQPLRSAAGAAAL |
| | | | ALTVDLPPASSEARNSAFGFQGRLLHLSAGQRLGV |
| | | | HLHTEARARHAWQLTQGATVLGLFRVTPEIPAGLPS |
| | | | PRSE |
| 93 | 41BBL- | WT 41BBL of | MSKSTGSWALVAGLLLLLLLAAACAVFLACPWAVS |
| | mincyto | which | GARASPGSAASPRLREGPELSPDDPAGLLDLRQGM |
| | | intracellular | FAQLVAQNVLLIDGPLSWYSDPGLAGVSLTGGLSYK |
| | | domain is | EDTKELVVAKAGVYYVFFQLELRRVVAGEGSGSVS |
| | | swapped for | LALHLQPLRSAAGAAALALTVDLPPASSEARNSAFG |
| | | small linker | FQGRLLHLSAGQRLGVHLHTEARARHAWQLTQGA |
| | | MSKSTGS | TVLGLFRVTPEIPAGLPSPRSE |
| 94 | 41BBL13W | WT 41BBL with | MEWALVAGLLLLLLLAAACAVFLACPWAVSGARAS |
| | | deletion of the | PGSAASPRLREGPELSPDDPAGLLDLRQGMFAQLV |
| | | first 12 amino | AQNVLLIDGPLSWYSDPGLAGVSLTGGLSYKEDTKE |
| | | acids | LVVAKAGVYYVFFQLELRRVVAGEGSGSVSLALHL |
| | | containing a | QPLRSAAGAAALALTVDLPPASSEARNSAFGFQGR |
| | | putative casein | LLHLSAGQRLGVHLHTEARARHAWQLTQGATVLGL |
| | | kinase I motif | FRVTPEIPAGLPSPRSE |
| 95 | 41BBL- | 41BBL WT | MLGIKALTSHADAQEEQIPTRFSGGGPPKHADPPLR |
| | OX40rev | including TM | QDRTSGSYASDASLDPEAPWPPAPRARACRVLPW |
| | | and IC domain | ALVAGLLLLLLLAAACAVFLACPWAVSGARASPGSA |
| | | without first 2 | ASPRLREGPELSPDDPAGLLDLRQGMFAQLVAQNV |
| | | amino acids | LLIDGPLSWYSDPGLAGVSLTGGLSYKEDTKELVVA |
| | | with inverted | KAGVYYVFFQLELRRVVAGEGSGSVSLALHLQPLR |
| | | OX40 | SAAGAAALALTVDLPPASSEARNSAFGFQGRLLHLS |
| | | intracellular | AGQRLGVHLHTEARARHAWQLTQGATVLGLFRVTP |
| | | signaling | EIPAGLPSPRSE |
| | | domain | |
| 96 | 41BBL- | 41BBL | MLLLVTSLLLCELPHPAFLLIPDQGMFAQLVAQNVLL |
| | rev extra- | ligand | IDGPLSWYSDPGLAGVSLTGGLSYKEDTKELVVAKA |
| | OX40tm- | binding domain | GVYYVFFQLELRRVVAGEGSGSVSLALHLQPLRSA |
| | cyto | core in a type I | AGAAALALTVDLPPASSEARNSAFGFQGRLLHLSAG |
| | | orientation with | QRLGVHLHTEARARHAWQLTQGATVLGLFRVTPEI |
| | | IgK leader | PAGLPSPRSERLDLLGAPDDPSLEPGERLRPSAAS |
| | | sequence and | GPSARAVAAILGLGLVLGLLGPLAILLALYLLRRDQR |
| | | inverted | LPPDAHKPPGGGSFRTPIQEEQADAHSTLAKI |
| | | extracellular N- | |
| | | terminal part | |
| | | with an OX40 | |
| | | TM and | |
| | | intracellular | |
| | | signaling | |
| | | domain | |
| 97 | 41BBLmincyto- | WT 41BBL EC | MLGRDQRLPPDAHKPPGGGSFRTPIQEEQADAHST |
| | OX40 | and TM linked | LAKITSGSWALVAGLLLLLLLAAACAVFLACPWAVS |
| | | to IC signaling | GARASPGSAASPRLREGPELSPDDPAGLLDLRQGM |
| | | domain of | FAQLVAQNVLLIDGPLSWYSDPGLAGVSLTGGLSYK |
| | | OX40 | EDTKELVVAKAGVYYVFFQLELRRVVAGEGSGSVS |
| | | separated by a | LALHLQPLRSAAGAAALALTVDLPPASSEARNSAFG |
| | | TSGS linker | FQGRLLHLSAGQRLGVHLHTEARARHAWQLTQGA |
| | | | TVLGLFRVTPEIPAGLPSPRSE |
| 98 | 41BBL13W- | WT 41BBL | MEIKALTSHADAQEEQIPTRFSGGGPPKHADPPLRQ |
| | OX40rev | ligand binding, | DRRLLWPPAPRARACRVLPWALVAGLLLLLLLAAAC |
| | | TM and IC | AVFLACPWAVSGARASPGSAASPRLREGPELSPDD |
| | | domain without | PAGLLDLRQGMFAQLVAQNVLLIDGPLSWYSDPGL |
| | | first 12 amino | AGVSLTGGLSYKEDTKELVVAKAGVYYVFFQLELRR |
| | | acids of the IC | VVAGEGSGSVSLALHLQPLRSAAGAAALALTVDLPP |
| | | domain linked | ASSEARNSAFGFQGRLLHLSAGQRLGVHLHTEARA |
| | | to the inverted | RHAWQLTQGATVLGLFRVTPEIPAGLPSPRSE |
| | | OX40 IC | |
| | | signaling | |
| | | domain | |
| 99 | 41BBL- | WT 41BBL EC | MLGIKALTSHADAQEEQIPTRFSGGGPPKHADPPLR |
| | mincyto- | and TM linked | QDRTSGSWALVAGLLLLLLLAAACAVFLACPWAVS |
| | OX40rev | to IC signaling | GARASPGSAASPRLREGPELSPDDPAGLLDLRQGM |
| | | domain of | FAQLVAQNVLLIDGPLSWYSDPGLAGVSLTGGLSYK |
| | | inverted OX40 | EDTKELVVAKAGVYYVFFQLELRRVVAGEGSGSVS |
| | | separated by a | LALHLQPLRSAAGAAALALTVDLPPASSEARNSAFG |
| | | TSGS linker | FQGRLLHLSAGQRLGVHLHTEARARHAWQLTQGA |
| | | | TVLGLFRVTPEIPAGLPSPRSE |
| 100 | 41BBL- | 41BBL WT EC, | MLGIKALTSHADAQEEQIPTRFSGGGPPKHADPPLR |
| | OX40ENrev | TM and IC | QDRRLLYLATSGSYASDASLDPEAPWPPAPRARAC |
| | | without first 2 | RVLPWALVAGLLLLLLLAAACAVFLACPWAVSGARA |
| | | amino acids | SPGSAASPRLREGPELSPDDPAGLLDLRQGMFAQL |
| | | linked to | VAQNVLLIDGPLSWYSDPGLAGVSLTGGLSYKEDTK |
| | | inverted OX40 | ELVVAKAGVYYVFFQLELRRVVAGEGSGSVSLALHL |
| | | enlarged | QPLRSAAGAAALALTVDLPPASSEARNSAFGFQGR |
| | | (including | LLHLSAGQRLGVHLHTEARARHAWQLTQGATVLGL |
| | | amino acids | FRVTPEIPAGLPSPRSE |
| | | ALYLLR) IC | |
| | | signaling | |
| | | domain | |
| 101 | 41BBL- | 41BBL WT EC, | MLGALYLLRRDQRLPPDAHKPPGGGSFRTPIQEEQ |
| | OX40EN | TM and IC | ADAHSTLAKITSGSYASDASLDPEAPWPPAPRARA |
| | | without first 2 | CRVLPWALVAGLLLLLLLAAACAVFLACPWAVSGAR |
| | | amino acids | ASPGSAASPRLREGPELSPDDPAGLLDLRQGMFAQ |
| | | linked to OX40 | LVAQNVLLIDGPLSWYSDPGLAGVSLTGGLSYKEDT |
| | | enlarged | KELVVAKAGVYYVFFQLELRRVVAGEGSGSVSLAL |
| | | (including | HLQPLRSAAGAAALALTVDLPPASSEARNSAFGFQ |
| | | amino acids | GRLLHLSAGQRLGVHLHTEARARHAWQLTQGATVL |
| | | ALYLLR) | GLFRVTPEIPAGLPSPRSE |
| | | intracellular | |
| | | signaling | |
| | | domain | |
| 102 | 41BBLmincyto- | WT 41BBL EC | MLGRDQRLPPDAHKPPGGGSFRTPIQEEQADAHST |
| | 13alink- | and TM linked | LAKISLNGGGGSGGGGSGGGGSGGGGSGGPWAL |
| | OX40 | with a 13 amino | VAGLLLLLLLAAACAVFLACPWAVSGARASPGSAAS |
| | | acid linker to | PRLREGPELSPDDPAGLLDLRQGMFAQLVAQNVLLI |
| | | the OX40 IC | DGPLSWYSDPGLAGVSLTGGLSYKEDTKELVVAKA |
| | | signaling | GVYYVFFQLELRRVVAGEGSGSVSLALHLQPLRSA |
| | | domain | AGAAALALTVDLPPASSEARNSAFGFQGRLLHLSAG |
| | | | QRLGVHLHTEARARHAWQLTQGATVLGLFRVTPEI |
| | | | PAGLPSPRSE |
| 103 | OX40Lmincyto- | OX40L WT EC | MLGLECGGEEEEPFRCSCGDEEQTTQVPRMFPQK |
| | 41BBrev | and TM linked | FIYLLKKRGRKLLLVASVIQGLGLLLCFTYICLHFSAL |
| | | to the inverted | QVSHRYPRIQSIKVQFTEYKKEKGFILTSQKEDEIMK |
| | | IC signaling | VQNNSVIINCDGFYLISLKGYFSQEVNISLHYQKDEE |
| | | domain by a | PLFQLKKVRSVNSLMVASLTYKDKVYLNVTTDNTSL |
| | | short linker | DDFHVNGGELILIHQNPGEFCVL |
| 104 | OX40L | OX40L WT | MERVQPLEENVGNAARPRFERNKLLLVASVIQGLGL |
| | WT | | LLCFTYICLHFSALQVSHRYPRIQSIKVQFTEYKKEK |
| | | | GFILTSQKEDEIMKVQNNSVIINCDGFYLISLKGYFSQ |
| | | | EVNISLHYQKDEEPLFQLKKVRSVNSLMVASLTYKD |
| | | | KVYLNVTTDNTSLDDFHVNGGELILIHQNPGEFCVL |
| 105 | OX40Lmincyto- | OX40L WT, EC | MLGKRGRKKLLYIFKQPFMRPVQTTQEEDGCSCRF |
| | 41BB | and TM linked | PEEEEGGCELGGSLLLVASVIQGLGLLLCFTYICLHF |
| | | to 41BB IC | SALQVSHRYPRIQSIKVQFTEYKKEKGFILTSQKEDE |
| | | signaling | IMKVQNNSVIINCDGFYLISLKGYFSQEVNISLHYQK |
| | | domain by a | DEEPLFQLKKVRSVNSLMVASLTYKDKVYLNVTTDN |
| | | GGS linker | TSLDDFHVNGGELILIHQNPGEFCVL |
| 106 | CD86 WT | CD86 WT | MDPQCTMGLSNILFVMAFLLSGAAPLKIQAYFNETA |
| | | | DLPCQFANSQNQSLSELVVFWQDQENLVLNEVYLG |
| | | | KEKFDSVHSKYMGRTSFDSDSWTLRLHNLQIKDKG |
| | | | LYQCIIHHKKPTGMIRIHQMNSELSVLANFSQPEIVPI |
| | | | SNITENVYINLTCSSIHGYPEPKKMSVLLRTKNSTIEY |
| | | | DGVMQKSQDNVTELYDVSISLSVSFPDVTSNMTIFC |
| | | | ILETDKTRLLSSPFSIELEDPQPPPDHIPWITAVLPTVI |
| | | | ICVMVFCLILWKWKKKKRPRNSYKCGTNTMEREES |
| | | | EQTKKREKIHIPERSDEAQRVFKSSKTSSCDKSDTC |
| | | | F |
| 107 | CD86- | CD86 with a | MDPQCTMGLSNILFVMAFLLSGAAPLKIQAYFNETA |
| | delP276 | deletion of the | DLPCQFANSQNQSLSELVVFWQDQENLVLNEVYLG |
| | | IC part up to the | KEKFDSVHSKYMGRTSFDSDSWTLRLHNLQIKDKG |
| | | point which is | LYQCIIHHKKPTGMIRIHQMNSELSVLANFSQPEIVPI |
| | | shown to be | SNITENVYINLTCSSIHGYPEPKKMSVLLRTKNSTIEY |
| | | involved in | DGVMQKSQDNVTELYDVSISLSVSFPDVTSNMTIFC |
| | | correcT-cellular | ILETDKTRLLSSPFSIELEDPQPPPDHIPWITAVLPTVI |
| | | localization of | ICVMVFCLILWKWKKKKRP |
| | | CD86 (deletion | |
| | | of amino acid | |
| | | 277-329 of | |
| | | CD86) | |
| 108 | CD86mincyto | CD86 WT | MDPQCTMGLSNILFVMAFLLSGAAPLKIQAYFNETA |
| | | without IC | DLPCQFANSQNQSLSELVVFWQDQENLVLNEVYLG |
| | | domain | KEKFDSVHSKYMGRTSFDSDSWTLRLHNLQIKDKG |
| | | | LYQCIIHHKKPTGMIRIHQMNSELSVLANFSQPEIVPI |
| | | | SNITENVYINLTCSSIHGYPEPKKMSVLLRTKNSTIEY |
| | | | DGVMQKSQDNVTELYDVSISLSVSFPDVTSNMTIFC |
| | | | ILETDKTRLLSSPFSIELEDPQPPPDHIPWITAVLPTVI |
| | | | ICVMVFCLILW |
| 109 | CD86mincyto- | CD86 WT EC | MDPQCTMGLSNILFVMAFLLSGAAPLKIQAYFNETA |
| | IL 18RAP | and TM linked | DLPCQFANSQNQSLSELVVFWQDQENLVLNEVYLG |
| | | to IC domain of | KEKFDSVHSKYMGRTSFDSDSWTLRLHNLQIKDKG |
| | | IL18RAP | LYQCIIHHKKPTGMIRIHQMNSELSVLANFSQPEIVPI |
| | | | SNITENVYINLTCSSIHGYPEPKKMSVLLRTKNSTIEY |
| | | | DGVMQKSQDNVTELYDVSISLSVSFPDVTSNMTIFC |
| | | | ILETDKTRLLSSPFSIELEDPQPPPDHIPWITAVLPTVI |
| | | | ICVMVFCLILWSALLYRHWIEIVLLYRTYQSKDQTLG |
| | | | DKKDFDAFVSYAKWSSFPSEATSSLSEEHLALSLFP |
| | | | DVLENKYGYSLCLLERDVAPGGVYAEDIVSIIKRSRR |
| | | | GIFILSPNYVNGPSIFELQAAVNLALDDQTLKLILIKFC |
| | | | YFQEPESLPHLVKKALRVLPTVTWRGLKSVPPNSRF |
| | | | WAKMRYHMPVKNSQGFTWNQLRITSRIFQWKGLS |
| | | | RTETTGRSSQPKEW |
| 110 | CD86- | CD86 WT EC, | MDPQCTMGLSNILFVMAFLLSGAAPLKIQAYFNETA |
| | IL18RAP | TM and IC | DLPCQFANSQNQSLSELVVFWQDQENLVLNEVYLG |
| | | domain linked | KEKFDSVHSKYMGRTSFDSDSWTLRLHNLQIKDKG |
| | | to IL18RAP IC | LYQCIIHHKKPTGMIRIHQMNSELSVLANFSQPEIVPI |
| | | domain | SNITENVYINLTCSSIHGYPEPKKMSVLLRTKNSTIEY |
| | | | DGVMQKSQDNVTELYDVSISLSVSFPDVTSNMTIFC |
| | | | ILETDKTRLLSSPFSIELEDPQPPPDHIPWITAVLPTVI |
| | | | ICVMVFCLILWKWKKKKRPRNSYKCGTNTMEREES |
| | | | EQTKKREKIHIPERSDEAQRVFKSSKTSSCDKSDTC |
| | | | FSALLYRHWIEIVLLYRTYQSKDQTLGDKKDFDAFV |
| | | | SYAKWSSFPSEATSSLSEEHLALSLFPDVLENKYGY |
| | | | SLCLLERDVAPGGVYAEDIVSIIKRSRRGIFILSPNYV |
| | | | NGPSIFELQAAVNLALDDQTLKLILIKFCYFQEPESLP |
| | | | HLVKKALRVLPTVTWRGLKSVPPNSRFWAKMRYH |
| | | | MPVKNSQGFTWNQLRITSRIFQWKGLSRTETTGRS |
| | | | SQPKEW |
| 111 | CD86lgV- | WT 41BBL EC, | MLGRDQRLPPDAHKPPGGGSFRTPIQEEQADAHST |
| | 41BBL- | TM and IC | LAKITSGSYASDASLDPEAPWPPAPRARACRVLPW |
| | OX40 | without first two | ALVAGLLLLLLLAAACAVFLACPWAVSGARASPGSA |
| | | amino acids. | ASPRLREGPELSPDDPAGLLDLRQGMFAQLVAQNV |
| | | An CD86 IgV | LLIDGPLSWYSDPGLAGVSLTGGLSYKEDTKELVVA |
| | | domain was | KAGVYYVFFQLELRRVVAGEGSGSVSLALHLQPLR |
| | | linked with a 30 | SAAGAAALALTVDLPPASSEARNSAFGFQGRLLHLS |
| | | amino acid | AGQRLGVHLHTEARARHAWQLTQGATVLGLFRVTP |
| | | linker to WT | EIPAGLPSPRSESLNGGGGSGGGGSGGGGSGGG |
| | | 41BBL EC | GSGGGGSTSAPLKIQAYFNETADLPCQFANSQNQS |
| | | domain. The | LSELVVFWQDQENLVLNEVYLGKEKFDSVHSKYMG |
| | | OX40 IC | RTSFDSDSWTLRLHNLQIKDKGLYQCIIHHKKPTGMI |
| | | signaling | RIHQMNSELSVLAN |
| | | domain was | |
| | | linked to the IC | |
| | | domain 01 | |
| | | 41BBLby TSGS | |
| | | linker | |
| 112 | RANK | RANK WT | MAPRARRRRPLFALLLLCALLARLQVALQIAPPCTSE |
| | WT | | KHYEHLGRCCNKCEPGKYMSSKCTTTSDSVCLPC |
| | | | GPDEYLDSWNEEDKCLLHKVCDTGKALVAVVAGNS |
| | | | TTPRRCACTAGYHWSQDCECCRRNTECAPGLGAQ |
| | | | HPLQLNKDTVCKPCLAGYFSDAFSSTDKCRPWTNC |
| | | | TFLGKRVEHHGTEKSDAVCSGSRKPPNEPHVYLPG |
| | | | LIILLLFASVALVAAIIFGVCYRKKGKALTANLWHWIN |
| | | | EACGRLSGDKESSGDSCVSTHTANFGQQGACEGV |
| | | | LLLTLEEKTFPEDMCYPDQGGVCQGTCVGGGPYA |
| | | | QGEDARMLSLVSKTEIEEDSFRQMPTEDEYMDRPS |
| | | | QPTDQLLFLTEPGSKSTPPFSEPLEVGENDSLSQCF |
| | | | TGTQSTVGSESCNCTEPLCRTDWTPMSSENYLQK |
| | | | EVDSGHCPHWAASPSPNWADVCTGCRNPPGEDC |
| | | | EPLVGSPKRGPLPQCAYGMGLPPEEEASRTEARD |
| | | | QPEDGADGRLPSSARAGAGSGSSPGGQSPASGNV |
| | | | TGNSNSTFISSGQVMNFKGDIIVVYVSQTSQEGAAA |
| | | | AAEPMGRPVQEETLARRDSFAGNGPRFPDPCGGP |
| | | | EGLREPEKASRPVQEQGGAKA |
| 113 | RANKmincyto | RANK WT | MAPRARRRRPLFALLLLCALLARLQVALQIAPPCTSE |
| | | without the IC | KHYEHLGRCCNKCEPGKYMSSKCTTTSDSVCLPC |
| | | domain | GPDEYLDSWNEEDKCLLHKVCDTGKALVAVVAGNS |
| | | | TTPRRCACTAGYHWSQDCECCRRNTECAPGLGAQ |
| | | | HPLQLNKDTVCKPCLAGYFSDAFSSTDKCRPWTNC |
| | | | TFLGKRVEHHGTEKSDAVCSGSRKPPNEPHVYLPG |
| | | | LIILLLFASVALVAAIIFGV |
| 114 | RANK- | EC domain of | MAPRARRRRPLFALLLLCALLARLQVALQIAPPCTSE |
| | OX40 | RANK linked to | KHYEHLGRCCNKCEPGKYMSSKCTTTSDSVCLPC |
| | | TM and IC of | GPDEYLDSWNEEDKCLLHKVCDTGKALVAVVAGNS |
| | | OX40 | TTPRRCACTAGYHWSQDCECCRRNTECAPGLGAQ |
| | | | HPLQLNKDTVCKPCLAGYFSDAFSSTDKCRPWTNC |
| | | | TFLGKRVEHHGTEKSDAVCSGSRKPPNEPHVYLPV |
| | | | AAILGLGLVLGLLGPLAILLALYLLRRDQRLPPDAHKP |
| | | | PGGGSFRTPIQEEQADAHSTLAKI |
| 115 | RANK- | EC domain of | MAPRARRRRPLFALLLLCALLARLQVALQIAPPCTSE |
| | 41BB | RANK linked to | KHYEHLGRCCNKCEPGKYMSSKCTTTSDSVCLPC |
| | | TM and IC | GPDEYLDSWNEEDKCLLHKVCDTGKALVAVVAGNS |
| | | domain of 41BB | TTPRRCACTAGYHWSQDCECCRRNTECAPGLGAQ |
| | | | HPLQLNKDTVCKPCLAGYFSDAFSSTDKCRPWTNC |
| | | | TFLGKRVEHHGTEKSDAVCSGSRKPPNEPHVYLPII |
| | | | SFFLALTSTALLFLLFFLTLRFSVVKRGRKKLLYIFKQ |
| | | | PFMRPVQTTQEEDGCSCRFPEEEEGGCEL |
| 116 | RANK- | EC domain of | MAPRARRRRPLFALLLLCALLARLQVALQIAPPCTSE |
| | IL18RAP | RANK linked to | KHYEHLGRCCNKCEPGKYMSSKCTTTSDSVCLPC |
| | | the TM and IC | GPDEYLDSWNEEDKCLLHKVCDTGKALVAVVAGNS |
| | | domain of | TTPRRCACTAGYHWSQDCECCRRNTECAPGLGAQ |
| | | IL 18RAP | HPLQLNKDTVCKPCLAGYFSDAFSSTDKCRPWTNC |
| | | | TFLGKRVEHHGTEKSDAVCSGSRKPPNEPHVYLPG |
| | | | VVLLYILLGTIGTLVAVLAASALLYRHWIEIVLLYRTYQ |
| | | | SKDQTLGDKKDFDAFVSYAKWSSFPSEATSSLSEE |
| | | | HLALSLFPDVLENKYGYSLCLLERDVAPGGVYAEDI |
| | | | VSIIKRSRRGIFILSPNYVNGPSIFELQAAVNLALDDQ |
| | | | TLKLILIKFCYFQEPESLPHLVKKALRVLPTVTWRGL |
| | | | KSVPPNSRFWAKMRYHMPVKNSQGFTWNQLRITS |
| | | | RIFQWKGLSRTETTGRSSQPKEW |
| 117 | OX40WT | WT OX40L | MERNKLLLVASVIQGLGLLLCFTYICLHFSALQVSHR |
| | mincyto | without IC | YPRIQSIKVQFTEYKKEKGFILTSQKEDEIMKVQNNS |
| | | domain | VIINCDGFYLISLKGYFSQEVNISLHYQKDEEPLFQLK |
| | | (deletion of first | KVRSVNSLMVASLTYKDKVYLNVTTDNTSLDDFHVN |
| | | 18 amino acids) | GGELILIHQNPGEFCVL |
| 118 | Q8 | CD34 epitope | MGLVRRGARAGPRIPRGWTALCLLSLLPSGFMAEL |
| | | linked CD8 | PTQGTFSNVSTNVSPAKPTTTPAPRPPTPAPTIASQ |
| | | stalk, TM and | PLSLRPEACRPAAGGAVHTRGLDFACDIYIWAPLAG |
| | | IC domain; | TCGVLLLSLVITLYCNHRNRRRVCKCPRPVV |
| | | tricistronic | |
| | | control | |
| 119 | eGFP | Enhanced GFP, | MVSKGEELFTGVVPILVELDGDVNGHKFSVSGEGE |
| | | tricistronic | GDATYGKLTLKFICTTGKLPVPWPTLVTTLTYGVQC |
| | | control protein | FSRYPDHMKQHDFFKSAMPEGYVQERTIFFKDDGN |
| | | without a | YKTRAEVKFEGDTLVNRIELKGIDFKEDGNILGHKLE |
| | | cellular function | YNYNSHNVYIMADKQKNGIKVNFKIRHNIEDGSVQL |
| | | | ADHYQQNTPIGDGPVLLPDNHYLSTQSALSKDPNE |
| | | | KRDHMVLLEFVTAAGITLGMDELYK |
| 124 | 41BBL- | WT 41BBL | MLGRDQRLPPDAHKPPGGGSFRTPIQEEQAD |
| | 13W- | ligand | AHSTLAKITSGS |
| | OX40 | binding, TM | WPPAPRARACRVLPWALVAGLLLLLLLAAACA |
| | | and IC | VFLACPWAVSGARASPGSAASPRLREGPELS |
| | | domain | PDDPAGLLDLRQGMFAQLVAQNVLLIDGPLSW |
| | | without first 12 | YSDPGLAGVSLTGGLSYKEDTKELVVAKAGVY |
| | | amino acids of | YVFFQLELRRVVAGEGSGSVSLALHLQPLRSA |
| | | the IC domain | AGAAALALTVDLPPASSEARNSAFGFQGRLLH |
| | | linked to the | LSAGQRLGVHLHTEARARHAWQLTQGATVLG |
| | | OX40 IC | LFRVTPEIPAGLPSPRSE |
| | | signaling | |
| | | domain | |
| 125 | 41BBL- | Extracellular | MLGVLHTPDQGQLEQLSLYADTNLPLLQTVKDRELV |
| | OX40- | 41BBL ligand | ELPSIEPALGGPSFSSSPFPSSLWKQVDGGHESSL |
| | IL2RB | domain, a | QSFFKSPDPTNCKLVKKLWPGTNRCNGSGRDQRL |
| | | transmembrane | PPDAHKPPGGGSFRTPIQEEQADAHSTLAKITSGSY |
| | | domain | ASDASLDPEAPWPPAPRARACRVLPWALVAGLLLL |
| | | from 41BBL, | LLLAAACAVFLACPWAVSGARASPGSAASPRLREG |
| | | and an OX40 | PELSPDDPAGLLDLRQGMFAQLVAQNVLLIDGPLS |
| | | and IL2RB | WYSDPGLAGVSLTGGLSYKEDTKELVVAKAGVYYV |
| | | heterologous | FFQLELRRVVAGEGSGSVSLALHLQPLRSAAGAAAL |
| | | intracellular | ALTVDLPPASSEARNSAFGFQGRLLHLSAGQRLGV |
| | | signaling | HLHTEARARHAWQLTQGATVLGLFRVTPEIPAGLPS |
| | | domain | PRSE |
| 126 | CD70 | WT CD70 | MLGPEEGSGCSVRRRPYGCVLRAALVPLVAG |
| | | | LVICLVVCIQRFAQAQQQLPLESLGWDVAELQ |
| | | | LNHTGPQQDPRLYWQGGPALGRSFLHGPELD |
| | | | KGQLRIHRDGIYMVHIQVTLAICSSTTASRHHP |
| | | | TTLAVGICSPASRSISLLRLSFHQGCTIASQRLT |
| | | | PLARGDTLCTNLTGTLLPSRNTDETFFGVQWV |
| | | | RP |
| 127 | CD70mincyto | CD70 EC and | MLGCVLRAALVPLVAGLVICLVVCIQRFAQAQQ |
| | | TM | QLPLESLGWDVAELQLNHTGPQQDPRLYWQ |
| | | | GGPALGRSFLHGPELDKGQLRIHRDGIYMVHI |
| | | | QVTLAICSSTTASRHHPTTLAVGICSPASRSISL |
| | | | LRLSFHQGCTIASQRLTPLARGDTLCTNLTGTL |
| | | | LPSRNTDETFFGVQWVRP |
| 128 | CD70mincyto- | CD70 EC and | MLGRDQRLPPDAHKPPGGGSFRTPIQEEQAD |
| | OX40 | TM linked to | AHSTLAKITSGSGCVLRAALVPLVAGLVICLVV |
| | | IC signaling | CIQRFAQAQQQLPLESLGWDVAELQLNHTGP |
| | | domain of | QQDPRLYWQGGPALGRSFLHGPELDKGQLRI |
| | | OX40 | HRDGIYMVHIQVTLAICSSTTASRHHPTTLAVGI |
| | | separated by | CSPASRSISLLRLSFHQGCTIASQRLTPLARGD |
| | | a TSGS linker | TLCTNLTGTLLPSRNTDETFFGVQWVRP |
| 129 | CD70- | CD70 EC | MLGRDQRLPPDAHKPPGGGSFRTPIQEEQAD |
| | 41BBL- | linked to | AHSTLAKITSGSYASDASLDPEAPWPPAPRAR |
| | OX40 | 41BBL TM | ACRVLPWALVAGLLLLLLLAAACAVFLQRFAQ |
| | | and IC with IC | AQQQLPLESLGWDVAELQLNHTGPQQDPRLY |
| | | signaling | WQGGPALGRSFLHGPELDKGQLRIHRDGIYM |
| | | domain of | VHIQVTLAICSSTTASRHHPTTLAVGICSPASRS |
| | | OX40 | ISLLRLSFHQGCTIASQRLTPLARGDTLCTNLT |
| | | separated by | GTLLPSRNTDETFFGVQWVRP |
| | | a TSGS linker | |

A chimeric protein can comprise, consist essentially of, or consist of an amino acid sequence with at least a minimal level of sequence identity compared to an amino acid sequence disclosed herein. For example, a chimeric protein can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to an amino acid sequence disclosed herein, for example, any one of SEQ ID NOs: 38, 83-91, 95-103, 105, 109-111, 114-116, 124-125, or 128-129.

In an embodiment, the chimeric bidirectional signaling transmembrane protein identified under a) is represented by an amino acid sequence having at least 80% identity with SEQ ID NO: 83 as identified in table 5. In an embodiment, the sequence identity may be at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, such chimeric bidirectional signaling transmembrane protein having at least a minimal level of sequence identity compared to a given amino acid sequence is functional and therefore encompassed by the invention as long as this chimeric protein is able to transduce at least two inducible intracellular signals and/or is able to induce an improvement of a biological parameter and/or function in a T-cell expressing it as earlier defined herein and/or is able to induce an improvement of a biological parameter and/or function induced by such a cell especially when such cell is used as exemplified herein. The transduction of these at least two inducible intracellular signals should be detectable using an assay known to the skilled person. Such biological parameter and/or function in a T-cell of the invention that may be further improved by the expression of a chimeric protein, may be the enhanced cellular proliferation (expansion), enhanced cellular survival, and greater magnitude and persistence of immune effector functions, such as anti-tumour or anti-infective response. The anti-tumour or anti-infective response may be assessed as earlier defined herein. The cytotoxicity and production of inflammatory mediators may be assessed. The wording “target biological outcome” or “biological outcome” may be replaced by “biological parameter”.

One or multiple biological functions and/or parameters of the cell may be modulated/improved. Multiple biological functions and/or parameters may be modulated, for example, any combination of induced or reduced biological functions and/or parameters that contributes to a target biological outcome such as an anti-tumour or anti-infective response. In this context, a target biological outcome may be the treatment, cure of a cancer or an infection. For example, multiple biological functions can be induced in the T-cell and/or a biological function can be induced and another one can be reduced.

Examples of suitable assays are western blotting, luminescence reporter or FACS assays. The improvement of a biological parameter and/or function should also be detectable using an assay known to the skilled person. Depending on the parameter and/or function, the skilled person would know which assay may be used.

The cellular proliferation (expansion), and/or survival of the T-cells may be assessed using any technique known to the skilled person. As an example, for assessment of expansion, T-cells may be optionally stimulated with anti-CD3/CD28 polymeric nanomatrix beads, in the presence of IL-7 and IL-15. This may be performed using commercially available kits as discussed earlier herein. The skilled person may measure the T-cell number prior to and post-stimulation and thus determine the proliferative ability of the cells. Alternatively, expansion may be monitored via e.g., cell trace violet (or any other suitable dye) dilution when T-cell are stained at the start of a proliferative assay.

Survival of T-cells may, for example, be monitored based on staining for various markers including, but not limited to CD4, CD8a, CD3, αβTCR, γδTCR, 4-1 BB, OX40, PD-1, TIM-3, LAG-3, 4-1BBL, OX40L, CD86, CD107a and CD69, for example staining with fluorescent-labeled antibodies targeting these markers in combination with flow cytometry.

Survival of T-cells may, for example, be monitored based on any cell viability assay known to the skilled person, many of which are commercially available (see for example the assays offered by ThermoFisher Scientific, WA, MA, USA). Non-limiting examples of cell viability assays involve the use of dyes such as calcein AM, ethidium-homodimer-1, SYTOX Deep Red, DiOC 19(3), propidium iodide, SYBR 14, SYTO 10, green ethidium homodimer-2, SYTOX Green, C-12 resazurin, BOBO-3 iodide, DAPI, and others. By knowing a starting number of T-cells, the skilled person may monitor their survival by measuring the number of viable cells over time.

In some embodiments, expansion of a T-cell is increased by at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, at least 10-fold, or more, relative to an otherwise comparable T-cell not expressing the chimeric bidirectional signaling transmembrane protein.

In some embodiments, survival of a T-cell is increased by at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, at least 10-fold, or more, relative to an otherwise comparable T-cell not expressing the chimeric bidirection signaling transmembrane protein. Survival may be measured over a defined period, for example over about 1 day, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, or about 10 weeks.

In some embodiments, a chimeric protein can comprise, consist essentially of, or consist of an amino acid sequence that is a wild type protein amino acid sequence or any other amino acid sequence disclosed herein, for example, any one of SEQ ID NOs: 38, 83-91, 95-103, 105, 109-111, 114-116, 124-125, or 128-129.

A chimeric protein can comprise an amino acid sequence with one or more amino acid insertions, deletions, or substitutions compared to an amino acid sequence disclosed herein.

For example, a chimeric protein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to an amino acid sequence disclosed herein, for example, any one of SEQ ID NOs: 38, 83-91, 95-103, 105, 109-111, 114-116, 124-125, or 128-129.

In some embodiments, a chimeric protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to an amino acid sequence disclosed herein, for example, any one of SEQ ID NOs: 38, 83-91, 95-103, 105, 109-111, 114-116, 124-125, or 128-129.

In some embodiments, a chimeric protein comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to an amino acid sequence disclosed herein, for example, any one of SEQ ID NOs: 38, 83-91, 95-103, 105, 109-111,114-116, 124-125, or 128-129.

The one or more insertions can be at the N-terminus, C-terminus, within the amino acid sequence, or a combination thereof. The one or more insertions can be contiguous, non-contiguous, or a combination thereof.

In some embodiments, a chimeric protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to an amino acid sequence disclosed herein, for example, any one of SEQ ID NOs: 38, 83-91, 95-103, 105, 109-111, 114-116, 124-125, or 128-129.

In some embodiments, a chimeric protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to an amino acid sequence disclosed herein, for example, any one of SEQ ID NOs: 38, 83-91, 95-103, 105, 109-111, 114-116, 124-125, or 128-129.

In some embodiments, a chimeric protein comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to an amino acid sequence disclosed herein, for example, any one of SEQ ID NOs: 38, 83-91, 95-103, 105, 109-111,114-116, 124-125, or 128-129.

The one or more deletions can be at the N-terminus, C-terminus, within the amino acid sequence, or a combination thereof. The one or more deletions can be contiguous, non-contiguous, or a combination thereof.

In some embodiments, a chimeric protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to an amino acid sequence disclosed herein, for example, any one of SEQ ID NOs: 38, 83-91, 95-103, 105, 109-111, 114-116, 124-125, or 128-129.

In some embodiments, a chimeric protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to an amino acid sequence disclosed herein, for example, any one of SEQ ID NOs: 38, 83-91, 95-103, 105, 109-111, 114-116, 124-125, or 128-129.

In some embodiments, a chimeric protein comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to an amino acid sequence disclosed herein, for example, any one of SEQ ID NOs: 38, 83-91, 95-103, 105, 109-111, 114-116, 124-125, or 128-129.

The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

Certain chimeric proteins (or chimeric bidirectional signaling transmembrane protein) disclosed herein combine an amino acid sequence from a type I transmembrane protein with an amino acid sequence from a type II transmembrane protein. In some embodiments, such chimeric proteins exhibit surprising and unexpected effects, as type I and type II transmembrane proteins cannot be readily combined into a functional protein. For example, many attempts to fuse an amino acid sequence from a type I transmembrane protein to an amino acid sequence from type II transmembrane protein fail to yield a functional protein, for example, due to an altered N-terminal or C-terminal location of one of the amino acid sequences, inability of the resulting protein to adopt a functional conformation, tertiary structure, transmembrane orientation, or a combination thereof.

In some embodiments, the extracellular ligand domain comprises an amino acid sequence that is from or derived from a type I transmembrane protein, and the heterologous intracellular signaling domain comprises an amino acid sequence that is from or derived from a type II transmembrane protein.

In some examples provided herein, the extracellular ligand domain comprises an amino acid sequence that is from or derived from a type II transmembrane protein, and the heterologous intracellular signaling domain comprises an amino acid sequence that is from or derived from a type I transmembrane protein (for example, an extracellular ligand domain from 41 BBL, and an intracellular signaling domain from OX40).

In some embodiments, part or all of an extracellular ligand domain and/or a heterologous intracellular signaling domain of a chimeric bidirectional signaling transmembrane protein comprises an amino acid sequence that is inverted compared to a wild type amino acid sequence (i.e. expressed as a retro-protein). In some embodiments, such chimeric bidirectional signaling transmembrane protein exhibit surprising and unexpected effects, as in many cases retro-proteins do not retain the functionality of the parent protein, e.g., due to a failure to adopt a functional conformation and/or tertiary structure.

In some embodiments, a chimeric bidirectional signaling transmembrane protein combines an amino acid sequence from a type I transmembrane protein with an amino acid sequence from a type II transmembrane protein, and contains at least one amino acid sequence that is inverted compared to a wild type amino acid sequence. Functionality of such a chimeric protein can be surprising and unexpected based on a lack of expectation of success combining sequences from type I and type II transmembrane proteins into a functioning fusion protein, and a lack of expectation of success in obtaining a functional retro-protein domain.

In an embodiment, an extracellular ligand domain is a tumour necrosis factor superfamily member or a molecule derived thereof and is derived from a type II transmembrane protein and is therefore a type II molecule.

In an embodiment, an extracellular ligand domain is an immunoglobulin superfamily member or is derived thereof and is derived from a type I transmembrane protein and is therefore a type I molecule.

In an embodiment, the T-cell, preferably γδT-cell or αβT-cell, more preferably αβT-cell, comprises (preferably expresses) a γδTCR or part thereof comprising a CDR3 region, comprising (A or B or C) and a chimeric protein wherein
A:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7 and/or 9, preferably at least 70% sequence identity with amino acid sequence SEQ ID NO: 7, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10 and/or 12, or with SEQ ID NO: 10, 12, and/or 131, preferably at least 85% sequence identity with amino acid sequence SEQ ID NO: 10,
B:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 13 and/or 15, or with amino acid sequence SEQ ID NO: 13, 15, 142, and/or 153, preferably at least 70% sequence identity with amino acid sequence SEQ ID NO: 13, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 16 and/or 18, or with amino acid sequence SEQ ID NO: 16, 18, 133, 143, 144, 154, 155, and/or 162, preferably at least 85% sequence identity with amino acid sequence SEQ ID NO: 16,
C:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 19 and/or 21, preferably at least 70% sequence identity with amino acid sequence SEQ ID NO: 19, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 22 and/or 24, or with amino acid sequence SEQ ID NO: 22, 24, and/or 135, preferably at least 85% sequence identity with amino acid sequence SEQ ID NO: 22,
      and
      wherein the chimeric protein has
its extracellular ligand domain comprising an amino acid sequence from 41BBL, OX40L, CD86, or RANK, and
its heterologous intracellular signaling domain comprising an amino acid sequence from OX40, 41BB, NKp80, or IL18RAP.

In an embodiment, the T-cell, preferably γδT-cell or αβT-cell, more preferably αβT-cell, comprises (preferably expresses) a γδTCR or part thereof comprising a CDR3 region, comprising (A or B or C) and a chimeric protein wherein
A:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 7 and/or 9, preferably at least 70% sequence identity with amino acid sequence SEQ ID NO: 7, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 10 and/or 12, or with amino acid sequence SEQ ID NO: 10, 12. and/or 131, preferably at least 85% sequence identity with amino acid sequence SEQ ID NO: 10,
B:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 13 and/or 15, or with amino acid sequence SEQ ID NO: 13, 15, 142, and/or 153, preferably at least 70% sequence identity with amino acid sequence SEQ ID NO: 13, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 16 and/or 18, or with amino acid sequence SEQ ID NO: 16, 18, 133, 143, 144, 154, 155, and/or 162, preferably at least 85% sequence identity with amino acid sequence SEQ ID NO: 16,
C:
   a δT-cell receptor chain or part thereof comprising a CDR3 region, said δT-cell receptor chain or part thereof being represented by an amino acid sequence, said amino acid sequence comprising at least 60% sequence identity or similarity with amino acid sequence SEQ ID NO: 19 and/or 21, preferably at least 70% sequence identity with amino acid sequence SEQ ID NO: 19, and/or
   a γT-cell receptor chain or part thereof comprising a CDR3 region, said γT-cell receptor chain or part thereof being represented by an amino acid sequence, said amino acid sequence comprising at least 80% sequence identity or similarity with amino acid sequence SEQ ID NO: 22 and/or 24, or with amino acid sequence SEQ ID NO: 22, 24, and/or 135, preferably at least 85% sequence identity with amino acid sequence SEQ ID NO: 22,
      and
      wherein the chimeric protein has
its extracellular ligand domain comprising an amino acid sequence from 41BBL, OX40L, CD86, RANK, or CD70, and
its heterologous intracellular signaling domain comprising an amino acid sequence from OX40, 41BB, NKp80, IL18RAP, or IL2RB.

Preferably, the chimeric bidirectional signaling transmembrane protein comprises the extracellular ligand domain that comprises an amino acid sequence from 41BBL and the heterologous intracellular signaling domain that comprises an amino acid sequence from OX40, preferably wherein the extracellular ligand domain is from or is derived from a type II transmembrane protein 41 BBL and the heterologous intracellular signaling domain is from or is derived from a type I transmembrane protein OX40 (type a).

More preferably, the chimeric bidirectional signaling transmembrane protein identified under a) is represented by an amino acid sequence having at least 80% identity with SEQ ID NO: 38, 83, 84, 95, 96, 97, 98, 99, 100, 101, or 102 as identified in table 5. More preferably, the chimeric bidirectional signaling transmembrane protein identified under a) is represented by an amino acid sequence having at least 80% identity with SEQ ID NO: 83 as identified in table 5. In an embodiment, the sequence identity may be at least 81%, 82%, 83%, 84%, 85%, 86%, 87%, 88%, 89%, 90%, 91%, 92%, 93%, 94%, 95%, 96%, 97%, 98%, 99% or 100%.

In an embodiment, a polynucleotide (comprising a polynucleotide encoding the γδTCR identified herein and a polynucleotide encoding the chimeric protein) is introduced into a T-cell utilizing a vector, electroporated into said cell, or otherwise introduced into said cell to generate a T-cell of the invention. In some embodiments, a bicistronic vector is used comprising a polynucleotide encoding the γδTCR. In another embodiment, two different vectors are utilized: one vector comprises a polynucleotide encoding the γδTCR and the second vector comprises a polynucleotide encoding the chimeric protein.

In yet another embodiment, a tricistronic vector system can be employed to deliver a polynucleotide encoding a γδTCR, and a polynucleotide encoding the chimeric protein. A bicistronic or tricistronic vector can be organized in various configurations such that the polynucleotide encoding the gamma chain of the γδTCR, the polynucleotide encoding the delta chain of the γδTCR and the polynucleotide encoding the chimeric protein are delivered at any position in the vector.

In an embodiment, the vector comprises:
the polynucleotide sequence encoding a gamma-chain of the γδTCR,
the polynucleotide that encodes the chimeric protein and
the polynucleotide sequence encoding a delta-chain of the γδTCR,

Each of these polynucleotide sequences being operably linked to each other.

### Therapy

A δT-cell receptor chain or a part thereof, a γT-cell receptor chain or a part thereof, a γδTCR or a part thereof, a conjugate, a nucleic acid molecule, a nucleic acid construct, a vector, a cell, a population of cells all as defined earlier herein are preferably for use as a medicament. In an embodiment, a δT-cell receptor chain or part thereof is a δ1T-cell or a δ3T-cell receptor chain or part thereof. In an embodiment, a γT-cell receptor chain or part thereof is a γ3T-cell, γ4T-cell, or a γ9T-cell receptor chain or part thereof. The medicament is preferably for the prevention, suppression, treatment of cancer or an infection. Accordingly the invention also relates to a composition, preferably a pharmaceutical composition comprising a δT-cell receptor chain or a part thereof, a γT-cell receptor chain or a part thereof, a γδTCR or a part thereof, a conjugate, a nucleic acid molecule, a nucleic acid construct, a vector, a cell or a population of cells all as defined earlier herein.

In a further aspect, the invention relates to a method for preventing, treating, regressing, curing and/or delaying cancer or an infection in a subject wherein a δT-cell receptor chain or a part thereof, a γT-cell receptor chain or a part thereof, a conjugate, a nucleic acid molecule, a nucleic acid construct, a vector, a cell or a population of cells all as defined earlier herein are administered to said subject. A preferred subject is a human being.

In a further aspect, the invention relates to a use of a δT-cell receptor chain or a part thereof, a γT-cell receptor chain, or a part thereof, a conjugate, a nucleic acid molecule a nucleic acid construct, a vector, a cell or a population of cells all as defined earlier herein for the manufacture of a medicament for preventing, treating, regressing, curing and/or delaying cancer in a subject. A preferred subject is a human being.

Each of these molecules or cell have been earlier defined herein. In an embodiment the medicament is a composition. A preferred composition is a pharmaceutical composition. In an embodiment, the medicament is for preventing, treating, regressing, curing and/or delaying cancer or an infection.

A subject in need thereof can have a disorder, for example, a cancer or an infection. In some cases, the cancer is a metastatic cancer. In other cases, the cancer is a relapsed or refractory cancer. In some cases, a cancer is a solid tumour or a hematologic malignancy. In some instances, the cancer is a solid tumour. In other instances, the cancer is a hematologic malignancy. In some embodiments, a cancer may be a liquid cancer. In some embodiments, a cancer may be Acute myeloid leukemia (AML). In some embodiments, a cancer may be Multiple Myeloma (MM). In some embodiments, a cancer may be a solid cancer. In some embodiments, a cancer may be an ovarian cancer. In some embodiments, a cancer may be a breast cancer. In some embodiments, a cancer may be a colon cancer. In some embodiments, a cancer may be a kidney cancer. In some embodiments, a cancer may be a renal cancer. In some embodiments, a cancer may be a skin cancer, for example melanoma. In some embodiments, a cancer may be a lung cancer. In some cases, a subject has an infection as described earlier herein.

The general part dedicated to the definitions provides more information as to the therapeutic aspect of the invention, especially the formulation and administration mode of the medicament.

### General Part Dedicated to Definitions

### Polypeptide/Nucleic Acid

A “wild type” protein/polypeptide amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide.

An amino acid sequence that is “derived from” a wild type sequence or other amino acid sequence disclosed herein can refer to an amino acid sequence that differs by one or more amino acids compared to the reference amino acid sequence, for example, containing one or more amino acid insertions, deletions, or substitutions as disclosed herein.

In the context of the invention, a polypeptide is represented by an amino acid sequence. Preferred polypeptides are δT-cell (or γT-cell) receptor chains or parts thereof or a γδTCR or parts thereof which mediates an anti-tumour or an anti-infective response as explained herein. A part of a δT-cell (or γT-cell) receptor chain or of a γδTCR may mean a functional part thereof. In the context of the invention, a nucleic acid molecule as a nucleic acid molecule encoding such a δT-cell (or γT-cell) receptor chain or part thereof or a γδTCR or a part thereof is represented by a nucleic acid or nucleotide sequence which encodes such a polypeptide. A nucleic acid molecule may comprise a regulatory region.

It is to be understood that each nucleic acid molecule or polypeptide or construct as identified herein by a given Sequence Identity Number (SEQ ID NO) is not limited to this specific sequence as disclosed. Throughout this application, each time one refers to a specific nucleotide sequence SEQ ID NO (take SEQ ID NO: X as example) encoding a given polypeptide, one may replace it by:
i. a nucleotide sequence comprising a nucleotide sequence that has at least 60% or at least 80% sequence identity with SEQ ID NO: X;
ii. a nucleotide sequences the complementary strand of which hybridizes to a nucleic acid molecule of sequence of (i);
iii. a nucleotide sequence the sequence of which differs from the sequence of a nucleic acid molecule of (i) or (ii) due to the degeneracy of the genetic code; or,
iv. a nucleotide sequence that encodes an amino acid sequence that has at least 60% or at least 80% amino acid identity or similarity with an amino acid sequence encoded by a nucleotide sequence SEQ ID NO: X.

Throughout this application, each time one refers to a specific amino acid sequence SEQ ID NO (take SEQ ID NO: Y as example), one may replace it by: a polypeptide comprising an amino acid sequence that has at least 60% or at least 80% sequence identity or similarity with amino acid sequence SEQ ID NO: Y.

In the context of the application, the minimum identity or similarity in relation with a δT-cell receptor chain or part thereof may mean an identity or a similarity of at least 60%, of at least 70%, or more.

In the context of the application, the minimum identity or similarity in relation with a γT-cell receptor chain or part thereof may mean an identity or a similarity of at least 80%, of at least 85%, or more.

Each nucleotide sequence or amino acid sequence described herein by virtue of its identity or similarity percentage (e.g. at least 60%) with a given nucleotide sequence or amino acid sequence respectively has in a further preferred embodiment an identity or a similarity of at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity or similarity with the given nucleotide or amino acid sequence respectively. In a preferred embodiment, sequence identity or similarity is determined by comparing the whole length of the sequences as identified herein. Unless otherwise indicated herein, identity or similarity with a given SEQ ID NO means identity or similarity based on the full length of said sequence (i.e. over its whole length or as a whole).

### Sequence Identity

“Sequence identity” is herein defined as a relationship between two or more amino acid (polypeptide or protein) sequences or two or more nucleic acid (polynucleotide) sequences, as determined by comparing the sequences. The identity between two amino acid or two nucleic acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. “Similarity” between two amino acid sequences is determined by comparing the amino acid sequence and its conserved amino acid substitutes of one polypeptide to the sequence of a second polypeptide. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, |
| | and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, P |
| formation | and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; IIe to Leu or Val; Leu to IIe or Val; Lys to Arg; Gln or Glu; Met to Leu or lie; Phe to Met, Leu or Tyr; Ser to Thr; Thr to Ser; Trp to Tyr; Tyr to Trp or Phe; and, Val to IIe or Leu.

### Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host cell. For example, to suit the codon preference of mammalian, insect, plant, or microbial cells, preferably human cells. Codon optimization also eliminates elements that potentially impact negatively RNA stability and/or translation (e. g. termination sequences, TATA boxes, splice sites, ribosomal entry sites, repetitive and/or GC rich sequences and RNA secondary structures or instability motifs). Codon optimization may be done according to standard methods available to skilled person.

### Antigen

An “antigen” is a molecule or molecular structure that an antigen receptor or an antigen-binding protein can recognize (for example, bind to). An antigen can be or can comprise, for example, a peptide, a polypeptide, a carbohydrate, a chemical, a moiety, a non-peptide antigen, a phosphoantigen, a tumour-associated antigen, a neoantigen, a tumour microenvironment antigen, a microbial antigen, a viral antigen, a bacterial antigen, an autoantigen, a glycan-based antigen, a peptide-based antigen, a lipid-based antigen, or any combination thereof. In some embodiments, an antigen is capable of inducing an immune response. In some examples, an antigen binds to an antigen receptor or antigen-binding protein, or induces an immune response, when present in a complex e.g., presented by MHC. In some cases, an antigen adopts a certain conformation in order to bind to an antigen receptor or antigen-binding protein, and/or to induce an immune response, e.g., adopts a conformation in response to the presence or absence of one or more metabolites. Antigen can refer to a whole target molecule, a whole complex, a or a fragment of a target molecule or complex that binds to an antigen receptor or an antigen-binding protein. Antigen receptors that recognize antigens include γδTCR disclosed herein and other receptors, such as endogenous T-cell receptors.

### Conjugate

A polypeptide comprising a δT-cell (or γT-cell) receptor chain, or γδTCR or part thereof which mediates an anti-tumour or anti-infective response as explained herein may be coupled or linked to an agent to form a conjugate. The agent may be selected from the group consisting of a diagnostic agent, a therapeutic agent, an anti-cancer agent, a chemical, a nanoparticle, a chemotherapeutic agent or a fluorochrome.

### Gene or Coding Sequence

“Gene” or “coding sequence” or “nucleic acid” or “nucleic” refers to a DNA or RNA region (the transcribed region) which “encodes” a particular polypeptide such as a δT-cell receptor or a γT-cell receptor or a γδTCR or parts thereof. A coding sequence is transcribed (DNA) and translated (RNA) into a polypeptide when placed under the control of an appropriate regulatory region, such as a promoter. A gene may comprise several operably linked fragments, such as a promoter, a 5′ leader sequence, an intron, a coding sequence and a 3′nontranslated sequence, comprising a polyadenylation site or a signal sequence. A chimeric or recombinant gene (such as the one encoding a δTCR or γTCR chain or a γδTCR comprising the polypeptide as identified herein and operably linked to a promoter) is a gene not normally found in nature, such as a gene in which for example the promoter is not associated in nature with part or all of the transcribed DNA region. “Expression of a gene” refers to the process wherein a gene is transcribed into an RNA and/or translated into an active protein.

### Promoter

As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is preferentially active in specific types of differentiated cells/tissues, such as preferably a T-cell. A preferred promoter is the MSCV promoter, an example of which is represented by SEQ ID NO: 120.

### Operably Linked

“Operably linked” is defined herein as a configuration in which a control sequence such as a promoter sequence or regulating sequence is appropriately placed at a position relative to the nucleotide sequence of interest, preferably coding for a δTCR (or a γTCR) chain or a γδTCR comprising the polypeptide as identified such that the promoter or control or regulating sequence directs or affects the transcription and/or production or expression of the nucleotide sequence of interest, preferably encoding a δTCR (or γTCR) chain or a γδTCR comprising the polypeptide as identified in a cell and/or in a subject. For instance, a promoter is operably linked to a coding sequence if the promoter is able to initiate or regulate the transcription or expression of a coding sequence, in which case the coding sequence should be understood as being “under the control of” the promoter.

### Nucleic Acid Construct

An “expression construct” or “nucleic acid construct” comprises a nucleic acid molecule, such as the ones described herein, and may be expressed in a host cell. In some cases, such a construct is a viral expression construct. A viral expression construct comprises parts of a virus' genome, as further described later herein.

An expression construct or nucleic acid construct carries a genome that is able to stabilize and remain episomal in a cell. Within the context of the invention, a cell may mean to encompass a cell used to make the construct or a cell wherein the construct will be administered. Alternatively, a construct is capable of integrating into a cell's genome, e.g. through homologous recombination or otherwise. A particularly preferred expression construct is one wherein a nucleotide sequence encoding a δTCR (or γTCR) chain or part thereof or a γδTCR is operably linked to a promoter as defined herein wherein said promoter is capable of directing expression of said nucleotide sequence (i.e. coding sequence) in a cell. Such a preferred expression construct is said to comprise an expression cassette. An expression cassette as used herein comprises or consists of a nucleotide sequence encoding a δTCR (or γTCR) chain or part thereof or a γδTCR or part thereof. An expression construct may comprise two expression cassettes to allow the expression of two polypeptides such as a δTCR and a γTCR chain or part thereof. A viral expression construct may be an expression construct which is intended to be used in gene therapy. It is designed to comprise part of a viral genome as later defined herein.

An expression construct may further comprise a sequence encoding a 2A-self cleaving peptide. These self-cleaving peptides are known to the skilled person and are further described, for example, in Xu Y., et al (2019), Cancer Immunology, Immunotherapy, 68: 1979-1993 and Pincha M., et al, (2011), Gene Therapy, 18: 750-764, both of which incorporated herein by reference in their entireties. Non-limiting examples of suitable 2A peptides are F2A (2A peptide derived from the foot-and-mouth disease virus), E2A (2A peptide derived from the equine rhinitis virus), P2A (2A peptide derived from the porcine teschovirus-1), or T2A (2A peptide derived from the Thosea asigna virus). In some embodiments, the 2A self-cleaving peptide is a F2A peptide. In some embodiments, the 2A self-cleaving peptide is an E2A peptide. In some embodiments, the 2A self-cleaving peptide is a P2A peptide. In some embodiments, the 2A self-cleaving peptide is a T2A peptide. The skilled person understands that an expression construct described herein may also comprise nucleotide sequences encoding different 2A self-cleaving peptides. In expression constructs encoding a γδTCR or part thereof, a sequence encoding a 2A self-cleaving peptide may in some cases be inserted between the sequence encoding the 6 T-cell receptor chain or part thereof and the γT-cell receptor chain or part thereof.

An expression construct may further comprise a sequence encoding a chimeric bidirectional signaling transmembrane protein as described earlier herein.

Expression constructs disclosed herein could be prepared using recombinant techniques in which nucleotide sequences encoding said δTCR (or γTCR) chain or part thereof or a γδTCR or part thereof are expressed in a suitable cell, e.g. cultured cells or cells of a multicellular organism, such as described in Ausubel et al. and in Sambrook and Green (supra). Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis). Typically, a nucleic acid or nucleotide sequence encoding a δTCR (or γTCR) chain or a γδTCR or part thereof is used in an expression construct or expression vector. The phrase “expression vector” generally refers to a nucleotide sequence that is capable of effecting expression of a gene in a host compatible with such sequences. These expression vectors typically include at least suitable promoter sequences and optionally, transcription termination signals. An additional factor necessary or helpful in effecting expression can also be used as described herein. A nucleic acid or DNA or nucleotide sequence encoding a δTCR (or γTCR) chain or a γδTCR or part thereof is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines such as human cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been defined herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Green, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Green, 2001, supra). An expression vector includes the replication system and transcriptional and translational regulatory sequences together with the insertion site for the polypeptide encoding segment can be employed. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insecT-cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media.

Alternatively, a host cell is a cell that is part of a multicellular organism such as a transgenic plant or animal.

### Viral Vector

A vector may comprise a nucleic acid construct or an expression construct as earlier defined herein. A vector as described herein may be selected from any genetic element known in the art which can facilitate transfer of nucleic acids between cells, such as, but not limited to, plasmids, transposons, cosmids, chromosomes, artificial chromosomes, viruses, virions, and the like. A vector may also be a chemical vector, such as a lipid complex or naked DNA. “Naked DNA” or “naked nucleic acid” refers to a nucleic acid molecule that is not contained in encapsulating means that facilitates delivery of a nucleic acid into the cytoplasm of a target host cell. Naked DNA may be circular or linear (linearized DNA sequence). Optionally, a naked nucleic acid can be associated with standard means used in the art for facilitating its delivery of the nucleic acid to the target host cell, for example to facilitate the transport of the nucleic acid through the cell membrane.

A vector may be a viral vector and/or a gene therapy vector. A viral vector is a vector that comprises an expression construct as defined above.

A gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol.10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

A viral vector and/or a gene therapy vector may be an adenoviral vector, an adeno-associated viral vector or a retroviral vector. These vectors may comprise a nucleic acid molecule or nucleic acid construct as described herein.

A particularly suitable vector includes an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications. (Russell, 2000, J. Gen. Virol. 81: 2573-2604; Goncalves, 2005, Virol J. 2(1):43) as indicated above. AAV vectors are even more preferred since they are known to result in very stable long term expression of transgene expression (up to 9 years in dog (Niemeyer et al, Blood. 2009 Jan. 22; 113(4):797-806) and ˜2 years in human (Nathwani et al, N Engl J Med. 2011 Dec. 22; 365(25):2357-65, Simonelli et al, Mol Ther. 2010 March; 18(3):643-50. Epub 2009 Dec. 1.)). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34.

Another suitable vector includes a retroviral vector. A preferred retroviral vector for application in the present invention is a lentiviral based viral vector. Lentiviral vectors have the ability to infect and to stably integrate into the genome of dividing and non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et aL. (2000, J Gene Med 2000; 2: 308-16).

In an embodiment, the vector is a viral vector, preferably a lentiviral vector. In an embodiment, a single bicistronic viral vector is used. In an embodiment, a single bicistronic lentiviral vector with a 2A self-cleaving peptide sequence (i.e. SEQ ID NO:25) is used as in the experimental part (Xu Y., et al (2019), Cancer Immunology, Immunotherapy, 68: 1979-1993 and Pincha M., et al, (2011), Gene Therapy, 18: 750-764).

Other suitable viral and/or gene therapy vectors include a herpes virus vector, a polyoma virus vector or a vaccinia virus vector.

A viral and/or gene therapy vector comprises a nucleotide encoding a δTCR (or γTCR) chain, or a γδTCR whereby each of said nucleotide sequence is operably linked to the appropriate regulatory sequences. Such regulatory sequence will at least comprise a promoter sequence. Suitable promoters for expression of such a nucleotide sequence from gene therapy vectors include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter, the MSCV promoter and the herpes simplex virus thymidine kinase promoter. Transposon or other non-viral delivery systems may also be used in this context. All systems can be used in vitro or in vivo.

A viral and/or gene therapy vector may optionally comprise a further nucleotide sequence coding for a further polypeptide. A further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Green (supra).

A viral and/or gene therapy vector is preferably formulated in a pharmaceutical composition as defined herein. In this context, a pharmaceutical composition may comprise a suitable pharmaceutical carrier as earlier defined herein.

### Transgene

A “transgene” is herein defined as a gene or a nucleic acid molecule (i.e. a molecule encoding a δTCR or a γTCR chain or a γδTCR or a part thereof) that has been newly introduced into a cell, i.e. a gene that may be present but may normally not be expressed or expressed at an insufficient level in a cell. The transgene may comprise sequences that are native to the cell, sequences that naturally do not occur in the cell and it may comprise combinations of both. A transgene may contain sequences coding for a δTCR or a γTCR chain or a γδTCR or parts thereof and comprising the polypeptide as identified and/or additional proteins as earlier identified herein that may be operably linked to appropriate regulatory sequences for expression of the sequences coding for a δTCR or a γTCR chain or a γδTCR or parts thereof. In some embodiments, the transgene is not integrated into the host cell's genome. In some embodiments, the transgene is integrated into the host cell's genome.

### Transduction

“Transduction” refers to the delivery of a δTCR chain or a γTCR chain or parts thereof or a γδTCR or parts thereof into a recipient host cell by a viral vector. For example, transduction of a cell by a retroviral or lentiviral vector of the invention leads to transfer of the genome contained in that vector into the transduced cell. In an embodiment, the vector is a lentiviral vector.

### Host Cell

“Host cell” refers to the cell into which the DNA delivery takes place, such as the T-cells of a donor. Cells or T-cells of the invention may be named engineered cells as further explained below.

### Engineered Cells

“Engineered cells” refers herein to cells having been engineered, e.g. by the introduction of an exogenous nucleic acid sequence as defined herein. Such a cell has been genetically modified for example by the introduction of for example one or more mutations, insertions and/or deletions in the endogenous gene and/or insertion of a genetic construct in the genome. The modification may have been introduced using recombinant DNA technology. An engineered cell may refer to a cell in isolation or in culture. Engineered cells may be “transduced cells” wherein the cells have been infected with e.g. a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplextransfection of nucleic acids. Such a transfection may be transient, but may also be a stable transfection wherein cells can be selected that have the gene construct integrated in their genome. In some cases genetic engineering systems such as CRISPR or Argonaute may be utilized to design engineered cells that express a polypeptide described herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA. Genome editing tools as described above may also be used to introduce a genomic modification which results in the reduction or elimination of surface expression of an endogenous αβTCR in an αβT-cell as discussed earlier herein.

In an embodiment, an “engineered cell” has been transformed, modified or transduced to comprise a heterologous or exogenous nucleic acid molecule (i.e. encoding a δTCR chain or a γTCR chain or parts thereof or a γδTCR or parts thereof). In the application, the wording “engineered cell” may be replaced by “modified cell” or “transformed cell” or “transduced cell”. In an embodiment, said cell expresses a protein encoded by said nucleic acid molecule. In an embodiment, said cell is a T-cell, preferably a γδT-cell or an αβT-cell, more preferably an αβT-cell. In an embodiment, said cell is a TEG. In an embodiment, said cell is from a human cell line, for example it is a HEK293 or a HEK293F or a derivative thereof.

### TEG

A “TEG” is a T-cell engineered to express a defined δTCR chain, a γTCR chain or parts thereof or a γδTCR or parts thereof as disclosed herein. In a non-limiting example, a TEG can be an alpha-beta T-cell that is engineered to express a defined γδTCR.

### Production of the Cells of the Invention

In some embodiments, the cells can be cultured for extended periods without stimulation or with stimulation. Stimulation may comprise contact with an anti-CD3 antibody or antigen binding fragment thereof immobilized on a surface. Stimulation may comprise contact with a target cell.

For co-stimulation of an accessory molecule on the surface of the T-cells, a ligand that binds the accessory molecule can be used. In some cases a population of T-cells can be CD3-CD28 co-stimulated, for example, contacted with an anti-CD3 antibody and an anti-CD28 antibody, under conditions that can stimulate proliferation of the T-cells.

Conditions appropriate for T-cell culture can include an appropriate media (e.g., Minimal Essential Media or RPMI Media 1640, TexMACS (Miltenyi) or, X-vivo 5, (Lonza)) that may contain factors necessary for proliferation and viability, including serum. In an aspect, cells can be maintained under conditions necessary to support growth; for example, an appropriate temperature (e.g., 37° C.) and atmosphere (e.g., air plus 5% CO2).

Cells can be obtained from any suitable source for the generation of engineered cells. Cells can be primary cells. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumours. Cells can be derived from a healthy donor or from a patient diagnosed with cancer. Cells can also be obtained from a cell therapy bank. Cells can also be obtained from whole blood, apheresis, or a tumour sample of a subject. A cell can be a tumour infiltrating lymphocytes (TIL). In some cases an apheresis can be a leukapheresis.

A desirable cell population can also be selected prior to modification. A selection can include at least one of: magnetic separation, flow cytometric selection, antibiotic selection. The one or more cells can be any blood cells, such as peripheral blood mononuclear cell (PBMC), lymphocytes, monocytes or macrophages. The one or more cells can be any immune cells such as a lymphocyte, an alpha-beta T-cell, a gamma-delta T-cell, CD4+ T-cell, CD8+ T-cell, a T effector cell, a lymphocyte, a B cell, an NK cell, an NKT-cell, a myeloid cell, a monocyte, a macrophage, or a neutrophil.

### Pharmaceutical Composition/Method of Treatment

In therapeutic applications, an effective amount of a δTCR or a γTCR chain or parts thereof or a γδTCR or a part thereof or nucleic acid molecule or conjugate or nucleic acid construct or viral vector or cell expressing these molecules as defined herein is administered to a subject. Accordingly, pharmaceutical compositions of the present disclosure comprise an effective amount of one or more molecules (i.e., a δT-cell receptor chain, a γT-cell receptor chain, a γδT-cell receptor, a part thereof, a conjugate, a nucleic acid molecule, a nucleic acid construct, a vector, or a cell such as a T-cell, as described herein), optionally dissolved or dispersed in a pharmaceutically acceptable carrier.

The term “effective amount” as used herein is defined as the amount of the molecules of the present invention that are necessary to result in the desired physiological change in the cell or tissue to which it is administered. The term “therapeutically effective amount” as used herein is defined as the amount of the molecules of the present invention that achieves a desired effect with respect to cancer. In this context, a “desired effect” is synonymous with “an anti-tumour activity” or an “anti-infective” as earlier defined herein. A skilled artisan readily recognizes that in many cases the molecules may not provide a cure but may provide a partial benefit, such as alleviation or improvement of at least one symptom or parameter. In some embodiments, a physiological change having some benefit is also considered therapeutically beneficial. Thus, in some embodiments, an amount of molecules that provides a physiological change is considered an “effective amount” or a “therapeutically effective amount.”

The phrases “pharmaceutical or pharmacologically acceptable” refers to molecular entities and compositions that do not produce or produce acceptable adverse, allergic or other untoward reaction when administered to an animal, such as, for example, a human, as appropriate. Whether certain adverse effects are acceptable is determined based on the severity of the disease. The preparation of a pharmaceutical composition that contains at least one active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. In certain embodiments, a pharmaceutical composition described herein comprising a population of cells described herein, further comprises a suitable amount of an antifungal agent. In some cases, a pharmaceutical composition described herein comprises an antifungal agent in an amount sufficient for the pharmaceutical composition to retain at least 20%, 30%, 40%, 50%, 60%, 70%, 80% or 90% of its desired activity for a period of at least 1 month, 2 months, 3 months, 6 months, 1 year, 1.5 years, 2 years, 2.5 years or 3 years.

The actual dosage amount of a composition of the present invention administered to an animal or a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### T-Cell as Pharmaceutical Composition

In some embodiments, a preferred pharmaceutical composition comprises a T-cell, preferably a γδT-cell or an αβT-cell, more preferably an αβT-cell, expressing a δT-cell receptor chain, a γT-cell receptor chain, a γδT-cell receptor, a part thereof, or comprising a nucleic acid molecule, a nucleic acid construct, or a vector as described herein. The T-cells may optionally express a chimeric bidirectional signaling protein as described earlier herein. The composition may comprise a population of said T-cells as described herein. These compositions may easily be obtained using the information of the disclosure.

As a non-limiting example, prior to expansion and genetic modification of the T-cells of the invention, a source of T-cells is obtained from a subject. T-cells can be obtained from a number of sources, including PBMCs, bone marrow, lymph node tissue, cord blood, thymus tissue, tissue from a site of infection, ascites, pleural effusion, spleen tissue, and tumours. In certain embodiments of the present invention, T-cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T-cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In a particular embodiment, the engineered cell can be a T-cell. The engineered cell can be an effector (T_{EFF}), effector-memory (T_{EM}), central-memory (T_{CM}), T memory stem (T_{SCM}), naive (T_{N}), or CD4+ or CD8+ T-cell. The T-cells can also be selected from a bulk population, for example, selecting T-cells from whole blood. The T-cells can also be expanded from a bulk population. The T-cells can also be skewed towards particular populations and phenotypes. The engineered cell can also be expanded ex vivo. The engineered cell can be formulated into a pharmaceutical composition. The engineered cell can be formulated into a pharmaceutical composition and used to treat a subject in need thereof as earlier explained herein. The engineered cell can be autologous to a subject in need thereof. The engineered cell can be allogeneic to a subject in need thereof. The engineered cell can also be a good manufacturing practices (GMP) compatible reagent. The engineered cell can be part of a combination therapy to treat a subject in need thereof. The engineered cell can be a human cell. The subject that is being treated can be a human.

A method of attaining suitable cells can comprise sorting cells. In some cases, a cell can comprise a marker that can be selected for the cell. For example, such marker can comprise GFP, a resistance gene, a cell surface marker, an endogenous tag. Cells can be selected using any endogenous marker. Suitable cells can be selected or sorted using any technology. Such technology can comprise flow cytometry and/or magnetic columns. The selected cells can then be infused into a subject. The selected cells can also be expanded to large numbers. The selected cells can be expanded prior to infusion.

Vectors can be delivered to cells ex vivo, such as cells explanted from an individual patient (e.g., lymphocytes, T-cells, bone marrow aspirates, tissue biopsy), followed by re-implantation of the cells into a patient, usually after selection for cells which have incorporated the vector. Prior to or after selection, the cells can be expanded.

Ex vivo cell transfection can also be used for diagnostics, research, or for gene therapy (e.g. via re-infusion of the transfected cells into the host organism). In some cases, cells are isolated from the subject organism, transfected with a nucleic acid (e.g., gene or DNA), and re-infused back into the subject organism (e.g. patient). Further, also in vivo cell transfection can be used for gene therapy, in order to reduced immune reactions of the patient.

In some cases, populations of engineered T-cells may be formulated for administration to a subject using techniques known to the skilled artisan. Formulations comprising populations of engineered T-cells may include pharmaceutically acceptable excipient(s). Excipients included in the formulations will have different purposes depending, for example, on the subpopulation of T-cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising populations of engineered T-cells will typically have been prepared and cultured in the absence of any non-human components, such as animal serum.

A formulation may include one population of engineered T-cells, or more than one, such as two, three, four, five, six or more population of engineered T-cells. The formulations comprising population(s) of engineered T-cells may be administered to a subject using modes and techniques known to the skilled artisan. Exemplary modes include, but are not limited to, intravenous injection. Other modes include, without limitation, intratumoural, intradermal, subcutaneous (s.c., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection of infusion of the formulations can be used to effect such administration. The formulations comprising population(s) of engineered T-cells that are administered to a subject comprise a number of engineered T-cells that is effective for the treatment and/or prophylaxis of the specific indication or disease. Thus, therapeutically-effective populations of engineered T-cells are administered to subjects when the methods of the present invention are practiced. In general, formulations are administered that comprise between about 1×10⁴ and about 1×10¹⁰ engineered T-cells. In most cases, the formulation will comprise between about 1×10⁵ and about 1×10⁹ engineered T-cells, from about 5×10⁵ to about 5×10⁸ engineered T-cells, or from about 1×10⁶ to about 1×10⁷ engineered T-cells. However, the number of engineered T-cells administered to a subject will vary between wide limits, depending upon the location, source, identity, extent and severity of the cancer, the age and condition of the individual to be treated etc. A physician will ultimately determine appropriate dosages to be used.

### Additional Methods of the Invention

The present inventors have further found that when a Cγ2 constant region or a part thereof in a γT-cell receptor chain or part thereof is replaced by a Cγ1 constant region or a part thereof, the resulting γT-cell receptor chain or part thereof, or γδT-cell receptor or part thereof comprising it, is able to mediate an improved anti-tumor or anti-infective response. The resulting γT-cell receptor chain or part thereof, or γδT-cell receptor or part thereof comprising it, may demonstrate improved expression, preferably surface expression, in a T-cell, preferably a γδT-cell or an αβT-cell, more preferably an αβT-cell.

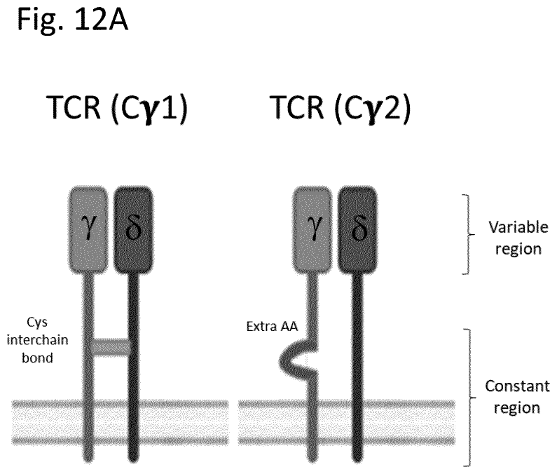

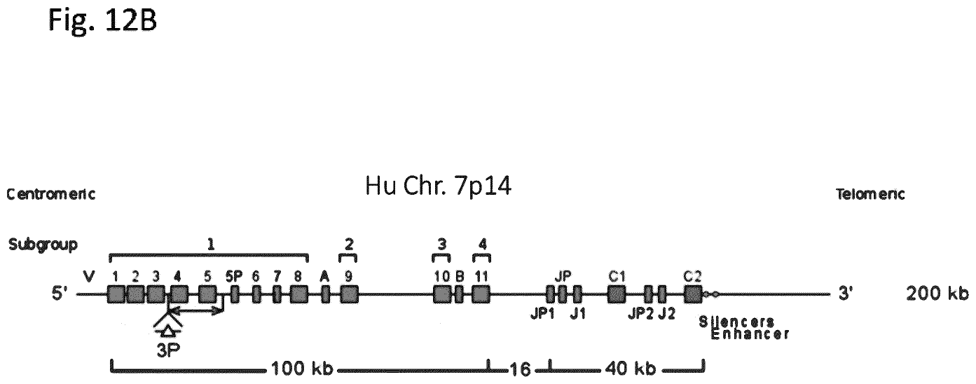

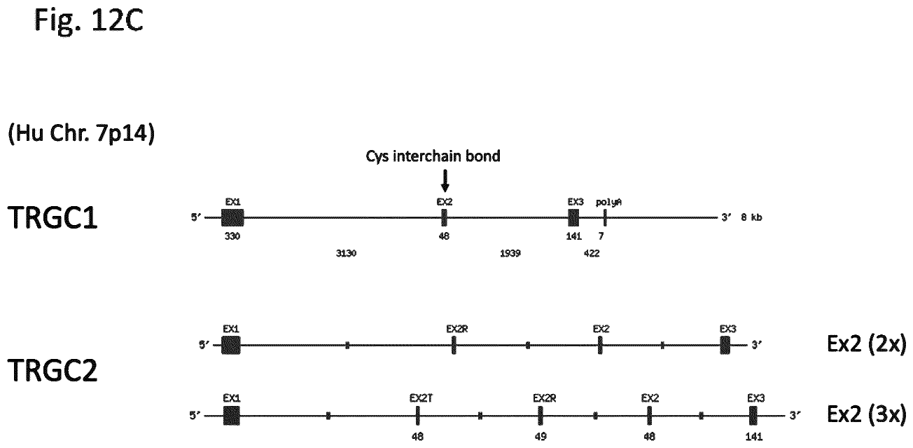

A “constant” region with respect to a γT-cell receptor or a δT-cell receptor chain (or parts thereof) as used herein corresponds to its general definition in the art. γδTCRs are cell-surface multimeric transmembrane complexes formed by two γ and δ glycoproteins non-covalently linked up to six different CD3 subunits. Each of the γ and δ chains of the γδ heterodimer are composed of a variable region, which protrudes into the extracellular milieu and is responsible for the cognate antigen-binding, and by a constant region, which anchors the receptor to the cell membrane and is involved in interactions with CD3 molecules and signal transduction (FIG. 12A). The modular structure of the γ and δ TCR chains is the result of both somatic rearrangements and splicing of variable (V), diversity (D), joining (J), and constant (C) region-encoding genes, each representing a multigene sub-family, which occurs during the development of T lymphocytes in the thymus. First, the variable region is assembled by the somatic rearrangements of V and J genes in TRG (γ chain) loci, and between V, D, and J genes in TRD (δ chain) loci. This event, together with N-insertions between recombined V (D) J, determines the clonal variability of the TCR repertoire. After transcription, the resulting rearranged V-(D)-J region is spliced to the C gene, which encodes the constant region of the receptor. The constant regions are generally conserved.

A Cγ2 constant region as used herein refers to a region encoded by a TRGC2 gene or variant thereof. Such a gene and region are known to the skilled person, for example see Uniprot Ref: P03986, SEQ ID NO: 161, and SEQ ID NO: 164 provided herein. A Cγ1 constant region as used herein refers to a region encoded by a TRGC1 gene or variant thereof. Such a gene and region are known to the skilled person, for example see Uniprot Ref: POCF51 and SEQ ID NO: 152 provided herein.

In the human genome, the TRD locus contains three variable genes (TRDV1-3), three diversity genes (TRDD1-3), fourjoining genes (TRDJ1-4) and one constant TRDC gene, which rearrange to encode a TCRδ chain. The TRG locus that rearranges to encode a TCRγ chain contains 14 variable genes, of which only 6 are functional (TRGV2-5, TRGV8, and TRGV9), five joining genes (TRGJ1, J2, JP, JP1, JP2), and two constant genes (TRGC1, TRGC2, as discussed above) (FIG. 12B). The two TRGC genes resulted, with their associated TRGJ genes, from a recent duplication in the locus (see Antonacci et al, 2020, Genes 1(6): 624, incorporated herein by reference in its entirety). However, there are structural differences: TRGJP1, TRGJ1, and TRGC1 cross-hybridize to TRGJP2, TRGJ, and TRGC2, respectively, whereas the TRGJP has no equivalent in the TRGJP2-J2-C2 cluster (see Lefranc M.-P et al, 1986, PNAS 83:9596-9600; Brenner M B, 1987, Nature 325: 689-694, both of which incorporated herein by reference in their entireties).

The TRGC genes encode the extracellular region of typically 110 amino acids (C-region), the connecting region (CO), the transmembrane region™, and the cytoplasmic region (CY). The TRGC1 gene comprises three exons and typically encodes a C-region of 173 AA (Cγ1), whereas the TRGC2 gene comprises four or five exons, owing to the duplication or triplication of a region that includes Exon 2 (EX2, EX2T and/or EX2R) and typically encodes a C-region (Cγ2) of 189 or 205 AA, respectively (see Le Franc M.-P, The T Cell Receptor FactsBook, 2001, Academic Press, incorporated herein by reference in its entirety) (FIGS. 12A and 12C).

Accordingly, a TRGC2 (Cγ2) constant region typically differs from a TRGC1 (Cγ1) constant region by having 16-32 extra amino acids in the connecting peptide. Additionally, Exon 2 of the TRGC1 gene has a cysteine involved in the interchain disulfide bridge, whereas the cysteine is not conserved in Exon 2 of the human TRGC2 gene.

The frequency of γδTCR comprising Cγ1 or Cγ2 constant regions differs among the different γδT-cell subsets. For example, the constant gamma region of the Vγ9Vδ2 TCR expressed by the most abundant γδT lymphocytes in human adult blood is exclusively encoded by TRGC1 gene, while the non-Vγ9Vδ2 TCRs tend to express a Cγ2 domain encoded by the TRGC2 gene (Casorati et al, 1989, JEM 170(5): 1521-35).

The inventors' finding is surprising, as the mediation of an anti-tumour or anti-infective response of γδTCRs is traditionally associated with the variable regions. The impact of the constant regions has hitherto not been explored. Accordingly, in a further aspect, there is provided a method for improving the anti-tumour or anti-infective response mediated by a γδT-cell receptor or a part thereof comprising a CDR3 region, wherein said receptor or part thereof comprises a Cγ2 constant region or a part thereof, said method comprising the step of replacing said Cγ2 constant region or part thereof by a Cγ1 constant region or a part thereof.

It is understood that said Cγ2 constant region or a part thereof is comprised in the γT-cell receptor chain or part thereof comprised in the γδT-cell receptor or part thereof.

A γT-cell receptor chain or part thereof comprised in a γδT-cell receptor or part thereof which comprises a Cγ2 constant region or a part thereof can be distinguished from a chain or part thereof which comprises a Cγ1 constant region or a part thereof based on the structural and sequence differences between Cγ2 and Cγ1 constant regions discussed above.

The skilled person understands that “replacing a Cγ2 constant region or a part thereof by a Cγ1 constant region or part thereof”, in addition to a complete replacement, also refers to the γT-cell receptor chain or part thereof comprising the Cγ2 constant region or part thereof being modified. Accordingly, the skilled person understands that modifying the amino acid sequence of a Cγ2 constant region or part thereof to arrive at the sequence of a Cγ1 constant region or part thereof (or an intermediate sequence), for example by introducing a modification such as an amino acid substitution and/or deletion to the Cγ2 constant region or part thereof, is also encompassed by the definition. This may be done since the structure and differences in the amino acid sequence of Cγ2 and Cγ1 regions are known in the art as described above.

As a non-limiting example, the skilled person starting from SEQ ID NO: 161 or SEQ ID NO: 164 may modify said sequences to arrive at SEQ ID NO: 152 using standard molecular toolbox techniques.

In some embodiments, “replacing a Cγ2 constant region or a part thereof by a Cγ1 constant region or part thereof” corresponds to introducing at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-one, or at least twenty-two amino acid modifications in a γT-cell receptor chain or a part thereof comprising SEQ ID NO: 161, SEQ ID NO: 164, or a part thereof.

In some embodiments, “replacing a Cγ2 constant region or a part thereof by a Cγ1 constant region or part thereof” corresponds to introducing at least one, at least two, at least three, at least four, at least five, or at least six amino acid substitutions in a γT-cell receptor chain or a part thereof comprising SEQ ID NO: 161, SEQ ID NO: 164, or a part thereof.

In some embodiments, “replacing a Cγ2 constant region or a part thereof by a Cγ1 constant region or part thereof” corresponds to introducing at least one, at least two, at least three, at least four, at least five, at least six, at least seven, at least eight, at least nine, at least ten, at least eleven, at least twelve, at least thirteen, at least fourteen, at least fifteen, or at least sixteen amino acid deletions in a γT-cell receptor chain or a part thereof comprising SEQ ID NO: 161, SEQ ID NO: 164, or a part thereof.

In some embodiments, “replacing a Cγ2 constant region or a part thereof by a Cγ1 constant region or part thereof” corresponds to the isoleucine corresponding to position 41 of SEQ ID NO: 161 or SEQ ID NO: 164, preferably of SEQ ID NO: 161, being substituted, preferably by a valine. In some embodiments, it corresponds to the glutamine corresponding to position 47 of SEQ ID NO: 161 or SEQ ID NO: 164, preferably of SEQ ID NO: 161, being substituted, preferably by a glutamic acid. In some embodiments, it corresponds to the glutamic acid corresponding to position 80 of SEQ ID NO: 161 or SEQ ID NO: 164, preferably of SEQ ID NO: 161, being substituted, preferably by a lysine. In some embodiments, it corresponds to the isoleucine corresponding to position 99 of SEQ ID NO: 161 or SEQ ID NO: 164, preferably of SEQ ID NO: 161, being substituted, preferably by a valine. In some embodiments, it corresponds to the tryptophan corresponding to position 137 of SEQ ID NO: 161 or SEQ ID NO: 164, preferably of SEQ ID NO: 161, being substituted, preferably by a cysteine. In some embodiments it corresponds to the threonine corresponding to position 157 of SEQ ID NO: 164 being substituted, preferably by a methionine. In some embodiments, it corresponds to the glycine corresponding to position 178 of SEQ ID NO: 161 being substituted, preferably by an arginine.

In some embodiments, “replacing a Cγ2 constant region or a part thereof by a Cγ1 constant region or part thereof” corresponds to the aspartic acid corresponding to position 111 of SEQ ID NO: 161 or SEQ ID NO: 164, preferably of SEQ ID NO: 161, being deleted. In some embodiments, it corresponds to the valine corresponding to position 112 of SEQ ID NO: 161 or SEQ ID NO: 164, preferably of SEQ ID NO: 161, being deleted. In some embodiments, it corresponds to the threonine corresponding to position 113 of SEQ ID NO: 161 or SEQ ID NO: 164, preferably of SEQ ID NO: 161, being deleted. In some embodiments, it corresponds to the threonine corresponding to position 114 of SEQ ID NO: 161 or SEQ ID NO: 164, preferably of SEQ ID NO: 161, being deleted. In some embodiments, it corresponds to the valine corresponding to position 115 of SEQ ID NO: 161 or SEQ ID NO: 164, preferably of SEQ ID NO: 161, being deleted. In some embodiments, it corresponds to the aspartic acid corresponding to position 116 of SEQ ID NO: 161 or SEQ ID NO: 164, preferably of SEQ ID NO: 161, being deleted. In some embodiments, it corresponds to the proline corresponding to position 117 of SEQ ID NO: 161 or SEQ ID NO: 164, preferably of SEQ ID NO: 161, being deleted. In some embodiments, it corresponds to the lysine corresponding to position 118 of SEQ ID NO: 161 or SEQ ID NO: 164, preferably of SEQ ID NO: 161, being deleted. In some embodiments, it corresponds to the tyrosine corresponding to position 119 of SEQ ID NO: 161 or SEQ ID NO: 164, preferably of SEQ ID NO: 161, being deleted. In some embodiments, it corresponds to the asparagine corresponding to position 120 of SEQ ID NO: 161 or SEQ ID NO: 164, preferably of SEQ ID NO: 161, being deleted. In some embodiments, it corresponds to the tyrosine corresponding to position 121 of SEQ ID NO: 161 or SEQ ID NO: 164, preferably of SEQ ID NO: 161, being deleted. In some embodiments, it corresponds to the serine corresponding to position 122 of SEQ ID NO: 161 or SEQ ID NO: 164, preferably of SEQ ID NO: 161, being deleted. In some embodiments, it corresponds to the lysine corresponding to position 123 of SEQ ID NO: 161 or SEQ ID NO: 164, preferably of SEQ ID NO: 161, being deleted. In some embodiments, it corresponds to the aspartic acid corresponding to position 124 of SEQ ID NO: 161 or SEQ ID NO: 164, preferably of SEQ ID NO: 161, being deleted. In some embodiments, it corresponds to the alanine corresponding to position 125 of SEQ ID NO: 161 or SEQ ID NO: 164, preferably of SEQ ID NO: 161, being deleted. In some embodiments, it corresponds to the asparagine corresponding to position 126 of SEQ ID NO: 161 or SEQ ID NO: 164, preferably of SEQ ID NO: 161, being deleted.

It is to be understood that combinations of the abovementioned amino acid substitutions and/or deletions are also encompassed.

The location of the Cγ2 constant region or part thereof can easily be identified in a γT-cell receptor chain or part thereof comprising it as this region is generally conserved as described above. For example, the location of the Cγ2 constant region or part thereof in a γT-cell receptor chain or part thereof in question can be obtained by aligning the amino acid sequence of said chain or part thereof with Uniprot Ref: P03986 or SEQ ID NO: 161, or part thereof, using sequence alignment tools described herein. Then, the Cγ2 constant region or part thereof may be replaced by a Cγ1 constant region or part thereof, for example by Uniprot Ref: POCF51 or SEQ ID NO: 152, or a part thereof, following the methods of the invention in combination with using standard molecular toolbox techniques, for example as described in standard handbooks such as Sambrook and Green (2012, supra) and Ausubel et al. (2003, supra) and others discussed herein. An example of such a replacement is provided in the experimental section herein. As another example, one or more amino acid modifications may be introduced in a Cγ2 constant region or part thereof as described earlier herein.

Similarly, the location of a Cγ1 constant region or part thereof in a γT-cell receptor chain or part thereof in question can be obtained by aligning the amino acid sequence of said chain or part thereof with Uniprot Ref: POCF51 or SEQ ID NO: 152, or a part thereof.

Using the above approach, it can (for example) be deduced that the Cγ2 constant region in SEQ ID NO: 12 starts at amino acid position D133 and ends at amino acid position S321 of that sequence. As another example, it can be deduced that the Cγ1 constant region in SEQ ID NO: 18 starts at amino acid position D134 and ends at amino acid position K305 of that sequence. As another example, it can be deduced that the Cγ1 constant region in SEQ ID NO: 162 starts at amino acid position D134 and ends at amino acid position S306 of that sequence. As another example, it can be deduced that the Cγ2 constant region in SEQ ID NO: 24 starts at amino acid position D140 and ends at amino acid position S328 of that sequence. As another example, it can be deduced that the Cγ1 constant region in SEQ ID NO: 131 starts at amino acid position D133 and ends at amino acid position S305 of that sequence. As another example, it can be deduced that the Cγ2 constant region in SEQ ID NO: 133 starts at amino acid position D134 and ends at amino acid position S322 of that sequence. As another example, it can be deduced that the Cγ1 constant region in SEQ ID NO: 135 starts at amino acid position D140 and ends at amino acid position S312 of that sequence.

The start and end amino acid positions of a Cγ2 or a Cγ1 region or part thereof in any other γT-cell receptor chain or part thereof may be similarly deduced.

A definition of “part thereof” has been provided earlier herein, and may correspond to at least 1%, at least 2%, at least 3%, at least 4%, at least 5%, at least 10%, at least 20%, at least 30%, at least 40% of the length of the Cγ2 or Cγ1 constant region, such as represented by an amino acid sequence with a specific SEQ ID NO, or it may correspond to at least 50% of the length of the Cγ2 or Cγ1 constant region, or at least 60%, or at least 70%, or at least 80%, or at least 85%, or at least 90%, or at least 91%, or at least 92%, or at least 93%, or at least 94%, or at least 95%, or at least 96%, or at least 97%, or at least 98%, or at least 99%. In some embodiments, a part of a Cγ1 or Cγ2 constant region corresponds to a Cγ1 or Cγ2 constant region lacking the transmembrane and/or cytoplasmic domain or a part thereof. In some embodiments, a part of a Cγ1 constant region does not comprise SEQ ID NO: 158 or a part thereof. In some embodiments, a part of a Cγ2 constant region does not comprise SEQ ID NO: 159 or a part thereof.

The γδT-cell receptor or part thereof in which the Cγ2 constant region or part thereof has been replaced by a Cγ1 constant region or part thereof may be expressed by a cell, for example a T-cell, a γδT-cell, or an αβT-cell, as described earlier herein. The γδT-cell receptor or part thereof in which the Cγ2 constant region or part thereof has been replaced by a Cγ1 constant region or part thereof may be a soluble polypeptide, as described earlier herein, for example it may be a γδTCR bispecific engager or a γδTCR-CD3 bispecific engager.

The γδT-cell receptor or part thereof in which the Cγ2 constant region or part thereof has been replaced by a Cγ1 constant region or part thereof may result in the γδT-cell receptor or part thereof demonstrating an improved expression, preferably surface expression, in a T-cell, preferably a γδT-cell or an αβT-cell, more preferably an αβT-cell.

Accordingly, in a further aspect, there is provided a method for improving the surface expression of a γδT-cell receptor or a part thereof comprising a CDR3 region in a T-cell, preferably a γδT-cell or an αβT-cell, more preferably an αβT-cell, wherein said receptor or part thereof comprises a Cγ2 constant region or a part thereof, said method comprising the step of replacing said Cγ2 constant region or part thereof by a Cγ1 constant region or a part thereof.

The skilled person understands that the step of “replacing a Cγ2 constant region or a part thereof by a Cγ1 constant region or a part thereof” in a γδT-cell receptor or a part thereof comprising a CDR3 region is to be understood as being equivalent to “replacing a nucleotide sequence encoding a Cγ2 constant region or a part thereof by a nucleotide sequence encoding a Cγ1 constant region or a part thereof” in a nucleic acid molecule encoding said γδT-cell receptor or a part thereof comprising a CDR3 region discussed in the aspects below. This is because the end results of the steps are the same as they both may result in a γδT-cell receptor or a part thereof, which previously comprised a Cγ2 constant region or a part thereof, comprising a Cγ1 constant region or a part thereof (in the latter step once the nucleic acid molecule is expressed).

Accordingly, in a further aspect, there is provided a method for identifying a γδT-cell receptor or a part thereof comprising a CDR3 region that mediates an improved anti-tumour or anti-infective response comprising the steps of:
a) providing a nucleic acid molecule comprising a nucleotide sequence encoding a γT-cell receptor chain or a part thereof comprising a CDR3 region, wherein said γT-cell receptor chain or a part thereof comprises a Cγ2 constant region or a part thereof;
b) replacing the nucleotide sequence encoding the Cγ2 constant region or a part thereof by a nucleotide sequence encoding a Cγ1 constant region or a part thereof in the nucleic acid molecule of step a);
c) introducing the nucleic acid molecule of step b) and a nucleic acid molecule encoding a δT-cell receptor chain in a T-cell, preferably a γδT-cell or an αβT-cell, more preferably an αβT-cell, to provide an engineered T-cell expressing a γδT-cell receptor or part thereof comprising the γT-cell receptor chain or part thereof encoded by the nucleotide sequence of step b);
d) determining the anti-tumour or anti-infective response of the engineered T-cell of step c);
e) identifying the γδT-cell receptor or part thereof that mediates the improved anti-tumour or anti-infective response.

In a further aspect, there is provided a method for identifying a γδT-cell receptor or a part thereof comprising a CDR3 region that mediates an improved anti-tumour or anti-infective response comprising the steps of:
a) providing a γδT-cell receptor or a part thereof comprising a CDR3 region, wherein said receptor or part thereof comprises a Cγ2 constant region or a part thereof
b) replacing said Cγ2 constant region or part thereof by a Cγ1 constant region or a part thereof;
c) expressing the γδT-cell receptor or a part thereof obtained in step b) in an engineered T-cell, preferably γδT-cell or αβT-cell, more preferably αβT-cell;
d) determining the anti-tumour or anti-infective response of the engineered T-cell of step c);
e) identifying the γδT-cell receptor or part thereof that mediates the improved anti-tumour or anti-infective response.

The skilled person understands that identification of the γδT-cell receptor or part thereof that mediates an improved anti-tumour or anti-infective response may, for example, be done by comparing the anti-tumour or anti-infective response of the engineered T-cell of step c) with an equivalent engineered T-cell expressing the γδT-cell receptor or a part thereof of step a), for example in step d) of the method.

In a further aspect, there is provided a method for identifying a γδT-cell receptor or a part thereof comprising a CDR3 region demonstrating improved surface expression in a T-cell, preferably a γδT-cell or an αβT-cell, more preferably an αβT-cell, comprising the steps of:
a) providing a nucleic acid molecule comprising a nucleotide sequence encoding a γT-cell receptor chain or a part thereof comprising a CDR3 region, wherein said γT-cell receptor chain or a part thereof comprises a Cγ2 constant region or a part thereof;
b) replacing the nucleotide sequence encoding the Cγ2 constant region or a part thereof by a nucleotide sequence encoding a Cγ1 constant region or a part thereof in the nucleic acid molecule of step a);
c) introducing the nucleic acid molecule of step b) and a nucleic acid molecule encoding a δT-cell receptor chain in a T-cell, preferably a γδT-cell or an αβT-cell, more preferably an αβT-cell, to provide an engineered T-cell expressing a γδT-cell receptor or part thereof comprising the γT-cell receptor chain or part thereof encoded by the nucleotide sequence of step b);
d) determining the surface expression of the γδT-cell receptor or part thereof by the engineered T-cell of step c);
e) identifying the γδT-cell receptor or part thereof that demonstrates improved surface expression.

In a further aspect, there is provided a method for identifying a γδT-cell receptor or a part thereof comprising a CDR3 region demonstrating improved surface expression in a T-cell, preferably a γδT-cell or an αβT-cell, more preferably an αβT-cell, comprising the steps of:
a) providing a γδT-cell receptor or a part thereof comprising a CDR3 region, wherein said receptor or part thereof comprises a Cγ2 constant region or a part thereof
b) replacing said Cγ2 constant region or part thereof by a Cγ1 constant region or a part thereof;
c) expressing the γδT-cell receptor or a part thereof obtained in step b) in an engineered T-cell, preferably γδT-cell or αβT-cell, more preferably αβT-cell;
d) determining the surface expression of the γδT-cell receptor or part thereof by the engineered T-cell of step c);
e) identifying the γδT-cell receptor or part thereof that demonstrates improved surface expression.

The skilled person understands that identification of the γδT-cell receptor or part thereof that demonstrates improved surface expression may, for example, be done by comparing the surface expression of the γδT-cell receptor or part thereof in the engineered T-cell of step c) with the one of the γδT-cell receptor or part thereof of step a) expressed in an equivalent engineered T-cell, for example in step d) of the method.

In further aspect, there is provided a method for identifying a soluble γδT-cell receptor or a part thereof comprising a CDR3 region that mediates an improved anti-tumour or anti-infective response comprising the steps of:
a) providing a nucleic acid molecule comprising a nucleotide sequence encoding a soluble γT-cell receptor chain or a part thereof comprising a CDR3 region, wherein said γT-cell receptor chain or a part thereof comprises a Cγ2 constant region or part thereof and a T-cell- and/or NK-cell-binding domain;
b) replacing the nucleotide sequence encoding the Cγ2 constant region or part thereof by a nucleotide sequence encoding a Cγ1 constant region or part thereof in the nucleic acid molecule of step a);
c) introducing the nucleic acid molecule of step b) and a nucleic acid molecule encoding a soluble δT-cell receptor chain in a host cell, preferably a human cell, to provide an engineered cell expressing a soluble γδT-cell receptor or part thereof comprising the γT-cell receptor chain or part thereof encoded by the nucleotide sequence of step b);
d) obtaining the soluble γδT-cell receptors or parts thereof expressed by the cells of step c);
e) contacting the soluble γδT-cell receptors or parts thereof obtained in step d) with a T-cell, preferably a γδT-cell or an αβT-cell, more preferably an αβT-cell, and a target cell;
f) determining the anti-tumour or anti-infective response of the T-cell of step e);
g) identifying the soluble γδT-cell receptor or part thereof that mediates the improved anti-tumour or anti-infective response.

In further aspect, there is provided a method for identifying a soluble γδT-cell receptor or a part thereof comprising a CDR3 region that mediates an improved anti-tumour or anti-infective response comprising the steps of:
a) providing a soluble γδT-cell receptor or a part thereof comprising a CDR3 region, wherein said receptor or part thereof comprises a Cγ2 constant region or a part thereof and a T-cell- and/or NK-cell-binding domain;
b) replacing said Cγ2 constant region or part thereof by a Cγ1 constant region or a part thereof;
c) expressing the soluble γδT-cell receptor or a part thereof obtained in step b) in a host cell, preferably a human cell;
d) obtaining the soluble γδT-cell receptors or parts thereof expressed by the cells of step c);
e) contacting the soluble γδT-cell receptors or parts thereof obtained in step d) with a T-cell, preferably a γδT-cell or an αβT-cell, more preferably an αβT-cell, and a target cell;
f) determining the anti-tumour or anti-infective response of the T-cell of step e);
g) identifying the soluble γδT-cell receptor or part thereof that mediates the improved anti-tumour or anti-infective response.

The skilled person understands that identification of the soluble γδT-cell receptor or part thereof that mediates an improved anti-tumour or anti-infective response may, for example, be done by comparing the anti-tumour or anti-infective response of the T-cell of step e) with an equivalent T-cell contacted with the soluble γδT-cell receptor or part thereof of step a), for example in step f of the method.

A description of a soluble γδT-cell receptor or a part thereof comprising a CDR3 region is provided earlier herein. In some embodiments, a soluble γδT-cell receptor or a part thereof is a γδTCR bispecific engager, preferably a γδTCR-CD3 bispecific engager, as described earlier herein. In some embodiments, a preferred T-cell-binding domain is a CD3-binding domain, preferably an scFv CD3-binding domain, more preferably represented by an amino acid sequence comprising or consisting of SEQ ID NO: 146 or a variant thereof. CD3-binding domains have been described earlier herein.

In some embodiments, a soluble γδT-cell receptor or a part thereof comprises additional domains as described earlier herein. In some embodiments, a soluble γδT-cell receptor or a part thereof comprises a linker, for example as shown in Table 4, preferably comprises an amino acid sequence comprising or consisting of SEQ ID NO: 145 and/or SEQ ID NO: 147. In some embodiments, a soluble γδT-cell receptor or a part thereof comprises a domain facilitating polypeptide excretion and/or polypeptide isolation and/or purification and/or stability. In some embodiments, a soluble γδT-cell receptor or a part thereof comprises a His-tag and/or AVI-tag, preferably comprises an amino acid sequence comprising or consisting of SEQ ID NO: 149 and/or SEQ ID NO: 148 or a variant thereof.

The host cell, preferably human cell, may be any of the suitable host cells described earlier herein. In some embodiments, the cell is from a human cell line, for example it is a HEK293 or a HEK293F or a derivative thereof.

In some embodiments, obtaining the soluble γδT-cell receptors or parts thereof in step d) involves isolation and/or purification of the polypeptides. Suitable solation and/or purification methods are described earlier herein.

Preferred target cells have been described earlier herein and may be selected from cancer or infected cells, for example: Caki-2, HT29, SK-OV-3, 769-P, 786-0, COV504, MDA-MB-231, BLM, Hs895.T, SW480, RKO, IgR39D, HAP-1, OVCAR-3, MZ1851 RC, NCI-226, or others.

In step c) of the methods involving replacing a nucleotide sequence encoding a Cγ2 constant region or a part thereof by a nucleotide sequence encoding a Cγ1 constant region or a part thereof, the nucleic acid molecule encoding the γT-cell receptor chain or part thereof and the nucleic acid molecule encoding the δT-cell receptor chain may correspond to distinct nucleic acid molecules or to the same nucleic acid molecule which encodes both polypeptides. The nucleic acid molecule may be comprised in a nucleic acid construct or a vector as described earlier herein. Preferred vectors are retroviral and lentiviral vectors, with lentiviral vectors being further preferred.

Replacing a Cγ2 constant region or a part thereof by a Cγ1 constant region or part thereof may result in an improvement of the anti-tumour or anti-infective response mediated by the γδT-cell receptor or part thereof (or soluble γδT-cell receptor or part thereof) of at least 5%, at least 10%, at least 20%, at least 30%, at least 40%, at least 50%, at least 60%, at least 70%, at least 80%, at least 90%, at least 100% higher, or more as compared to the γδT-cell receptor or part thereof it is derived from.

Determination of the anti-tumour or anti-infective response of engineered T-cells expressing γδT-cell receptors or T-cells that have been contacted with soluble γδT-cell receptors or parts thereof and a target cell in the methods involving replacement of a Cγ2 constant region or part thereof by a Cγ1 constant region or part thereof may be performed using any of the assays described earlier herein. Optionally, control T-cells as described earlier herein may be used. Further examples are provided in the experimental section herein.

As discussed earlier herein, the ability of a γδTCR and/or part thereof described herein to mediate an anti-tumor or anti-infective response may in some cases also be assessed by assessing their surface expression in a T-cell, preferably a γδT-cell or an αβT-cell, more preferably an αβT-cell, as improved surface expression may in some cases correlate with an improved anti-tumour or anti-infective response.

Replacing a Cγ2 constant region or a part thereof by a Cγ1 constant region or part thereof may result in an improvement of expression, preferably surface expression, of the γδT-cell receptor or part thereof in a T-cell, preferably γδT-cell or αβT-cell, more preferably αβT-cell. Expression, preferably surface expression, may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100% higher, or more relative to expression of the γδT-cell receptor or part thereof it is derived from in an equivalent cell. Expression and surface expression may be assessed by routine methods, described earlier herein. Further examples are provided in the experimental section herein.

Identifying the γδT-cell receptor or part thereof that mediates the anti-tumour or anti-infective response in the methods involving replacement of a Cγ2 constant region or part thereof by a Cγ1 constant region or part thereof may be done, for example, by cross-referencing the engineered T-cells exhibiting an anti-tumour or anti-infective response with the γδT-cell receptor or part thereof they express. As another example, in the case of soluble γδT-cell receptors or parts thereof, identification may be done by identifying the soluble γδT-cell receptor or part thereof that resulted in the T-cell it was contacted with exhibiting an anti-tumour or anti-infective response. The amino acid sequence of the γT-cell receptor chain or part thereof and the δT-cell receptor chain or part thereof, or the nucleotide sequence of nucleic acid molecules encoding them, may be obtained using any sequencing method known to the skilled person.

In some embodiments of the methods involving replacement of a Cγ2 constant region or part thereof by a Cγ1 constant region or part thereof, the Cγ1 constant region or part thereof is represented by an amino acid sequence comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100%, preferably at least 95%, sequence identity or similarity with SEQ ID NO: 152.

In some embodiments of the methods involving replacement of a Cγ2 constant region or part thereof by a Cγ1 constant region or part thereof, the Cγ1 constant region or part thereof is represented by an amino acid sequence which does not comprise SEQ ID NO: 163.

In some embodiments of the methods involving replacement of a Cγ2 constant region or part thereof by a Cγ1 constant region or part thereof, the Cγ1 constant region or part thereof is represented by an amino acid sequence which does not comprise SEQ ID NO: 165.

In some embodiments of the methods involving replacement of a Cγ2 constant region or part thereof by a Cγ1 constant region or part thereof, the Cγ1 constant region or part thereof is represented by an amino acid sequence which comprises at least 80% sequence identity or similarity with SEQ ID NO: 152 and does not comprise SEQ ID NO: 163 or SEQ ID NO: 165.

In some embodiments of the methods involving replacing a nucleotide sequence encoding a Cγ2 constant region or a part thereof by a nucleotide sequence encoding a Cγ1 constant region or a part thereof, the nucleic acid molecule encoding the Cγ1 constant region or part thereof is represented by a sequence comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100%, preferably at least 95%, sequence identity with SEQ ID NO: 151.

In some embodiments of the methods involving replacement of a Cγ2 constant region or part thereof by a Cγ1 constant region or part thereof, the Cγ1 constant region or part thereof is represented by an amino acid sequence that does not comprise SEQ ID NO: 158 or part thereof.

In some embodiments of the methods involving replacement of a Cγ2 constant region or part thereof by a Cγ1 constant region or part thereof, the Cγ2 constant region or part thereof is represented by an amino acid sequence comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100%, preferably at least 95%, sequence identity or similarity with SEQ ID NO: 161 or SEQ ID NO: 164, preferably with SEQ ID NO: 161.

In some embodiments of the methods involving replacement of a Cγ2 constant region or part thereof by a Cγ1 constant region or part thereof, the Cγ2 constant region or part thereof comprises SEQ ID NO: 163.

In some embodiments of the methods involving replacement of a Cγ2 constant region or part thereof by a Cγ1 constant region or part thereof, the Cγ2 constant region or part thereof comprises SEQ ID NO: 165.

In some embodiments of the methods involving replacement of a Cγ2 constant region or part thereof by a Cγ1 constant region or part thereof, the Cγ2 constant region or part thereof is represented by an amino acid sequence that does not comprise SEQ ID NO: 159 or part thereof.

In a further aspect, there is provided a γT-cell receptor chain or a part thereof (or a γδT-cell receptor or a part thereof comprising it) obtained by or obtainable by a method involving replacement of a Cγ2 constant region or part thereof by a Cγ1 constant region or part thereof described herein.

Accordingly, the invention further provides a γT-cell receptor chain or part thereof comprising a Cγ1 constant region or part thereof. The invention further provides a γδT-cell receptor or part thereof comprising a γT-cell receptor chain or part thereof, wherein said chain or part thereof comprises a Cγ1 constant region or part thereof. In some embodiments, the Cγ1 region or part thereof is represented by an amino acid sequence comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100%, preferably at least 95%, sequence identity or similarity with SEQ ID NO: 152. In some embodiments, the Cγ1 constant region or part thereof is represented by an amino acid sequence that does not comprise SEQ ID NO: 158 or part thereof. In some embodiments, the Cγ1 constant region or part thereof is represented by an amino acid sequence which does not comprise SEQ ID NO: 163. In some embodiments, the Cγ1 constant region or part thereof is represented by an amino acid sequence which does not comprise SEQ ID NO: 165.

In some embodiments, the Cγ1 constant region or part thereof is represented by an amino acid sequence which does not comprise an isoleucine in a position corresponding to position 41 of SEQ ID NO: 161 or SEQ ID NO: 164.

In some embodiments, the Cγ1 constant region or part thereof is represented by an amino acid sequence which comprises a valine in a position corresponding to position 41 of SEQ ID NO: 152.

In some embodiments, the Cγ1 constant region or part thereof is represented by an amino acid sequence which does not comprise a glutamine in a position corresponding to position 47 of SEQ ID NO: 161 or SEQ ID NO: 164.

In some embodiments, the Cγ1 constant region or part thereof is represented by an amino acid sequence which comprises a glutamic acid in a position corresponding to position 47 of SEQ ID NO: 152.

In some embodiments, the Cγ1 constant region or part thereof is represented by an amino acid sequence which does not comprise a glutamic acid in a position corresponding to position 80 of SEQ ID NO: 161 or SEQ ID NO: 164.

In some embodiments, the Cγ1 constant region or part thereof is represented by an amino acid sequence which comprises a lysine in a position corresponding to position 80 of SEQ ID NO: 152.

In some embodiments, the Cγ1 constant region or part thereof is represented by an amino acid sequence which does not comprise an isoleucine in a position corresponding to position 99 of SEQ ID NO: 161 or SEQ ID NO: 164.

In some embodiments, the Cγ1 constant region or part thereof is represented by an amino acid sequence which comprises a valine in a position corresponding to position 99 of SEQ ID NO: 152.

In some embodiments, the Cγ1 constant region or part thereof is represented by an amino acid sequence which does not comprise a tryptophan in a position corresponding to position 137 of SEQ ID NO: 161 or SEQ ID NO: 164.

In some embodiments, the Cγ1 constant region or part thereof is represented by an amino acid sequence which comprises a cysteine in a position corresponding to position 121 of SEQ ID NO: 152.

In some embodiments, the Cγ1 constant region or part thereof is represented by an amino acid sequence which does not comprise a threonine in a position corresponding to position 157 of SEQ ID NO: 164.

In some embodiments, the Cγ1 constant region or part thereof is represented by an amino acid sequence which comprises a methionine in a position corresponding to position 141 of SEQ ID NO: 152.

In some embodiments, the Cγ1 constant region or part thereof is represented by an amino acid sequence which does not comprise a glycine in a position corresponding to position 178 of SEQ ID NO: 161.

In some embodiments, the Cγ1 constant region or part thereof is represented by an amino acid sequence which comprises an arginine in a position corresponding to position 162 of SEQ ID NO: 152.

In some embodiments, the Cγ1 constant region or part thereof is represented by an amino acid sequence which does not comprise an aspartic acid in a position corresponding to position 111 of SEQ ID NO: 161 or SEQ ID NO: 164.

In some embodiments, the Cγ1 constant region or part thereof is represented by an amino acid sequence which does not comprise a valine in a position corresponding to position 112 of SEQ ID NO: 161 or SEQ ID NO: 164.

In some embodiments, the Cγ1 constant region or part thereof is represented by an amino acid sequence which does not comprise a threonine in a position corresponding to position 113 of SEQ ID NO: 161 or SEQ ID NO: 164.

In some embodiments, the Cγ1 constant region or part thereof is represented by an amino acid sequence which does not comprise a threonine in a position corresponding to position 114 of SEQ ID NO: 161 or SEQ ID NO: 164.

In some embodiments, the Cγ1 constant region or part thereof is represented by an amino acid sequence which does not comprise a valine in a position corresponding to position 115 of SEQ ID NO: 161 or SEQ ID NO: 164.

In some embodiments, the Cγ1 constant region or part thereof is represented by an amino acid sequence which does not comprise an aspartic acid in a position corresponding to position 116 of SEQ ID NO: 161 or SEQ ID NO: 164.

In some embodiments, the Cγ1 constant region or part thereof is represented by an amino acid sequence which does not comprise a proline in a position corresponding to position 117 of SEQ ID NO: 161 or SEQ ID NO: 164.

In some embodiments, the Cγ1 constant region or part thereof is represented by an amino acid sequence which does not comprise a lysine in a position corresponding to position 118 of SEQ ID NO: 161 or SEQ ID NO: 164.

In some embodiments, the Cγ1 constant region or part thereof is represented by an amino acid sequence which does not comprise a tyrosine in a position corresponding to position 119 of SEQ ID NO: 161 or SEQ ID NO: 164.

In some embodiments, the Cγ1 constant region or part thereof is represented by an amino acid sequence which does not comprise an asparagine in a position corresponding to position 120 of SEQ ID NO: 161 or SEQ ID NO: 164.

In some embodiments, the Cγ1 constant region or part thereof is represented by an amino acid sequence which does not comprise a tyrosine in a position corresponding to position 121 of SEQ ID NO: 161 or SEQ ID NO: 164.

In some embodiments, the Cγ1 constant region or part thereof is represented by an amino acid sequence which does not comprise a serine in a position corresponding to position 122 of SEQ ID NO: 161 or SEQ ID NO: 164.

In some embodiments, the Cγ1 constant region or part thereof is represented by an amino acid sequence which does not comprise a lysine in a position corresponding to position 123 of SEQ ID NO: 161 or SEQ ID NO: 164.

In some embodiments, the Cγ1 constant region or part thereof is represented by an amino acid sequence which does not comprise an aspartic acid in a position corresponding to position 124 of SEQ ID NO: 161 or SEQ ID NO: 164.

In some embodiments, the Cγ1 constant region or part thereof is represented by an amino acid sequence which does not comprise an alanine in a position corresponding to position 125 of SEQ ID NO: 161 or SEQ ID NO: 164.

In some embodiments, the Cγ1 constant region or part thereof is represented by an amino acid sequence which does not comprise an asparagine in a position corresponding to position 126 of SEQ ID NO: 161 or SEQ ID NO: 164.

The γT-cell receptor chain or part thereof (or the γδT-cell receptor or part thereof comprising it) may be soluble. The γT-cell receptor chain or part thereof (or the γδT-cell receptors or parts thereof comprising it) may be comprised in a soluble polypeptide, and may be fused with a T-cell- and/or NK-cell-binding domain as described earlier herein. Optionally, additional domains may be comprised in the soluble polypeptide (such as, but not limited to, signal peptides, tags, and/or linkers), as described earlier herein. A preferred T-cell-binding domain is a CD3-binding domain, preferably an scFv CD3-binding domain, more preferably represented by an amino acid sequence comprising or consisting of SEQ ID NO: 146 or a variant thereof.

The γT-cell receptor chain or part thereof (or the γδT-cell receptor or part thereof comprising it) may be expressed in a cell, preferably a T-cell, more preferably a γδT-cell or an αβT-cell, most preferably an αβT-cell, said cell optionally expressing additional proteins such as the chimeric bidirectional signaling transmembrane proteins described earlier herein. A preferred chimeric bidirectional signaling transmembrane protein comprises an extracellular ligand domain comprising an amino acid sequence from 41BBL, OX40L, CD86, RANK, or CD70, and a heterologous intracellular signaling domain comprising an amino acid sequence from OX40, 41BB, NKp80, IL18RAP, or IL2RB. A further preferred chimeric bidirectional signaling transmembrane protein is represented by an amino acid sequence comprising at least 80%, at least 85%, at least 90%, at least 95%, or 100% identity or similarity with the amino acid sequence SEQ ID NO: 83.

The γT-cell receptor chain or part thereof (or the γδT-cell receptor or parts thereof comprising it) may mediate an anti-tumour or anti-infective response. Accordingly, the γT-cell receptor chain or part thereof (or the γδT-cell receptor or part thereof comprising it) and/or the cells expressing it may be used in any of the therapeutic applications described herein, for example they may be used for preventing, treating, regressing, curing and/or delaying a cancer or an infection as described earlier herein. In some embodiments, the cancer is a metastatic cancer. In some embodiments, the cancer is a relapsed or refractory cancer. In some embodiments, a cancer is a solid tumour or a hematologic malignancy. In some embodiments, the cancer is a solid tumour. In some embodiments, the cancer is a hematologic malignancy. In some embodiments, a cancer may be a liquid cancer. In some embodiments, a cancer may be Acute myeloid leukemia (AML). In some embodiments, a cancer may be Multiple Myeloma (MM). In some embodiments, a cancer may be a solid cancer. In some embodiments, a cancer may be an ovarian cancer. In some embodiments, a cancer may be a breast cancer. In some embodiments, a cancer may be a colon cancer. In some embodiments, a cancer may be a kidney cancer. In some embodiments, a cancer may be a renal cancer. In some embodiments, a cancer may be a skin cancer, for example melanoma. In some embodiments, a cancer may be a lung cancer. In some cases, a subject has an infection as described earlier herein.

The γT-cell receptor chain or part thereof (or the γδT-cell receptor or parts thereof comprising it) and/or the cells expressing it be present in a composition, preferably a pharmaceutical composition, as described earlier herein.

### General

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a method, respectively component as defined herein may comprise additional step(s), respectively component(s) than the ones specifically identified, said additional step(s), respectively component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” when used in association with an integer (about 10) preferably means that the value may be the given value of 10 more or less 1 of the value: about 10 preferably means from 9 to 11. The word “about” when used in association with a numerical value (about 10.6) preferably means that the value may be the given value of 10.6 more or less 1% of the value 10.6.

All patent and literature references cited in the present specification are hereby incorporated by reference in their entirety. The following examples are offered for illustrative purposes only and are not intended to limit the scope of the present invention in any way.

TABLE 6
Overview of the sequences of the sequence listing

| | | Amino acid |
|---|---|---|
| SEQ ID NO | Name | (aa) or DNA |
| 1 | TRD CDR3 clone 1 | aa |
| 2 | TRD codon optimised clone 1 | DNA |
| 3 | TRD full length clone 1 | aa |
| 4 | TRG CDR3 clone 1 | aa |
| 5 | TRG codon optimised clone 1 | DNA |
| 6 | TRG full length clone 1 | aa |
| 7 | TRD CDR3 clone 2 | aa |
| 8 | TRD codon optimised clone 2 | DNA |
| 9 | TRD full length clone 2 | aa |
| 10 | TRG CDR3 clone 2 | aa |
| 11 | TRG codon optimised clone 2 | DNA |
| 12 | TRG full length clone 2 | aa |
| 13 | TRD CDR3 clone 3 | aa |
| 14 | TRD codon optimised clone 3 | DNA |
| 15 | TRD full length clone 3 | aa |
| 16 | TRG CDR3 clone 3 | aa |
| 17 | TRG codon optimised clone 3 | DNA |
| 18 | TRG full length clone 3 | aa |
| 19 | TRD CDR3 clone 4 | aa |
| 20 | TRD codon optimised clone 4 | DNA |
| 21 | TRD full length clone 4 | aa |
| 22 | TRG CDR3 clone 4 | aa |
| 23 | TRG codon optimised clone 4 | DNA |
| 24 | TRG full length clone 4 | aa |
| 25 | pLenti 6.3 | DNA |
| 26 | TRD full length wild type clone 1 | DNA |
| 27 | TRG full length wild type clone 1 | DNA |
| 28 | TRD full length wild type clone 2 | DNA |
| 29 | TRG full length wild type clone 2 | DNA |
| 30 | TRD full length wild type clone 3 | DNA |
| 31 | TRG full length wild type clone 3 | DNA |
| 32 | TRD full length wild type clone 4 | DNA |
| 33 | TRG full length wild type clone 4 | DNA |
| 34 | TRG clone 2-41BBL-OX40-TRD clone 2 | DNA |
| 35 | TRG clone 4-41BBL-OX40-TRD clone 4 | DNA |
| 36 | 41BBL-OX40 | DNA |
| 37 | 41BBL-OX40 GSG | DNA |
| 39-44 | Table 1 | aa |
| 45-57 | Table 2 | aa |
| 58-65 | Table 3 | aa |
| 66-82 | Table 4 | aa |
| 38, 83-119 | Table 5 | aa |
| 120 | MSCV promoter | DNA |
| 121 | CD70 | aa |
| 122 | IL2RB ICD | aa |
| 123 | CD70-TM | aa |
| 124 | 41BBL-13W-OX40 | aa |
| 125 | 41BBL-OX40-IL2RB | aa |
| 126 | CD70 | aa |
| 127 | CD70mincyto | aa |
| 128 | CD70mincyto-OX40 | aa |
| 129 | CD70-41BBL-OX40 | aa |
| 130 | TRG codon optimised clone 2 Cg1 | DNA |
| 131 | TRG full length clone 2 Cg1 | aa |
| 132 | TRG codon optimised clone 3 Cg2 | DNA |
| 133 | TRG full length clone 3 Cg2 | aa |
| 134 | TRG codon optimised clone 4 Cg1 | DNA |
| 135 | TRG full length clone 4 Cg1 | aa |
| 136 | clone 5 TRG CDR3 | aa |
| 137 | TRG codon optimised clone 5 Cg2 | DNA |
| 138 | TRG full length clone 5 Cg2 | aa |
| 139 | C132 TRG CDR3 | aa |
| 140 | TRG codon optimised C132 Cg2 | DNA |
| 141 | TRG full length C132 Cg2 | aa |
| 142 | TCR clone 3 TRD soluble | aa |
| 143 | TCR clone 3 TRG Cg1 soluble | aa |
| 144 | TCR clone 3 TRG Cg2 soluble | aa |
| 145 | linker | aa |
| 146 | scFv CD3 -binding | aa |
| 147 | linker | aa |
| 148 | AVI-TAG | aa |
| 149 | His-TAG | aa |
| 150 | pHCAG-L2EOP | DNA |
| 151 | Cg1 | DNA |
| 152 | Cg1 | aa |
| 153 | TCR clone 3 TRD soluble no signal pept | aa |
| 154 | TCR clone 3 TRG Cg1 soluble no signal | aa |
| | pept | |
| 155 | TCR clone 3 TRG Cg2 soluble no signal | aa |
| | pept | |
| 156 | signal pept gamma | aa |
| 157 | signal pept delta | aa |
| 158 | TRGC1 TM domain | aa |
| 159 | TRGC2 TM domain | aa |
| 160 | TRDC TM domain | aa |
| 161 | Cg2 | aa |
| 162 | TRG full length clone 3 | aa |
| 163 | Cg2 domain | aa |
| 164 | Cg2 | aa |
| 165 | Cg2 domain | aa |

TABLE 7
Exemplary sequences

| SEQ ID | | |
|---|---|---|
| NO | Name | Sequence |
| 7 | TRD CDR3 | CACGLPRSLADKLIF |
| | clone 2 | |
| 9 | TRD full length | MILTVGFSFLFFYRGTLCDKVTQSSPDQTVASGSEVVLLCTYD |
| | clone 2 | TVYSNPDLFWYRIRPDYSFQFVFYGDNSRSEGADFTQGRFS |
| | | VKHILTQKAFHLVISPVRTEDSATYYCACGLPRSLADKLIFGKG |
| | | TRVTVEPRSQPHTKPSVFVMKNGTNVACLVKEFYPKDIRINLV |
| | | SSKKITEFDPAIVISPSGKYNAVKLGKYEDSNSVTCSVQHDNK |
| | | TVHSTDFEVKTDSTDHVKPKETENTKQPSKSCHKPKAIVHTE |
| | | KVNMMSLTVLGLRMLFAKTVAVNFLLTAKLFFL |
| 10 | TRG CDR3 | CATWDLHKKLF |
| | clone 2 | |
| 12 | TRG full length | MEWALAVLLAFLSPASQKSSNLEGRTKSVIRQTGSSAEITCDL |
| | clone 2 | AEGSTGYIHWYLHQEGKAPQRLLYYDSYTSSVVLESGISPGK |
| | | YDTYGSTRKNLRMILRNLIENDSGVYYCATWDLHKKLFGSGT |
| | | TLVVTDKQLDADVSPKPTIFLPSIAETKLQKAGTYLCLLEKFFP |
| | | DIIKIHWQEKKSNTILGSQEGNTMKTNDTYMKFSWLTVPEESL |
| | | DKEHRCIVRHENNKNGIDQEIIFPPIKTDVTTVDPKYNYSKDAN |
| | | DVITMDPKDNWSKDANDTLLLQLTNTSAYYMYLLLLLKSVVYF |
| | | AIITCCLLGRTAFCCNGEKS |
| 13 | TRD CDR3 | CALGEFPYWGIPDKLIF |
| | clone 3 | |
| 15 | TRD full length | MVFSSLLCVFVAFSYSGSSVAQKVTQAQSSVSMPVRKAVTLN |
| | clone 3 | CLYETSWWSYYIFWYKQLPSKEMIFLIRQGSDEQNAKSGRYS |
| | | VNFKKAAKSVALTISALQLEDSAKYFCALGEFPYWGIPDKLIFG |
| | | KGTRVTVEPRSQPHTKPSVFVMKNGTNVACLVKEFYPKDIRI |
| | | NLVSSKKITEFDPAIVISPSGKYNAVKLGKYEDSNSVTCSVQH |
| | | DNKTVHSTDFEVKTDSTDHVKPKETENTKQPSKSCHKPKAIV |
| | | HTEKVNMMSLTVLGLRMLFAKTVAVNFLLTAKLFFL |
| 16 | TRG CDR3 | CATWDGYYKKLF |
| | clone 3 | |
| 18 | TRG full length | MRWALLVLLAFLSPASQKSSNLEGRTKSVTRQTGSSAEITCD |
| | clone 3 | LTVTNTFYIHWYLHQEGKAPQRLLYYDVSTARDVLESGLSPG |
| | | KYYTHTPRRWSWILRLQNLIENDSGVYYCATWDGYYKKLFGS |
| | | GTTLVVTDKQLDADVSPKPTIFLPSIAETKLQKAGTYLCLLEKF |
| | | FPDVIKIHWEEKKSNTILGSQEGNTMKTNDTYMKFSWLTVPE |
| | | KSLDKEHRCIVRHENNKNGVDQEIIFPPIKTDVITMDPKDNCSK |
| | | DANDTLLLQLTNTSAYYMYLLLLLKSVVYFAIITCCLLRRTAFC |
| | | CNGEK |
| 19 | TRD CDR3 | CALGGLGWGYAGDEGGGKLTDKLIF |
| | clone 4 | |
| 21 | TRD full length | MVFSSLLCVFVAFSYSGSSVAQKVTQAQSSVSMPVRKAVTLN |
| | clone 4 | CLYETSWWSYYIFWYKQLPSKEMIFLIRQGSDEQNAKSGRYS |
| | | VNFKKAAKSVALTISALQLEDSAKYFCALGGLGWGYAGDEGG |
| | | GKLTDKLIFGKGTRVTVEPRSQPHTKPSVFVMKNGTNVACLV |
| | | KEFYPKDIRINLVSSKKITEFDPAIVISPSGKYNAVKLGKYEDSN |
| | | SVTCSVQHDNKTVHSTDFEVKTDSTDHVKPKETENTKQPSKS |
| | | CHKPKAIVHTEKVNMMSLTVLGLRMLFAKTVAVNFLLTAKLFF |
| | | L |
| 22 | TRG CDR3 | CALWEVHPNYKKLF |
| | clone 4 | |
| 24 | TRG full length | MVSLLHASTLAVLGALCVYGAGHLEQPQISSTKTLSKTARLEC |
| | clone 4 | VVSGITISATSVYWYRERPGEVIQFLVSISYDGTVRKESGIPSG |
| | | KFEVDRIPETSTSTLTIHNVEKQDIATYYCALWEVHPNYKKLF |
| | | GSGTTLVVTDKQLDADVSPKPTIFLPSIAETKLQKAGTYLCLLE |
| | | KFFPDIIKIHWQEKKSNTILGSQEGNTMKTNDTYMKFSWLTVP |
| | | EESLDKEHRCIVRHENNKNGIDQEIIFPPIKTDVTTVDPKYNYS |
| | | KDANDVITMDPKDNWSKDANDTLLLQLTNTSAYYMYLLLLLKS |
| | | VVYFAIITCCLLGRTAFCCNGEKS |
| 131 | TRG full length | MEWALAVLLAFLSPASQKSSNLEGRTKSVIRQTGSSAEITCDL |
| | clone 2_Cg1 | AEGSTGYIHWYLHQEGKAPQRLLYYDSYTSSVVLESGISPGK |
| | | YDTYGSTRKNLRMILRNLIENDSGVYYCATWDLHKKLFGSGT |
| | | TLVVTDKQLDADVSPKPTIFLPSIAETKLQKAGTYLCLLEKFFP |
| | | DVIKIHWEEKKSNTILGSQEGNTMKTNDTYMKFSWLTVPEKS |
| | | LDKEHRCIVRHENNKNGVDQEIIFPPIKTDVITMDPKDNCSKD |
| | | ANDTLLLQLTNTSAYYMYLLLLLKSVVYFAIITCCLLRRTAFCC |
| | | NGEKS |
| 133 | TRG full length | MRWALLVLLAFLSPASQKSSNLEGRTKSVTRQTGSSAEITCD |
| | clone 3_Cg2 | LTVTNTFYIHWYLHQEGKAPQRLLYYDVSTARDVLESGLSPG |
| | | KYYTHTPRRWSWILRLQNLIENDSGVYYCATWDGYYKKLFGS |
| | | GTTLVVTDKQLDADVSPKPTIFLPSIAETKLQKAGTYLCLLEKF |
| | | FPDIIKIHWQEKKSNTILGSQEGNTMKTNDTYMKFSWLTVPEE |
| | | SLDKEHRCIVRHENNKNGIDQEIIFPPIKTDVTTVDPKYNYSKD |
| | | ANDVITMDPKDNWSKDANDTLLLQLTNTSAYYMYLLLLLKSVV |
| | | YFAIITCCLLGRTAFCCNGEKS |
| 135 | TRG full length | MVSLLHASTLAVLGALCVYGAGHLEQPQISSTKTLSKTARLEC |
| | clone 4_Cg1 | VVSGITISATSVYWYRERPGEVIQFLVSISYDGTVRKESGIPSG |
| | | KFEVDRIPETSTSTLTIHNVEKQDIATYYCALWEVHPNYKKLF |
| | | GSGTTLVVTDKQLDADVSPKPTIFLPSIAETKLQKAGTYLCLLE |
| | | KFFPDVIKIHWEEKKSNTILGSQEGNTMKTNDTYMKFSWLTV |
| | | PEKSLDKEHRCIVRHENNKNGVDQEIIFPPIKTDVITMDPKDNC |
| | | SKDANDTLLLQLTNTSAYYMYLLLLLKSVVYFAIITCCLLRRTAF |
| | | CCNGEKS |
| 142 | TCR clone 3 | MACPGFLWALVISTCLEFSMASAAQKVTQAQSSVSMPVRKA |
| | TRD_soluble | VTLNCLYETSWWSYYIFWYKQLPSKEMIFLIRQGSDEQNAKS |
| | | GRYSVNFKKAAKSVALTISALQLEDSAKYFCALGEFPYWGIPD |
| | | KLIFGKGTRVTVEPRSQPHTKPSVFVMKNGTNVACLVKEFYP |
| | | KDIRINLVSSKKITEFDPAIVISPSGKYNAVKLGKYEDSNSVTCS |
| | | VQHDNKTVHSTDFEVKTDSTDHVKPKETENTKQPSKS |
| 143 | TCR clone 3 | MACPGFLWALVISTCLEFSMARTSSNLEGRTKSVTRQTGSSA |
| | TRG_Cg1_ | EITCDLTVTNTFYIHWYLHQEGKAPQRLLYYDVSTARDVLESG |
| | soluble | LSPGKYYTHTPRRWSWILRLQNLIENDSGVYYCATWDGYYK |
| | | KLFGSGTTLVVTDKQLDADVSPKPTIFLPSIAETKLQKAGTYLC |
| | | LLEKFFPDVIKIHWEEKKSNTILGSQEGNTMKTNDTYMKFSWL |
| | | TVPEKSLDKEHRCIVRHENNKNGVDQEIIFPPIKTDVITMDPKD |
| | | N |
| 144 | TCR clone 3 | MACPGFLWALVISTCLEFSMARTSSNLEGRTKSVTRQTGSSA |
| | TRG_Cg2_ | EITCDLTVTNTFYIHWYLHQEGKAPQRLLYYDVSTARDVLESG |
| | soluble | LSPGKYYTHTPRRWSWILRLQNLIENDSGVYYCATWDGYYK |
| | | KLFGSGTTLVVTDKQLDADVSPKPTIFLPSIAETKLQKAGTYLC |
| | | LLEKFFPDIIKIHWQEKKSNTILGSQEGNTMKTNDTYMKFSWL |
| | | TVPEESLDKEHRCIVRHENNKNGIDQEIIFPPIKTDVTTVDPKY |
| | | NYSKDANDVITMDPKDN |
| 146 | scFv CD3- | QVQLVQSGGGVVQPGRSLRLSCKASGYTFTRYTMHWVRQA |
| | binding | PGKGLEWIGYINPSRGYTNYNQKFKDRFTISRDNSKNTAFLQ |
| | | MDSLRPEDTGVYFCARYYDDHYCLDYWGQGTPVTVSSGGG |
| | | GSGGGGSGGGGSDIQMTQSPSSLSASVGDRVTITCSASSSV |
| | | SYMNWYQQTPGKAPKRWIYDTSKLASGVPSRFSGSGSGTD |
| | | YTFTISSLQPEDIATYYCQQWSSNPFTFGQGTKLQITRVDGS |
| 152 | Cg1 | DKQLDADVSPKPTIFLPSIAETKLQKAGTYLCLLEKFFPD |
| | | VIKIHWEEKKSNTILGSQEGNTMKTNDTYMKFSWLTVPEK |
| | | SLDKEHRCIVRHENNKNGVDQEIIFPPIKTDVITMDPKDN |
| | | CSKDANDTLLLQLTNTSAYYMYLLLLLKSVVYFAIITCCL |
| | | LRRTAFCCNG EKS |
| 153 | TCR clone 3 | KVTQAQSSVSMPVRKAVTLNCLYETSWWSYYIFWYKQLPSK |
| | TRD_soluble_ | EMIFLIRQGSDEQNAKSGRYSVNFKKAAKSVALTISALQLEDS |
| | no signal pept | AKYFCALGEFPYWGIPDKLIFGKGTRVTVEPRSQPHTKPSVF |
| | | VMKNGTNVACLVKEFYPKDIRINLVSSKKITEFDPAIVISPSGK |
| | | YNAVKLGKYEDSNSVTCSVQHDNKTVHSTDFEVKTDSTDHV |
| | | KPKETENTKQPSKS |
| 154 | TCR clone 3 | NLEGRTKSVTRQTGSSAEITCDLTVTNTFYIHWYLHQEGKAP |
| | TRG_Cg1_ | QRLLYYDVSTARDVLESGLSPGKYYTHTPRRWSWILRLQNLI |
| | soluble_no | ENDSGVYYCATWDGYYKKLFGSGTTLVVTDKQLDADVSPKP |
| | signal pept | TIFLPSIAETKLQKAGTYLCLLEKFFPDVIKIHWEEKKSNTILGS |
| | | QEGNTMKTNDTYMKFSWLTVPEKSLDKEHRCIVRHENNKNG |
| | | VDQEIIFPPIKTDVITMDPKDN |
| 155 | TCR clone 3 | NLEGRTKSVTRQTGSSAEITCDLTVTNTFYIHWYLHQEGKAP |
| | TRG_Cg2_ | QRLLYYDVSTARDVLESGLSPGKYYTHTPRRWSWILRLQNLI |
| | soluble_no | ENDSGVYYCATWDGYYKKLFGSGTTLVVTDKQLDADVSPKP |
| | signal pept | TIFLPSIAETKLQKAGTYLCLLEKFFPDIIKIHWQEKKSNTILGSQ |
| | | EGNTMKTNDTYMKFSWLTVPEESLDKEHRCIVRHENNKNGID |
| | | QEIIFPPIKTDVTTVDPKYNYSKDANDVITMDPKDN |
| 161 | Cg2 | DKQLDADVSPKPTIFLPSIAETKLQKAGTYLCLLEKFFPDIIKIH |
| | | WQEKKSNTILGSQEGNTMKTNDTYMKFSWLTVPEESLDKEH |
| | | RCIVRHENNKNGIDQEIIFPPIKTDVTTVDPKYNYSKDANDVIT |
| | | MDPKDNWSKDANDTLLLQLTNTSAYYMYLLLLLKSVVYFAIIT |
| | | CCLLGRTAFCCNGEKS |
| 162 | TRG full length | MRWALLVLLAFLSPASQKSSNLEGRTKSVTRQTGSSAEITCD |
| | clone 3 | LTVTNTFYIHWYLHQEGKAPQRLLYYDVSTARDVLESGLSPG |
| | | KYYTHTPRRWSWILRLQNLIENDSGVYYCATWDGYYKKLFGS |
| | | GTTLVVTDKQLDADVSPKPTIFLPSIAETKLQKAGTYLCLLEKF |
| | | FPDVIKIHWEEKKSNTILGSQEGNTMKTNDTYMKFSWLTVPE |
| | | KSLDKEHRCIVRHENNKNGVDQEIIFPPIKTDVITMDPKDNCSK |
| | | DANDTLLLQLTNTSAYYMYLLLLLKSVVYFAIITCCLLRRTAFC |
| | | CNGEKS |
| 163 | Cg2 domain | DVTTVDPKYNYSKDAN |
| 164 | Cg2 | DKQLDADVSPKPTIFLPSIAETKLQKAGTYLCLLEKFFPDIIKIH |
| | | WQEKKSNTILGSQEGNTMKTNDTYMKFSWLTVPEESLDKEH |
| | | RCIVRHENNKNGIDQEIIFPPIKTDVTTVDPKYNYSKDANDVIT |
| | | MDPKDNWSKDANDTLLLQLTNTSAYYTYLLLLLKSVVYFAIITC |
| | | CLLRRTAFCCNGEKS |
| 165 | Cg2 domain | TTVDPKYNYSKDAN |

### EXAMPLES

It is understood that the examples and embodiments described herein are for illustrative purposes only and that various modifications or changes in light thereof will be suggested to persons skilled in the art and are to be included within the spirit and purview of this application and scope of the appended claims.

Unless specified, reagents employed in the examples are commercially available or can be prepared using commercially available instrumentation, methods, protocols, or reagents known in the art. The examples illustrate various aspects of the invention and practice of the methods of the invention. The examples are not intended to provide an exhaustive description of the many different embodiments of the invention. Thus, although the invention has been described in some detail by way of illustration and example for purposes of clarity of understanding, those of ordinary skill in the art will realize readily that many changes and modifications can be made thereto without departing from the spirit or scope of the appended claims. Numerous variations, changes, and substitutions will now occur to those skilled in the art without departing from the invention. It is intended that the claims define the scope of the invention and that methods and structures within the scope of these claims and their equivalents be covered thereby.

### Material & Methods Pertaining to the Examples Discussed Herein

γδTCR DNA subcloning and lentivirus preparation Codon optimized DNA encoding for the full length γ- and δ-chains of four putatively tumour targeting γδTCRs was generated (SEQ ID NO: 2, 5, 8, 11, 14, 17, 20, 23) and subcloned by gBlock gene assembly (IDT—Integrated DNA Technologies) into pLenti 6.3 lentiviral bicistronic vector (SEQ ID NO: 25), separated by a T2A self-cleaving peptide. Bicistronic expression of both γ and δTCR chains is driven by a MSCV promoter (SEQ ID NO: 120). This promoter has been disclosed in Jones S., et al (2009) (Joens S., et al (2009), Gene therapy, 20:630-640). Viral genome packaging and transgene expression enhancement are achieved by LTR/T and WPRE regulatory elements, respectively. Lentiviral particles were produced using the LV-Max system from Thermo Fisher Scientific. LV-MAX producer cells (A35827) were transfected with pLenti 6.3 γδTCR transfer construct and packaging mix (pLP1, pLP2, pLP-VSVG). Lentiviral titers were assessed in αβTCR-deficient Jurkat-76 cells by flow cytometry analysis, measuring the percentage of CD3/γδTCR among live cells.

For comparison experiments, the γδTCRs of clones Fe11 and Zil1, both as described in WP2017/212074 (refer to sequence id numbers 17, 18, 33, and 34 of WO2017/212074), were used.

For generation of the tested two soluble γδTCR-CD3 bispecific engagers, codon optimized DNA sequences encoding two soluble variants of the clone 3 γ3-chain (both encoding a part of TRGC1 (Cγ1 constant region) or TRGC2 (Cγ2 constrant region) genes; SEQ ID NOs: 143, 144) and a soluble variant of the clone 3 δ1-chain (SEQ ID NO: 142) were generated. Each γ3-chain-encoding sequence was paired with the δ1-chain-encoding sequence.

Both γ3-chain variants were connected to an anti-CD3 scFv derived from OKT3 antibody clone (SEQ ID NO: 146) via a linker (SEQ ID NO: 145), followed by a second linker (SEQ ID NO: 147), an AVI-TAG (suitable for biotinylation purposes; SEQ ID NO: 148) and His-tag (suitable for purification purposes; SEQ ID NO: 149).

The nucleotide sequences encoding the above were subcloned into pHCAG-L2EOP vector (SEQ ID NO: 150) by gBlock gene assembly (Addgene, MA, USA).

### TEG Production

TEGs were manufactured starting from αβT-cells enriched by MACS separation from healthy donor leukapheresis material, according to manufacturer instructions. Purified αβT-cells were cultured in TEXMACS medium supplemented with 2.5% human serum (Sanquin), rhlL-7 (20-2000 IU/mL) and rh IL15 (20-200 IU/mL) (both from Miltenyi), and 1% Penicillin/Streptomycin, and activated using TransAct (Miltenyi Biotec) per manufacturer's recommendations. Cells were transduced with γδTCR LV particles (MOI 5) and then expanded for 12 days in TEXMACS medium, 2.5% human serum, rhlL-7 (20-2000 IU/mL) and rh IL15 (20-200 IU/mL), 1% Penicillin/Streptomycin. At the end of the production, transduction efficiency (% γδTCR, >40% in all cases), T-cell purity (>90% in all cases), and relative expression of T-cell markers CD4 and CD8 were measured by flow cytometry. Cells were then cryopreserved in 1 volume of NaCl 0.9%/5% human serum albumin and 1 volume of Cryostor CS10 (Sigma-Aldrich).

### Soluble γδTCR-CD3 Bispecific Engager Production

Soluble γδTCR CD3 bispecific engagers derived from clone 3 γδTCR and comprising Cγ1 or Cγ2 constant region parts were produced by co-transfecting both plasmid constructs encoding the soluble γ3-chain of TCR clone 3 (either Cγ1- or Cγ2-versions) and the soluble δ1-chain of TCR clone 3 (as discussed above) into HEK293F cells, using the transfection reagent PEImax (Polysciences Inc.) at PEImax:DNA ratio of 3:1. The transfection was carried out using Optimem solution (Thermo Fisher Scientific) and in the presence of Biotin (final concentration 25ug/ml, Thermo Fisher Scientific). After 7 days post-transfection, supernatants were collected and soluble γδTCR clone 3-CD3 bispecific engagers were purified via His-TAG using Histrap columns (Sigma-Aldrich) following the manufacturer's protocol. Purity was assessed by SDS-PAGE gel (in denaturating/non denaturating conditions) and Coomassie staining.

xCELLigence Cytotoxicity Assay

TEG anti-tumour activity towards several tumour cell lines was evaluated in vitro by measuring the killing of tumour target cells in a xCELLigence co-culture assay (Agilent, Santa Clara, CA, USA). First, cell lines were harvested, counted and seeded to the appropriate number of cells per well in triplicate in 96-well E-plates, and then placed in the xCELLigence cradles. Target cell adhesion and proliferation was measured for 24 hours. TEG or negative control untransduced αβT-cells were then harvested, counted, resuspended in IMDM medium, 5% human serum, and 1% Penicillin/Streptomycin, and added to the tumour target cells at Effector/Target ratio of 1:1. Loss of target cell adherence, as a readout for cytotoxicity, was measured for 48 hours. Cytotoxicity was calculated as percentage of cytolysis relative to maximum cytolysis induced by treatment of the target cells with the detergent Triton-X-100.

### Luciferase-Based Serial Cytotoxicity Assay

The positive effect of the presence of 41BBL-OX40 protein (SEQ ID NO: 83) on the anti-tumour activity of two of the three γδTCRs of the disclosure was assessed by luciferase-based serial cytotoxicity assays. Briefly, Luc-Tom MDA-MB-231 tumour cells were harvested, counted and seeded to the appropriate number of cells per well, in triplicate in 96-well E-plates, and cultured for 24 hours at 37° C. TEGs expressing γδTCR clone 2 (SEQ ID NO: 9, 12 with CDR3 regions represented by SEQ ID NO: 7, 10) or γδTCR clone 4 (SEQ ID NO: 21, 24 with CDR3 regions represented by SEQ ID NO: 19, 22), with or without 41BBL-OX40 protein were then added at effector to target (E:T) ratios of 1:1, 1:2 and 1:4, and the resulting co-culture maintained at 37° C. for 48 hours. At the end of the co-culture, effector T-cells were harvested and transferred to a new cell culture plate containing fresh Luc-Tom MDA-MB-231 tumour cells, for a new round of target exposure/stimulation, and luciferase activity measurements were performed on the old co-culture plate, by adding D-luciferine substrate (ThermoFisher Scientific, Waltham, MA, USA) and reading the luminescence in endpoint mode using Glomax luminometer according to the manufacturer's instructions (Promega, Madison, WI, USA). Cytolysis/cytotoxicity was calculated using the following formula: 100x [1-(Luminescence from target cells in co-culture with effector T-cells/Luminescence from target cells cultured alone)]. The co-culture assay was repeated for a total of three rounds of stimulation.

### IFN-γ ELISA Assay

Cell culture supernatants from xCELLigence cytotoxicity assays were harvested at the end of the 48-hour co-culture to measure IFN-γ secretion using a commercial Human IFN-gamma DuoSet ELISA assay (cat nr. DY285B-05, R&D Systems, Minneapolis, MN, US), according to manufacturer's instructions. This is a standard sandwich ELISA using a plate-bound capture antibody and a detection antibody both specific for IFN-γ. The detection antibody is linked to an enzyme which can convert a substrate into an absorbance signal which is measured with a plate reader. The internal standard curve allows absorbance values to be calculated into the IFN-γ concentration (pg/mL) released into the supernatants.

### Example 1

In this example, we isolated four novel γδTCRs from human subjects and tested the capacity of αβT-cells engineered to express these γδTCRs (TEGs) to recognize and kill a panel of tumour cell lines using an impedance-based cytotoxicity assay. TEG reactivity towards the same tumour cell lines was also assessed by measuring the release of the cytokine IFN-γ into the cell culture supernatants.

TCR clone 1 is a Vγ3Vδ3TCR (CDR3 regions represented by SEQ ID NO: 1, 4) and, as depicted in FIG. 2A-2B, TEGs expressing this γδTCR do not recognize any of the 4 analyzed tumour cell lines, as there is no significant increase in both cytolysis and IFN-γ secretion when compared to the negative control untransduced αβT-cells.

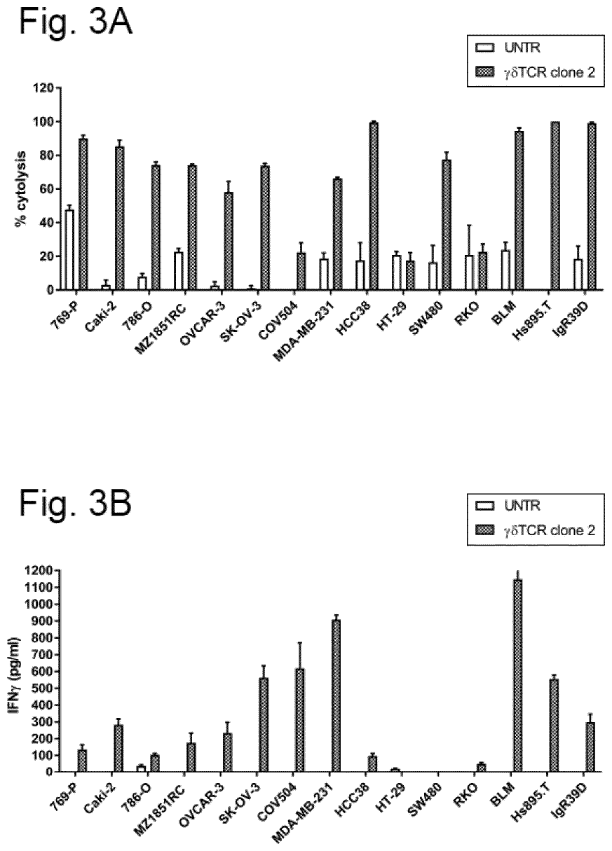

TCR clone 2 is a Vγ4Vδ3TCR (CDR3 regions represented by SEQ ID NO: 7, 10) and, as depicted in FIG. 3A-3B, TEGs expressing this γδTCR display a potent and broad recognition of tumour cell lines, as they display a significant increase in cytolytic activity and IFN-γ secretion towards 12 out of 15 tumour cell lines of different tissue origin, when compared to the negative control untransduced αβT-cells.

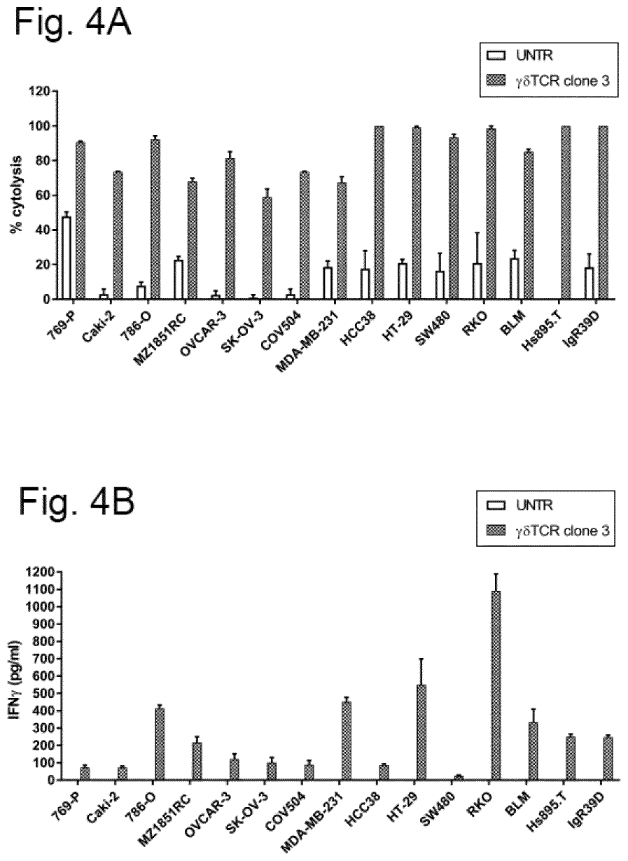

TCR clone 3 is a Vγ3Vδ1TCR (CDR3 regions represented by SEQ ID NO: 13, 16) and, as depicted in FIG. 4A-4B, TEGs expressing this γδTCR display a potent and broad recognition of tumour cell lines, as they display a significant increase in cytolytic activity and IFN-γ secretion towards 14 out of 15 tumour cell lines of different tissue origin, when compared to the negative control untransduced αβT-cells.

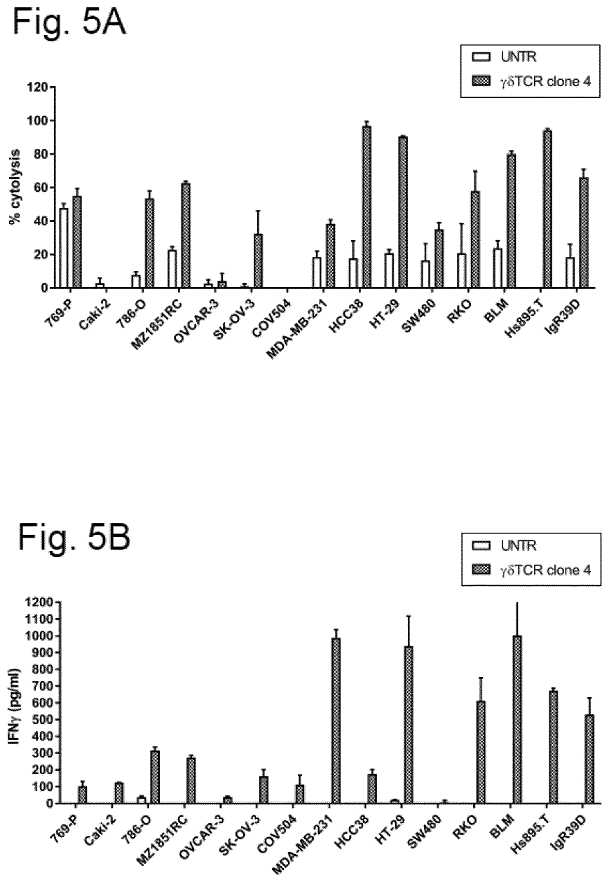

TCR clone 4, is a Vγ9Vδ1TCR (CDR3 regions represented by SEQ ID NO: 19, 22) and, as depicted in FIG. 5A-5B, TEGs expressing this γδTCR display a potent and broad recognition of tumour cell lines, as they display a significant increase in cytolytic activity and IFN-γ secretion towards 10 out of 15 tumour cell lines of different tissue origin, when compared to the negative control untransduced αβT-cells.

Furthermore, as described in FIG. 6A-6B, co-expression of the 41BBL-OX40 protein (SEQ ID NO: 83 enhances and prolongs the anti-tumour activity of the cells expressing γδTCRs of the disclosure.

In conclusion, three out of four evaluated γδTCRs display a potent and broad anti-tumour reactivity in the TEG format.

### Example 2

In this example, we compared the cytolytic activity and IFN-γ secretion of TEGs expressing γδTCRs from clone 2 (SEQ ID NO: 9, 12 with CDR3 regions represented by SEQ ID NO: 7, 10), clone 3 (SEQ ID NO: 15, 162 with CDR3 regions represented by SEQ ID NO: 13, 16), and clone 4 (SEQ ID NO: 21, 24 with CDR3 regions represented by SEQ ID NO: 19, 22) with TEGs expressing γδTCRs from clones Fe11 and Zi11 described in WO2017/212074.

For cytolytic activity comparisons, a luciferase-based serial cytotoxicity assay as discussed in the Materials & Methods was performed using Luc-Tom HT-29 or Luc-Tom NCI-226 tumour cells as targets, and the co-cultures were performed at effector to target (E:T) ratio of 1:1. Cells were co-cultured at 37° C. for 48 hours. At the end of the co-culture, effector T-cells were harvested and transferred to a new cell culture plate containing fresh tumour cells, for a new round of target exposure/stimulation, and luciferase activity measurements were performed as above described. IFN-γ production was measured as described in the Materials & Methods.

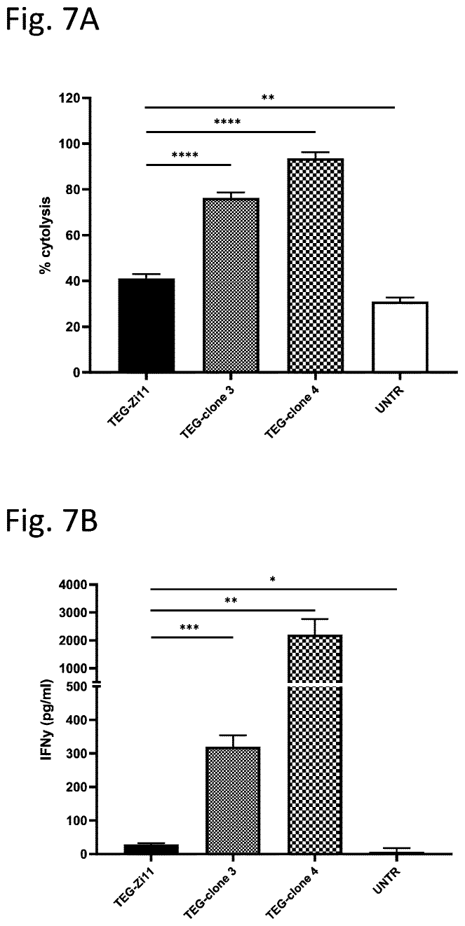

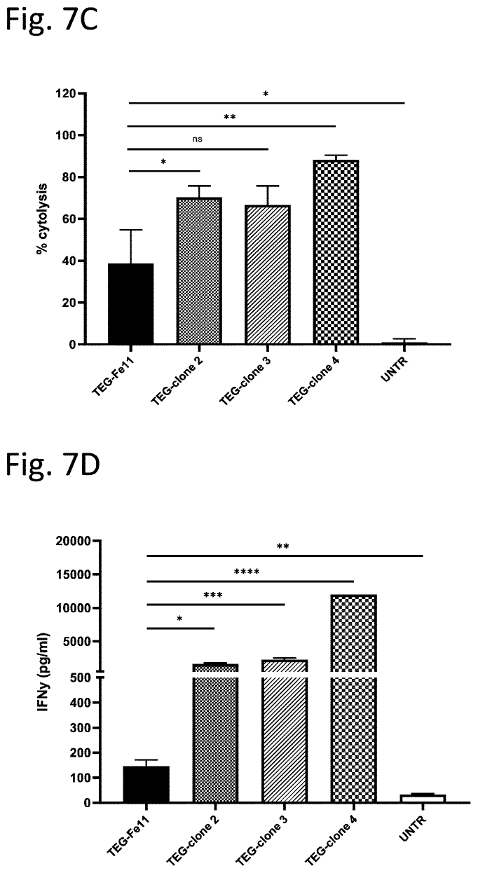

As depicted in FIG. 7A-7D, TEGs expressing γδTCRs from clones 2, 3, and 4 as described herein display more potent tumour reactivity compared to previously characterized γδTCRs.

### Example 3

In this example, we compared the surface expression of γδTCRs of clones 2, 3, and 4 comprising Cγ1 or Cγ2 constant regions in TEGs expressing them, as well as the cytolytic activity and IFN-γ secretion of the TEGs.

The γ-chain of clone 2 naturally comprises a Cγ2 constant region. For comparisons, the Cγ2 constant region in the γ-chain was replaced by a Cγ1 constant region, resulting in SEQ ID NO: 131. The chain comprising the Cγ1 constant region was paired with the δ-chain of clone 2 (SEQ ID NO: 9).

The γ-chain of clone 3 naturally comprises a Cγ1 constant region. For comparisons, the Cγ1 constant region in the γ-chain was replaced by a Cγ2 constant region, resulting in SEQ ID NO: 133. The chain comprising the Cγ2 constant region was paired with the δ-chain of clone 3 (SEQ ID NO: 15).

The γ-chain of clone 4 naturally comprises a Cγ2 constant region. For comparisons, the Cγ2 constant region in the γ-chain was replaced by a Cγ1 constant region, resulting in SEQ ID NO: 135. The chain comprising the Cγ1 constant region was paired with the δ-chain of clone 4 (SEQ ID NO: 21).

For additional comparisons, the Cγ1 constant regions of the γδTCRs of clones c15 and C132 described in WO2017/212074 (see sequence id numbers 15, 16, 37, and 38 in WO2017/212074) were replaced by a Cγ2 constant region, resulting in SEQ ID NO: 138 (c15) and SEQ ID NO: 141 (C132).

The original γδTCRs as well as the γδTCRs comprising a Cγ1 region (or Cγ2 region where applicable) generated herein were expressed in TEGs.

Lentivirus preparation and TEG production were performed as described in the Materials & Methods. For cytolytic activity comparisons in the case of clones 2, 3, and 4, a luciferase-based serial cytotoxicity assay as described in the Materials & Methods was performed using Luc-Tom MDA-MB-231 tumour cells, and the co-cultures were performed at effector to target (E:T) ratio of 1:1. Cells were co-cultured at 37° C. for 48 hours. At the end of the co-culture, effector T-cells were harvested and transferred to a new cell culture plate containing fresh tumour cells, for a new round of target exposure/stimulation, and luciferase activity measurements were performed as above described. IFN-γ production was measured as described in the Materials & Methods. Cytolytic activity comparisons in the case of c15 and C132 were made using xCELLigence as described in the Materials & Methods, using RKO colon carcinoma cells, HT-29 colon carcinoma cells, or SW480 colon carcinoma cells as targets.

Surface expression analysis was performed using flow cytometry using antibodies specific to αβTCR (clone IP26 (Biolegend, CA, USA) and γδTCR (clone IMMU510, Beckman Coulter, CA, USA). Acquisition was performed with BD FACS Fortessa and data analysized with FlowJo v10.

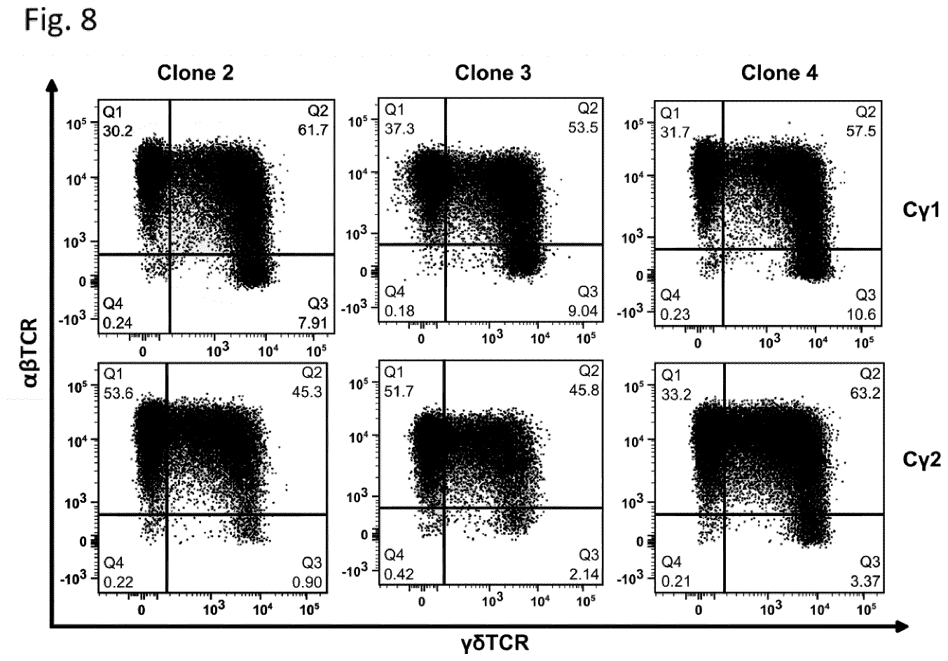

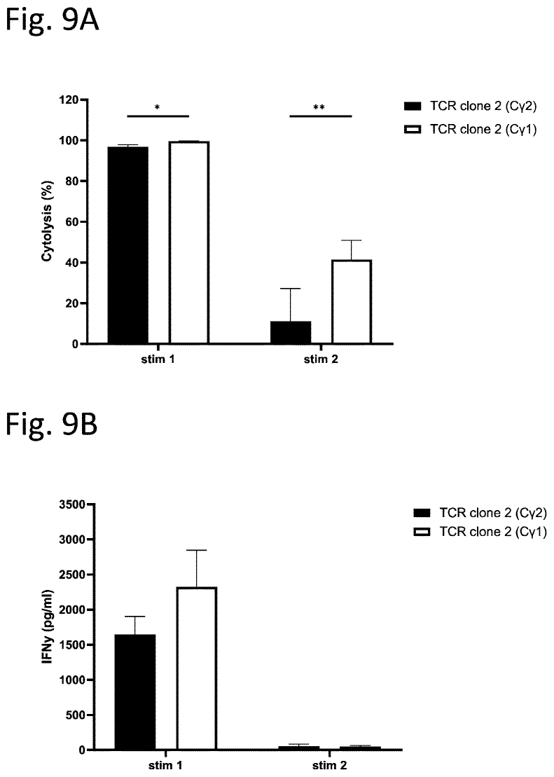

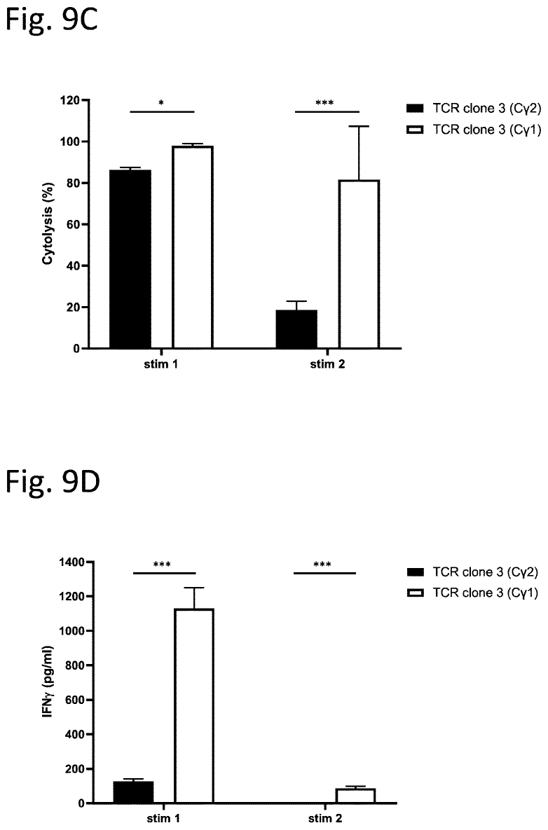

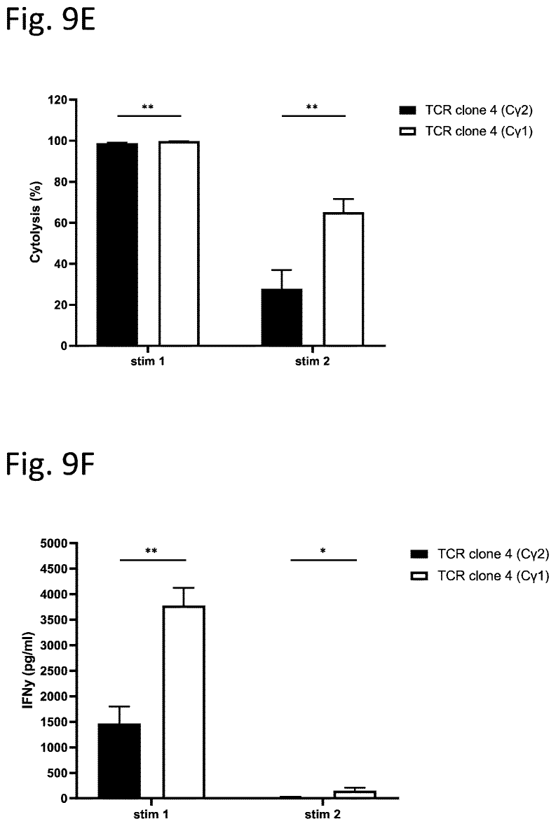

As shown in FIG. 8, a clear increase in cell surface expression of the transgene γδTCR with particular improvement in the γδTCR double positive population is observed in the case of TEGs expressing γδTCRs of clones 2, 3, and 4 comprising a Cγ1 constant region relative to γδTCRs comprising the same variable regions but comprising a Cγ2 constant region. More importantly, we observed a clear superior anti-tumour response of TEGs expressing γδTCRs comprising a Cγ1 constant region compared to their Cγ2 constant region-comprising counterparts (FIG. 9A-9F), and a clear correlation between improved surface expression of tested γδTCRs and improved anti-tumour response of TEGs expressing them.

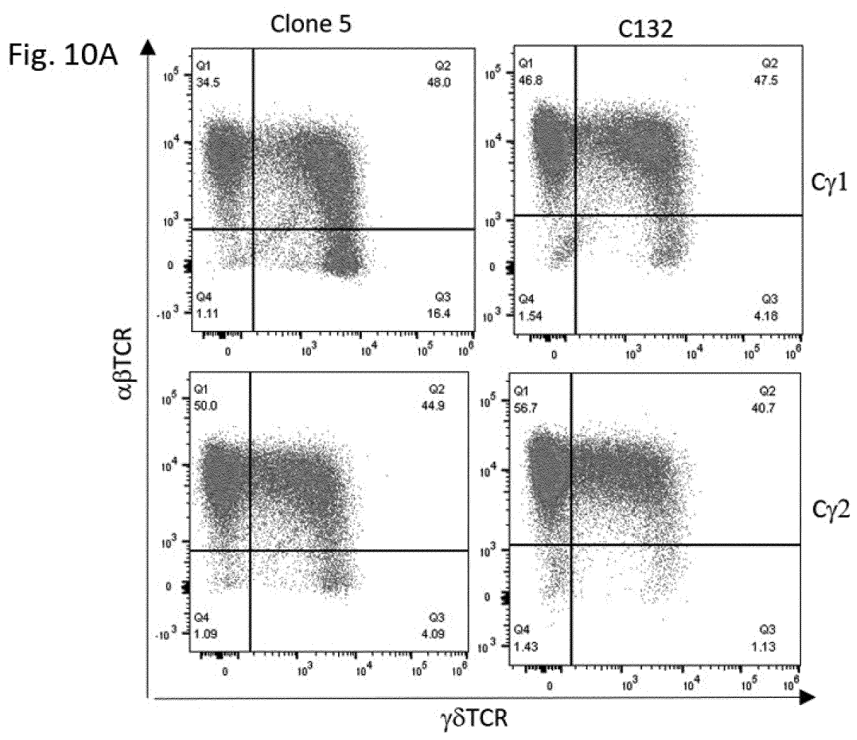

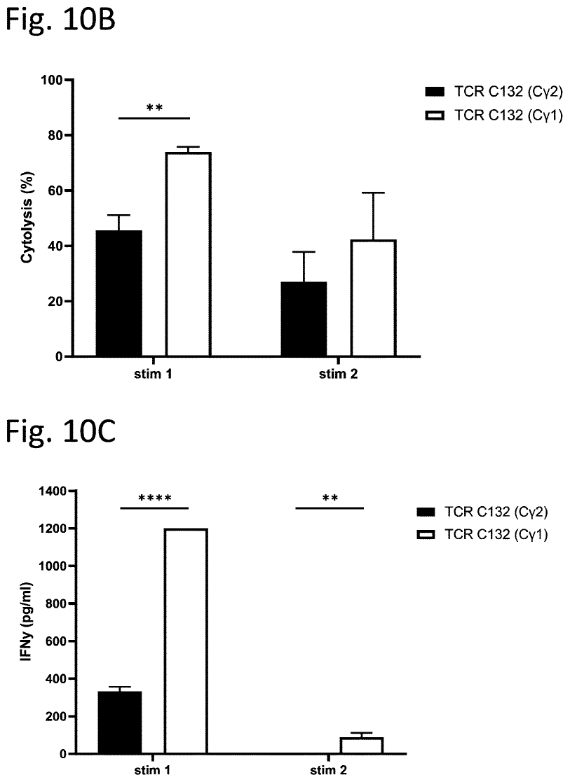

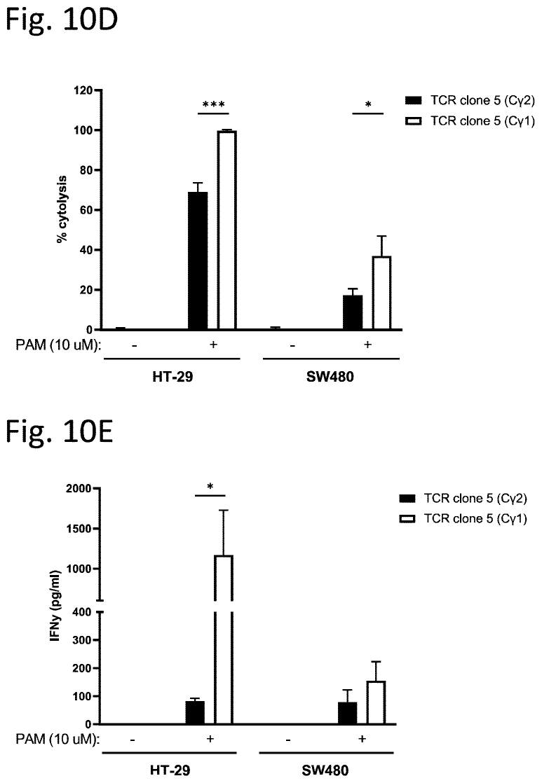

Additionally, the same trend was observed when Cγ1 constant region- and Cγ2 constant region-comprising variants of the previously characterized c15 and C132 γδTCRs (comprising the same variable regions) were compared in TEGs expressing them (FIG. 10A-10E).

These results demonstrate that the anti-tumour response mediated by γδTCRs may be improved by replacement of the Cγ2 constant region by a Cγ1 constant region.

### Example 4

In this example we tested whether soluble γδTCR-CD3 bispecific engagers can mediate anti-tumour responses.

Soluble γδTCR CD3 bispecific engagers derived from clone 3 γδTCR and comprising Cγ1 or Cγ2 constant region parts were generated as described in the Materials & Methods.

The mediated anti-tumour activity was evaluated by luciferase-based cytotoxicity assay by adding different concentrations of soluble γδTCR-CD3 bispecific engagers (1, 3 and 10 μg, respectively) into co-cultures of human PBMC-derived αβT-cells and Luc-Tom RKO or Luc-Tom MDA-MB-231 tumour cells (effector to target ratio of 1:1). Co-coltures without addition of soluble γδTCR clone 3-CD3 bispecific engagers were used as negative controls. After incubation for 48 hours at 37° C., luciferase activity measurements were performed as described in the Materials & Methods. IFN-γ production measurements were performed as described in the Materials & Methods.

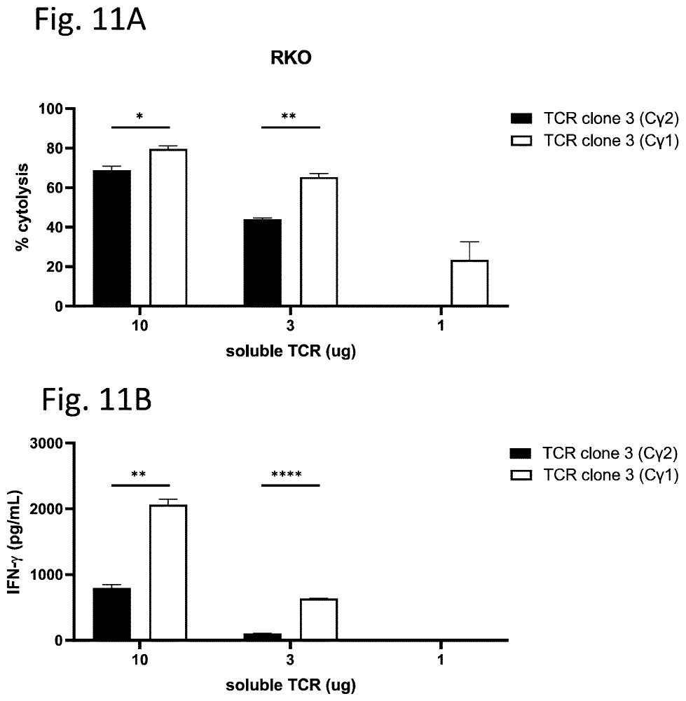

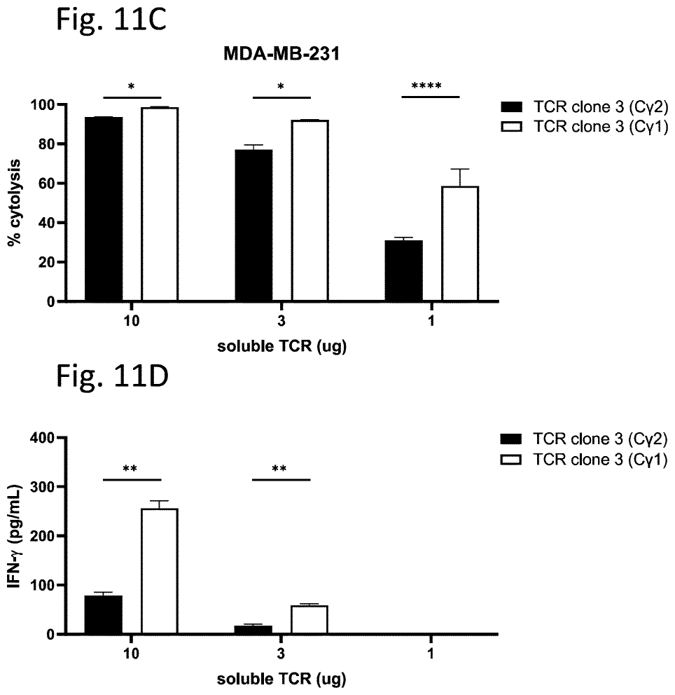

As shown in FIG. 11A-11D, γδTCR-CD3 bispecific engagers are able to mediate a strong anti-tumor response. Additionally, the anti-tumour response is further increased in γδTCR-CD3 bispecific engagers comprising a Cγ1 constant region part, relative to γδTCR-CD3 bispecific engagers comprising the same variable regions but comprising a Cγ2 constant region part.

1. A δT-cell receptor chain or a part thereof comprising a CDR3 region, said δT-cell receptor chain or part thereof being represented by an amino acid sequence, said amino acid sequence comprising at least 70% sequence identity with amino acid sequence SEQ ID NO: 7, 9, 13, 15, 19, and/or 21.

2. A γT-cell receptor chain or a part thereof comprising a CDR3 region, said γT-cell receptor chain or part thereof being represented by an amino acid sequence, said amino acid sequence comprising at least 85% sequence identity with amino acid sequence SEQ ID NO: 10, 12, 16, 18, 22, and/or 24.

3. A nucleic acid molecule encoding an amino acid sequence as defined in claim 1, wherein said nucleic acid molecule is represented by a nucleotide sequence comprising a nucleotide sequence that has at least 60% sequence identity with SEQ ID NO: 8, 14, 20, 28, 30 and/or 32 and/or a nucleotide sequence that encodes an amino acid sequence that has at least 60% amino acid identity with an amino acid sequence encoded by a nucleotide sequence SEQ ID NO: 8, 14, 20, 28, 30 and/or 32.

4. A nucleic acid molecule encoding an amino acid sequence as defined in claim 2, wherein said nucleic acid molecule is represented by a nucleotide sequence comprising a nucleotide sequence that has at least 80% sequence identity with SEQ ID NO: 11, 17, 23, 29, 31, and/or 33 and/or a nucleotide sequence that encodes an amino acid sequence that has at least 80% amino acid identity with an amino acid sequence encoded by a nucleotide sequence SEQ ID NO: 11, 17, 23, 29, 31, and/or 33.

5. (canceled)

6. (canceled)

7. (canceled)

8. (canceled)

9. (canceled)

10. (canceled)

11. (canceled)

12. (canceled)

13. (canceled)

14. A nucleic acid construct comprising a nucleic acid molecule encoding the amino acid sequence as identified in claim 1.

15. (canceled)

16. A cell comprising the nucleic acid construct of claim 14.

17. (canceled)

18. (canceled)

19. (canceled)

20. (canceled)

21. (canceled)

22. (canceled)

23. (canceled)

24. (canceled)

25. (canceled)

26. (canceled)

27. A method for improving the anti-tumour or anti-infective response mediated by a γδT-cell receptor or a part thereof comprising a CDR3 region, wherein said receptor or part thereof comprises a Cγ2 constant region or a part thereof, said method comprising the step of replacing said Cγ2 constant region or part thereof by a Cγ1 constant region or a part thereof.

28. A method for identifying a γδT-cell receptor or a part thereof comprising a CDR3 region that mediates an improved anti-tumour or anti-infective response comprising the steps of:
a) providing a γδT-cell receptor or a part thereof comprising a CDR3 region, wherein said receptor or part thereof comprises a Cγ2 constant region or a part thereof;
b) replacing said Cγ2 constant region or part thereof by a Cγ1 constant region or a part thereof;
c) expressing the γδT-cell receptor or a part thereof obtained in step b) in an engineered T-cell, preferably an αβT-cell;
d) determining the anti-tumour or anti-infective response of the engineered T-cell of step c);
e) identifying the γδT-cell receptor or part thereof that mediates the improved anti-tumour or anti-infective response.

29. (canceled)

30. (canceled)

31. (canceled)

32. (canceled)

33. The method according to claim 27, wherein the Cγ2 constant region or part thereof is represented by an amino acid sequence comprising at least 95% sequence identity or similarity with SEQ ID NO: 161 or SEQ ID NO: 164, preferably with SEQ ID NO: 161.

34. The method according to claim 27, wherein the Cγ1 constant region or part thereof is represented by an amino acid sequence comprising at least 95% sequence identity or similarity with SEQ ID NO: 152.

35. The method according to claim 27, wherein the Cγ1 constant region or part thereof is encoded by a nucleic acid molecule represented by a nucleotide sequence comprising at least 95% sequence identity with SEQ ID NO: 151.

36. The method according to claim 27, wherein the Cγ1 constant region or part thereof is represented by an amino acid sequence that does not comprise SEQ ID NO: 158 or part thereof.

37. (canceled)

38. A nucleic acid construct comprising a nucleic acid molecule encoding the amino acid sequence as identified in claim 2.

39. A nucleic acid construct comprising a nucleic acid molecule as identified in claim 3.

40. A nucleic acid construct comprising a nucleic acid molecule as identified in claim 4.

41. A cell comprising the nucleic acid construct of claim 38.

42. A cell comprising the nucleic acid construct of claim 39.

43. A cell comprising the nucleic acid construct of claim 40.


SEQUENCE LISTING (165 sequences)

SEQ ID NO: 1 - protein, 21 aa, Artificial sequence
  TRD CDR3 clone 1
CAFIVYDFLGELGEDTDKLIF

SEQ ID NO: 2 - DNA, 885 bp, Artificial sequence
  TRD codon optimised clone 1
atgatcctgaccgtgggcttcagcttcctgttcttctaccggggcaccctgtgcgacaaagtgacacagtctagccccgatcagacagtggcctctggatctgaagtggtgctgctgtgcacctacgacaccgtgtacagcaaccccgacctgttctggtacagaatcagacccgactacagcttccagttcgtgttctacggcgacaacagcagaagcgagggcgccgatttcacccagggcagattttccgtgaagcacatcctgacacagaaagccttccacctggtcatcagccctgtgcggacagaggatagcgccacctactattgcgcctttatcgtgtacgacttcctgggcgagctgggcgaagataccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 3 - protein, 295 aa, Artificial Sequence
  TRD full length clone 1
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCAFIVYDFLGELGEDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 4 - protein, 15 aa, Artificial sequence
  TRG CDR3 clone 1
CATWDSKPHYYKKLF

SEQ ID NO: 5 - DNA, 975 bp, Artificial sequence
  TRG codon optimised clone 1
atgagatgggctctgctggtgctgctggcctttctgtctcctgccagccagaagtccagcaacctggaaggcagaacaaagagcgtgaccagacagaccggaagcagcgccgagatcacctgtgatctgaccgtgaccaacaccttctacatccactggtatctgcaccaagagggcaaagcccctcagcggctgctgtactacgatgtgtctaccgccagggacgtgctggaatctggactgagccctggcaagtactacacccacacacctcggcggtggtcctggattctgagactgcagaacctgatcgagaacgactccggcgtgtactactgtgccacctgggatagcaagccccactactacaagaagctgttcggcagcggcaccacactggtggttacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagtcc

SEQ ID NO: 6 - protein, 325 aa, Artificial Sequence
  TRG full length clone 1
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDSKPHYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 7 - protein, 15 aa, Artificial sequence
  TRD CDR3 clone 2
CACGLPRSLADKLIF

SEQ ID NO: 8 - DNA, 867 bp, Artificial sequence
  TRD codon optimised clone 2
atgatcctgaccgtgggcttcagcttcctgttcttctaccggggcaccctgtgcgacaaagtgacacagtctagccccgatcagacagtggcctctggatctgaagtggtgctgctgtgcacctacgacaccgtgtacagcaaccccgacctgttctggtacagaatcagacccgactacagcttccagttcgtgttctacggcgacaacagcagaagcgagggcgccgatttcacccagggcagattttccgtgaagcacatcctgacacagaaagccttccacctggtcatcagccctgtgcggacagaggatagcgccacctactattgtgcttgtggcctgcctagaagcctggccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 9 - protein, 289 aa, Artificial Sequence
  TRD full length clone 2
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCACGLPRSLADKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 10 - protein, 11 aa, Artificial sequence
  TRG CDR3 clone 2
CATWDLHKKLF

SEQ ID NO: 11 - DNA, 963 bp, Artificial sequence
  TRG codon optimised clone 2
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgtgccacatgggacctgcacaagaagctgttcggcagcggcacaaccctggtggttacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagtcc

SEQ ID NO: 12 - protein, 321 aa, Artificial Sequence
  TRG full length clone 2
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDLHKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 13 - protein, 17 aa, Artificial sequence
  TRD CDR3 clone 3
CALGEFPYWGIPDKLIF

SEQ ID NO: 14 - DNA, 873 bp, Artificial sequence
  TRD codon optimised clone 3
atggtgttcagcagcctgctgtgcgtgttcgtggccttcagctacagcggcagcagcgtggcccagaaagtgacccaggcccagagcagcgtgtccatgcctgtgcggaaggccgtgaccctgaactgcctgtacgagacaagctggtggtcctactacatcttctggtacaagcagctgcccagcaaagagatgatcttcctgatccggcagggcagcgacgagcagaacgccaagagcggccggtacagcgtgaacttcaagaaagccgccaagtccgtggccctgaccatcagcgccctgcagctggaagatagcgccaagtatttctgtgccctgggcgagtttccttattggggcatccccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 15 - protein, 291 aa, Artificial Sequence
  TRD full length clone 3
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGEFPYWGIPDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 16 - protein, 12 aa, Artificial sequence
  TRG CDR3 clone 3
CATWDGYYKKLF

SEQ ID NO: 17 - DNA, 915 bp, Artificial sequence
  TRG codon optimised clone 3
atgagatgggctctgctggtgctgctggcctttctgtctcctgccagccagaagtccagcaacctggaaggcagaacaaagagcgtgaccagacagaccggaagcagcgccgagatcacctgtgatctgaccgtgaccaacaccttctacatccactggtatctgcaccaagagggcaaagcccctcagcggctgctgtactacgatgtgtctaccgccagggacgtgctggaatctggactgagccctggcaagtactacacccacacacctcggcggtggtcctggattctgagactgcagaacctgatcgagaacgactccggcgtgtactactgtgctacctgggacggctactacaagaagctgttcggcagcggcaccacactggtggttacagacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaag

SEQ ID NO: 18 - protein, 305 aa, Artificial Sequence
  TRG full length clone 3
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEK

SEQ ID NO: 19 - protein, 25 aa, Artificial sequence
  TRD CDR3 clone 4
CALGGLGWGYAGDEGGGKLTDKLIF

SEQ ID NO: 20 - DNA, 897 bp, Artificial sequence
  TRD codon optimised clone 4
atggtgttcagcagcctgctgtgcgtgttcgtggccttcagctacagcggcagcagcgtggcccagaaagtgacccaggcccagagcagcgtgtccatgcctgtgcggaaggccgtgaccctgaactgcctgtacgagacaagctggtggtcctactacatcttctggtacaagcagctgcccagcaaagagatgatcttcctgatccggcagggcagcgacgagcagaacgccaagagcggccggtacagcgtgaacttcaagaaagccgccaagtccgtggccctgaccatcagcgccctgcagctggaagatagcgccaagtatttctgtgcccttggaggactcggatggggatatgctggcgacgaaggcggaggcaagctgaccgataagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 21 - protein, 299 aa, Artificial Sequence
  TRD full length clone 4
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGGLGWGYAGDEGGGKLTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 22 - protein, 14 aa, Artificial sequence
  TRG CDR3 clone 4
CALWEVHPNYKKLF

SEQ ID NO: 23 - DNA, 984 bp, Artificial sequence
  TRG codon optimised clone 4
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgtgctctgtgggaagtgcaccccaactacaagaagctgttcggcagcggcaccacactggtggttacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagtcc

SEQ ID NO: 24 - protein, 328 aa, Artificial Sequence
  TRG full length clone 4
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVHPNYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 25 - DNA, 6539 bp, Artificial sequence
  pLenti 6.3
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatgcatgctgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcactagcggccctaatacgactcactatagcccggctcgaggctaggcgcgaattcgccacccggatccacgcgtaccggttagtaatgatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggcgatggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 26 - DNA, 885 bp, Artificial Sequence
  TRD full length wild type clone 1
atgattcttactgtgggctttagctttttgtttttctacaggggcacgctgtgtgacaaagtaacccagagttccccggaccagacggtggcgagtggcagtgaggtggtactgctctgcacttacgacactgtatattcaaatccagatttattctggtaccggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctttatcgtttatgacttcctaggagaactgggggaagacaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 27 - DNA, 975 bp, Artificial Sequence
  TRG full length wild type clone 1
atgcggtgggccctactggtgcttctagctttcctgtctcctgccagtcagaaatcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccaccgcaagggatgtgttggaatcaggactcagtccaggaaagtattatactcatacacccaggaggtggagctggatattgagactgcaaaatctaattgaaaatgattctggggtctattactgtgccacctgggacagtaaacctcattattataagaaactctttggcagtggaacaacacttgttgtcacagataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaatacaattattcaaaagatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatca

SEQ ID NO: 28 - DNA, 867 bp, Artificial Sequence
  TRD full length wild type clone 2
atgattcttactgtgggctttagctttttgtttttctacaggggcacgctgtgtgacaaagtaacccagagttccccggaccagacggtggcgagtggcagtgaggtggtactgctctgcacttacgacactgtatattcaaatccagatttattctggtaccggataaggccagattattcctttcagtttgtcttttatggggataacagcagatcagaaggtgcagattttactcaaggacggttttctgtgaaacacattctgacccagaaagcctttcacttggtgatctctccagtaaggactgaagacagtgccacttactactgtgcctgtggccttccccgttctctggccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 29 - DNA, 963 bp, Artificial Sequence
  TRG full length wild type clone 2
atgcagtgggccctagcggtgcttctagctttcctgtctcctgccagtcagaaatcttccaacttggaagggagaacgaagtcagtcatcaggcagactgggtcatctgctgaaatcacttgtgatcttgctgaaggaagtaccggctacatccactggtacctacaccaggaggggaaggccccacagcgtcttctgtactatgactcctacacctccagcgttgtgttggaatcaggaatcagcccagggaagtatgatacttacggaagcacaaggaagaacttgagaatgatactgcgaaatcttattgaaaatgactctggagtctattactgtgccacctgggatctgcataagaaactctttggcagtggaacaacacttgttgtcacagataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaatacaattattcaaaggatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatca

SEQ ID NO: 30 - DNA, 873 bp, Artificial Sequence
  TRD full length wild type clone 3
atgctgttctccagcctgctgtgtgtatttgtggccttcagctactctggatcaagtgtggcccagaaggttactcaagcccagtcatcagtatccatgccagtgaggaaagcagtcaccctgaactgcctgtatgaaacaagttggtggtcatattatattttttggtacaagcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggaattcccctactgggggatccccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 31 - DNA, 915 bp, Artificial Sequence
  TRG full length wild type clone 3
atgcggtgggccctactggtgcttctagctttcctgtctcctgccagtcagaaatcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccaccgcaagggatgtgttggaatcaggactcagtccaggaaagtattatactcatacacccaggaggtggagctggatattgagactgcaaaatctaattgaaaatgattctggggtctattactgtgccacctgggacggctattataagaaactctttggcagtggaacaacacttgttgtcacagataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacagatgtcatcacaatggatcccaaagacaattgttcaaaagatgcaaatgatacactactgctgcagctcacaaacacctctgcatattacatgtacctcctcctgctcctcaagagtgtggtctattttgccatcatcacctgctgtctgcttagaagaacggctttctgctgcaatggagagaaa

SEQ ID NO: 32 - DNA, 897 bp, Artificial Sequence
  TRD full length wild type clone 4
atgctgttctccagcctgctgtgtgtatttgtggccttcagctactctggatcaagtgtggcccagaaggttactcaagcccagtcatcagtatccatgccagtgaggaaagcagtcaccctgaactgcctgtatgaaacaagttggtggtcatattatattttttggtacaagcaacttcccagcaaagagatgattttccttattcgccagggttctgatgaacagaatgcaaaaagtggtcgctattctgtcaacttcaagaaagcagcgaaatccgtcgccttaaccatttcagccttacagctagaagattcagcaaagtacttttgtgctcttggggggctgggctggggatacgcaggcgatgaagggggtggaaaactaaccgataaactcatctttggaaaaggaacccgtgtgactgtggaaccaagaagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttg

SEQ ID NO: 33 - DNA, 984 bp, Artificial Sequence
  TRG full length wild type clone 4
atgctgtcactgctccacacatcaacgctggcagtccttggggctctgtgtgtatatggtgcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggtgcatcccaattataagaaactctttggcagtggaacaacacttgttgtcacagataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaatacaattattcaaaggatgcaaatgatgtcatcacaatggatcccaaagacaattggtcaaaagatgcaaatgatacactactgctgcagctcacaaacacctctgcatattacatgtacctcctcctgctcctcaagagtgtggtctattttgccatcatcacctgctgtctgcttggaagaacggctttctgctgcaatggagagaaatca

SEQ ID NO: 34 - DNA, 2844 bp, Artificial sequence
  TRG clone 2-41BBL-OX40-TRD clone 2
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgtgccacatgggacctgcacaagaagctgttcggcagcggcacaaccctggtggttacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagagcggatctggcgccaccaatttcagcctgctgaaacaggctggcgacgtggaagagaaccctgggcccatgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaaggcagcggcgaaggccgcggcagcctgctgacctgcggagacgtggaagaaaaccctggcccgatgatcctgaccgtgggcttcagcttcctgttcttctaccggggcaccctgtgcgacaaagtgacacagtctagccccgatcagacagtggcctctggatctgaagtggtgctgctgtgcacctacgacaccgtgtacagcaaccccgacctgttctggtacagaatcagacccgactacagcttccagttcgtgttctacggcgacaacagcagaagcgagggcgccgatttcacccagggcagattttccgtgaagcacatcctgacacagaaagccttccacctggtcatcagccctgtgcggacagaggatagcgccacctactattgtgcttgtggcctgcctagaagcctggccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 35 - DNA, 2895 bp, Artificial sequence
  TRG clone 4-41BBL-OX40-TRD clone 4
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgtgctctgtgggaagtgcaccccaactacaagaagctgttcggcagcggcaccacactggtggttacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagagcggatctggcgccaccaatttcagcctgctgaaacaggctggcgacgtggaagagaaccctgggcccatgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaaggcagcggcgaaggccgcggcagcctgctgacctgcggagacgtggaagaaaaccctggcccgatggtgttcagcagcctgctgtgcgtgttcgtggccttcagctacagcggcagcagcgtggcccagaaagtgacccaggcccagagcagcgtgtccatgcctgtgcggaaggccgtgaccctgaactgcctgtacgagacaagctggtggtcctactacatcttctggtacaagcagctgcccagcaaagagatgatcttcctgatccggcagggcagcgacgagcagaacgccaagagcggccggtacagcgtgaacttcaagaaagccgccaagtccgtggccctgaccatcagcgccctgcagctggaagatagcgccaagtatttctgtgcccttggaggactcggatggggatatgctggcgacgaaggcggaggcaagctgaccgataagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 36 - DNA, 885 bp, Artificial sequence
  41BBL-OX40
atgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 37 - DNA, 894 bp, Artificial sequence
  41BBL-OX40_GSG
atgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaaggcagcggc

SEQ ID NO: 38 - protein, 298 aa, Artificial sequence
  41BBL-OX40_GSG
MLGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSG

SEQ ID NO: 39 - protein, 205 aa, Artificial sequence
  41BBL-EC
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 40 - protein, 133 aa, Artificial sequence
  OX40L
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 41 - protein, 247 aa, Artificial sequence
  CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 42 - protein, 212 aa, Artificial sequence
  RANK
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLP

SEQ ID NO: 43 - protein, 220 aa, Artificial sequence
  Reverse 41BBL
MLLLVTSLLLCELPHPAFLLIPDQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSERLDLLGAPDDPSLEPGERLRPSAASGPSARA

SEQ ID NO: 44 - protein, 347 aa, Artificial sequence
  41BBL  CD86 IgV domain
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESLNGGGGSGGGGSGGGGSGGGGSGGGGSTSAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAN

SEQ ID NO: 45 - protein, 36 aa, Artificial sequence
  OX40-ICD
RDQRLPPDAHKPPGGGSFRTPIQEEQADAHSTLAKI

SEQ ID NO: 46 - protein, 36 aa, Artificial sequence
  OX40-ICD-REV
IKALTSHADAQEEQIPTRFSGGGPPKHADPPLRQDR

SEQ ID NO: 47 - protein, 63 aa, Artificial sequence
  OX40-ICD-TM
VAAILGLGLVLGLLGPLAILLALYLLRRDQRLPPDAHKPPGGGSFRTPIQEEQADAHSTLAKI

SEQ ID NO: 48 - protein, 42 aa, Artificial sequence
  41BB-ICD
KRGRKKLLYIFKQPFMRPVQTTQEEDGCSCRFPEEEEGGCEL

SEQ ID NO: 49 - protein, 45 aa, Artificial sequence
  41BB-ICD-REV
MLGKRGRKKLLYIFKQPFMRPVQTTQEEDGCSCRFPEEEEGGCEL

SEQ ID NO: 50 - protein, 36 aa, Artificial sequence
  NKp80-ICD
MQDEDGYMTLNVQSKKRSSAQTSQLTFKDYSVTLHW

SEQ ID NO: 51 - protein, 224 aa, Artificial sequence
  IL18RAP-ICD
AASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 52 - protein, 69 aa, Artificial sequence
  41BB-ICD-TM
IISFFLALTSTALLFLLFFLTLRFSVVKRGRKKLLYIFKQPFMRPVQTTQEEDGCSCRFPEEEEGGCEL

SEQ ID NO: 53 - protein, 42 aa, Artificial sequence
  OX40-ICD-ALYLLR
IKALTSHADAQEEQIPTRFSGGGPPKHADPPLRQDRRLLYLA

SEQ ID NO: 54 - protein, 42 aa, Artificial sequence
  OX40-ICD-ALYLLR-REV
ALYLLRRDQRLPPDAHKPPGGGSFRTPIQEEQADAHSTLAKI

SEQ ID NO: 55 - protein, 6 aa, Artificial sequence
  TRAF domain OX40
PIQEEQ

SEQ ID NO: 56 - protein, 5 aa, Artificial sequence
  YMTLN motif
YMTLN

SEQ ID NO: 57 - protein, 18 aa, Artificial sequence
  TRAF domain 41BB
QTTQEEDGCSCRFPEEEE

SEQ ID NO: 58 - protein, 21 aa, Artificial sequence
  OX40-TM
VAAILGLGLVLGLLGPLAILL

SEQ ID NO: 59 - protein, 27 aa, Artificial sequence
  OX40L-TM
LLLVASVIQGLGLLLCFTYICLHFSAL

SEQ ID NO: 60 - protein, 27 aa, Artificial sequence
  41BB-TM
IISFFLALTSTALLFLLFFLTLRFSVV

SEQ ID NO: 61 - protein, 21 aa, Artificial sequence
  41BBL-TM
WALVAGLLLLLLLAAACAVFL

SEQ ID NO: 62 - protein, 21 aa, Artificial sequence
  NKp80-TM
ILLGISGTVNGILTLTLISLI

SEQ ID NO: 63 - protein, 21 aa, Artificial sequence
  CD86-TM
WITAVLPTVIICVMVFCLILW

SEQ ID NO: 64 - protein, 19 aa, Artificial sequence
  IL18RAP-TM
GVVLLYILLGTIGTLVAVL

SEQ ID NO: 65 - protein, 21 aa, Artificial sequence
  RANK-TM
GLIILLLFASVALVAAIIFGV

SEQ ID NO: 66 - protein, 4 aa, Artificial sequence
  Flexible linker
TSGS

SEQ ID NO: 67 - protein, 5 aa, Artificial sequence
  Flexible linker
GGGGS

SEQ ID NO: 68 - protein, 4 aa, Artificial sequence
  Flexible linker
GGGS

SEQ ID NO: 69 - protein, 2 aa, Artificial sequence
  Flexible linker
GG

SEQ ID NO: 70 - protein, 18 aa, Artificial sequence
  Flexible linker
KESGSVSSEQLAQFRSLD

SEQ ID NO: 71 - protein, 14 aa, Artificial sequence
  Flexible linker
EGKSSGSGSESKST

SEQ ID NO: 72 - protein, 12 aa, Artificial sequence
  Flexible linker
GSAGSAAGSGEF

SEQ ID NO: 73 - protein, 5 aa, Artificial sequence
  Rigid linker
EAAAK

SEQ ID NO: 74 - protein, 5 aa, Artificial sequence
  Rigid linker
EAAAR

SEQ ID NO: 75 - protein, 5 aa, Artificial sequence
  Rigid linker
PAPAP

SEQ ID NO: 76 - protein, 12 aa, Artificial sequence
  Rigid linker
AEAAAKEAAAKA

SEQ ID NO: 77 - protein, 51 aa, Artificial sequence
  Rigid linker
ILTHDSSIRYLQEIYNSNNQKIVNLKEKVAQLEAQCQEPCKDTVQIHDITG

SEQ ID NO: 78 - protein, 3 aa, Artificial sequence
  Flexible linker
GGS

SEQ ID NO: 79 - protein, 30 aa, Artificial sequence
  Flexible linker
SLNGGGGSGGGGSGGGGSGGGGSGGGGSTS

SEQ ID NO: 80 - protein, 20 aa, Artificial sequence
  Flexible linker
SGGSGGGGSGGGSGGGGSLQ

SEQ ID NO: 81 - protein, 26 aa, Artificial sequence
  Flexible linker
SGGGSGGGGSGGGGSGGGGSGGGSLQ

SEQ ID NO: 82 - protein, 15 aa, Artificial sequence
  Flexible linker
GGGGSGGGGSGGGGS

SEQ ID NO: 83 - protein, 295 aa, Artificial sequence
  41BBL-OX40
MLGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 84 - protein, 283 aa, Artificial sequence
  41BBL13W-OX40rev
MLGIKALTSHADAQEEQIPTRFSGGGPPKHADPPLRQDRTSGSWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 85 - protein, 288 aa, Artificial sequence
  41BBL-NKp80
MQDEDGYMTLNVQSKKRSSAQTSQLTFKDYSVTLHWYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 86 - protein, 264 aa, Artificial sequence
  41BBLmincyto-NKp80
MQDEDGYMTLNVQSKKRSSAQTSQLTFKDYSVTLHWYKWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 87 - protein, 231 aa, Artificial sequence
  OX40L-41BB_1
MLGKRGRKKLLYIFKQPFMRPVQTTQEEDGCSCRFPEEEEGGCELTSGSERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 88 - protein, 231 aa, Artificial sequence
  OX40L- 41BB_2
MLGKRGRKKLLYIFKQPFMRPVQTTQEEDGCSCRFPEEEEGGCELGGSAERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 89 - protein, 232 aa, Artificial sequence
  OX40L-41BBrev
MLGLECGGEEEEPFRCSCGDEEQTTQVPRMFPQKFIYLLKKRGRKLGGSAERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 90 - protein, 368 aa, Artificial sequence
  CD86-OX40
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCFGSGRDQRLPPDAHKPPGGGSFRTPIQEEQADAHSTLAKI

SEQ ID NO: 91 - protein, 313 aa, Artificial sequence
  CD86delP276-OX40
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPGRDQRLPPDAHKPPGGGSFRTPIQEEQADAHSTLAKI

SEQ ID NO: 92 - protein, 252 aa, Artificial sequence
  41BBL
YASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 93 - protein, 233 aa, Artificial sequence
  41BBL-mincyto
MSKSTGSWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 94 - protein, 228 aa, Artificial sequence
  41BBL13W
MEWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 95 - protein, 295 aa, Artificial sequence
  41BBL-OX40rev
MLGIKALTSHADAQEEQIPTRFSGGGPPKHADPPLRQDRTSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 96 - protein, 283 aa, Artificial sequence
  41BBL-rev extra-OX40tm-cyto
MLLLVTSLLLCELPHPAFLLIPDQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSERLDLLGAPDDPSLEPGERLRPSAASGPSARAVAAILGLGLVLGLLGPLAILLALYLLRRDQRLPPDAHKPPGGGSFRTPIQEEQADAHSTLAKI

SEQ ID NO: 97 - protein, 269 aa, Artificial sequence
  41BBLmincyto-OX40
MLGRDQRLPPDAHKPPGGGSFRTPIQEEQADAHSTLAKITSGSWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 98 - protein, 281 aa, Artificial sequence
  41BBL13W-OX40rev
MEIKALTSHADAQEEQIPTRFSGGGPPKHADPPLRQDRRLLWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 99 - protein, 269 aa, Artificial sequence
  41BBL-mincyto-OX40rev
MLGIKALTSHADAQEEQIPTRFSGGGPPKHADPPLRQDRTSGSWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 100 - protein, 301 aa, Artificial sequence
  41BBL-OX40ENrev
MLGIKALTSHADAQEEQIPTRFSGGGPPKHADPPLRQDRRLLYLATSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 101 - protein, 301 aa, Artificial sequence
  41BBL-OX40EN
MLGALYLLRRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 102 - protein, 291 aa, Artificial sequence
  41BBLmincyto-13alink-OX40
MLGRDQRLPPDAHKPPGGGSFRTPIQEEQADAHSTLAKISLNGGGGSGGGGSGGGGSGGGGSGGPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 103 - protein, 205 aa, Artificial sequence
  OX40Lmincyto-41BBrev
MLGLECGGEEEEPFRCSCGDEEQTTQVPRMFPQKFIYLLKKRGRKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 104 - protein, 183 aa, Artificial sequence
  OX40L WT
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - protein, 208 aa, Artificial sequence
  OX40Lmincyto-41BB
MLGKRGRKKLLYIFKQPFMRPVQTTQEEDGCSCRFPEEEEGGCELGGSLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 106 - protein, 329 aa, Artificial sequence
  CD86 WT
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 107 - protein, 276 aa, Artificial sequence
  CD86-delP276
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRP

SEQ ID NO: 108 - protein, 268 aa, Artificial sequence
  CD86mincyto
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILW

SEQ ID NO: 109 - protein, 490 aa, Artificial sequence
  CD86mincyto-IL18RAP
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWSALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 110 - protein, 551 aa, Artificial sequence
  CD86-IL18RAP
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCFSALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 111 - protein, 437 aa, Artificial sequence
  CD86IgV-41BBL-OX40
MLGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESLNGGGGSGGGGSGGGGSGGGGSGGGGSTSAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAN

SEQ ID NO: 112 - protein, 613 aa, Artificial sequence
  RANK WT
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 113 - protein, 230 aa, Artificial sequence
  RANKmincyto
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPGLIILLLFASVALVAAIIFGV

SEQ ID NO: 114 - protein, 272 aa, Artificial sequence
  RANK-OX40
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPVAAILGLGLVLGLLGPLAILLALYLLRRDQRLPPDAHKPPGGGSFRTPIQEEQADAHSTLAKI

SEQ ID NO: 115 - protein, 278 aa, Artificial sequence
  RANK-41BB
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPIISFFLALTSTALLFLLFFLTLRFSVVKRGRKKLLYIFKQPFMRPVQTTQEEDGCSCRFPEEEEGGCEL

SEQ ID NO: 116 - protein, 452 aa, Artificial sequence
  RANK-IL18RAP
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 117 - protein, 165 aa, Artificial sequence
  OX40WT mincyto
MERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 118 - protein, 138 aa, Artificial sequence
  Q8
MGLVRRGARAGPRIPRGWTALCLLSLLPSGFMAELPTQGTFSNVSTNVSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 119 - protein, 239 aa, Artificial sequence
  eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 120 - DNA, 367 bp, Artificial sequence
  MSCV promoter
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcact

SEQ ID NO: 121 - protein, 155 aa, Artificial sequence
  CD70
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 122 - protein, 94 aa, Artificial sequence
  IL2RB ICD
VLHTPDQGQLEQLSLYADTNLPLLQTVKDRELVELPSIEPALGGPSFSSSPFPSSLWKQVDGGHESSLQSFFKSPDPTNCKLVKKLWPGTNRCN

SEQ ID NO: 123 - protein, 21 aa, Artificial sequence
  CD70-TM
VLRAALVPLVAGLVICLVVCI

SEQ ID NO: 124 - protein, 283 aa, Artificial sequence
  41BBL-13W-OX40
MLGRDQRLPPDAHKPPGGGSFRTPIQEEQADAHSTLAKITSGSWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 125 - protein, 392 aa, Artificial sequence
  41BBL-OX40-IL2RB
MLGVLHTPDQGQLEQLSLYADTNLPLLQTVKDRELVELPSIEPALGGPSFSSSPFPSSLWKQVDGGHESSLQSFFKSPDPTNCKLVKKLWPGTNRCNGSGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 126 - protein, 195 aa, Artificial sequence
  CD70
MLGPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 127 - protein, 180 aa, Artificial sequence
  CD70mincyto
MLGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 128 - protein, 221 aa, Artificial sequence
  CD70mincyto-OX40
MLGRDQRLPPDAHKPPGGGSFRTPIQEEQADAHSTLAKITSGSGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 129 - protein, 245 aa, Artificial sequence
  CD70-41BBL-OX40
MLGRDQRLPPDAHKPPGGGSFRTPIQEEQADAHSTLAKITSGSYASDASLDPEAPWPPAPRARACRVLPWALVAGLLLLLLLAAACAVFLQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 130 - DNA, 915 bp, Artificial sequence
  TRG codon optimised clone 2_Cg1
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgtgccacatgggacctgcacaagaagctgttcggcagcggcacaaccctggtggttacagacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagc

SEQ ID NO: 131 - protein, 305 aa, Artificial sequence
  TRG full length clone 2_Cg1
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDLHKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 132 - DNA, 966 bp, Artificial sequence
  TRG codon optimised clone 3_Cg2
atgagatgggctctgctggtgctgctggcctttctgtctcctgccagccagaagtccagcaacctggaaggcagaacaaagagcgtgaccagacagaccggaagcagcgccgagatcacctgtgatctgaccgtgaccaacaccttctacatccactggtatctgcaccaagagggcaaagcccctcagcggctgctgtactacgatgtgtctaccgccagggacgtgctggaatctggactgagccctggcaagtactacacccacacacctcggcggtggtcctggattctgagactgcagaacctgatcgagaacgactccggcgtgtactactgtgctacctgggacggctactacaagaagctgttcggcagcggcaccacactggtggttacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagtcc

SEQ ID NO: 133 - protein, 322 aa, Artificial sequence
  TRG full length clone 3_Cg2
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 134 - DNA, 936 bp, Artificial sequence
  TRG codon optimised clone 4_Cg1
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgtgctctgtgggaagtgcaccccaactacaagaagctgttcggcagcggcaccacactggtggttacagacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagc

SEQ ID NO: 135 - protein, 312 aa, Artificial sequence
  TRG full length clone 4_Cg1
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVHPNYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 136 - protein, 16 aa, Artificial sequence
  clone 5 TRG CDR3
CALWEIQELGKKIKVF

SEQ ID NO: 137 - DNA, 990 bp, Artificial sequence
  TRG codon optimised clone 5_Cg2
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagtcc

SEQ ID NO: 138 - protein, 330 aa, Artificial sequence
  TRG full length clone 5_Cg2
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 139 - protein, 13 aa, Artificial sequence
  C132 TRG CDR3
CATWDGFYYKKLF

SEQ ID NO: 140 - DNA, 969 bp, Artificial sequence
  TRG codon optimised C132_Cg2
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacggcttctactacaagaagctgttcggcagcggcaccaccctggtggtgacagacaagcagctggacgccgacgtgtcccccaagcctaccatcttcctgccctcaattgctgagacaaagctgcagaaggccggcacctacctgtgcctgctggaaaagttcttcccagacatcatcaagatccactggcaagaaaagaagtccaacaccatcctgggcagccaagaaggcaacaccatgaagaccaacgacacctacatgaagttcagctggctgaccgtgcccgaggagtccctggacaaagaacaccggtgcatcgtgcggcacgagaacaacaagaacggcatcgaccaagaaatcatcttcccacccatcaagaccgacgtgacaacagtggaccccaagtacaactacagcaaggacgccaacgacgtgatcacaatggaccccaaggacaactggagcaaggacgccaacgataccctgctgctgcagctgacaaacaccagcgcctactacatgtacttgttgctgctgctgaagtccgtggtgtacttcgccatcatcacatgctgcctgctgggccggaccgccttctgctgcaacggcgagaagtcc

SEQ ID NO: 141 - protein, 323 aa, Artificial sequence
  TRG full length C132_Cg2
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 142 - protein, 250 aa, Artificial sequence
  TCR clone 3  TRD_soluble
MACPGFLWALVISTCLEFSMASAAQKVTQAQSSVSMPVRKAVTLNCLYETSWWSYYIFWYKQLPSKEMIFLIRQGSDEQNAKSGRYSVNFKKAAKSVALTISALQLEDSAKYFCALGEFPYWGIPDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 143 - protein, 258 aa, Artificial sequence
  TCR clone 3  TRG_Cg1_soluble
MACPGFLWALVISTCLEFSMARTSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 144 - protein, 274 aa, Artificial sequence
  TCR clone 3  TRG_Cg2_soluble
MACPGFLWALVISTCLEFSMARTSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDN

SEQ ID NO: 145 - protein, 17 aa, Artificial sequence
  linker
RTGGGGSGGGGSGGGGS

SEQ ID NO: 146 - protein, 245 aa, Artificial sequence
  scFv CD3 -binding
QVQLVQSGGGVVQPGRSLRLSCKASGYTFTRYTMHWVRQAPGKGLEWIGYINPSRGYTNYNQKFKDRFTISRDNSKNTAFLQMDSLRPEDTGVYFCARYYDDHYCLDYWGQGTPVTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRVDGS

SEQ ID NO: 147 - protein, 3 aa, Artificial sequence
  linker
GSG

SEQ ID NO: 148 - protein, 21 aa, Artificial sequence
  AVI-TAG
GLNDIFEAQKIEWHEGSGGGG

SEQ ID NO: 149 - protein, 7 aa, Artificial sequence
  His-TAG
HHHHHHH

SEQ ID NO: 150 - DNA, 13120 bp, Artificial sequence
  pHCAG-L2EOP
ggggaggagcggggggaggagcggggggaggagcggggggaggagcggccagaccccgaaaacgggccccccccaaaacacaccccccgggggtcgcgcgcggccctttaaagcggcggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacgactctaggaattaattcttgaagacgaaagggcctcgtgatacgcctatttttataggttaatgtcatgataataatggtttcttagggtcgaccggtcgaccggtcgaccggtcgaccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggtcgaccggtcgaccggtcgaccggtcgaccggtcgaccatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcatgaattattccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacaggcagggggcggggcccgggccccgacttcccggttcggcggtaatgagatacgagccccgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgataagctgatccatcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaacttgtttattgcagcttataatggttacaaataaagcaatagcatcacaaatttcacaaataaagcatttttttcactgcattctagttgtggtttgtccaaactcatcaatgtatcttatcatgtctggatcggtttgaagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagcgtttaaacttaagcttggtaccgagctcggatccactagtccagtgtggtggaattctgcagatatccagcacagtggcggccgctcgagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtggtgaaacagactttgaattttgaccttctcaagttggcgggagacgtcgagtccaaccctgggcccatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaagggctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccatttaaatgttaattaaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggaacttgtttattgcagcttataatggttacaaataaagcaatagcatcacaaatttcacaaataaagcatttttttcactgcattctagttgtggtttgtccaaactcatcaatgtatcttatcatgtctggatctctgacctgagattggcggcactgaggtagagatgcccgaacccccccgagggagcgcgggacgcggccggggagggctggggccggggagggctggggccggggagggctggggccggggagggctggggccggggagggctggggccggggagggctggggctggggaggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcaggtgtggtggatgacgtgacgtgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcgg

SEQ ID NO: 151 - DNA, 516 bp, Artificial sequence
  Cg1
gacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaag

SEQ ID NO: 152 - protein, 173 aa, Artificial sequence
  Cg1
DKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 153 - protein, 225 aa, Artificial sequence
  TCR clone 3  TRD_soluble_no signal pept
KVTQAQSSVSMPVRKAVTLNCLYETSWWSYYIFWYKQLPSKEMIFLIRQGSDEQNAKSGRYSVNFKKAAKSVALTISALQLEDSAKYFCALGEFPYWGIPDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 154 - protein, 233 aa, Artificial sequence
  TCR clone 3  TRG_Cg1_soluble_no signal pept
NLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 155 - protein, 249 aa, Artificial sequence
  TCR clone 3  TRG_Cg2_soluble_no signal pept
NLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDN

SEQ ID NO: 156 - protein, 25 aa, Artificial sequence
  signal pept gamma
MACPGFLWALVISTCLEFSMARTSS

SEQ ID NO: 157 - protein, 25 aa, Artificial sequence
  signal pept delta
MACPGFLWALVISTCLEFSMASAAQ

SEQ ID NO: 158 - protein, 23 aa, Artificial sequence
  TRGC1 TM domain
YYMYLLLLLKSVVYFAIITCCLL

SEQ ID NO: 159 - protein, 23 aa, Artificial sequence
  TRGC2 TM domain
YYTYLLLLLKSVVYFAIITCCLL

SEQ ID NO: 160 - protein, 23 aa, Artificial sequence
  TRDC TM domain
LGLRMLFAKTVAVNFLLTAKLFF

SEQ ID NO: 161 - protein, 189 aa, Artificial sequence
  Cg2
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYMYLLLLLKSVVYFAIITCCLLGRTAFCCNGEKS

SEQ ID NO: 162 - protein, 306 aa, Artificial Sequence
  TRG full length clone 3
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 163 - protein, 16 aa, Artificial sequence
  Cg2 domain
DVTTVDPKYNYSKDAN

SEQ ID NO: 164 - protein, 189 aa, Artificial sequence
  Cg2
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 165 - protein, 14 aa, Artificial sequence
  Cg2 domain
TTVDPKYNYSKDAN